SEQUENCE LISTING (167 sequences)

SEQ ID NO: 1 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 2 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 3 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEIALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALNDVCTGGNPREATLEDIVELYHTAW

SEQ ID NO: 4 - DNA, 1152 bp, Escherichia coli
atgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaatccccaccacagcaggcactgcggcagaagtgaccattaactacgtgatcactgacgaagaaaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatagcgctcgggcagtatgttgcgggtatgggcttctcgaatgttgggttagggttggtgcatggtatggcgcatccactgggcgcgttttataacactccacacggtgttgcaaacgccatcctgctaccgcatgtcatgcgctataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggggtacgcaaggaagacattccggcactggcgcaggcggcactgaatgatgtttgtaccggtggcaacccgcgtgaagcaacgcttgaggatattgtagagctttaccataccgcctggtaa

SEQ ID NO: 5 - protein, 336 aa, Escherichia coli
MKAAVVTKDHHVDVTYKTLRSLKHGEALLKMECCGVCHTDLHVKNGDFGDKTGVILGHEGIGVVAEVGPGVTSLKPGDRASVAWFYEGCGHCEYCNSGNETLCRSVKNAGYSVDGGMAEECIVVADYAVKVPDGLDSAAASSITCAGVTTYKAVKLSKIRPGQWIAIYGLGGLGNLALQYAKNVFNAKVIAIDVNDEQLKLATEMGADLAINSHTEDAAKIVQEKTGGAHAAVVTAVAKAAFNSAVDAVRAGGRVVAVGLPPESMSLDIPRLVLDGIEVVGSLVGTRQDLTEAFQFAAEGKVVPKVALRPLADINTIFTEMEEGKIRGRMVIDFRH

SEQ ID NO: 6 - DNA, 1011 bp, Escherichia coli
atgaaggctgcagttgttacgaaggatcatcatgttgacgttacgtataaaacactgcgctcactgaaacatggcgaagccctgctgaaaatggagtgttgtggtgtatgtcataccgatcttcatgttaagaatggcgattttggtgacaaaaccggcgtaattctgggccatgaaggcatcggtgtggtggcagaagtgggtccaggtgtcacctcattaaaaccaggcgatcgtgccagcgtggcgtggttctacgaaggatgcggtcattgcgaatactgtaacagtggtaacgaaacgctctgccgttcagttaaaaatgccggatacagcgttgatggcgggatggcggaagagtgcatcgtggtcgccgattacgcggtaaaagtgccagatggtctggactcggcggcggccagcagcattacctgtgcgggagtcaccacctacaaagccgttaagctgtcaaaaattcgtccagggcagtggattgctatctacggtcttggcggtctgggtaacctcgccctgcaatacgcgaagaatgtctttaacgccaaagtgatcgccattgatgtcaatgatgagcagttaaaactggcaaccgaaatgggcgcagatttagcgattaactcacacaccgaagacgccgccaaaattgtgcaggagaaaactggtggcgctcacgctgcggtggtaacagcggtagctaaagctgcgtttaactcggcagttgatgctgtccgtgcaggcggtcgtgttgtggctgtcggtctaccgccggagtctatgagcctggatatcccacgtcttgtgctggatggtattgaagtggtcggttcgctggtcggcacgcgccaggatttaactgaagccttccagtttgccgccgaaggtaaagtggtgccgaaagtcgccctgcgtccgttagcggacatcaacaccatctttactgagatggaagaaggcaaaatccgtggccgcatggtgattgatttccgtcactaa

SEQ ID NO: 7 - protein, 269 aa, Escherichia coli
MTHKATEILTGKVMQKSVLITGCSSGIGLESALELKRQGFHVLAGCRKPDDVERMNNMGFTGVLIDLDSPESVDRAADEVIALTDNCLYGIFNNAGFGMYGPLSTISRAQMEQQFSANFFGAHQLTMRLLPAMLPHGEGRIVMTSSVMGLISTPGRGAYAASKYALEAWSDALRMELRHSGIKVSLIEPGPIRTRFTDNVNQTQSDKPVENPGIAARFTLGPEAVVDKVRHAFISEKPKMRYPVTLVTWAVMVLKRLLPGRVMDKILQG

SEQ ID NO: 8 - DNA, 810 bp, Escherichia coli
atgactcataaagcaacggagatcctgacaggtaaagttatgcaaaaatcggtcttaattaccggatgttccagtggaattggcctggaaagcgcgctcgaattaaaacgccagggttttcatgtgctggcaggttgccgaaaaccggatgatgttgagcgtatgaacaacatgggatttaccggcgtgttgatcgatctggattcaccagaaagtgttgatcgcgcagcagacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagacgcgctgcgcatggagctacgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtaacgctggtgacctgggcagtaatggtgcttaagcgcctgctgccggggcgcgtgatggacaaaatattgcaggggtga

SEQ ID NO: 9 - protein, 395 aa, Escherichia coli
MQNELQTALFQAFDTLNLQRVKTFSVPPVTLCGPGAVSSCGQQAQTRGLKHLFVMADSFLHQAGMTAGLTRSLAVKGIAMTLWPCPVGEPCITDVCAAVAQLRESGCDGVIAFGGGSVLDAAKAVALLVTNPDSTLAEMSETSVLQPRLPLIAIPTTAGTGSETTNVTVIIDAVSGRKQVLAHASLMPDVAILDAALTEGVPSHVTAMTGIDALTHAIEAYSALNATPFTDSLAIGAIAMIGKSLPKAVGYGHDLAARESMLLASCMAGMAFSSAGLGLCHAMAHQPGAALHIPHGLANAMLLPTVMEFNRMVCRERFSQIGRALRTKKSDDRDAINAVSELIAEVGIGKRLGDVGATSAHYGAWAQAALEDICLRSNPRTASLEQIVGLYAAAQ

SEQ ID NO: 10 - DNA, 1188 bp, Escherichia coli
atgcaaaatgaattgcagaccgcgctctttcaggcgttcgataccctgaatctgcaacgggtaaaaacatttagcgttccaccggtgacgctttgcggtccgggcgcggtgagcagttgcgggcagcaagcgcaaacgcgtgggctgaaacatctgttcgtgatggcagacagctttttgcatcaggcggggatgaccgccgggctgacgcgcagcctggctgttaaaggcatcgccatgacgctctggccatgtccggtgggcgaaccgtgcattaccgacgtgtgtgcagccgtggcgcagttgcgtgagtcaggctgtgatggggtgatcgcatttggcggcggctcggtgctggatgcggcgaaagccgtggcgttgctggtgacgaaccccgatagcacgctggcagagatgtcagaaaccagcgttctgcaaccgcgcttgccgctgattgccattccaacgaccgccggaaccggctctgaaaccaccaatgtaacggtgattatcgacgcggtgagcgggcgcaagcaggtgttagcccatgcctcgctgatgccggatgtggcgatcctcgacgccgcattgaccgaaggtgtgccgtcgcatgtcacggcgatgaccggcattgatgcgttaacccatgccattgaagcatacagcgccctgaacgctacaccgtttaccgacagcctggcgattggtgccattgcgatgattggcaaatcgctgccgaaagcggtgggctacggtcacgaccttgccgcgcgcgagagcatgttactggcttcatgtatggcgggaatggcgttttccagtgcgggtcttgggttgtgccacgcgatggcgcatcagccgggcgcggcgctgcatattccgcacggtctcgcgaacgccatgttgctgccaacggtgatggaatttaaccggatggtttgtcgtgaacgctttagtcagattggtcgggcactgcgaactaaaaaatccgacgatcgtgacgctattaacgcggtaagtgagctgattgcggaagttgggattggtaaacgactgggcgatgttggtgcgacatctgcgcattacggcgcatgggcgcaggccgcgctggaagatatttgtctgcgcagtaacccgcgtaccgccagcctggagcagattgtcggcctgtacgcagcggcgcaataa

SEQ ID NO: 11 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADY

SEQ ID NO: 12 - DNA, 1020 bp, Escherichia coli
atgtcgatgataaaaagctatgccgcaaaagaagcgggcggagaactggaagtttatgagtacgatcccggtgagctgaggccacaagatgttgaagtgcaggtggattactgcgggatctgccattccgatctgtcgatgatcgataacgaatggggattttcacaatatccgctggttgccgggcatgaggtgattgggcgcgtggtggcactcgggagcgccgcgcaggataaaggtttgcaggtcggtcagcgtgtcgggattggctggacggcgcgtagctgtggtcactgcgacgcctgtattagcggtaatcagatcaactgcgagcaaggtgcggtgccgacgattatgaatcgcggtggctttgccgagaagttgcgtgcggactggcaatgggtgattccactgccagaaaatattgatatcgagtccgccgggccgctgttgtgcggcggtatcacggtctttaaaccactgttgatgcaccatatcactgctaccagccgcgttggggtaattggtattggcgggctggggcatatcgctataaaacttctgcacgcaatgggatgcgaggtgacagcctttagttctaatccggcgaaagagcaggaagtgctggcgatgggtgccgataaagtggtgaatagccgcgatccgcaggcactgaaagcactggcggggcagtttgatctcattatcaacaccgtcaacgtcagcctcgactggcagccctattttgaggcgctgacctatggcggtaatttccatacggtcggtgcggttctcacgccgctgtctgttccggcctttacgttaattgcgggcgatcgcagcgtctctggttctgctaccggcacgccttatgagctgcgtaagctgatgcgttttgccgcccgcagcaaggttgcgccgaccaccgaactgttcccgatgtcgaaaattaacgacgccatccagcatgtgcgcgacggtaaggcgcgttaccgcgtggtgttgaaagccgattattga

SEQ ID NO: 13 - protein, 349 aa, Escherichia coli
MKIKAVGAYSAKQPLEPMDITRREPGPNDVKIEIAYCGVCHSDLHQVRSEWAGTVYPCVPGHEIVGRVVAVGDQVEKYAPGDLVGVGCIVDSCKHCEECEDGLENYCDHMTGTYNSPTPDEPGHTLGGYSQQIVVHERYVLRIRHPQEQLAAVAPLLCAGITTYSPLRHWQAGPGKKVGVVGIGGLGHMGIKLAHAMGAHVVAFTTSEAKREAAKALGADEVVNSRNADEMAAHLKSFDFILNTVAAPHNLDDFTTLLKRDGTMTLVGAPATPHKSPEVFNLIMKRRAIAGSMIGGIPETQEMLDFCAEHGIVADIEMIRADQINEAYERMLRGDVKYRFVIDNRTLTD

SEQ ID NO: 14 - DNA, 1050 bp, Escherichia coli
atgaagatcaaagctgttggtgcatattccgctaaacaaccacttgaaccgatggatatcacccggcgtgaaccgggaccgaatgatgtcaaaatcgaaatcgcttactgtggcgtttgccattccgatctccaccaggtccgttccgagtgggcggggacggtttacccctgcgtgccgggtcatgaaattgtggggcgtgtggtagccgttggtgatcaggtagaaaaatatgcgccgggcgatctggtcggtgtcggctgcattgtcgacagttgtaaacattgcgaagagtgtgaagacgggttggaaaactactgtgatcacatgaccggcacctataactcgccgacgccggacgaaccgggccatactctgggcggctactcacaacagatcgtcgttcatgagcgatatgttctgcgtattcgtcacccgcaagagcagctggcggcggtggctcctttgttgtgtgcagggatcaccacgtattcgccgctacgtcactggcaggccgggccgggtaaaaaagtgggcgtggtcggcatcggcggtctgggacatatggggattaagctggcccacgcgatgggggcacatgtggtggcatttaccacttctgaggcaaaacgcgaagcggcaaaagccctgggggccgatgaagttgttaactcacgcaatgccgatgagatggcggctcatctgaagagtttcgatttcattttgaatacagtagctgcgccacataatctcgacgattttaccaccttgctgaagcgtgatggcaccatgacgctggttggtgcgcctgcgacaccgcataaatcgccggaagttttcaacctgatcatgaaacgccgtgcgatagccggttctatgattggcggcattccagaaactcaggagatgctcgatttttgcgccgaacatggcatcgtggctgatatagagatgattcgggccgatcaaattaatgaagcctatgagcgaatgctgcgcggtgatgtgaaatatcgttttgttatcgataatcgcacactaacagactga

SEQ ID NO: 15 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 16 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagatcctgctggatacctactacggtcgtgattatgtagaaggtgaaactgcagcgaaaaaagaagccgctccggctaaagctgagaaaaaagcgaaaaaatccgcttaa

SEQ ID NO: 17 - protein, 412 aa, Escherichia coli
MKALTYHGPHHVQVENVPDPGIEQADDIILRITATAICGSDLHLYRGKIPQVKHGDIFGHEFMGEVVETGKDVKNLQKGDRVVIPFVIACGDCFFCRLQQYAACENTNAGKGAALNKKQIPAPAALFGYSHLYGGVPGGQAEYVRVPKGNVGPFKVPPLLSDDKALFLSDILPTAWQAAKNAQIQQGSSVAVYGAGPVGLLTIACARLLGAEQIFVVDHHPYRLHFAADRYGAIPINFDEDSDPAQSIIEQTAGHRGVDAVIDAVGFEAKGSTTETVLTNLKLEGSSGKALRQCIAAVRRGGIVSVPGVYAGFIHGFLFGDAFDKGLSFKMGQTHVHAWLGELLPLIEKGLLKPEEIVTHYMPFEEAARGYEIFEKREEECRKVILVPGAQSAEAAQKAVSGLVNAMPGGTI

SEQ ID NO: 18 - DNA, 1239 bp, Escherichia coli
atgaaagcattgacttatcacggcccacatcacgttcaggtagaaaatgttcccgatccgggcattgaacaggcagatgatattattctgcgtattacggcaacggcgatctgtggctctgacctccatctttatcgaggcaaaataccccaggttaaacatggcgatatttttggtcatgaatttatgggggaagtcgttgaaaccggaaaggacgtaaaaaatttgcaaaaaggcgaccgggtggtaattccgtttgtcattgcttgtggcgactgttttttctgtcgattacagcaatatgccgcctgcgaaaataccaatgcgggtaaaggcgctgcgctcaataaaaaacagataccagctcccgcggcattgtttggttatagtcacctgtatggcggcgttcctggtgggcaggcggaatatgtccgcgtccctaaagggaatgtggggccgtttaaagtaccgcctttgctttcagatgataaagcgcttttcctttctgatattctgccaacggcatggcaggcagcaaaaaatgcgcagatccaacaaggttcaagcgttgcagtctatggtgctggtcctgtgggattgttgacaatcgcctgtgcacggttgctcggtgcggaacagatttttgttgttgatcatcatccctaccgcttgcatttcgccgccgaccgctacggcgcgatcccgattaattttgatgaagacagcgatccggcacagtcaattattgaacaaacggcaggtcaccggggcgtggatgcagtaatagacgccgtcggttttgaagcgaaaggcagcaccacggaaacggtgctgactaacctgaaactggagggcagcagcggtaaagcgttgcgtcagtgtattgcggcggtcaggcgtggcggcattgttagcgtaccgggcgtctacgctggatttattcacggtttcctgtttggcgacgcctttgataaagggttgtcgtttaaaatgggacagacccacgttcacgcatggctgggagaattattaccgttaattgagaaaggattactgaaaccagaagaaattgttacccactatatgccgtttgaagaggccgcccggggatatgagattttcgaaaaacgtgaagaggagtgccgtaaggtgattctggtacccggtgcacaaagcgcagaggcggcgcagaaggcggtttcaggtctagtgaatgcgatgccggggggaacaatatga

SEQ ID NO: 19 - protein, 275 aa, Escherichia coli
MANPTVIKLQDGNVMPQLGLGVWQASNEEVITAIQKALEVGYRSIDTAAAYKNEEGVGKALKNASVNREELFITTKLWNDDHKRPREALLDSLKKLQLDYIDLYLMHWPVPAIDHYVEAWKGMIELQKEGLIKSIGVCNFQIHHLQRLIDETGVTPVINQIELHPLMQQRQLHAWNATHKIQTESWSPLAQGGKGVFDQKVIRDLADKYGKTPAQIVIRWHLDSGLVVIPKSVTPSRIAENFDVWDFRLDKDELGEIAKLDQGKRLGPDPDQFGG

SEQ ID NO: 20 - DNA, 828 bp, Escherichia coli
atggctaatccaaccgttattaagctacaggatggcaatgtcatgccccagctgggactgggcgtctggcaagcaagtaatgaggaagtaatcaccgccattcaaaaagcgttagaagtgggttatcgctcgattgataccgccgcggcctacaagaacgaagaaggtgtcggcaaagccctgaaaaatgcctcagtcaacagagaagaactgttcatcaccactaagctgtggaacgacgaccacaagcgcccccgcgaagccctgctcgacagcctgaaaaaactccagcttgattatatcgacctctacttaatgcactggcccgttcccgctatcgaccattatgtcgaagcatggaaaggcatgatcgaattgcaaaaagagggattaatcaaaagcatcggcgtgtgcaacttccagatccatcacctgcaacgcctgattgatgaaactggcgtgacgcctgtgataaaccagatcgaacttcatccgctgatgcaacaacgccagctacacgcctggaacgcgacacacaaaatccagaccgaatcctggagcccattagcgcaaggagggaaaggcgttttcgatcagaaagtcattcgcgatctggcagataaatacggcaaaaccccggcgcagattgttatccgctggcatctggatagcggcctggtggtgatcccgaaatcggtcacaccttcacgtattgccgaaaactttgatgtctgggatttccgtctcgacaaagacgaactcggcgaaattgcaaaactcgatcagggcaagcgtctcggtcccgatcctgaccagttcggcggctaa

SEQ ID NO: 21 - protein, 383 aa, Escherichia coli
MASSTFFIPSVNVIGADSLTDAMNMMADYGFTRTLIVTDNMLTKLGMAGDVQKALEERNIFSVIYDGTQPNPTTENVAAGLKLLKENNCDSVISLGGGSPHDCAKGIALVAANGGDIRDYEGVDRSAKPQLPMIAINTTAGTASEMTRFCIITDEARHIKMAIVDKHVTPLLSVNDSSLMIGMPKSLTAATGMDALTHAIEAYVSIAATPITDACALKAVTMIAENLPLAVEDGSNAKAREAMAYAQFLAGMAFNNASLGYVHAMAHQLGGFYNLPHGVCNAVLLPHVQVFNSKVAAARLRDCAAAMGVNVTGKNDAEGAEACINAIRELAKKVDIPAGLRDLNVKEEDFAVLATNALKDACGFTNPIQATHEEIVAIYRAAM

SEQ ID NO: 22 - DNA, 1152 bp, Escherichia coli
atggcatcttcaactttctttattccttctgtgaatgtcatcggcgctgattcattgactgatgcaatgaatatgatggcagattatggatttacccgtaccttaattgtcactgacaatatgttaacgaaattaggtatggcgggtgatgtgcaaaaagcactggaagaacgcaatatttttagcgttatttatgatggcacccaacctaacccaaccacggaaaacgtcgccgcaggtttgaaattacttaaagaaaataattgcgatagcgtgatttccttaggcggtggttctccgcatgactgtgcaaaaggtattgcgctggtggcagccaatggtggtgatatccgtgattatgaaggcgttgaccgctctgcaaaaccgcagctgccgatgatcgccatcaataccactgcgggtacagcatcagaaatgactcgtttctgcatcatcaccgacgaagcgcgtcacatcaaaatggcgattgttgataagcacgtgactccgctgctttctgtcaatgactcctcgctgatgatcggtatgccgaagtcactgaccgccgccactggtatggacgccttaacgcacgctatcgaagcgtatgtttctattgccgccacgccgatcactgacgcttgtgcactgaaagccgtgaccatgattgccgaaaacctgccgttagccgttgaagatggcagtaatgcgaaagcgcgtgaagcaatggcttatgcccagttcctcgccggtatggcgttcaataatgcttctctgggttatgttcatgcgatggcgcaccagctgggcggtttctacaacctgccacacggtgtatgtaacgccgttttgctgccgcatgttcaggtattcaacagcaaagtcgccgccgcacgtctgcgtgactgtgccgctgcaatgggcgtgaacgtgacaggtaaaaacgatgcggaaggtgctgaagcctgcattaacgccatccgtgaactggcgaagaaagtggatatcccggcaggcctacgcgacctgaacgtgaaagaagaagatttcgcggttctggcgactaatgccctgaaagatgcctgtggttttactaacccgatccaggcaactcacgaagaaattgtggcgatttatcgcgcagcgatgtaa

SEQ ID NO: 23 - protein, 369 aa, Escherichia coli
MKSRAAVAFAPGKPLEIVEIDVAPPKKGEVLIKVTHTGVCHTDAFTLSGDDPEGVFPVVLGHEGAGVVVEVGEGVTSVKPGDHVIPLYTAECGECEFCRSGKTNLCVAVRETQGKGLMPDGTTRFSYNGQPLYHYMGCSTFSEYTVVAEVSLAKINPEANHEHVCLLGCGVTTGIGAVHNTAKVQPGDSVAVFGLGAIGLAVVQGARQAKAGRIIAIDTNPKKFDLARRFGATDCINPNDYDKPIKDVLLDINKWGIDHTFECIGNVNVMRAALESAHRGWGQSVIIGVAGAGQEISTRPFQLVTGRVWKGSAFGGVKGRSQLPGMVEDAMKGDIDLEPFVTHTMSLDEINDAFDLMHEGKSIRTVIRY

SEQ ID NO: 24 - DNA, 1110 bp, Escherichia coli
atgaaatcacgtgctgccgttgcatttgctcccggtaaaccgctggaaatcgttgaaattgacgttgcaccaccgaaaaaaggtgaagtgctgattaaagtcacccataccggcgtttgccataccgacgcatttaccctctccggggatgacccggaaggtgtattcccggtggttctcggtcacgaaggggccggcgttgtggttgaagtcggtgaaggcgtaaccagcgtcaaacctggcgaccatgtgatcccgctttacaccgcggagtgcggcgagtgtgagttctgtcgttctggcaaaactaacctctgtgttgcggttcgcgaaacccagggtaaaggcttgatgccagacggcaccacccgtttttcttacaacgggcagccgctttatcactacatgggatgctcaacattcagtgaatacaccgtggtcgcggaagtgtctctggccaaaattaatccagaagcaaaccatgaacacgtctgcctgctgggctgtggcgtgaccaccggtattggcgcggtgcacaacacagctaaagtccagccaggtgattctgttgccgtgtttggtcttggcgcgattggtctggcagtggttcagggcgcgcgtcaggcgaaagcgggacggattatcgctatcgataccaacccgaagaaattcgatctggctcgtcgcttcggtgctaccgactgcattaacccgaatgactacgacaaaccgattaaagatgtcctgctggatatcaacaaatggggtatcgaccatacctttgaatgcatcggtaacgtcaacgtgatgcgtgcggcgctggaaagtgcgcaccgcggctggggtcagtcggtgatcatcggggtagcaggtgccggtcaggaaatctccacccgaccattccagttggtcaccggtcgcgtatggaaaggttccgcgtttggcggcgtgaaaggtcgttcccagttaccgggtatggttgaagatgcgatgaaaggtgatatcgatctggaaccgtttgtcacgcataccatgagccttgatgaaattaatgacgccttcgacctgatgcatgaaggcaaatccattcgaaccgtaattcgttactga

SEQ ID NO: 25 - protein, 267 aa, Escherichia coli
MAIPAFGLGTFRLKDDVVISSVKTALELGYRAIDTAQIYDNEAAVGQAIAESGVPRHELYITTKIWIENLSKDKLIPSLKESLQKLRTDYVDLTLIHWPSPNDEVSVEEFMQELLEAKKEGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD

SEQ ID NO: 26 - DNA, 804 bp, Escherichia coli
atggctatccctgcatttggtttaggtactttccgtctgaaagacgacgttgttatttcatctgtgaaaacggcgcttgaacttggttatcgcgcaattgatactgcacaaatctatgataacgaagccgcagtaggtcaggcgattgcagaaagtggcgtgccacgtcatgaactctacatcaccactaaaatctggattgaaaatctcagcaaagacaaattgatcccgagtctgaaagagagcctgcaaaaattgcgtactgattatgttgatctgactctaatccactggccgtcaccaaacgatgaagtctctgttgaagagtttatgcaggagctgctggaagccaaaaaagaagggttgacgcgtgagatcggtatttccaacttcacgatcccattgatggaaaaggcgattgctgctgttggcgctgaaaacatcgctactaaccagattgaactctctccttatctgcaaaaccgtaaagtggttgcctgggctaaacagcacggcatccatattacttcctatatgacgctggcgtatggtaaggccctgaaagatgaggttattgctcgtatcgcagctaaacacaatgcgactccggcacaagtgattctggcgtgggctatgggggaaggttactcagtaattccttcttctactaaacgtaaaaacctggaaagtaatcttaaggcacaaaatttacagcttgatgccgaagataaaaaagcgatcgccgcactggattgcaacgaccgcctggttagcccggaaggtctggctcctgaatgggattaa

SEQ ID NO: 27 - protein, 294 aa, Escherichia coli
MSHLKDPTTQYYTGEYPKQKQPTPGIQAKMTPVPDCGEKTYVGSGRLKDRKALVTGGDSGIGRAAAIAYAREGADVAISYLPVEEEDAQDVKKIIEECGRKAVLLPGDLSDEKFARSLVHEAHKALGGLDIMALVAGKQVAIPDIADLTSEQFQKTFAINVFALFWLTQEAIPLLPKGASIITTSSIQAYQPSPHLLDYAATKAAILNYSRGLAKQVAEKGIRVNIVAPGPIWTALQISGGQTQDKIPQFGQQTPMKRAGQPAELAPVYVYLASQESSYVTAEVHGVCGGEHLG

SEQ ID NO: 28 - DNA, 885 bp, Escherichia coli
atgtctcatttaaaagacccgaccacgcagtattacactggtgaatatcccaaacagaaacaaccgacgccaggcatccaggcgaagatgacaccggtaccggattgcggcgagaaaacctatgttggtagcggtcgcctgaaagatcgtaaagcactggtgacagggggcgattccggaataggtcgcgctgccgccatcgcttacgcgcgtgaaggggctgacgtggcgatcagttatcttcccgtggaagaagaagacgctcaggatgtgaaaaagatcattgaagaatgcggacgcaaagccgttctgctgccaggcgatttaagcgatgagaaatttgcccgttcgctggttcacgaagcgcacaaggcgttaggcgggctggatattatggcgctggtcgccgggaaacaggttgccattccggatattgcagacctcaccagcgaacagtttcaaaagacctttgccattaacgttttcgcgctgttctggctaacccaggaagcgatccccctgctaccgaaaggtgcaagtatcatcaccacttcgtcaatccaggcataccagccaagtccgcatttactggactatgcggctacgaaggcggcgattctgaactacagccgtggcttggcaaaacaggtcgcggagaaaggtattcgggtgaatattgtcgcgccaggcccgatctggacagcactgcaaatttccggcggacaaacgcaggataagatcccgcagtttggtcagcaaacgccgatgaaacgtgcggggcaaccggcggaactggcccctgtatatgtttatctggcaagtcaggagtcgagctacgtcaccgcagaagtgcacggcgtgtgcggcggcgagcatttaggttaa

SEQ ID NO: 29 - protein, 326 aa, Escherichia coli
MKKIPLGTTDITLSRMGLGTWAIGGGPAWNGDLDRQICIDTILEAHRCGINLIDTAPGYNFGNSEVIVGQALKKLPREQVVVETKCGIVWERKGSLFNKVGDRQLYKNLSPESIREEVEASLQRLGIDYIDIYMTHWQSVPPFFTPIAETVAVLNELKAEGKIRAIGAANVDADHIREYLQYGELDIIQAKYSILDRAMENELLPLCRDNGIVVQVYSPLEQGLLTGTITRDYVPGGARANKVWFQRENMLKVIDMLEQWQPLCARYQCTIPTLALAWILKQSDLISILSGATAPEQVRENVAALNINLSDADATLMREMAEALER

SEQ ID NO: 30 - DNA, 981 bp, Escherichia coli
atgaaaaaaatacctttaggcacaacggatattacgctttcgcgaatggggttggggacatgggccattggcggcggtcctgcatggaatggcgatctcgatcggcaaatatgtattgatacgattcttgaagcccatcgttgcggcattaatctgattgatactgcaccaggatataactttggcaatagtgaagttatcgtcggtcaggcgttaaaaaaactgccccgtgaacaggttgtagtagaaaccaaatgcggcattgtctgggaacgaaaaggaagtttattcaacaaagttggcgatcggcagttgtataaaaacctttccccggaatctatccgcgaagaggtagaagccagcttgcaacgtctgggtattgattacatcgatatctacatgacgcactggcagtcggtgccgccattttttacgccgatagctgaaactgtcgcagtgcttaatgagttaaaagccgaagggaaaattcgcgcgataggcgctgctaacgtcgatgctgaccatatccgcgagtatctgcaatatggtgaactggatattattcaggcgaaatacagtatcctcgaccgggcaatggaaaacgaactgctgccgctatgtcgtgataatggcattgtggttcaggtttattccccgctagagcagggattgttgaccggcaccatcactcgtgattacgttccgggcggcgctcgggcaaataaagtctggttccagcgtgaaaacatgctgaaagtgattgatatgcttgaacagtggcagccactttgtgctcgttatcagtgcacaattcccactctggcactggcgtggatattaaaacagagtgatttaatctccattcttagtggggctactgcaccggaacaggtacgcgaaaatgtcgcggcactgaatatcaacttatcggatgcagacgcaacattgatgagggaaatggcagaggccctggagcgttaa

SEQ ID NO: 31 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 32 - DNA, 1104 bp, Escherichia coli
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaacgcgccggtggaggaaatcgaaaccgtagctgcgcttagccatgcggtaggtttgccaataactctcgctcaactggatattaaagaagatgtcccggcgaaaatgcgaattgtggcagaagcggcatgtgcagaaggtgaaaccatccacaacatgcctggcggcgcgacgccagatcaggtttacgccgctctgctggtagccgaccagtacggtcagcgtttcctgcaagagtgggaataa

SEQ ID NO: 33 - protein, 253 aa, Escherichia coli
MAQVAIITASDSGIGKECALLLAQQGFDIGITWHSDEEGAKDTAREVVSHGVRAEIVQLDLGNLPEGALALEKLIQRLGRIDVLVNNAGAMTKAPFLDMAFDEWRKIFTVDVDGAFLCSQIAARQMVKQGQGGRIINITSVHEHTPLPDASAYTAAKHALGGLTKAMALELVRHKILVNAVAPGAIATPMNGMDDSDVKPDAEPSIPLRRFGATHEIASLVVWLCSEGANYTTGQSLIVDGGFMLANPQFNPE

SEQ ID NO: 34 - DNA, 762 bp, Escherichia coli
atggcacaggttgcgattattaccgcctccgattcggggatcggcaaagagtgcgcgttattactggcgcagcaggggtttgatattggtattacctggcactcagatgaagaaggggcaaaagataccgcgcgtgaggtagttagccacggcgtacgtgcggagatcgtgcagctggatctcggcaatctaccagaaggggcactggcgctggagaaactcattcaacggctggggcgcattgatgtgctggtgaataatgcgggtgcaatgaccaaagcgccgtttcttgatatggcttttgatgagtggcgcaagatttttaccgttgatgtcgatggtgcattcttatgctcgcaaattgcggctcgtcagatggtgaaacaagggcagggcggtcgcatcatcaacattacgtcggtacatgaacatacgccgctgccggatgccagcgcctacacagccgctaaacatgcgctcggtgggttaaccaaagcgatggcgctggagctggtcaggcataagattttggtgaacgcagtcgcgcctggggcgatcgccacgccaatgaatggcatggatgacagcgacgtgaagcccgacgcggagccttcgattcccttgcggcgttttggcgcaacgcatgagattgccagcctggtggtgtggctttgttcggagggcgcaaattacaccaccgggcagtcgttgatagtggatggcggctttatgttggcgaatccacagttcaacccagaatag

SEQ ID NO: 35 - protein, 284 aa, Escherichia coli
MQQKMIQFSGDVSLPAVGQGTWYMGEDASQRKTEVAALRAGIELGLTLIDTAEMYADGGAEKVVGEALTGLREKVFLVSKVYPWNAGGQKAINACEASLRRLNTDYLDLYLLHWSGSFAFEETVAAMEKLIAQGKIRRWGVSNLDYADMQELWQLPGGNQCATNQVLYHLGSRGIEYDLLPWCQQQQMPVMAYSPLAQAGRLRNGLLKNAVVNEIAHAHNISAAQVLLAWVISHQGVMAIPKAATIAHVQQNAAVLEVELSSAELAMLDKAYPAPKGKTALDMV

SEQ ID NO: 36 - DNA, 855 bp, Escherichia coli
atgcaacaaaaaatgattcaatttagtggcgatgtctcactgccagccgtagggcagggaacatggtatatgggcgaagatgccagtcagcgcaaaacagaagttgctgcactacgcgcgggcattgaactcggtttaaccctcattgataccgccgaaatgtatgccgatggcggtgccgaaaaggtggttggggaagcattaaccggtctgcgagagaaggtctttctcgtctctaaagtctatccgtggaatgctggcgggcaaaaagcgataaatgcatgcgaagccagtttacgccgtctcaatactgattatctcgatctttacttattacactggtctggcagtttcgcttttgaagagactgtcgcagcgatggaaaaattgatcgcccagggaaaaatccgccgctggggcgtttctaaccttgattatgctgatatgcaggaactctggcagctgccggggggaaatcagtgtgccactaatcaggtgctttaccatctcggttcacgaggaattgagtacgatctactcccctggtgccagcaacagcagatgccggtgatggcttacagtccgttagcccaggccgggcggttgcgcaatggactgttaaaaaacgcggtagtcaacgaaattgcacatgctcacaatatcagcgcggcacaagtattgttggcgtgggtgatcagtcatcagggtgtgatggcgattccaaaagcggccacgattgcccatgtccaacaaaatgcggctgtgcttgaggtcgaactttcttcagcggaattagctatgctggataaggcatatccggcaccaaaaggaaaaactgcgctggatatggtgtga

SEQ ID NO: 37 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 38 - DNA, 1047 bp, Saccharomyces cerevisiae
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 39 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 40 - DNA, 1047 bp, Saccharomyces cerevisiae
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 41 - protein, 375 aa, Saccharomyces cerevisiae [sequence omitted]

SEQ ID NO: 42 - DNA, 1128 bp, Saccharomyces cerevisiae
atgttgagaacgtcaacattgttcaccaggcgtgtccaaccaagcctattttctagaaacattcttagattgcaatccacagctgcaatccctaagactcaaaaaggtgtcatcttttatgagaataaggggaagctgcattacaaagatatccctgtccccgagcctaagccaaatgaaattttaatcaacgttaaatattctggtgtatgtcacaccgatttacatgcttggcacggcgattggccattacctgttaaactaccattagtaggtggtcatgaaggtgctggtgtagttgtcaaactaggttccaatgtcaagggctggaaagtcggtgatttagcaggtatcaaatggctgaacggttcttgtatgacatgcgaattctgtgaatcaggtcatgaatcaaattgtccagatgctgatttatctggttacactcatgatggttctttccaacaatttgcgaccgctgatgctattcaagccgccaaaattcaacagggtaccgacttggccgaagtagccccaatattatgtgctggtgttactgtatataaagcactaaaagaggcagacttgaaagctggtgactgggttgccatctctggtgctgcaggtggcttgggttccttggccgttcaatatgcaactgcgatgggttacagagttctaggtattgatgcaggtgaggaaaaggaaaaacttttcaagaaattggggggtgaagtattcatcgactttactaaaacaaagaatatggtttctgacattcaagaagctaccaaaggtggccctcatggtgtcattaacgtttccgtttctgaagccgctatttctctatctacggaatatgttagaccatgtggtaccgtcgttttggttggtttgcccgctaacgcctacgttaaatcagaggtattctctcatgtggtgaagtccatcaatatcaagggttcttatgttggtaacagagctgatacgagagaagccttagacttctttagcagaggtttgatcaaatcaccaatcaaaattgttggattatctgaattaccaaaggtttatgacttgatggaaaagggcaagattttgggtagatacgtcgtcgatactagtaaataa

SEQ ID NO: 43 - protein, 382 aa, Saccharomyces cerevisiae
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 44 - DNA, 1149 bp, Saccharomyces cerevisiae
atgtcttccgttactgggttttacattccaccaatctctttctttggtgaaggtgctttagaagaaaccgctgattacatcaaaaacaaggattacaaaaaggctttgatcgttactgatcctggtattgcagctattggtctctccggtagagtccaaaagatgttggaagaacgtgacttaaacgttgctatctatgacaaaactcaaccaaacccaaatattgccaatgtcacagctggtttgaaggttttgaaggaacaaaactctgaaattgttgtttccattggtggtggttctgctcacgacaatgctaaggccattgctttattggctactaacggtggggaaatcggagactatgaaggtgtcaatcaatctaagaaggctgctttaccactatttgccatcaacactactgctggtactgcttccgaaatgaccagattcactattatctctaatgaagaaaagaaaatcaagatggctatcattgacaacaacgtcactccagctgttgctgtcaacgatccatctaccatgtttggtttgccacctgctttgactgctgctactggtctagatgctttgactcactgtatcgaagcttatgtttccaccgcctctaacccaatcaccgatgcctgtgctttgaagggtattgatttgatcaatgaaagcttagtcgctgcatacaaagacggtaaagacaagaaggccagaactgacatgtgttacgctgaatacttggcaggtatggctttcaacaatgcttctctaggttatgttcatgcccttgctcatcaacttggtggtttctaccacttgcctcatggtgtttgtaacgctgtcttgttgcctcatgttcaagaggccaacatgcaatgtccaaaggccaagaagagattaggtgaaattgctttgcatttcggtgcttctcaagaagatccagaagaaaccatcaaggctttgcacgttttaaacagaaccatgaacattccaagaaacttgaaagaattaggtgttaaaaccgaagattttgaaattttggctgaacacgccatgcatgatgcctgccatttgactaacccagttcaattcaccaaagaacaagtggttgccattatcaagaaagcctatgaatattaa

SEQ ID NO: 45 - protein, 351 aa, Saccharomyces cerevisiae
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

SEQ ID NO: 46 - DNA, 1056 bp, Saccharomyces cerevisiae
atgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaatga

SEQ ID NO: 47 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 48 - DNA, 1083 bp, Saccharomyces cerevisiae
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 49 - protein, 361 aa, Saccharomyces cerevisiae
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 50 - DNA, 1086 bp, Saccharomyces cerevisiae
atgctttacccagaaaaatttcagggcatcggtatttccaacgcaaaggattggaagcatcctaaattagtgagttttgacccaaaaccctttggcgatcatgacgttgatgttgaaattgaagcctgtggtatctgcggatctgattttcatatagccgttggtaattggggtccagtcccagaaaatcaaatccttggacatgaaataattggccgcgtggtgaaggttggatccaagtgccacactggggtaaaaatcggtgaccgtgttggtgttggtgcccaagccttggcgtgttttgagtgtgaacgttgcaaaagtgacaacgagcaatactgtaccaatgaccacgttttgactatgtggactccttacaaggacggctacatttcacaaggaggctttgcctcccacgtgaggcttcatgaacactttgctattcaaataccagaaaatattccaagtccgctagccgctccattattgtgtggtggtattacagttttctctccactactaagaaatggctgtggtccaggtaagagggtaggtattgttggcatcggtggtattgggcatatggggattctgttggctaaagctatgggagccgaggtttatgcgttttcgcgaggccactccaagcgggaggattctatgaaactcggtgctgatcactatattgctatgttggaggataaaggctggacagaacaatactctaacgctttggaccttcttgtcgtttgctcatcatctttgtcgaaagttaattttgacagtatcgttaagattatgaagattggaggctccatcgtttcaattgctgctcctgaagttaatgaaaagcttgttttaaaaccgttgggcctaatgggagtatcaatctcaagcagtgctatcggatctaggaaggaaatcgaacaactattgaaattagtttccgaaaagaatgtcaaaatatgggtggaaaaacttccgatcagcgaagaaggcgtcagccatgcctttacaaggatggaaagcggagacgtcaaatacagatttactttggtcgattatgataagaaattccataaatag

SEQ ID NO: 51 - protein, 386 aa, Saccharomyces cerevisiae
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 52 - DNA, 1161 bp, Saccharomyces cerevisiae
atgtccgccgctactgttggtaaacctattaagtgcattgctgctgttgcgtatgatgcgaagaaaccattaagtgttgaagaaatcacggtagacgccccaaaagcgcacgaagtacgtatcaaaattgaatatactgctgtatgccacactgatgcgtacactttatcaggctctgatccagaaggacttttcccttgcgttctgggccacgaaggagccggtatcgtagaatctgtaggcgatgatgtcataacagttaagcctggtgatcatgttattgctttgtacactgctgagtgtggcaaatgtaagttctgtacttccggtaaaaccaacttatgtggtgctgttagagctactcaagggaaaggtgtaatgcctgatgggaccacaagatttcataatgcgaaaggtgaagatatataccatttcatgggttgctctactttttccgaatatactgtggtggcagatgtctctgtggttgccatcgatccaaaagctcccttggatgctgcctgtttactgggttgtggtgttactactggttttggggcggctcttaagacagctaatgtgcaaaaaggcgataccgttgcagtatttggctgcgggactgtaggactctccgttatccaaggtgcaaagttaaggggcgcttccaagatcattgccattgacattaacaataagaaaaaacaatattgttctcaatttggtgccacggattttgttaatcccaaggaagatttggccaaagatcaaactatcgttgaaaagttaattgaaatgactgatgggggtctggattttacttttgactgtactggtaataccaaaattatgagagatgctttggaagcctgtcataaaggttggggtcaatctattatcattggtgtggctgccgctggtgaagaaatttctacaaggccgttccagctggtcactggtagagtgtggaaaggctctgcttttggtggcatcaaaggtagatctgaaatgggcggtttaattaaagactatcaaaaaggtgccttaaaagtcgaagaatttatcactcacaggagaccattcaaagaaatcaatcaagcctttgaagatttgcataacggtgattgcttaagaaccgtcttgaagtctgatgaaataaaatag

SEQ ID NO: 53 - protein, 363 aa, Saccharomyces cerevisiae
MIGSASDSSSKLGRLRFLSETAAIKVSPLILGEVSYDGARSDFLKSMNKNRAFELLDTFYEAGGNFIDAANNCQNEQSEEWIGEWIQSRRLRDQIVIATKFIKSDKKYKAGESNTANYCGNHKRSLHVSVRDSLRKLQTDWIDILYVHWWDYMSSIEEFMDSLHILVQQGKVLYLGVSDTPAWVVSAANYYATSYGKTPFSIYQGKWNVLNRDFERDIIPMARHFGMALAPWDVMGGGRFQSKKAMEERRKNGEGIRSFVGASEQTDAEIKISEALAKIAEEHGTESVTAIAIAYVRSKAKNFFPSVEGGKIEDLKENIKALSIDLTPDNIKYLESIVPFDIGFPNNFIVLNSLTQKYGTNNV

SEQ ID NO: 54 - DNA, 1092 bp, Saccharomyces cerevisiae
atgattgggtccgcgtccgactcatctagcaagttaggacgcctccgatttctttctgaaactgccgctattaaagtatccccgttaatcctaggagaagtctcatacgatggagcacgttcggattttctcaaatcaatgaacaagaatcgagcttttgaattgcttgatactttttacgaggcaggtggaaatttcattgatgccgcaaacaactgccaaaacgagcaatcagaagaatggattggtgaatggatacagtccagaaggttacgtgatcaaattgtcattgcaaccaagtttataaaaagcgataaaaagtataaagcaggtgaaagtaacactgccaactactgtggtaatcacaagcgtagtttacatgtgagtgtgagggattctctccgcaaattgcaaactgattggattgatatactttacgttcactggtgggattatatgagttcaatcgaagaatttatggatagtttgcatattctggtccagcagggcaaggtcctctatttgggtgtatctgatacacctgcttgggttgtttctgcggcaaactactacgctacatcttatggtaaaactccctttagtatctaccaaggtaaatggaacgtgttgaacagagattttgagcgtgatattattccaatggctaggcatttcggtatggccctcgccccatgggatgtcatgggaggtggaagatttcagagtaaaaaagcaatggaggaacggaggaagaatggagagggtattcgttctttcgttggcgcctccgaacaaacagatgcagaaatcaagattagtgaagcattggccaagattgctgaggaacatggcactgagtctgttactgctattgctattgcctatgttcgctctaaggcgaaaaatttttttccgtcggttgaaggaggaaaaattgaggatctcaaagagaacattaaggctctcagtatcgatctaacgccagacaatataaaatacttagaaagtatagttccttttgacatcggatttcctaataattttatcgtgttaaattccttgactcaaaaatatggtacgaataatgtttag

SEQ ID NO: 55 - protein, 329 aa, Saccharomyces cerevisiae
MGSMNKEQAFELLDAFYEAGGNCIDTANSYQNEESEIWIGEWMKSRKLRDQIVIATKFTGDYKKYEVGGGKSANYCGNHKHSLHVSVRDSLRKLQTDWIDILYVHWWDYMSSIEEVMDSLHILVQQGKVLYLGVSDTPAWVVSAANYYATSHGKTPFSIYQGKWNVLNRDFERDIIPMARHFGMALAPWDVMGGGRFQSKKAMEERKKNGEGLRTVSGTSKQTDKEVKISEALAKVAEEHGTESVTAIAIAYVRSKAKNVFPLVGGRKIEHLKQNIEALSIKLTPEQIEYLESIIPFDVGFPTNFIGDDPAVTKKASLLTAMSAQISFD

SEQ ID NO: 56 - DNA, 990 bp, Saccharomyces cerevisiae
atgggctctatgaataaggaacaggcttttgaacttcttgatgctttttatgaagcaggaggtaattgcattgatactgcaaacagttaccaaaatgaagagtcagagatttggataggtgaatggatgaaatcaagaaagttgcgtgaccaaattgtaattgccaccaagtttaccggagattataagaagtatgaagtaggtggcggtaaaagtgccaactattgtggtaatcacaagcatagtttacatgtgagtgtgagggattctctccgcaaattgcaaactgattggattgatatactttacgttcactggtgggattatatgagttcaatcgaagaagttatggatagtttgcatattttagttcagcagggcaaagtcctctatttgggtgtgtctgatacacctgcttgggttgtttctgcggcaaactactacgccacatctcatgggaaaactccttttagtatctatcaaggtaaatggaatgtgttgaacagggactttgagcgcgatatcattccaatggccagacattttggtatggctctagccccatgggatgttatgggaggtggaagatttcagagtaaaaaagcaatggaggaacggaagaagaatggagagggtctgcgtactgtttcgggtacttctaaacagacggataaagaggttaagatcagtgaagcattggccaaggttgctgaggaacatggcactgagtctgttactgctattgctattgcctatgttcgctctaaggcgaaaaatgttttcccattggttggtggaaggaaaattgaacacctcaaacagaacattgaggctttaagtatcaaactgacaccagaacagatagaatacttagaaagtattattccttttgatgttggttttcctactaattttatcggtgatgatccggctgttaccaagaaggcttcacttctcacggcaatgtctgcgcagatttccttcgattaa

SEQ ID NO: 57 - protein, 288 aa, Saccharomyces cerevisiae
MASRKLRDQIVIATKFTTDYKGYDVGKGKSANFCGNHKRSLHVSVRDSLRKLQTDWIDILYVHWWDYMSSIEEVMDSLHILVQQGKVLYLGVSDTPAWVVSAANYYATSHGKTPFSIYQGKWNVLNRDFERDIIPMARHFGMALAPWDVMGGGRFQSKKAVEERKKKGEGLRTFFGTSEQTDMEVKISEALLKVAEEHGTESVTAIAIAYVRSKAKHVFPLVGGRKIEHLKQNIEALSIKLTPEQIKYLESIVPFDVGFPTNFIGDDPAVTKKPSFLTEMSAKISFED

SEQ ID NO: 58 - DNA, 867 bp, Saccharomyces cerevisiae
atggcatcaagaaaactgcgtgaccagattgtaattgccactaaatttaccacggattataaggggtatgatgtaggcaaggggaagagtgccaatttctgtgggaatcacaagcgcagtttgcatgtaagtgtgagagattcccttcgtaagttgcaaactgattggattgatattctttacgttcactggtgggattatatgagctccattgaggaagttatggatagtttgcacattcttgtgcagcagggcaaagtactctatctaggtgtgtctgatactcctgcctgggttgtttctgcagcaaattactacgccacatctcatggtaaaactccctttagtatctatcaaggtaaatggaatgtattgaacagggactttgaacgtgatatcattccaatggctaggcattttggtatggctcttgctccatgggatgttatgggaggcgggagatttcagagtaaaaaggcagtggaagagcggaagaagaaaggagaaggcttgcgtaccttttttggtacttcggaacagacggatatggaggttaaaatcagcgaagcattgttaaaagttgcggaagaacatggcactgagtctgtcactgctattgccatagcttatgttcggtctaaagcgaaacatgttttcccattagtgggaggaagaaagatcgaacatctcaaacagaacattgaggctttgagcattaaattaacaccagaacaaataaagtacttagaaagtattgttccttttgatgtcggatttcccactaattttattggagatgacccagctgttaccaagaaaccttcatttctcaccgaaatgtctgccaagattagcttcgaagattag

SEQ ID NO: 59 - protein, 376 aa, Saccharomyces cerevisiae
MTDLFKPLPEPPTELGRLRVLSKTAGIRVSPLILGGASIGDAWSGFMGSMNKEQAFELLDAFYEAGGNCIDTANSYQNEESEIWIGEWMASRKLRDQIVIATKFTGDYKKYEVGGGKSANYCGNHKRSLHVSVRDSLRKLQTDWIDILYIHWWDYMSSIEEVMDSLHILVQQGKVLYLGVSDTPAWVVSAANYYATSHGKTPFSVYQGKWNVLNRDFERDIIPMARHFGMALAPWDVMGGGRFQSKKAMEERKKNGEGLRTFVGGPEQTELEVKISEALTKIAEEHGTESVTAIAIAYVRSKAKNVFPLIGGRKIEHLKQNIEALSIKLTPEQIEYLESIVPFDVGFPKSLIGDDPAVTKKLSPLTSMSARIAFDN

SEQ ID NO: 60 - DNA, 1131 bp, Saccharomyces cerevisiae
atgactgacttgtttaaacctctacctgaaccacctaccgaattgggacgtctcagggttctttctaaaactgccggcataagggtttcaccgctaattctgggaggagcttcaatcggcgacgcatggtcaggctttatgggctctatgaataaggaacaggcctttgaacttcttgatgctttttatgaagctggaggtaattgtattgatactgcaaacagttaccaaaatgaagagtcagagatttggataggtgaatggatggcatcaagaaaactgcgtgaccagattgtaattgccaccaagtttaccggagattataagaagtatgaagtaggtggtggtaaaagtgccaactactgtggtaatcacaagcgtagtttacatgtgagtgtgagggattctctccgcaaattgcaaactgattggattgatatactttacattcactggtgggattatatgagttcaatcgaagaagttatggatagtttgcatattttagttcagcagggcaaggtcctatatttaggagtatctgatacacctgcttgggttgtttctgcggcaaattactacgctacatctcatggtaaaactccttttagcgtctatcaaggtaaatggaatgtattgaacagggactttgagcgtgatattattccaatggctaggcattttggtatggctctagccccatgggatgtcatgggaggtggaagatttcagagtaaaaaagcaatggaagaacggaagaagaatggagagggtctgcgtacttttgtgggtggccccgaacaaacagaattggaggttaaaatcagcgaagcattgactaaaattgctgaggaacatggaacagagtctgttactgctatcgctattgcctatgttcgctctaaagcgaaaaatgttttcccattgattggaggaaggaaaattgaacatctcaagcagaacattgaggctttgagtattaaattaacaccggaacaaatagaatacctggaaagtattgttccttttgatgttggctttcccaaaagtttaataggagatgacccagcggtaaccaagaagctttcacccctcacatcgatgtctgccaggatagcttttgacaattag

SEQ ID NO: 61 - protein, 143 aa, Saccharomyces cerevisiae
MARHFGMALAPWDVMGGGRFQSKKAMEERRKNGECIRSFVGASEQTDAEIKISEALAKVAEEHGTESVTAIAIAYVRSKAKNVFPSVEGGKIEDLKENIKALSIDLTPDNIKYLENVVPFDIGFPNTFIVLNSLTQKYGTNNV

SEQ ID NO: 62 - DNA, 432 bp, Saccharomyces cerevisiae
atggctaggcatttcggtatggccctcgccccatgggatgtcatgggaggtggaagatttcagagtaaaaaagcaatggaggaacggaggaagaatggagagtgtattcgttctttcgttggcgcctccgaacaaacagatgcagaaatcaagattagtgaagcattagccaaggttgctgaggaacatggcactgagtctgttactgctattgctattgcctatgttcgctctaaggcgaaaaatgtttttccgtcggttgaaggaggaaaaattgaggatctcaaagagaacattaaggctctcagtatcgatctaacgccggacaatataaaatacttggaaaatgtagttccttttgacatcggatttcctaacacttttatcgtgttaaattccttgactcaaaaatatggtacgaataatgtttag

SEQ ID NO: 63 - protein, 312 aa, Saccharomyces cerevisiae
MPATLKNSSATLKLNTGASIPVLGFGTWRSVDNNGYHSVIAALKAGYRHIDAAAIYLNEEEVGRAIKDSGVPREEIFITTKLWGTEQRDPEAALNKSLKRLGLDYVDLYLMHWPVPLKTDRVTDGNVLCIPTLEDGTVDIDTKEWNFIKTWELMQELPKTGKTKAVGVSNFSINNIKELLESPNNKVVPATNQIEIHPLLPQDELIAFCKEKGIVVEAYSPFGSANAPLLKEQAIIDMAKKHGVEPAQLIISWSIQRGYVVLAKSVNPERIVSNFKIFTLPEDDFKTISNLSKVHGTKRVVDMKWGSFPIFQ

SEQ ID NO: 64 - DNA, 939 bp, Saccharomyces cerevisiae
atgcctgctacgttaaagaattcttctgctacattaaaactaaatactggtgcctccattccagtgttgggtttcggcacttggcgttccgttgacaataacggttaccattctgtaattgcagctttgaaagctggatacagacacattgatgctgcggctatctatttgaatgaagaagaagttggcagggctattaaagattccggagtccctcgtgaggaaatttttattactactaagctttggggtacggaacaacgtgatccggaagctgctctaaacaagtctttgaaaagactaggcttggattatgttgacctatatctgatgcattggccagtgcctttgaaaaccgacagagttactgatggtaacgttctgtgcattccaacattagaagatggcactgttgacatcgatactaaggaatggaattttatcaagacgtgggagttgatgcaagagttgccaaagacgggcaaaactaaagccgttggtgtctctaatttttctattaacaacattaaagaattattagaatctccaaataacaaggtggtaccagctactaatcaaattgaaattcatccattgctaccacaagacgaattgattgccttttgtaaggaaaagggtattgttgttgaagcctactcaccatttgggagtgctaatgctcctttactaaaagagcaagcaattattgatatggctaaaaagcacggcgttgagccagcacagcttattatcagttggagtattcaaagaggctacgttgttctggccaaatcggttaatcctgaaagaattgtatccaattttaagattttcactctgcctgaggatgatttcaagactattagtaacctatccaaagtgcatggtacaaagagagtcgttgatatgaagtggggatccttcccaattttccaatga

SEQ ID NO: 65 - protein, 353 aa, Corynebacterium glutamicum
MSISVKALQKSGPEAPFEVKIIERRDPRADDVVIDIKAAGICHSDIHTIRNEWGEAHFPLTVGHEIAGVVSAVGSDVTKWKVGDRVGVGCLVNSCGECEQCVAGFENNCLRGNVGTYNSNDVDGTITQGGYAEKVVVNERFLCSIPEELNFDVAAPLLCAGITTYSPIARWNVKEGDKVAVMGLGGLGHMGVQIAAAKGAEVTVLSRSLRKAELAKELGAARTLATSDEDFFTEHAGEFDFILNTISASIPVDKYLSLLKPHGVMAVVGLPPEKQPLSFGALIGGGKVLTGSNIGGIPETQEMLDFCAKHGLGAMIETVGVNDVDAAYDRVVAGDVQFRVVIDTASFAEVEAV

SEQ ID NO: 66 - DNA, 1062 bp, Corynebacterium glutamicum
gtgagtatctcagtaaaagcactacaaaagtccggcccagaagcacctttcgaggtcaagatcattgaacgccgtgacccacgcgcagatgatgtggttattgatatcaaagctgcgggcatctgccacagcgatatccacaccatccgcaacgaatggggcgaggcgcacttcccgctcaccgtcggccacgaaatcgcaggcgttgtctctgcggttggatccgatgtaaccaaatggaaagtcggcgaccgcgtgggcgtcggctgcctcgttaactcctgcggcgaatgcgaacagtgcgtcgcaggatttgaaaacaactgccttcgcggaaacgtcggaacctacaactctaacgacgtcgacggcaccatcacccaaggcggctacgctgaaaaggtagtggtcaacgaacgtttcctgtgcagcatcccagaggaacttaacttcgatgtcgcagcaccactgctgtgcgcaggcatcaccacctactccccaatcgctcgctggaacgttaaagaaggcgacaaagtagcagtcatgggcctcggcggactcggacacatgggtgtccagatcgctgcagccaagggtgctgaggttaccgttctgtcccgttccctgcgcaaggcagaacttgccaaggaactcggcgcagctcgcacgcttgcgacttctgatgaggatttcttcaccgaacacgccggtgaattcgacttcatcctcaacaccattagcgcatccatcccagtcgacaagtacctgagccttctcaagccacacggtgtcatggctgttgtcggtctgccaccagagaagcagccactgagcttcggtgcgctcatcggcggcggaaaagtcctcaccggatccaacattggcggcatccctgaaacccaggaaatgctcgacttctgtgcaaaacacggcctcggtgcgatgatcgaaactgtcggcgtcaacgatgttgatgcagcctacgaccgtgttgttgccggcgacgttcagttccgcgttgtcattgatactgcttcgtttgctgaggttgaggcggtttag

SEQ ID NO: 67 - protein, 368 aa, Corynebacterium glutamicum
MSTVVPGIVALSKGAPVEKVNVVVPDPGANDVIVKIQACGVCHTDLAYRDGDISDEFPYLLGHEAAGIVEEVGESVTHVEVGDFVILNWRAVCGECRACKKGEPKYCFNTHNASKKMTLEDGTELSPALGIGAFLEKTLVHEGQCTKVNPEEDPAAAGLLGCGIMAGLGAAVNTGDIKRGESVAVFGLGGVGMAAIAGAKIAGASKIIAVDIDEKKLEWAKEFGATHTINSSGLGGEGDASEVVAKVRELTDGFGTDVSIDAVGIMPTWQQAFYSRDHAGRMVMVGVPNLTSRVDVPAIDFYGRGGSVRPAWYGDCLPERDFPTYVDLHLQGRFPLDKFVSERIGLDDVEEAFNTMKAGDVLRSVVEI

SEQ ID NO: 68 - DNA, 1107 bp, Corynebacterium glutamicum
atgagcactgtagtgcctggaattgtcgcactgtccaagggtgcaccggtagaaaaagtaaacgttgttgtccctgatccaggtgctaacgatgtcatcgtcaagattcaggcctgcggtgtgtgccacaccgacttggcctaccgcgatggcgatatttcagatgagttcccttacctcctcggccacgaggcagcaggcattgttgaggaggtaggcgagtccgtcacccacgttgaggtcggcgatttcgtcatcttgaactggcgtgcagtgtgcggcgagtgccgtgcatgtaagaagggcgagccaaagtactgctttaacacccacaacgcctctaagaagatgaccctggaagacggcaccgagctgtccccagcactgggtattggcgcgttcttggaaaagaccctggtccacgaaggccagtgcaccaaggttaaccctgaggaagatccagcagcagctggccttctgggttgtggcatcatggcaggccttggcgctgcggtgaacaccggtgatattaagcgtggcgagtccgtagcagtcttcggccttggtggcgtgggcatggcagctattgctggcgccaagattgctggcgcttccaagatcattgctgttgatatcgatgagaagaagctggagtgggcgaaggaattcggcgcaacccacaccattaattcctctggtcttggtggcgaaggtgatgcctctgaggtcgtggcaaaggttcgtgagctcaccgatggtttcggcaccgatgtctccatcgatgcggtaggcatcatgccgacctggcagcaggcgttttactcccgtgaccatgcaggccgcatggtgatggtgggcgttccaaacctgacgtctcgcgtagatgttcctgcgattgatttttacggtcgcggtggatccgtgcgccctgcatggtacggcgactgcctgcctgagcgtgatttcccaacttatgtggatctgcacctgcagggtcgtttcccactggataagtttgtttctgagcgtattggtcttgatgatgttgaagaggctttcaacaccatgaaggctggcgacgtgctgcgttctgtggtggagatctaa

SEQ ID NO: 69 - protein, 339 aa, Corynebacterium glutamicum
MPKYIAMQVSESGAPLAANLVQPAPLKSREVRVEIAASGVCHADIGTAAASGKHTVFPVTPGHEIAGTIAEIGENVSRWTVGDRVAIGWFGGNCGDCAFCRAGDPVHCRERKIPGVSYAGGWAQNIVVPAEALAAIPDGMDFYEAAPMGCAGVTTFNALRNLKLDPGAAVAVFGIGGLVRLAIQFAAKMGYRTITIARGLEREELARQLGANHYIDSNDLHPGQALFELGGADLILSTASTTEPLSELSTGLSIGGQLTIIGVDGGDITVSAAQLMMNRQIITGHLTGSANDTEQTMKFAHLHGVKPLIERMPLDQANEAIARISAGKPRFRIVLEPNS

SEQ ID NO: 70 - DNA, 1020 bp, Corynebacterium glutamicum
atgcccaaatacattgccatgcaggtatccgaatccggtgcaccgttagccgcgaatctcgtgcaacctgctccgttgaaatcgagggaagtccgcgtggaaatcgctgctagtggtgtgtgccatgcagatattggcacggcagcagcatcggggaagcacactgtttttcctgttacccctggtcatgagattgcaggaaccatcgcggaaattggtgaaaacgtatctcggtggacggttggtgatcgcgttgcaatcggttggtttggtggcaattgcggtgactgcgctttttgtcgtgcaggtgatcctgtgcattgcagagagcggaagattcctggcgtttcttatgcgggtggttgggcacagaatattgttgttccagcggaggctcttgctgcgattccagatggcatggacttttacgaggccgccccgatgggctgcgcaggtgtgacaacattcaatgcgttgcgaaacctgaagctggatcccggtgcggctgtcgcggtctttggaatcggcggtttagtgcgcctagctattcagtttgctgcgaaaatgggttatcgaaccatcaccatcgcccgcggtttagagcgtgaggagctagctaggcaacttggcgccaaccactacatcgatagcaatgatctgcaccctggccaggcgttatttgaacttggcggggctgacttgatcttgtctactgcgtccaccacggagcctctttcggagttgtctaccggtctttctattggcgggcagctaaccattatcggagttgatgggggagatatcaccgtttcggcagcccaattgatgatgaaccgtcagatcatcacaggtcacctcactggaagtgcgaatgacacggaacagactatgaaatttgctcatctccatggcgtgaaaccgcttattgaacggatgcctctcgatcaagccaacgaggctattgcacgtatttcagctggtaaaccacgtttccgtattgtcttggagccgaattcataa

SEQ ID NO: 71 - protein, 345 aa, Corynebacterium glutamicum
MTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIADDKLELARKHGAEFTVNARNEDSGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRF

SEQ ID NO: 72 - DNA, 1038 bp, Corynebacterium glutamicum
atgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagatcgatggtcgtgtggcgattcgtttctaa

SEQ ID NO: 73 - protein, 356 aa, Corynebacterium glutamicum
MSLQFDHETLGQRVLFGSGEAAQNLAAEISRLDAKNVMVVAGDFELPMARQVAADIDVKVWHSNVVMHVPIETAEEARSVAKENDIDVVVCVGGGSTTGLAKAIAMTTALPIIAVPTTYAGSEATNVWGLTEAARKTTGVDNKVLPVTVIYDSALTMSLPVEMSVASGLNGLAHCIDSLWGPKADPINAAMAAEGIRALSAGLPKIVADAQDVDGRDEALYGAYLAAVSFASAGSGLHHKICHVLGGTFNLPHAQTHATVLPYVLAFNAPYAPQAEQRAAAAFGSATALEGLQQLRAQVGAPQRLSDYGFTAAGIPEAVEIILEKVPANNPRTVTEENLTALLTTALNGDDPATLN

SEQ ID NO: 74 - DNA, 1071 bp, Corynebacterium glutamicum
atgtctttacagttcgatcatgaaaccctcggtcaacgagttctgttcggttcaggtgaggcggcgcaaaatctcgccgctgaaattagccgactcgatgccaaaaacgtcatggtggttgccggtgatttcgagcttcccatggcacggcaagtagcagcagatattgatgtcaaggtgtggcattcaaatgtcgtgatgcatgtgcccatcgaaacagcagaagaagcacgcagtgttgcgaaagaaaacgacattgatgttgtggtgtgtgtgggcggtggatccacaacaggtctagctaaagcgattgccatgaccaccgcattgccgatcattgcggtacccactacttatgcaggttctgaagcaacaaatgtgtggggattgaccgaagccgcgcgcaaaacaactggtgttgataacaaagtgctgccagtgacagttatctacgattcagcgttaaccatgtctttgccggtagaaatgtcggttgcttctggtctcaatggtttggctcactgcattgattctttgtggggaccgaaggcggatcccatcaatgcggctatggctgctgagggaattcgagcactttctgctggccttcccaagattgtggcagatgctcaggacgtagatggtcgcgatgaagcgctctacggtgcctacctggctgcggtgtcttttgcctctgctggctctggtctccaccacaagatctgccacgtgttgggtggaacttttaaccttccacacgcgcaaacccatgcaacagtactgccttatgttcttgccttcaacgcgccatatgcgccacaggcagaacaacgcgcagcggcagctttcggttctgcgacagcacttgaaggattgcaacagctgcgtgcccaagtgggagcaccacagcgactatccgattacggattcaccgcagcaggaatcccagaggcagtggaaatcatcttggagaaagtaccggcgaataatccacggacggtcacagaagaaaacctcactgcgctgcttaccacagcgctcaacggcgacgatccagcaactttgaattaa

SEQ ID NO: 75 - protein, 292 aa, Corynebacterium glutamicum
MQTLAAIVRATKQPFEITTIDLDAPRPDEVQIRVIAAGVRHTDAIVRDQIYPTFLPAVFGHEGAGVVVAVGSAVTSVKPDDKVVLGFNSCGQCLKCLGGKPAYCEKFYDRNFACTRDAGHTTLFTRATKEQAEAIIDTLDDVFYDADAGFLAYPATPPEASGVSVLVVAAGTSDLPQAKEALHTASYLGRSTSLIVDFGVAGIHRLLSYEEELRAAGVLIVAAGMDGALPGVVAGLVSAPVVALPTSVGYGAGAGGIAPLLTMLNACAPGVGVVNIDNGYGAGHLAAQIAAR

SEQ ID NO: 76 - DNA, 879 bp, Corynebacterium glutamicum
atgcaaacccttgctgctattgttcgtgccacgaagcaaccttttgagatcaccaccattgatctggatgcaccacgaccagatgaagttcaaatccgtgttattgctgccggagtgcgccacactgacgcaattgttcgtgatcagatttacccaacttttcttcccgcagttttcggccacgaaggcgccggagtagttgtcgccgtgggttctgcagtcacctcggtgaaaccagatgacaaggtagtgctgggattcaactcttgtggccagtgcttgaagtgtttgggcggtaagcctgcgtactgtgagaaattctatgaccgcaacttcgcatgcacccgcgatgccgggcacactactttgtttacccgtgcaacaaaagagcaggcagaggccatcatcgacacccttgatgatgttttctacgatgcggatgcgggtttcctggcatacccagcaactcccccagaggcttcgggagtaagcgtgttggttgtcgcggctggtacctctgatctcccccaagcaaaggaagcactacacactgcctcctacttggggcgctccacctcactgattgttgattttggagtggctggcatccaccgcctgctttcatacgaagaagaactccgcgctgcgggcgtgctcatcgttgccgctggaatggatggtgcgctacccggagttgtcgcaggcttagtgtccgcacctgtcgtcgcactgccaacctccgtgggatacggcgcaggtgctggaggaatcgcaccacttctgaccatgcttaacgcctgcgcgccgggagttggagtggtcaacattgataacggctatggagcaggacacctggctgcgcagattgcggcgaggtaa

SEQ ID NO: 77 - protein, 253 aa, Corynebacterium glutamicum
MLNAVGKAQNILLLGGTSEIGISIVSRFLKQGPSHVTLAARKDSPRVDAAVAEIKAAGAASVAVVDFDALDTESHPAAIDAAFENGDVDVAIVAFGILGDNEAQWRDQALAVEATTVNYTAGVSVGVLLGQKFEQQGHGTIVALSSVAGQRVRRSNFVYGSAKAGFDGFYTQLGEALRGSGANVLVVRPGQVRTKMSADGGEAPLTVNREDVADAVYDAVVNKKDIIFVHPLFQYVSFAFQFIPRAIFRKLPF

SEQ ID NO: 78 - DNA, 762 bp, Corynebacterium glutamicum
atgcttaacgcagtgggcaaagcccaaaacattctccttcttggtggaacctctgagatcggtatttccattgtctcccgcttcctcaagcagggtccatcccatgtgaccttggcagcgcgtaaagattccccacgcgtggacgcagcagtcgcagagatcaaagcagctggcgctgcttccgttgctgttgttgatttcgatgcgctcgacaccgaatcccaccctgcagccatcgacgcagcctttgaaaacggcgacgttgacgtagcaatcgtggctttcggcatcctcggcgacaacgaagcacagtggcgcgaccaagcactagcagtggaagcaaccaccgtgaactacaccgccggcgtttccgtaggtgtactgctgggccagaaatttgagcagcagggccacggcaccatcgtggcattgtcctctgtggcaggccagcgagtccgccgctccaactttgtctacggctccgccaaggcaggtttcgacggtttctacacccagctcggcgaagccctgcgtggatccggtgccaacgtattggtggttcgcccaggccaggtacgcaccaagatgtccgcagatggtggcgaagccccactgaccgtcaaccgcgaagacgtggcagatgctgtttatgatgcagtggtgaacaagaaggacatcatctttgtccacccactgttccagtacgtctcttttgcgttccaattcattccgcgagcaatcttccgcaagctgccgttctaa

SEQ ID NO: 79 - protein, 315 aa, Corynebacterium glutamicum
MEHGVTVIKGTEFDVFPLNLGGNTFGWTSNREQTFAVLDAFVAAGGNFVDTADSYSAWVEGNEGGESERELGAWIKERGADKLIIATKSGALEPVAGRSREATFKAVEGSLERLGVESIDIFYYHYDDEAVSIDEQVAIANDLIAQGKIKHLALSNYSAERLAEFFEKSVGTPAQPVALQPHYNLVSRKDYEENVQPLAEKHGVAVFPYFALAAGLLTGKYTSKEDISGKARAGQLDRYASDEAFAVVTELRAVADELGVAPTTVALAWLVAHGVTAPIASVSKVEQLKDLMAVKDVELSAEQLARLDKVSEPFA

SEQ ID NO: 80 - DNA, 948 bp, Corynebacterium glutamicum
atggagcacggcgtgaccgttattaaaggcactgaatttgatgttttcccactaaacctcggtggaaatacctttggctggacctcgaatagggaacagaccttcgcggttttggatgcattcgtggcagcgggaggaaactttgttgacaccgccgattcttattctgcatgggttgaaggcaatgagggtggcgagtcggagcgggagctcggcgcgtggattaaggaacgtggcgcagacaagctgatcattgctaccaagtctggtgcgttggagcctgttgctggtcgttcccgtgaggcaactttcaaggctgtcgagggttccctggagcgtttgggcgtggaatcgatcgatattttttactaccactacgacgatgaggcagtcagcattgatgagcaggttgctatcgctaatgatctgattgcacagggcaagattaagcacctcgcattgtctaactacagcgcggagcgtttagctgagttctttgagaagtctgtaggcactccagcgcagccggttgctctgcaaccgcactacaacctggtgtcgaggaaggattatgaggagaacgtgcagccactcgccgagaagcatggcgttgcagtcttcccttatttcgcgcttgccgcgggtcttttgaccggaaagtacacctccaaggaggatatttcgggtaaagcgcgtgcggggcagttggatcgttacgccagcgatgaggcgtttgccgtggtgacagagttgcgtgctgttgccgatgagttgggtgttgcgccaacgactgtggcgcttgcgtggttggttgcgcatggtgtgaccgcaccgattgcgtccgtgtccaaggtagagcagttgaaggatttgatggctgtgaaggatgtggagctgagcgctgagcagcttgcacgtttggataaggtttcggagcctttcgcttaa

SEQ ID NO: 81 - protein, 363 aa, Corynebacterium glutamicum
MNNSLAFNHDTLPQKVMFGYGKSSAFLKQEVERRGSAKVMVIAGEREMSIAHKVASEIEVAIWHDEVVMHVPIEVAERARAVATDNEIDLLVCVGGGSTIGLAKAIAMTTALPIVAIPTTYAGSEATNVWGLTEAARKTTGVDLKVLPETVIYDSELTMSLPVEMSVASGLNGLAHCIDSLWGPNADPINAVLAAEGIRALNQGLPKIVANPHSIEGRDEALYGAYLAAVSFASAGSGLHHKICHTLGGTFNLPHAQTHATVLPYVLAFNAGDAPEAERRAAAAFGTDTALEGLQRLRLSVNAPKRLSDYGFEASGIAEAVDVTLEKVPANNPRPVTRENLSRLLEAALNGEDPAVLSAVLSN

SEQ ID NO: 82 - DNA, 1092 bp, Corynebacterium glutamicum
gtgaacaactcactcgcattcaaccacgacaccctcccacagaaagtcatgtttggatatggcaagtccagtgcattcttaaagcaggaagttgaacgccgcggctcagccaaggtcatggtcattgcgggtgaacgagaaatgtcgatcgcccataaggtggcctcagaaattgaggtggcgatctggcacgacgaagttgtcatgcacgtgcccatcgaagtagccgaacgtgcgcgtgcagtggcaaccgacaatgagattgatctgctggtgtgtgttggcggcggatccaccataggtttggccaaagcaattgccatgaccactgccctgcccatcgtcgcgatccccaccacctacgcaggatcggaagcaaccaacgtgtggggtctgacggaagcagcgcgcaaaacaaccggtgttgatctgaaggtgctccccgaaacagtcatttacgattccgaactcaccatgtcgcttccagtggagatgtccgtggcatccggactcaacggcctggcgcactgcattgattctttgtggggacccaacgccgatcccatcaacgcagtgcttgcagccgaaggaatccgcgcactcaaccagggactgccgaaaattgttgcgaacccgcacagcatcgaaggacgcgacgaagccctctacggcgcctacctcgcagcagtatccttcgcctccgcaggctccggactacaccacaaaatctgccacaccttgggaggcaccttcaacctcccccacgcccaaacccacgcaaccgtgctgccgtatgttttggcattcaacgcaggcgacgcaccagaagctgaacgccgcgcagccgcagcctttggaactgacaccgcactagaaggcctgcaacgcctccgcttgtcagtcaacgcaccgaaacgactttccgactacggcttcgaggcttcaggaattgctgaggcagtggacgtcacgttggagaaagttcccgccaacaatcctcgcccagtgacccgggaaaacctcagcagattgctcgaagcagcactcaacggtgaggatccggcagttcttagcgcagtactcagtaactaa

SEQ ID NO: 83 - protein, 316 aa, Corynebacterium glutamicum
MKQRMVGSSGLRVSRLGLGTSTWGSGTELAEAGDIFKAFINSGGTLIDVSPNYTTGVAEEMLGTMLDAEVSRSAVVISSSAGVNPALPLGRRVDCSRRNLIAQLDVTLRALNTDYLDLWSVGYWDEGTPPHEVADTLDYAVRTGRVRYAGVRGYSGWQLAVTHAASNHAAASARPVVVAQNEYSLLERRAEQELLPATQHLGVGFFAGAPLGQGVLTAKYRSEIPHDSRAASTGRDAEVQSYLDNRGRIIVDALDTAAKGLGISPAVTATTWVRDRPGVTAVIVGARTHEQLSHLLKAESVTLPTPITQALDDVSL

SEQ ID NO: 84 - DNA, 951 bp, Corynebacterium glutamicum
gtgaaacagcgaatggtcggttcaagtggtttgcgggtatccaggctcggtttgggcacctcaacatggggctcgggcaccgagctggctgaggcaggcgatatctttaaggcgttcatcaattctggtggcacgcttatcgacgtctcccccaactacaccaccggcgtcgcggaagaaatgctcggcacgatgttggatgcggaagtctctcgttcggctgtcgtcatttcctccagcgcaggtgtcaaccccgctctgccgctcggccgacgtgtggattgctcccgccgcaatttgattgcccaattagatgtcaccctgcgggcattaaacactgactatttggatttgtggtctgtgggctattgggatgagggcaccccaccgcatgaggtggccgatactttggattacgccgtgcgcaccggccgagtccgatatgccggtgtccgaggatattccggttggcagttagcggtcacccacgctgcatccaatcatgcagcggcctccgcccgccccgtggtcgttgcacaaaatgaatacagcctgctggaacgccgcgcagaacaagaactcctccctgccacccaacacctaggtgtcggattctttgctggcgctccgctggggcaaggcgtgctgactgctaaataccgctccgaaattccccatgattccagagctgcatccacaggacgcgacgcagaagtccaaagctacctagataatcgaggccgcatcattgtcgatgctcttgatactgcagccaaaggattaggcattagccccgctgtcacagccaccacctgggtgcgtgatcgtcccggagtgacagctgtcatcgtgggcgctcgcacacatgaacagctgtcacatcttctcaaggcggaatcggtgactttgccaacaccaatcacacaagcccttgatgatgtctccctgtga

SEQ ID NO: 85 - protein, 329 aa, Bacillus subtilis
MKAVIHNGKAGLLGLSVQDVPSTKPGYGEVKVKLKSAGLNHRDLFLMKNKSEQDPHMILGSDGAGIIEEIGEGVKNVTVQTEVVIFPTLNWDLTENVPPVPEILGGPSDGTLAEYVIIPSQNAIKKPAYLSWEEAGVLPLSALTAYRALFTKGQLKKGEHLLIPGIGSGVATYALFMAKAIGATVSVTSRSEEKRKKALKLGADYAFDSYSNWDEQLQGKKIDVVLDSIGPALFSEYFRHVKPNGRIVSFGASSGDNLSFPVRSLFFPQVNVLGTSMGSGEEFQAMLAFIDKHKLRPVIDRIYPLEKACEAYKRMQEGRQFGNIGIVME

SEQ ID NO: 86 - DNA, 990 bp, Bacillus subtilis
atgaaagctgtaattcacaacggaaaagccggtcttctggggttatcagttcaggacgttccatcaacaaagcctggatacggagaggtaaaggttaaattaaaatctgcaggcctgaatcatcgtgacttgtttcttatgaaaaacaaatctgaacaagatcctcacatgatactgggttctgacggcgcgggtatcatcgaagagattggtgaaggcgtgaaaaatgttactgttcagacagaagtagtcattttcccgacattgaactgggatttgacagaaaatgttccacctgtacctgagattctgggaggtccttcggacggaacacttgctgaatatgtgatcattccttcacaaaatgcaatcaaaaaacctgcttatttatcttgggaagaagcgggcgttttacctttatccgctttaactgcatatcgggctctgtttacaaaggggcaattaaaaaaaggcgagcatctattgatacccggcatcggcagcggtgtagcaacctacgctttatttatggctaaggcgattggggcaacagtaagcgtgacctcccgcagtgaggagaaaagaaaaaaggcgctgaaattaggtgctgattacgcatttgacagctacagcaattgggatgagcagttgcagggaaaaaagatagatgttgttcttgacagcataggacctgccctcttttcggaatacttccgccatgtaaaaccaaatggccgtattgtcagctttggggcaagctcaggggataatctcagttttccggtgcgttctttattctttcctcaggtcaatgttttgggaacctcgatgggaagcggtgaggaatttcaagctatgctcgctttcattgacaaacataagctgcggcctgtaattgaccggatatatcctttagaaaaagcatgtgaagcatataaaagaatgcaggaaggcagacagtttggaaacatcgggatcgtaatggaataa

SEQ ID NO: 87 - protein, 390 aa, Bacillus subtilis
MENFTYYNPTKLIFGKGQLEQLRKEFKRYGKNVLLVYGGGSIKRNGLYDQVTGILKEEGAVVHELSGVEPNPRLATVEKGIGLCREHDIDFLLAVGGGSVIDCTKAIAAGVKYDGDAWDIFSKKVTAEDALPFGTVLTLAATGSEMNPDSVITNWETNEKFVWGSNVTHPRFSILDPENTFTVPENQTVYGMVDMMSHVFEQYFHNVENTPLQDRMCFAVLQTVIETAPKLLEDLENYELRETILYAGTIALNGTLQMGYFGDWASHTMEHAVSAVYDIPHAGGLAILFPNWMRYTLDTNVGRFKNLMLNMFDIDTEGKTDKEIALEGIDKLSAFWTSLGAPSRLADYNIGEEKLELIADIAAKEMEHGGFGNFQKLNKDDVLAILRASL

SEQ ID NO: 88 - DNA, 1173 bp, Bacillus subtilis
atggaaaatttcacttattataatccgacaaagctgatttttggaaaaggtcagcttgaacaattaagaaaagaattcaaacgatacggcaagaatgtactgcttgtttacgggggcggcagcattaaacgcaacggcctttatgatcaagtcacaggcattttaaaagaagagggcgctgttgttcatgagctgtcaggtgtagagccaaacccgcgtcttgcgacagtggaaaaaggcataggactttgcagagagcatgacattgattttctgcttgctgtcggcggaggcagtgtgattgactgtacaaaggcaatcgcagctggcgtcaaatatgacggtgacgcttgggatattttcagcaaaaaagtaacagcggaggatgcgctgccgtttggcactgttttaactcttgctgcaacagggtctgaaatgaaccctgattccgtgattacaaactgggaaacaaacgagaaatttgtatggggcagcaatgtcactcatccgcgtttctctattttagaccctgaaaatacgttcaccgttccagaaaatcaaacagtatacggcatggttgacatgatgagccacgtattcgaacaatacttccataatgttgaaaacacgccgcttcaggatagaatgtgctttgctgttttgcagacggtcatcgaaacagctcctaagcttcttgaagatctggaaaactacgaacttcgtgaaacgattttgtatgctggtactattgctttaaacggcacgctccaaatgggatacttcggtgactgggcttctcatacaatggaacacgctgtttcagctgtatatgatattccgcacgcgggcggtttggcaatactgttcccaaattggatgagatacacgcttgatacaaatgtaggccgttttaaaaaccttatgctcaacatgtttgacattgatactgaaggcaaaacagataaagaaattgcgcttgaaggaatcgataaactgtctgcgttctggacaagcctcggtgcaccttctcgtcttgctgattacaatattggagaagaaaagcttgagctgattgctgatatcgcagccaaggaaatggaacacggcggcttcggcaacttccaaaaactgaacaaagatgacgtgcttgccatccttcgcgcgtctctataa

SEQ ID NO: 89 - protein, 387 aa, Bacillus subtilis
MQNFTYWNPTKLIFGRGEVERLPEELKPYGKNVLLVYGGGSIKRSGLYDQVIEQLNKAGATVHELAGVEPNPRVSTVNKGVAICKEQNIDFLLAVGGGSVIDCTKAIAAGAKYDGDAWDIVTKKHQPKDALPFGTVLTLAATGSEMNSGSVITNWETKEKYGWGSPLVFPKFSILDPVNTFTVPKNHTIYGMVDMMSHVFEQYFHHVSNTPYQDRMCESLLRTVIETAPKLINDLENYELRETILYTGTIALNGMLSMGARGDWATHNIEHAVSAVYDIPHAGGLAILFPNWMRHTLSENPARMKQLAVRVFDVEEAGKTDEEIALEGIDKLSAFWTSLGAPNRLADYDINDEQLDTIADKAMANGTFGQFKSLNKEDVLSILKASL

SEQ ID NO: 90 - DNA, 1164 bp, Bacillus subtilis
atgcaaaactttacatactggaatccgaccaaattaattttcgggcgcggcgaagtggaaagacttccggaggaactcaaaccttacggaaaaaacgtattgcttgtgtacggaggcggcagcattaaacgcagcggcctgtatgatcaagtgattgaacagctgaataaagccggagcgaccgtgcatgaattagcaggtgtggaaccgaatcctcgtgtgtcgactgttaataaaggtgttgccatctgtaaagaacaaaacattgatttcttgctggctgtcggaggcggaagcgtaatcgactgtacaaaagcgattgccgcaggagcgaagtatgatggtgatgcgtgggatatcgttacgaaaaagcatcagccaaaagatgctttgccattcggaacggtattgactctcgctgcaactggttcagaaatgaactcaggatctgttattacaaactgggaaacaaaagaaaaatacggctggggcagcccgctcgtattccctaaattctcgattcttgatccggtgaatacattcaccgtacctaaaaaccacacgatctacgggatggttgacatgatgagccacgtattcgaacaatacttccatcatgtatcaaacacgccgtatcaggaccgcatgtgtgaatcacttttgcgtacagtcattgaaacagcgcctaagctgatcaatgatctcgaaaattacgaattgcgtgagacgatcctgtacacaggaacaatcgcattaaacggcatgctttctatgggggcaagaggggattgggctactcataatattgaacatgcagtatcagccgtttatgatattccgcatgccggcggactggcgattctgttcccgaattggatgagacacacattgtctgaaaaccctgcccgcatgaaacagcttgcagttcgcgtgtttgatgttgaagaagcaggtaaaacggatgaagaaattgcccttgaaggtatcgataagctgtccgcattctggacaagtcttggcgctccgaaccgtcttgctgattatgatattaatgatgagcagcttgacacaatcgctgacaaggcaatggctaacggtacattcggccaatttaaatcactcaacaaagaagatgtgctgtcaattttgaaagcatcactataa

SEQ ID NO: 91 - protein, 331 aa, Bacillus subtilis
MEYTSIADTGIEASRIGLGTWAIGGTMWGGTDEKTSIETIRAALDQGITLIDTAPAYGFGQSEEIVGKAIKEYGKRDQVILATKTALDWKNNQLFRHANRARIVEEVENSLKRLQTDYIDLYQVHWPDPLVPIEETAEVMKELYDAGKIRAIGVSNFSIEQMDTFRAVAPLHTIQPPYNLFEREMEESVLPYAKDNKITTLLYGSLCRGLLTGKMTEEYTFEGDDLRNHDPKFQKPRFKEYLSAVNQLDKLAKTRYGKSVIHLAVRWILDQPGADIALWGARKPGQLEALSEITGWTLNSEDQKDINTILENTISDPVGPEFMAPPTREEI

SEQ ID NO: 92 - DNA, 996 bp, Bacillus subtilis
atggaatataccagtatagcagatacaggaatagaagcctccagaatcggcctcggcacatgggccattggcggaacgatgtggggaggcactgacgaaaaaacatcgattgaaacaatccgcgccgctcttgatcaggggattacactgattgacaccgcaccggcttacggcttcgggcagtccgaggaaattgtcggaaaggcaatcaaagagtacggcaaaagagaccaggtgattctcgcaacgaaaacggctctggactggaagaacaaccagctgttccgccatgcgaacagagcgagaattgtagaggaagttgagaattctttgaagcggcttcaaacagactatattgatctttatcaggtgcattggcccgatccgcttgtgccaattgaagaaacggctgaagtcatgaaggaattatatgatgcgggaaaaatccgggcgattggcgtcagcaatttttcaattgagcaaatggatacatttcgcgccgtcgcacctctccatacgattcagcctccatataatctgtttgaaagagagatggaagagagtgtccttccttatgcgaaagataacaagataacaacattattatacggcagtttatgcagagggctgttaacaggcaaaatgactgaagaatatacatttgagggcgatgatctgcgtaatcacgatccaaaattccagaagccccgctttaaagagtatctttctgctgtgaatcaattggataagctggcgaagacacgttatggaaaatcagtgattcacttggctgtcagatggatcttagatcagccgggagcggatatcgctctttggggagcaagaaagcctgggcagcttgaggccctatctgagattacaggctggacgctgaacagtgaagatcagaaagatatcaatactatattggaaaatacgatatcagaccctgtcggaccggagtttatggccccgccgaccagagaggaaatataa

SEQ ID NO: 93 - protein, 306 aa, Bacillus subtilis
MRKRKLGTSDLDISEVGLGCMSLGTEKNKALSILDEAIELGINYLDTADLYDRGRNEEIVGDAIQNRRHDIILATKAGNRWDDGSEGWYWDPSKAYIKEAVKKSLTRLKTDYIDLYQLHGGTIEDNIDETIEAFEELKQEGVIRYYGISSIRPNVIKEYVKKSNIVSIMMQFSLFDRRPEEWLPLLEEHQISVVARGPVAKGLLTEKPLDQASESMKQNGYLSYSFEELTNARKAMEEVAPDLSMTEKSLQYLLAQPAVASVITGASKIEQLRENIQAANARRLTEEEIKALQSHTKQDIYKAHRS

SEQ ID NO: 94 - DNA, 921 bp, Bacillus subtilis
atgagaaagcgcaaattgggtacatctgatttagacattagcgaagtcggactcggctgtatgtctcttggaactgaaaaaaacaaagcattgtccattctggatgaagcgatcgagcttggcatcaactatttggacacagcggatttgtatgaccggggacgcaatgaagaaattgtcggtgatgcgatccaaaacagacgccatgatattattctggcaacaaaagcgggaaaccgttgggatgacggaagcgagggctggtattgggacccttcaaaagcttacataaaagaggcggtaaaaaagagccttacacgtctgaaaacagattatatcgacctttatcagctccacggcggcacgatagaggacaacattgatgaaacgattgaagcgtttgaggaattaaaacaagaaggtgtcatccgctactacggcatttcttccatccgcccgaatgtgattaaagaatatgtaaaaaaatcaaacatcgtcagcattatgatgcaattcagcctgtttgacagacgccctgaggaatggctcccgcttttagaggaacatcaaatcagcgtagtcgccagaggtcctgttgccaaagggctcttaactgaaaaaccgcttgatcaagcttcagaaagtatgaaacaaaacgggtacttgtcctattcattcgaggaactgacaaatgcccgtaaggcaatggaggaagtggctcccgatctttccatgacagaaaagtcgctgcagtatctgctagcacagccggctgtcgcttcagtgattacaggcgccagtaagattgagcagttacgggaaaatattcaggcagcaaatgcacggcgtttaaccgaagaggaaatcaaagcgctccaatctcatacgaaacaagacatttacaaagctcatcgctcatag

SEQ ID NO: 95 - protein, 310 aa, Bacillus subtilis
MDQTRTLGKTKLKVKRIGFGANAVGGHNLFPNLNDETGKDLVRTALDGGVNFIDTAFIYGLGRSEELIGEVVQERGVRNELIIATKGAHKEVDGSIELDNSREFLRSEVEKSLKRLKTDYIDLYYVHFPDGKTPLAEVAGTLKELKDEGKIKAIGASNLDYQQLQDFNADGYLEVFQAEYSLIQRDAEKELLPYCEKQGISFIPYFPLASGLLTGKFTQDTVFDDFRKDKPQFQGETFIHNLKKVDKLKAVAEEKQADTAHVALAWLLTRPAIDAIIPGAKRPEQLQDNLKTLNIELTEDEVNFISDIFK

SEQ ID NO: 96 - DNA, 933 bp, Bacillus subtilis
atggatcaaacacgtacactcggcaaaacgaagctgaaggtgaagcggatcggattcggcgcgaatgcggtcggcgggcataatctatttccaaatctaaatgatgaaacagggaaggatttagtgcgcacggcattggatggcggcgtcaattttatcgataccgcctttatatatggattggggcgatctgaagaattaatcggcgaagtcgtacaggaacgcggcgtgcggaatgagctcatcatcgccaccaaaggagctcataaagaagtggacggcagcattgaattagacaatagccgggagtttcttcgcagcgaggtggagaagagcctgaagcggctgaaaacagattacattgatttgtattatgttcactttccggatggaaaaacacctctcgctgaagtggcgggcactttgaaagagctgaaggatgaggggaaaatcaaagcgatcggcgcttcgaacctcgattatcagcaattgcaggattttaatgctgacggctatttggaggtcttccaggccgaatattctctcatacagcgtgatgccgagaaagagcttcttccatactgtgaaaaacaaggcatctcctttattccttactttccgcttgcgtccggactgctgacaggaaaattcacgcaagacacagtctttgatgatttcagaaaggataaacctcaatttcagggtgaaacgtttatccacaatctcaaaaaagtagataagctgaaagcagtagcggaggaaaaacaagcggatacggcacatgtcgccttggcgtggctgttaacgagaccggcgattgatgccattattccaggagctaaacgaccggagcaattacaggataacctgaaaaccttgaacattgaactgaccgaagatgaagtgaatttcatcagcgacattttcaaataa

SEQ ID NO: 97 - protein, 310 aa, Bacillus subtilis
MKKAKLGKSDLQVFPIGLGTNAVGGHNLYPNLNEETGKELVREAIRNGVTMLDTAYIYGIGRSEELIGEVLREFNREDVVIATKAAHRKQGNDFVFDNSPDFLKKSVDESLKRLNTDYIDLFYIHFPDEHTPKDEAVNALNEMKKAGKIRSIGVSNFSLEQLKEANKDGLVDVLQGEYNLLNREAEKTFFPYTKEHNISFIPYFPLVSGLLAGKYTEDTTFPEGDLRNEQEHFKGERFKENIRKVNKLAPIAEKHNVDIPHIVLAWYLARPEIDILIPGAKRADQLIDNIKTADVTLSQEDISFIDKLFA

SEQ ID NO: 98 - DNA, 933 bp, Bacillus subtilis
atgaaaaaagcgaagctcggaaaatcagacttgcaggtattccctatcggattaggaacaaatgctgtcggaggacataacctctacccgaacctaaatgaagaaaccggaaaagaattggtgcgcgaggcgatccgtaatggcgtgacaatgttagacaccgcttacatttacgggatcggccgttccgaagaattaattggtgaagtgctgcgtgaattcaaccgtgaagatgttgtcatcgcgacaaaagccgctcacagaaaacaaggcaatgactttgtctttgataattcaccagattttcttaaaaaatcagttgatgaaagcctgaagcgcttgaataccgattatattgatttgttctacattcacttccctgacgaacatacgcctaaggatgaagccgttaacgcgctgaatgagatgaagaaagccggaaaaatccgttccatcggtgtatccaacttctctttagagcaattgaaagaagcaaacaaagacggtttggtagatgtattgcaaggcgaatacaacctgttaaaccgtgaagcggaaaaaacattcttcccgtatacgaaggagcataatatttcatttatcccttacttccctctcgtatcaggtttattggcaggaaagtatacagaagatacaacgttcccagaaggcgacctgcgaaacgaacaggaacacttcaagggtgagcgtttcaaagaaaatatcagaaaggtcaacaagcttgcgccgattgccgaaaaacacaacgtggatatccctcacatcgtattggcctggtatttagcaagaccggaaattgatattttaatcccaggagcaaaacgtgccgatcagctgattgataacatcaaaacagctgacgtgacgctttctcaagaggatatttcatttattgataagctgttcgcataa

SEQ ID NO: 99 - protein, 326 aa, Bacillus subtilis
MEYTYLGRTGLRVSRLCLGTMNFGVDTDEKTAFRIMDEALDNGIQFFDTANIYGWGKNAGLTESIIGKWFAQGGQRREKVVLATKVYEPISDPNDGPNDMRGLSLYKIRRHLEGSLKRLQTDHIELYQMHHIDRRTPWDEIWEAFETQVRSGKVDYIGSSNFAGWHLVKAQAEAEKRRFMGLVTEQHKYSLLERTAEMEVLPAARDLGLGVVAWSPLAGGLLGGKALKSNAGTRTAKRADLIEKHRLQLEKFSDLCKELGEKEANVALAWVLANPVLTAPIIGPRTVEQLRDTIKAVEISLDKEILRMLNDIFPGPGGETPEAYAW

SEQ ID NO: 100 - DNA, 981 bp, Bacillus subtilis
gtggagtatacctatttagggagaacaggattgcgggtgagccgtttatgtttaggcacgatgaattttggagttgatacagacgaaaagactgcgttccgtatcatggatgaagcacttgataacggcattcaattttttgatactgccaatatttacggctggggcaaaaacgcaggattgacagagagcatcattggaaaatggtttgcacaaggaggacagcgccgcgagaaagttgttctggcgacaaaagtatatgaaccgatttctgatccgaatgacggaccaaatgatatgaggggcttgtctctatacaaaatcagacgtcatctggaaggatcactgaagcggcttcagacagatcatatcgaattgtaccaaatgcatcatatcgataggcggacaccgtgggatgagatatgggaagcttttgagactcaggttcgctccggcaaagtagactatattggatccagtaattttgcaggctggcatttagttaaagcgcaagctgaagctgaaaaacggcgattcatgggactcgtcactgaacagcataagtatagtttattagaacgaacagctgaaatggaagtgctgccggctgcacgggatcttggtttaggagtagtggcgtggagtccccttgcaggagggcttcttggcgggaaggcattgaaaagcaatgccggaactcgtacagcaaaaagagcagatttaattgaaaaacatcgtttgcaactcgagaaattttcagatttatgcaaagaactaggagaaaaagaagcaaatgtggctttggcatgggtgctggcaaatccagttttaactgcgccgatcatcggaccacgaacggttgagcagctgcgtgatacgataaaagccgttgaaatcagtctggataaggagattctccgcatgttaaatgatatctttcccggacctggaggagagacacctgaggcatacgcctggtga

SEQ ID NO: 101 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSSVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 102 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 103 - protein, 1178 aa, Mycobacterium abcessus
MTETISTAAVPTTDLEEQVKRRIEQVVSNDPQLAALLPEDSVTEAVNEPDLPLVEVIRRLLEGYGDRPALGQRAFEFVTGDDGATVIALKPEYTTVSYRELWERAEAIAAAWHEQGIRDGDFVAQLGFTSTDFASLDVAGLRLGTVSVPLQTGASLQQRNAILEETRPAVFAASIEYLDAAVDSVLATPSVRLLSVFDYHAEVDSQREALEAVRARLESAGRTIVVEALAEALARGRDLPAAPLPSADPDALRLLIYTSGSTGTPKGAMYPQWLVANLWQKKWLTDDVIPSIGVNFMPMSHLAGRLTLMGTLSGGGTAYYIASSDLSTFFEDIALIRPSEVLFVPRVVEMVFQRFQAELDRSLAPGESNSEIAERIKVRIREQDFGGRVLSAGSGSAPLSPEMTEFMESLLQVPLRDGYGSTEAGGVWRDGVLQRPPVTDYKLVDVPELGYFTTDSPHPRGELRLKSETMFPGYYKRPETTADVFDDEGYYKTGDVVAELGPDHLKYLDRVKNVLKLAQGEFVAVSKLEAAYTGSPLVRQIFVYGNSERSFLLAVVVPTPEVLERYADSPDALKPLIQDSLQQVAKDAELQSYEIPRDFIVETVPFTVESGLLSDARKLLRPKLKDHYGERLEALYAELAESQNERLRQLAREAATRPVLETVTDAAAALLGASSSDLAPDVRFIDLGGDSLSALSYSELLRDIFEVDVPVGVINSVANDLAAIARHIEAQRTGAATQPTFASVHGKDATVITAGELTLDKFLDESLLKAAKDVQPATADVKTVLVTGGNGWLGRWLVLDWLERLAPNGGKVYALIRGADAEAARARLDAVYESGDPKLSAHYRQLAQQSLEVIAGDFGDQDLGLSQEVWQKLAKDVDLIVHSGALVNHVLPYSQLFGPNVAGTAEIIKLAISERLKPVTYLSTVGIADQIPVTEFEEDSDVRVMSAERQINDGYANGYGNSKWAGEVLLREAHDLAGLPVRVFRSDMILAHSDYHGQLNVTDVFTRSIQSLLLTGVAPASFYELDADGNRQRAHYDGVPGDFTAASITAIGGVNVVDGYRSFDVFNPHHDGVSMDTFVDWLIDAGYKIARIDDYDQWLARFELALKGLPEQQRQQSVLPLLKMYEKPQPAIDGSALPTAEFSRAVHEAKVGDSGEIPHVTKELILKYASDIQLLGLV

SEQ ID NO: 104 - protein, 1193 aa, Mycobacterium smegmatis
MHQLTVTGMNICEVQRLFPRMTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 105 - DNA, 3537 bp, Mycobacterium abscessus
atgactgaaacgatctccacagcggctgtccccactacggatctcgaagagcaggtgaagcgacgcatcgagcaggtcgtgtccaacgatccgcagctggcggcgcttctcccggaagattcggtcaccgaggcggtcaacgagcccgatctaccgctggtcgaggtgatcaggcgactgctggagggctacggtgaccgcccggcactcggccagcgcgccttcgagttcgtcaccggggacgacggtgcgaccgtgatcgcgctgaagcccgaatacaccaccgtctcctaccgcgagttgtgggaacgtgccgaggctatcgctgccgcgtggcacgagcagggcatccgtgacggcgacttcgtcgctcagttgggtttcaccagcacggacttcgcgtcgctcgacgtcgcgggattgcgtctgggcaccgtctcggtgcccctgcagacgggcgcgtcgctgcagcagcgcaacgcgattctcgaagagacccggcccgcagtctttgccgcgagtatcgaataccttgatgccgccgtcgattcggtgcttgcgaccccctcggtgcgactcctctcggttttcgactatcacgcggaggtcgacagccagcgcgaagcgctggaggctgtgcgggcccggcttgagagtgccggccggacgatcgtcgtcgaggccctggcggaggctctcgcgcgggggcgggacctgcccgccgcgccgctgcccagtgcagatcccgatgccttgcgtctgctcatctacacctccggcagcaccggtacccccaagggcgccatgtatccgcaatggctggtcgccaacttgtggcagaagaagtggctcaccgacgatgtgattccgtccataggcgtgaacttcatgcccatgagccacctggcgggtcgcctcactctcatgggcaccctttccggtggcggaaccgcctactacatcgcttcgagcgatctttcgactttcttcgaggacatcgcgctcatccgcccctccgaagtgctcttcgtgccgcgtgtggtggagatggtgttccagcgttttcaggcagaattggaccggtcccttgccccgggtgagagcaactccgagatcgcggagcgaatcaaggtccgcatccgggaacaggacttcggcgggcgtgtgctcagtgctggctccgggtcggccccgttgtctcctgagatgacggagttcatggagtcgctgctgcaggtgccgttgcgcgacgggtatgggtccaccgaggccggtggtgtgtggcgtgacggagtcctgcagcgtccgcccgtcaccgactacaagctggttgacgttccggaactcggatacttcaccacagattcgccgcatccccgtggcgagctgcggttgaagtcggagacgatgttccccggctactacaagcgcccggagaccactgccgatgtcttcgatgacgaggggtactacaagaccggtgacgtggtcgccgagctcgggccggatcacctcaagtacctcgaccgcgtcaagaacgtcctcaagctcgcgcagggagagtttgtcgcggtgtcaaagctggaggccgcttacaccggcagcccgctggtccggcagatctttgtgtacgggaacagtgaacgctcgttcctgctggctgtcgtggtcccgacacccgaagtccttgagcggtacgcagattcgccagatgcgctcaagcccttgatccaggattcgctgcagcaggtcgccaaggacgcggagctgcaatcctatgagataccgcgcgacttcatcgttgagacggtgccgttcaccgtcgagtccggattgctatcggacgcgcgaaagctgctgcgccccaagctgaaggatcactacggagagaggctggaggcgctgtacgccgaactggcggaaagccagaatgagcggctgcgccagttggccagggaggcagccacgcgcccggtcctggagacggtgaccgatgcggccgccgcgctgctgggcgcatcgtcctcggatctggctcctgatgtgcgattcatcgacctcggtggcgactcactgtcggcgctgtcgtactccgagctgctgcgcgacatctttgaggtggacgttccggtgggcgtcatcaacagcgtcgccaacgaccttgccgcgatcgcccggcacatcgaggcgcagcggaccggcgccgctacgcagccgacctttgcgtcggtccacggcaaggacgcgacggtcatcaccgccggtgaactcaccctcgacaagttcttggacgagtcactgttgaaagcggccaaggacgttcagccggcaacggccgatgtcaagaccgttctagtgaccggcggcaacggctggttgggtcgttggctggtgctcgattggctggagcggttggcacccaatggtggcaaggtctacgccctcattcgtggcgccgatgccgaagcagcccgggcacggttggacgccgtgtacgaatcgggtgatcccaagctgtccgcgcattatcgtcagctggcgcaacagagtctggaagttatcgccggcgatttcggcgaccaggatctcggtctatcccaggaagtttggcagaagctggccaaggacgtggacctgatcgtgcactccggtgccttggtgaaccacgtgctgccgtacagccagttgttcggtccgaatgtggcgggtaccgccgagatcatcaagctggcaatttcggagcggctcaagccggtcacctacctgtcgacggtgggcatcgccgaccagattccggtgacggagttcgaggaagactccgatgttcgtgtgatgtcggccgagcgccagatcaatgacggctacgcgaacggatacggcaactcaaaatgggccggcgaggtgctgttgcgggaggctcatgacctagcggggctgccggtgcgtgtgttccgctccgacatgatcctggcgcacagtgactaccacggacagctcaacgtcaccgacgtgttcacccggagcatccagagtctgctgctcaccggtgttgcaccggccagcttctatgaattggatgccgacggcaatcggcagcgcgctcactatgacggtgtgcccggcgatttcaccgccgcatcgatcaccgccatcggcggtgtgaacgtggtagacggttaccgcagcttcgacgtgttcaacccgcaccatgacggtgtctcgatggataccttcgtcgactggctgatcgacgcaggctacaagatcgcgcggatcgacgattacgaccagtggctcgcccggttcgagctggccctcaagggattgcccgagcagcagcggcaacagtcggtgttgccacttctcaagatgtacgagaagccgcaaccggcgatcgacggaagtgcacttccgaccgcagaattcagtcgcgccgtgcacgaggcgaaggtcggagacagcggtgagataccgcacgtcaccaaggagctgatcctcaagtacgccagcgatattcagctgttgggcctggtgtag

SEQ ID NO: 106 - protein, 224 aa, Bacillus subtilis
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 107 - protein, 222 aa, Nocardia iowensis
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 108 - protein, 272 aa, Saccharomyces cerevisiae
MVKTTEVVSEVSKVAGVRPWAGIFVVEIQEDILADEFTFEALMRTLPLASQARILNKKSFHDRCSNLCSQLLQLFGCSIVTGLNFQELKFDKGSFGKPFLDNNRFLPFSMTIGEQYVAMFLVKCVSTDEYQDVGIDIASPCNYGGREELELFKEVFSEREFNGLLKASDPCTIFTYLWSLKESYTKFTGTGLNTDLSLIDFGAISFFPAEGASMCITLDEVPLIFHSQWFNNEIVTICMPKSISDKINTNRPKLYNISLSTLIDYFIENDGL

SEQ ID NO: 109 - DNA, 669 bp, Nocardia iowensis
atgatcgagacaattttgcctgctggtgtcgagtcggctgagctgctggagtatccggaggacctgaaggcgcatccggcggaggagcatctcatcgcgaagtcggtggagaagcggcgccgggacttcatcggggccaggcattgtgcccggctggcgctggctgagctcggcgagccgccggtggcgatcggcaaaggggagcggggtgcgccgatctggccgcgcggcgtcgtcggcagcctcacccattgcgacggatatcgggccgcggcggtggcgcacaagatgcgcttccgttcgatcggcatcgatgccgagccgcacgcgacgctgcccgaaggcgtgctggattcggtcagcctgccgccggagcgggagtggttgaagaccaccgattccgcactgcacctggaccgtttactgttctgcgccaaggaagccacctacaaggcgtggtggccgctgaccgcgcgctggctcggcttcgaggaagcgcacatcaccttcgagatcgaagacggctccgccgattccggcaacggcacctttcacagcgagctgctggtgccgggacagacgaatgacggtgggacgccgctgctttcgttcgacggccggtggctgatcgccgacgggttcatcctcaccgcgatcgcgtacgcctga

SEQ ID NO: 110 - protein, 468 aa, Escherichia coli
MIREPPEHILNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 111 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 112 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 113 - protein, 421 aa, Escherichia coli
MSNNEFHQRRLSATPRGVGVMCNFFAQSAENATLKDVEGNEYIDFAAGIAVLNTGHRHPDLVAAVEQQLQQFTHTAYQIVPYESYVTLAEKINALAPVSGQAKTAFFTTGAEAVENAVKIARAHTGRPGVIAFSGGFHGRTYMTMALTGKVAPYKIGFGPFPGSVYHVPYPSDLHGISTQDSLDAIERLFKSDIEAKQVAAIIFEPVQGEGGFNVAPKELVAAIRRLCDEHGIVMIADEVQSGFARTGKLFAMDHYADKPDLMTMAKSLAGGMPLSGVVGNANIMDAPAPGGLGGTYAGNPLAVAAAHAVLNIIDKESLCERANQLGQRLKNTLIDAKESVPAIAAVRGLGSMIAVEFNDPQTGEPSAAIAQKIQQRALAQGLLLLTCGAYGNVIRFLYPLTIPDAQFDAAMKILQDALSD

SEQ ID NO: 114 - protein, 464 aa, Ruegeria pomeroyi
MATITNHMPTAELQALDAAHHLHPFSANNALGEEGTRVITRARGVWLNDSEGEEILDAMAGLWCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGMAGMHAQSGLIPDVHHINQPNWWAEGGDMDPEEFGLARARELEEAILELGENRVAAFIAEPVQGAGGVIVAPDSYWPEIQRICDKYDILLIADEVICGFGRTGNWFGTQTMGIRPHIMTIAKGLSSGYAPIGGSIVCDEVAHVIGKDEFNHGYTYSGHPVAAAVALENLRILEEENILDHVRNVAAPYLKEKWEALTDHPLVGEAKIVGMMASIALTPNKASRAKFASEPGTIGYICRERCFANNLIMRHVGDRMIISPPLVITPAEIDEMFVRIRKSLDEAQAEIEKQGLMKSAA

SEQ ID NO: 115 - DNA, 1407 bp, Escherichia coli
atgatacgcgagcctccggagcatattttgaacaggttaccttcgagcgcatcggctttagcgtgcagcgcccacgccctgaatctcattgagaagcgaacgctggatcatgaggagatgaaagcacttaaccgagaggtgattgaatacttcaaagagcatgtcaatccggggtttttagagtatcgcaaatctgttaccgccggcggggattacggagccgtagagtggcaagcgggaagtttaaatacgcttgtcgacacccagggccaggagtttatcgactgcctgggaggttttggaattttcaacgtggggcaccgtaatccagttgtggtttccgccgtacagaatcaacttgcgaaacaaccgctgcacagccaggagctgctcgatccgttacgggcgatgttggcgaaaacccttgctgcgctaacgcccggtaaactgaaatacagcttcttctgtaatagcggcaccgagtccgttgaagcagcgctgaagctggcgaaagcttaccagtcaccgcgcggcaagtttacttttattgccaccagcggcgcgttccacggtaaatcacttggcgcgctgtcggccacggcgaaatcgaccttccgcaaaccgtttatgccgttactgccgggcttccgtcatgtgccgtttggcaatatcgaagccatgcgcacggctcttaacgagtgcaaaaaaaccggtgatgatgtggctgcggtgatcctcgaaccgattcagggtgaaggtggcgtaattctgccgccgccgggctatctcaccgccgtacgtaagctatgcgatgagttcggcgcactgatgatcctcgatgaagtacaaacgggcatggggcgcacgggcaagatgttcgcctgcgagcatgagaacgtacagccggatatcctctgccttgccaaagcgctcggcggcggcgtgatgccgattggcgcgaccatcgccactgaagaggtgttttcagttctgttcgacaacccattcctgcataccaccacctttggcggcaacccgctggcctgtgcggcggcgctggcgaccatcaatgtgttgctggagcagaacttaccggctcaggctgagcaaaaaggcgatatgttgctggacggtttccgtcaactggcgcgggaatatcccgatctggtacaggaagcgcgtggtaaagggatgttgatggcgattgagtttgttgataacgaaatcggctataactttgccagcgagatgttccgccagcgcgtactggtggccggaacgctcaataacgccaaaacgatccgcattgaaccgccactgacactgaccattgaacagtgtgaactggtgatcaaagcggcgcgtaaggcgctggcggccatgcgagtaagtgtcgaagaagcgtaa

SEQ ID NO: 116 - DNA, 36 bp, Artificial
  forward primer
ttgttgaataaatcgtctgtttgccgagaatacgcg

SEQ ID NO: 117 - DNA, 35 bp, Artificial
  reverse primer
tatcgcatgcagatctgcgctaatgggctccagga

SEQ ID NO: 118 - DNA, 35 bp, Artificial
  forward primer
ttgttgaataaatcgcgcgccagcgctggctgttt

SEQ ID NO: 119 - DNA, 35 bp, Artificial
  reverse primer
tatcgcatgcagatcccgccaatgccggaagagtt

SEQ ID NO: 120 - DNA, 35 bp, Artificial
  forward primer
ttgttgaataaatcgcgatcgtgatgccgctgtct

SEQ ID NO: 121 - DNA, 35 bp, Artificial
  reverse primer
tatcgcatgcagatcgacaacgtaggctttgttca

SEQ ID NO: 122 - DNA, 35 bp, Artificial
  forward primer
ttgttgaataaatcgcgccatctcgacactcttga

SEQ ID NO: 123 - DNA, 35 bp, Artificial
  reverse primer
tatcgcatgcagatcgcgaacatcgatgggttagc

SEQ ID NO: 124 - DNA, 35 bp, Artificial
  forward primer
ttgttgaataaatcgtggcatttgcccttcctgtt

SEQ ID NO: 125 - DNA, 35 bp, Artificial
  reverse primer
tatcgcatgcagatcgtcctgatcctgcaacggaa

SEQ ID NO: 126 - DNA, 35 bp, Artificial
  forward primer
ttgttgaataaatcgctcataacggtactgcaaac

SEQ ID NO: 127 - DNA, 35 bp, Artificial
  reverse primer
tatcgcatgcagatctcgcagcaggtaagatgatt

SEQ ID NO: 128 - DNA, 35 bp, Artificial
  forward primer
ttgttgaataaatcgatattcgtcctaacgaacag

SEQ ID NO: 129 - DNA, 35 bp, Artificial
  reverse primer
tatcgcatgcagatctttttgattttcaagtatgt

SEQ ID NO: 130 - DNA, 35 bp, Artificial
  primer
gatctgcatgcgatatctctagaacgcgtaagctt

SEQ ID NO: 131 - DNA, 29 bp, Artificial
  primer
tctcgagccgatttattcaacaaagccgc

SEQ ID NO: 132 - DNA, 25 bp, Artificial
  forward primer
actttcgttttcgggcatttcgtcc

SEQ ID NO: 133 - DNA, 24 bp, Artificial
  reverse primer
ccaatatgagggcagagaacgatc

SEQ ID NO: 134 - DNA, 20 bp, Artificial
  forward primer
atgcgctgatgtgataatgc

SEQ ID NO: 135 - DNA, 19 bp, Artificial
  reverse primer
ccttctccttgttgcttta

SEQ ID NO: 136 - DNA, 20 bp, Artificial
  forward primer
gaggcctttgctgcgactgc

SEQ ID NO: 137 - DNA, 21 bp, Artificial
  reverse primer
agttcctccttttcggatgat

SEQ ID NO: 138 - DNA, 18 bp, Artificial
  forward primer
atgccggatgcgacgctt

SEQ ID NO: 139 - DNA, 20 bp, Artificial
  reverse primer
tcattttgcatatagcccct

SEQ ID NO: 140 - DNA, 20 bp, Artificial
  forward primer
tgacctgggcagtaatggtg

SEQ ID NO: 141 - DNA, 25 bp, Artificial
  reverse primer
caggatctccgttgctttatgagtc

SEQ ID NO: 142 - DNA, 24 bp, Artificial
  forward primer
cgtggtgttgaaagccgattattg

SEQ ID NO: 143 - DNA, 23 bp, Artificial
  reverse primer
cataaacttccagttctccgccc

SEQ ID NO: 144 - DNA, 20 bp, Artificial
  forward primer
tcgcacactaacagactgaa

SEQ ID NO: 145 - DNA, 20 bp, Artificial
  reverse primer
tgtgtttactcctgattagc

SEQ ID NO: 146 - DNA, 25 bp, Artificial
  forward primer
tattctcaatccgtttcagcacgcg

SEQ ID NO: 147 - DNA, 21 bp, Artificial
  reverse primer
ttcgggatcaccaccaggccg

SEQ ID NO: 148 - DNA, 20 bp, Artificial
  forward primer
cggaaatggacgaacagtgg

SEQ ID NO: 149 - DNA, 22 bp, Artificial
  reverse primer
cgtcatcagcgtttaccagatt

SEQ ID NO: 150 - DNA, 20 bp, Artificial
  forward primer
gcataaacactgtccgcgtc

SEQ ID NO: 151 - DNA, 24 bp, Artificial
  reverse primer
gaaatcgagaaggcagaagcgaaa

SEQ ID NO: 152 - DNA, 20 bp, Artificial
  forward primer
ggtgcggtcaccattgttcg

SEQ ID NO: 153 - DNA, 20 bp, Artificial
  reverse primer
catatcgcacgccagcagtg

SEQ ID NO: 154 - DNA, 21 bp, Artificial
  forward primer
ggtgaggatggagagttcatg

SEQ ID NO: 155 - DNA, 21 bp, Artificial
  reverse primer
cagttcgatttgcgccaccag

SEQ ID NO: 156 - DNA, 20 bp, Artificial
  forward primer
tccgctagtgtgatttcagg

SEQ ID NO: 157 - DNA, 23 bp, Artificial
  reverse primer
gaaattattatgccgccaggcgt

SEQ ID NO: 158 - DNA, 20 bp, Artificial
  forward primer
ttatggtctgggcgacatgc

SEQ ID NO: 159 - DNA, 22 bp, Artificial
  reverse primer
gcatcatcctggtcatataccc

SEQ ID NO: 160 - DNA, 35 bp, Artificial
  primer
ataaggagatataccatgatacgcgagcctccgga

SEQ ID NO: 161 - DNA, 35 bp, Artificial
  primer
atgcggccgcaagctttacgcttcttcgacactta

SEQ ID NO: 162 - DNA, 35 bp, Artificial
  primer
taagaaggagatatacatatgactgaaacgatctc

SEQ ID NO: 163 - DNA, 35 bp, Artificial
  primer
gtggcagcagcctagctacaccaggcccaacagct

SEQ ID NO: 164 - DNA, 35 bp, Artificial
  primer
tttaaggagttcgatatgatcgagacaattttgcc

SEQ ID NO: 165 - DNA, 35 bp, Artificial
  primer
ggtggcagcagcctagtcaggcgtacgcgatcgcg

SEQ ID NO: 166 - DNA, 20 bp, Artificial
  primer
ctaggctgctgccaccgctg

SEQ ID NO: 167 - DNA, 40 bp, Artificial
  primer
atcgaactccttaaatttatctacaccaggcccaacagct

(19) United States
(12) Patent Application Publication
(10) Pub. No.: US 2022/0396800 A1
(43) Pub. Date: Dec. 15, 2022

(54) GENETICALLY MODIFIED MICROORGANISM AND METHOD FOR PRODUCING DIAMINE COMPOUND

(71) Applicant: Asahi Kasei Kabushiki Kaisha, Tokyo (JP)

(72) Inventors: Yutaro Yamada, Tokyo (JP); Hisanari Yoneda, Tokyo (JP)

(73) Assignee: Asahi Kasei Kabushiki Kaisha, Tokyo (JP)

(21) Appl. No.: 17/623,542

(22) PCT Filed: Jul. 22, 2020
(86) PCT No.: PCT/JP2020/028456

§ 371 Date: Dec. 28, 2021

(30) Foreign Application Priority Data
Jul. 22, 2019 (JP) 2019-134306

(51) Int. Cl. C12N 15/52; C12N 15/70; C12N 9/04; C12N 9/02; C12P 13/00

(52) U.S. Cl. CPC C12N 15/52; C12N 15/70; C12N 9/0006; C12N 9/0008; C12P 13/001; C12N 9/001; C12Y 101/01001; C12Y 102/99006; C12R 2001/19

(57) ABSTRACT

Provided are a microorganism that produces a diamine compound and a method of producing a diamine compound.

The genetically modified microorganism expresses an enzyme involved in synthesis of a diamine compound, in which the diamine compound is represented by Formula: H₂N—R—NH₂ (wherein, R is a chain or cyclic organic group comprised of one or more atoms selected from the group consisting of C, H, O, N, and S), and the genetically modified microorganism is modified to reduce an activity of an alcohol dehydrogenase compared to a non-reduced strain.

### TECHNICAL FIELD

The present invention relates to a genetically modified microorganism that produces a diamine compound and a method of producing a diamine compound.

### BACKGROUND ART

A diamine compound is widely used as a raw material of a polymer such as a polyamide resin. As the diamine compound that is industrially used, examples of representative compounds include hexamethylenediamine (1,6-diaminohexane), heptamethylenediamine (1,7-diaminoheptane), octamethylenediamine (1,8-diaminooctane), decamethylenediamine (1,10-diaminodecane), and dodecamethylenediamine (1,12-diaminododecane).

For example, hexamethylenediamine is synthesized by obtaining adiponitrile through hydrogenation of butadiene, electrolytic dimerization of acrylonitrile, or nitridation of adipic acid, and further performing hydrogenation using nickel as a catalyst. (Non Patent Literature 1) Hexamethylenediamine is industrially produced by this method, but adiponitrile is once synthesized, and then, a hydrogenation reaction is performed. In addition, as for decanediamine, octanediamine, dodecanediamine, or the like, similarly to the above adipic acid raw material, a method of obtaining a corresponding dinitrile and synthesizing the dinitrile by hydrogenation is known. (Patent Literatures 1 and 2)

In recent years, in a chemical product producing process, it is desired to switch from a fossil fuel-derived raw material which may be depleted and contributes to global warming to a renewable raw material such as a biomass-derived raw material. In order to solve this problem, a method of producing a diamine compound such as 1,3-diaminopropane (Non Patent Literature 2), 1,4-diaminobutane or 1,5-diaminopentane (Non Patent Literature 3), or 4-aminophenylethylamine (Non Patent Literature 4) using a microorganism metabolically modified by a genetic modification is disclosed.

Among them, a method of producing diamine obtained by combining an exogeneous enzyme, for example, a carboxylic acid decarboxylase or aminotransferase from a dicarboxylic acid or an aminocarboxylic acid, a dialdehyde, and the like in cells using a genetically modified microorganism has wide applicability of a compound as a substrate, and for example, hexamethylenediamine (Patent Literatures 3 and 4) and heptamethylenediamine (Patent Literature 5) have been reported.

Patent Literature 3 expects and exemplifies an enzyme gene whose yield is expected to improve due to deletion or disruption in a microbial host modified to have a hexamethylenediamine production pathway based on a metabolic simulation in in silico. However, neither a by-product derived from an intermediate in the hexamethylenediamine production pathway nor a method of suppressing the same is mentioned.

Patent Literature 4 describes a method of producing hexamethylenediamine by an enzymatic reaction pathway via 6-hydroxyhexanoic acid. However, neither production of a by-product derived from an intermediate in a hexamethylenediamine production pathway newly constructed by a genetic modification nor a suppression method thereof is mentioned.

Patent Literature 5 describes a method of producing heptamethylenediamine using an enzymatic reaction pathway via pimelic acid or the like. However, neither a by-product derived from an intermediate in a reaction pathway nor a suppression method thereof is mentioned.

Therefore, the prior arts do not disclose production of a by-product due to a conversion pathway to a diamine compound or a suppression method thereof, at all. In the production of the diamine compound by the genetically modified microorganism, a technique capable of more efficiently suppressing a by-product and efficiently producing a diamine compound is required.

### CITATION LIST

### Patent Literatures

Patent Literature 1: JP 57-70842 A
Patent Literature 2: JP 49-24446 B
Patent Literature 3: JP 2015-146810 A
Patent Literature 4: JP 2017-544854 A
Patent Literature 5: JP 2014-525741 A

### Non Patent Literatures

Non Patent Literature 1: Process Economics Program Report 31B (IHS market)
Non Patent Literature 2: Chae, T. et al., Metabolic engineering of Escherichia coli for the production of 1,3-diaminopropane, a three carbon diamine, Sci Rep. 2015 Aug. 11; 5:13040
Non Patent Literature 3: Tsuge, Y. et al., Engineering cell factories for producing building block chemicals for bio-polymer synthesis, Microb. Cell Fact., Vol., 15, 19 (2016)
Non Patent Literature 4: Masuo, S., et al, Bacterial fermentation platform for producing artificial aromatic amines, Scientific Reports volume 6, Article number: 25764 (2016)

### SUMMARY OF INVENTION

### Technical Problem

An object of the present invention is to provide a microorganism that produces a diamine compound and a method of producing a diamine compound.

### Solution to Problem

In conducting the studies, the present inventors found that an alcohol form derived from a diamine biosynthetic pathway intermediate is generated as a by-product by an endogenous alcohol dehydrogenase activity in a microorganism having a diamine compound production pathway. As a result of conducting intensive studies, the present inventors achieved the present invention based on the findings that production of an alcohol form that is a by-product can be suppressed and/or a production amount of a diamine compound can be increased by performing a modification so as to reduce an alcohol dehydrogenase activity of a host microorganism.

That is, the present invention provides the following:

[1] A genetically modified microorganism that expresses an enzyme involved in synthesis of a diamine compound, in which

the diamine compound is represented by Formula: H₂N—R—NH₂

(wherein, R is a chain or cyclic organic group comprised of one or more atoms selected from the group consisting of C, H, O, N, and S), and

the genetically modified microorganism is modified to reduce an activity of an alcohol dehydrogenase compared to a non-reduced strain;

[2] The genetically modified microorganism according to [1], in which the modification performed to reduce the activity of the alcohol dehydrogenase compared to the non-reduced strain is

a modification to suppress expression of a gene encoding an alcohol dehydrogenase or

a modification to suppress expression of a gene encoding an alcohol dehydrogenase and to suppress an activity of an alcohol dehydrogenase;

[3] The modified microorganism according to [1] or [2], in which the alcohol dehydrogenase is a protein encoded by

DNA consisting of a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100,

DNA consisting of a base sequence having 85% or more of sequence identity with a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100 and encoding a protein having an alcohol dehydrogenase activity,

DNA consisting of a base sequence encoding a protein consisting of an amino acid sequence obtained by deleting, substituting, inserting, and/or adding 1 to 10 amino acids with respect to an amino acid sequence of a protein encoded by a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100 and encoding a protein having an alcohol dehydrogenase activity, or

DNA consisting of a degenerate isomer of a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100;

[4] The modified microorganism according to any one of [1] to [3], in which the alcohol dehydrogenase is a protein containing an amino acid sequence having 80% or more of sequence identity with an amino acid sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, and 99 and having an alcohol dehydrogenase activity;

[5] The genetically modified microorganism according to any one of [1] to [4], in which the alcohol dehydrogenase is a protein encoded by at least one gene selected from the group consisting of yqhD, fucO, adhP, ybbO, eutG, ahr, yahK, adhE, ybdR, dkgA, yiaY, frmA, dkgB, yghA, ydjG, gldA, yohF, yeaE, ADH1, ADH2, ADH3, ADH4, ADH5, ADH6, ADH7, SFA1, AAD3, AAD4, AAD10, AAD14, AAD15, YPR1, NCg10324, NCg10313, NCg10219, NCg12709, NCg11112, NCg12382, NCg10186, NCg10099, NCg12952, NCg11459, yogA, bdhK, bdhJ, akrN, yqkF, yccK, iolS, and yrpG;

[6] The genetically modified microorganism according to any one of [1] to [5], in which the alcohol dehydrogenase is a protein encoded by at least one gene selected from the group consisting of yqhD, fucO, adhP, eutG, ybbO, ahr, and yahK;

[7] The genetically modified microorganism according to any one of [1] to [6], in which the alcohol dehydrogenase is a protein encoded by at least one gene selected from the group consisting of yqhD and adhP;

[8] The genetically modified microorganism according to [7], in which the alcohol dehydrogenase is a protein encoded by an adhP gene;

[9] The genetically modified microorganism according to any one of [1] to [6], in which the alcohol dehydrogenase is a protein encoded by at least one gene selected from the group consisting of yqhD, fucO, eutG, ybbO, ahr, and yahK;

[10] The genetically modified microorganism according to [9], in which the alcohol dehydrogenase is a protein encoded by at least one gene selected from the group consisting of eutG, ybbO, ahr, and yahK;

[11] The genetically modified microorganism according to any one of [1] to [6], in which the alcohol dehydrogenase is a protein encoded by two or more genes selected from the group consisting of yqhD, fucO, adhP, eutG, ybbO, ahr, and yahK;

[12] The genetically modified microorganism according to any one of [1] to [6], in which the alcohol dehydrogenase is a protein encoded by a gene of one combination selected from the group consisting of:
yqhD and fucO,
yqhD and adhP,
yqhD and eutG,
yqhD and ybbO,
yqhD and ahr,
yqhD and yahK,
yqhD, fucO, and adhP,
yqhD, fucO, adhP, and eutG,
yqhD, fucO, adhP, eutG, and ybbO,
yqhD, fucO, adhP, eutG, ybbO, and ahr,
   and
yqhD, fucO, adhP, eutG, ybbO, ahr, and yahK;

[13] The modified microorganism according to any one of [1] to [12], in which the modification performed to reduce the activity of the alcohol dehydrogenase compared to the non-reduced strain is performed by one or more selected from the group consisting of

a reduction in transcription amount and/or translation amount of a gene encoding the alcohol dehydrogenase in the microorganism and

a disruption of a gene encoding the alcohol dehydrogenase in the microorganism;

[14] The genetically modified microorganism according to any one of [1] to [13], in which the genetically modified microorganism belongs to a genus selected from the group consisting of the genus Escherichia, the genus Corynebacterium, the genus Bacillus, the genus Acinetobacter, the genus Burkholderia, the genus Pseudomonas, the genus Clostridium, the genus Saccharomyces, the genus Schizosaccharomyces, the genus Yarrowia, the genus Candida, the genus Pichia, and the genus Aspergillus;

[15] The genetically modified microorganism according to any one of [1] to [14], in which the genetically modified microorganism is Escherichia coli;

[16] The genetically modified microorganism according to any one of [1] to [15], in which the genetically modified microorganism expresses an aminotransferase as the enzyme involved in the synthesis of the diamine compound;

[17] The genetically modified microorganism according to any one of [1] to [16], in which the genetically modified microorganism expresses a carboxylic acid reductase as the enzyme involved in the synthesis of the diamine compound;

[18] The genetically modified microorganism according to [17], in which the carboxylic acid reductase has an activity of converting a carboxyl group of a carboxylic acid semialdehyde, a dicarboxylic acid, or an aminocarboxylic acid into an aldehyde;

[19] The genetically modified microorganism according to any one of [1] to [18], in which the genetically modified microorganism

has an ability of producing a dicarboxylic acid, a carboxylic acid semialdehyde, or an aminocarboxylic acid, and

further expresses an aminotransferase and a carboxylic acid reductase;

[20] The genetically modified microorganism according to any one of [1] to [19], in which the genetically modified microorganism

has an ability of producing adipic acid, adipic acid semialdehyde, or 6-aminohexanoic acid, and

further expresses an aminotransferase and a carboxylic acid reductase;

[21] The genetically modified microorganism according to any one of [11] to [20], in which the genetically modified microorganism is further modified to increase an activity of a phosphopantetheinyl transferase;

[22] The genetically modified microorganism according to any one of [16] to [21], in which a gene encoding the aminotransferase is ygjG;

[23] The genetically modified microorganism according to any one of [17] to [22], in which a gene encoding the carboxylic acid reductase is MaCar;

[24] The genetically modified microorganism according to any one of [21] to [23], in which a gene encoding the phosphopantetheinyl transferase is Npt;

[25] The modified microorganism according to any one of [1] to [24], in which the modified microorganism contains

a base sequence having 85% or more of sequence identity with a base sequence set forth in SEQ ID NO: 115 and encoding a protein having an aminotransferase activity or

a base sequence having 85% or more of sequence identity with a base sequence encoding an amino acid sequence set forth in any one of SEQ ID NOs: 110 to 114 and encoding a protein having an aminotransferase activity;

[26] The modified microorganism according to any one of [1] to [25], in which the modified microorganism contains

a base sequence having 85% or more of sequence identity with a base sequence set forth in SEQ ID NO: 105 and encoding a protein having a carboxylic acid reductase activity or

a base sequence having 85% or more of sequence identity with a base sequence encoding an amino acid sequence set forth in any one of SEQ ID NOs: 101 to 104 and encoding a protein having a carboxylic acid reductase activity;

[27] The modified microorganism according to any one of [21] to [26], in which the modified microorganism contains

a base sequence having 85% or more of sequence identity with a base sequence set forth in SEQ ID NO: 109 and encoding a protein having a phosphopantetheinyl transferase activity or

a base sequence having 80% or more of sequence identity with a base sequence encoding an amino acid sequence set forth in any one of SEQ ID NOs: 106 to 108 and encoding a protein having a phosphopantetheinyl transferase activity;

[28] The genetically modified microorganism according to any one of [1] to [27], in which the genetically modified microorganism expresses one or more enzymes selected from the group consisting of

acyl-(acyl carrier protein (ACP)) reductase (AAR),

an enzyme that produces an aldehyde from acyl-CoA, and

an enzyme that produces an aldehyde from acyl phosphate;

[29] A method of producing a diamine compound using the genetically modified microorganism according to any one of [1] to [28];

[30] A method of producing a diamine compound, the method including a culture step of culturing the genetically modified microorganism according to [1] to [28] in a medium containing a carbon source and a nitrogen source to obtain a culture medium containing bacterial cells;

[31] The method of producing a diamine compound according to [30], in which the medium further contains a precursor of a diamine compound, or

in the culture step, the precursor is added to the medium;

[32] The method of producing a diamine compound according to [30] or [31], further including a reaction step of bringing the culture medium and/or the bacterial cells into contact with an aqueous solution containing a precursor of a diamine compound to obtain a reaction solution containing a diamine compound; and

[33] The method of producing a diamine compound according to [31] or [32], in which the precursor is selected from the group consisting of a dicarboxylic acid, a carboxylic acid semialdehyde, an aminocarboxylic acid, an aminoaldehyde, a dialdehyde, acyl-ACP, acyl-CoA, and acyl phosphate.

### Advantageous Effects of Invention

According to the present invention, a diamine compound can be produced.

### BRIEF DESCRIPTION OF DRAWINGS

FIG. 1 schematically illustrates conversion of functional groups from various precursors into an amine as an example of a production pathway for a diamine compound in a genetically modified microorganism of the present invention.

FIG. 2 illustrates a concentration of 1,6-hexanediol in a culture supernatant after culturing an E. coli strain in which an ADH gene is disrupted in a medium containing 1,6-hexanediol for 48 hours.

FIG. 3 is a plasmid map of pDA56, in which “lacI” represents an lad gene, “T7 Promoter” represents a T7 promotor, “T7 Terminator” represents a T7 terminator, “ygjG” represents a ygjG gene derived from Escherichia coli, “MaCar” represents a carboxylic acid reductase gene derived from Mycobacterium abcessus, “Npt” represents a phosphopantetheinyl transferase gene derived from Nocardia iowensis, “CAT” represents a chloramphenicol acetyltransferase gene, and “P15Aori” represents a replication point.

### DESCRIPTION OF EMBODIMENTS

Hereinafter, embodiments of the present invention will be described in detail.

A genetically modified microorganism according to the present invention is a genetically modified microorganism that has a diamine compound production pathway and, at the same time, is modified to reduce an alcohol dehydrogenase activity. Here, the modification includes substitution, deletion, insertion, and/or addition. Hereinafter, the “genetically modified microorganism” is simply referred to as a “modified microorganism”.

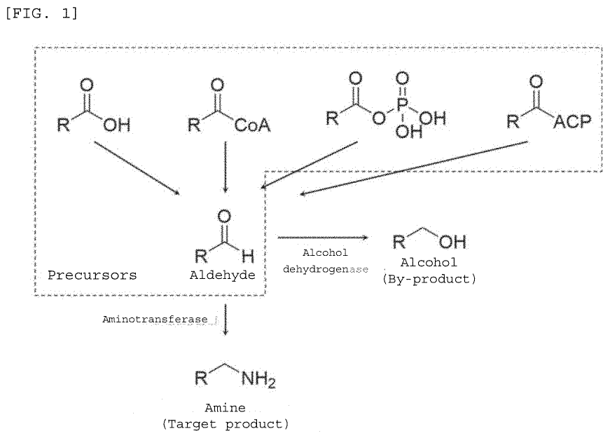

The modified microorganism expresses an enzyme involved in synthesis of a diamine compound or an enzyme group involved in synthesis of a diamine compound. Examples of the enzyme involved in synthesis of a diamine compound include a carboxylic acid reductase and an aminotransferase. As illustrated in FIG. 1, the carboxylic acid reductase has an activity of converting, for example, a carboxyl group of a carboxylic acid semialdehyde, a dicarboxylic acid, or an aminocarboxylic acid into an aldehyde. As illustrated in FIG. 1, the aminotransferase has an activity of converting an aldehyde into an amine. In the present invention, the microorganism “that expresses an enzyme involved in synthesis of a diamine compound or an enzyme group involved in synthesis of a diamine compound” means that a host microorganism itself may have an ability of expressing an enzyme or an enzyme group or a host microorganism may be modified to express an enzyme or an enzyme group.

The “diamine compound” (hereinafter, simply referred to as a “diamine”) in the present invention is represented by Formula: H₂N—R—NH₂. In the formula, R is a chain or cyclic divalent organic group comprised of one or more atoms selected from the group consisting of C, H, O, N, and S. A chain organic group includes a linear organic group and a branched organic group. A cyclic organic group includes an alicyclic organic group, a heterocyclic organic group, a polycyclic organic group, and an aromatic organic group.

Examples of the organic group constituting R include, but are not limited to, an aliphatic hydrocarbon group such as a methylene group, an ethylene group, a vinylene group, a trimethylene group, a propylene group, a propenylene group, a tetramethylene group, an isobutylene group, a pentamethylene group, a hexamethylene group, and an octamethylene group; an alicyclic hydrocarbon group such as a cyclobutylene group, a cyclopentylene group, a cyclohexylene group, a cycloheptylene group, a cyclooctylene group, a cyclohexenylene group, and a cyclohexadienylene group; an aromatic hydrocarbon group such as an o-phenylene group, an m-phenylene group, a p-phenylene group, a diphenylene group, a naphthylene group, a 1,2-phenylenedimethylene group, a 1,3-phenylenedimethylene group, a 1,4-phenylenedimethylene group, a 1,4-phenylylenediethylene group, a methylene diphenylene group, and an ethylene diphenylene group; an oxygen-containing characteristic group such as an oxy group and a carbonyl group; an ether group such as a methylenedioxy group and an ethylenedioxy group; an acyl group such as an oxalyl group, a malonyl group, a succinyl group, a glutalyl group, an adipoyl group, a speroyl group, an o-phthaloyl group, an m-phthaloyl group, and a p-phthaloyl group; a sulfur-containing characteristic group such as a thio group and a thiocarbonyl group; a nitrogen-containing characteristic group such as an imino group and an azo group; and a combination thereof.

In addition, one or more substituents may be included in R. Examples of the substituent that can be included in R include, but are not limited to, an amino group, a carboxy group, a cyano group, a nitro group, a hydroxy group, and a thiol group.

In one aspect, R is a chain or cyclic hydrocarbon, and a linear and branched, saturated and unsaturated hydrocarbons are included in the chain hydrocarbon. In a preferred aspect, R is a hydrocarbon group having 3 to 20 carbon atoms. More preferably, R is a linear saturated hydrocarbon group represented by Formula: CH₂(CH₂)ₙCH₂, wherein n is 1, 2, 3, 4, 5, 6, 7, 8, 9, or 10. Still more preferably, n is 2, 3, 4, 5, 6, 7, or 8, and particularly preferably, n is 4, 5, 6, 7, or 8.

Examples of a typical diamine compound include, but are not limited to, 1,3-diaminopropane (trimethylenediamine), 1,4-diaminobutane (tetramethylenediamine (putrescine)), 1,5-diaminopentane (pentamethylenediamine (cadaverine)), 1,6-diaminohexane (hexamethylenediamine), 1,7-diaminoheptane (heptamethylenediamine), 1,8-diaminooctane (octamethylenediamine), 1,9-diaminononane (nonamethylenediamine), 1,10-diaminodecane (decamethylenediamine), 1,11-diaminoundecane (undecamethylenediamine), 1,12-diaminododecane (dodecamethylenediamine), 3-aminobenzylamine, 4-aminobenzylamine, 2-methylpentamethylenediamine, 2-methylpentamethylenediamine, 2,2,4-trimethylhexamethylenediamine, 2,4,4-trimethylhexamethylenediamine, 1,3-bis(aminomethyl)cyclohexane, 1,4-bis(aminomethyl)cyclohexane, m-xylene diamine, p-xylene diamine, 1-amino-3-aminomethyl-3,5,5-trimethylcyclohexane, bis(4-aminocyclohexyl)methane, bis(4-amino-3-methylcyclohexyl)methane, 1,4-bis(aminopropyl)piperazine, 1,3-diaminocyclohexane, 1,4-diaminocyclohexane, 1,3-phenylenediamine, 1,4-phenylenediamine, N-(3-aminopropyl)1,4-butanediamine (spermidine), 3,3′-diaminodipropylamine, N,N-bis(3-aminopropyl)methylamine, N,N′-bis(3-aminopropyl)ethylenediamine, N,N′-bis(2-aminoethyl)-1,3-propanediamine, N,N′-bis(3-aminopropyl)-1,4-butanediamine (spermine), 2,2′-dithiobis(ethylamine), and dipropylenetriamine. It is appreciated by those skilled in the art that the diamine has a neutral or ionized form including an arbitrary salt form and that the form depends on pH.

The “dicarboxylic acid” in the present specification refers to a compound having a structure represented by a chemical formula HOOC—R—COOH (wherein, R is as described above). An aliphatic dicarboxylic acid and an aromatic carboxylic acid are included in the dicarboxylic acid. Examples of a typical dicarboxylic acid include, but are not limited to, oxalic acid, malonic acid, succinic acid, fumaric acid, itaconic acid, glutaric acid, adipic acid, muconic acid, pimelic acid, suberic acid, azelaic acid, sebacic acid, dodecanedioic acid, malic acid, 2,5-furandicarboxylic acid, phthalic acid, isophthalic acid, terephthalic acid, maleic acid, tartaric acid, and muconic acid. It is appreciated by those skilled in the art that the dicarboxylic acid has a neutral or ionized form including an arbitrary salt form and the form depends on pH.

The “carboxylic acid semialdehyde” in the present specification refers to a compound having a structure represented by a chemical formula HOOC—R—CHO (wherein, R is as described above). Examples of a typical carboxylic acid semialdehyde include, but are not limited to, succinic acid semialdehyde, glutaric acid semialdehyde, adipic acid semialdehyde, pimelic acid semialdehyde, suberic acid semialdehyde, azelaic acid semialdehyde, and sebacic acid semialdehyde. It is appreciated by those skilled in the art that the carboxylic acid semialdehyde has a neutral or ionized form including an arbitrary salt form and that the form depends on pH.

The “aminocarboxylic acid” in the present specification refers to a compound having a structure represented by a chemical formula H₂N—R—COOH (wherein, R is as described above). Examples of a typical aminocarboxylic acid include, but are not limited to, glycine, β-alanine, 4-aminobutanoic acid, 5-aminopentanoic acid, 6-aminohexanoic acid, 7-aminoheptanoic acid, 8-aminooctanoic acid, 9-aminononanoic acid, 10-aminodecanoic acid, and 12-aminododecanoic acid. It is appreciated by those skilled in the art that the aminocarboxylic acid has a neutral or ionized form including an arbitrary salt form and that the form depends on pH.

In the present specification, the term “endogenous” or “endogenous property” is used to mean that the host microorganism without a genetic modification has the gene referred to or the protein encoded by the same (typically, an enzyme), regardless of whether the gene or the protein is functionally expressed to the extent that a predominant biochemical reaction can proceed in the host cell.

In the present specification, the term “exogeneous” or “exogeneous property” is used to mean that a gene or a nucleic acid sequence according to the present invention is introduced into a host in a case where a pre-genetically modified host microorganism does not have a gene to be introduced according to the present invention, in a case where the pre-genetically modified host microorganism does not substantially express an enzyme by the gene, or in a case where the pre-genetically modified host microorganism does not express an endogenous enzyme activity corresponding to after the genetic modification although an amino acid sequence of the enzyme is encoded by a different gene. The term “exogeneous property” and “external property” are used interchangeably in the present specification.

FIG. 1 illustrates an example of conversion of functional groups of synthesis pathways for a diamine compound in the present invention. A diamine is synthesized using an aldehyde-inducible compound and/or an aldehyde as a precursor. The aldehyde is converted into an amine by an aminotransferase. The genetically modified microorganism according to the present invention is modified to reduce an activity of an alcohol dehydrogenase, thereby suppressing the conversion of the aldehyde that is an intermediate in the pathway into an alcohol. Here, the alcohol dehydrogenase includes one or more proteins having an alcohol dehydrogenase activity.

The host microorganism used in the present invention is not particularly limited, and may be either a prokaryote or a eukaryote. Any one of a microorganism isolated and preserved in advance, a microorganism newly isolated from nature, a microorganism subjected to a genetic modification, and a microorganism modified so that the compound can be metabolized can be arbitrarily selected. Examples thereof include, but are not limited to, a bacterium, for example, the genus Escherichia such as Escherichia coli (E. coli), the genus Pseudomonas such as Pseudomonas putida, the genus Bacillus such as Bacillus subtilis, the genus Corynebacterium such as Corynebacterium glutamicum, the genus Clostridium such as Clostridium acetobutylicum, the genus Acinetobacter, and the genus Burkholderia; a yeast, for example, the genus Saccharomyces such as Saccharomyces cerevisiae, the genus Schizosaccharomyces such as Schizosaccharomyces pombe, the genus Pichia such as Pichia pastoris, and the genus Yarrowia such as Yarrowia lipolytica; and a filamentous fungus, for example, the genus Aspergillus such as Aspergillus oryzae. In the present invention, E. coli is preferably used as a host microorganism.

The genetically modified microorganism according to the present invention is further modified to reduce an endogenous alcohol dehydrogenase (ADH) activity compared to a non-reduced strain. The present inventors found that in a host microorganism having a diamine compound production pathway, an alcohol form derived from a diamine biosynthetic pathway intermediate is produced as a by-product due to an endogenous alcohol dehydrogenase activity. After conducting further intensive studies, the present inventors found that production of an alcohol form that is a by-product can be suppressed and/or a production amount of a diamine compound is increased by modifying a host microorganism to reduce an activity of an alcohol dehydrogenase compared to a non-reduced strain, resulting in efficient production of a diamine compound.

The alcohol dehydrogenase is an enzyme having an activity of reducing an aldehyde and a ketone to be converted into an alcohol in the presence of an electron donor. Here, the alcohol dehydrogenase also includes a protein containing an amino acid sequence in which one or more amino acids are deleted, substituted, inserted, and/or added in an amino acid sequence of the enzyme, the protein being functionally equivalent to the enzyme. Here, the “functionally equivalent protein” is a protein having the equivalent activity to the activity of the enzyme. For example, the “functionally equivalent protein” includes a protein having 80%, 85%, 90%, 95%, 97%, 98%, or 99% or more of sequence identity with the amino acid sequence of the enzyme. Specifically, the term “alcohol dehydrogenase” includes a protein containing an amino acid sequence having 80%, 85%, 90%, 95%, 97%, 98%, or 99% or more of sequence identity with the amino acid sequence set forth in the following specific sequence number, and having an alcohol dehydrogenase activity.

The gene encoding an alcohol dehydrogenase contains:

DNA consisting of a base sequence set forth in the following specific sequence number,

DNA hybridizing to DNA containing a base sequence complementary to a base sequence set forth in the following specific sequence number under a stringent condition and encoding a protein having an alcohol dehydrogenase activity,

DNA consisting of a base sequence having 85%, 90%, 95%, 97%, 98%, or 99% or more of sequence identity with a base sequence set forth in the following specific sequence number and encoding a protein having an alcohol dehydrogenase activity,

DNA consisting of a base sequence encoding a protein consisting of an amino acid sequence in which one or more (for example, 1 to 10, preferably 1 to 7, more preferably 1 to 5, still more preferably 1 to 3, and still further preferably 1 or 2) of amino acids are deleted, substituted, inserted, and/or added in an amino acid sequence of a protein encoded by a base sequence set forth in the following sequence number, and encoding a protein having an alcohol dehydrogenase activity,

and

DNA consisting of a degenerate isomer of a base sequence set forth in the following specific sequence number.

The “stringent condition” is, for example, a condition of about “1×SSC, 0.1% SDS, 60° C.”, a more severe condition of about “0.1×SSC, 0.1% SDS, 60° C.”, and a still more severe condition of about “0.1×SSC, 0.1% SDS, 68° C.”.

In a preferred aspect of the present invention, an enzyme represented by EC 1.1.1.m (wherein, m is an integer of 1 or more) is included in the alcohol dehydrogenase. Examples of the alcohol dehydrogenase include, but are not limited to, enzymes classified as EC 1.1.1.1, EC 1.1.1.2, and EC 1.1.1.71.

In the case of E. coli, examples of the alcohol dehydrogenase include proteins encoded by yqhD, fucO, adhP, ybbO, eutG, ahr, yahK, adhE, ybdR, dkgA, yiaY, frmA, dkgB, yghA, ydjG, gldA, yohF, and yeaE genes.

In the case of Saccharomyces cerevisiae, examples of the alcohol dehydrogenase include proteins encoded by ADH1, ADH2, ADH3, ADH4, ADH5, ADH6, ADH7, SFA1, AAD3, AAD4, AAD10, AAD14, AAD15, and YPR1 genes. In the case of Corynebacterium glutamicum, examples of the alcohol dehydrogenase include proteins encoded by NCg10324, NCg10313, NCg10219, NCg12709, NCg11112, NCg12382, NCg10186, NCg10099, NCg12952, and NCg11459 genes. In the case of Bacillus subtilis, examples of the alcohol dehydrogenase include proteins encoded by a yogA, bdhK, bdhJ, akrN, yqkF, yccK, iolS, and yrpG genes. However, the alcohol dehydrogenase is not limited thereto as long as it has an alcohol dehydrogenase activity.

The alcohol dehydrogenase is a protein encoded by, for example, at least one gene selected from the group consisting of yqhD, fucO, adhP, eutG, ybbO, ahr, and yahK. Modification of at least one gene selected from the group consisting of the above genes such that the activity of the alcohol dehydrogenase is reduced compared to a non-reduced strain can suppress production of an alcohol form that is a by-product and/or increase a production amount of a diamine compound, resulting in efficient production of a diamine compound.

The alcohol dehydrogenase is preferably encoded by at least one gene selected from the group consisting of yqhD, fucO, adhP, ybbO, eutG, ahr, and yahK genes, more preferably encoded by at least one gene selected from the group consisting of yqhD, ahr, and yahK genes, and still more preferably encoded by at least one gene selected from the group consisting of ahr and yahK genes.

The alcohol dehydrogenase is preferably encoded by at least one gene selected from the group consisting of yqhD and adhP genes, and more preferably encoded by an adhP gene. In the production of the diamine compound, by modifying the microorganism to reduce the activity of at least one of these genes, the production amount of the diamine compound can be increased in the genetically modified microorganism.

The alcohol dehydrogenase is preferably encoded by at least one gene selected from the group consisting of yqhD, fucO, eutG, ybbO, ahr, and yahK, and more preferably encoded by at least one gene selected from the group consisting of eutG, ybbO, ahr, and yahK. In the production of the diamine compound, by modifying the microorganism to reduce the activity of at least one of these genes, the production of an alcohol form that is a by-product can be suppressed in the genetically modified microorganism.

The alcohol dehydrogenase is preferably encoded by two or more genes selected from the group consisting of yqhD, fucO, adhP, eutG, ybbO, ahr, and yahK, and more preferably encoded by a yqhD gene and one or more genes selected from the group consisting of fucO, adhP, eutG, ybbO, ahr, and yahK. In the production of the diamine compound, by modifying the microorganism to reduce the activities of two or more of these genes, the production amount of the diamine compound can be significantly increased, and production of an alcohol form that is a by-product can also be suppressed in the genetically modified microorganism.

The alcohol dehydrogenase is preferably encoded by a gene of one combination selected from the group consisting of
yqhD and fucO,
yqhD and adhP,
yqhD and eutG,
yqhD and ybbO,
yqhD and ahr,
yqhD and yahK,
yqhD, fucO, and adhP,
yqhD, fucO, adhP, and eutG,
yqhD, fucO, adhP, eutG, and ybbO,
yqhD, fucO, adhP, eutG, ybbO, and ahr,
and
yqhD, fucO, adhP, eutG, ybbO, ahr, and yahK. As described above, in the production of the diamine compound, the production amount of the diamine compound can be significantly increased, and production of an alcohol form that is a by-product can also be significantly suppressed, by modifying the microorganism to reduce the activities of two or more genes.

Amino acid sequences of typical proteins encoded by the aforementioned alcohol dehydrogenase genes and base sequences of coding regions are shown in Tables 1-1 to 1-50. In the first row of each table, genes, protein names, accession numbers, and origins are shown.

TABLE 1-1
yqhD/ACT44688.1 Escherichia coli BL21(DE3)

| | |
|---|---|
| Amino acid | MNNFNLHTPTRILFGKGAIAGLREQIPHDARVLITYGGGSVKKTGVLDQVLDALKGM |
| sequence | DVLEFGGIEPNPAYETLMNAVKLVREQKVTFLLAVGGGSVLDGTKFIAAAANYPENID |
| | PWHILQTGGKEIKSAIPMGCVLTLPATGSESNAGAVISRKTTGDKQAFHSAHVQPVFA |
| | VLDPVYTYTLPPRQVANGVVDAFVHTVEQYVTKPVDAKIQDRFAEGILLTLIEDGPK |
| | ALKEPENYDVRANVMWAATQALNGLIGAGVPQDWATHMLGHELTAMHGLDHAQT |
| | LAIVLPALWNEKRDTKRAKLLQYAERVWNITEGSDDERIDAAIAATRNFFEQLGVPTH |
| | LSDYGLDGSSIPALLKKLEEHGMTQLGENHDITLDVSRRIYEAAR |
| | (SEQ ID NO: 1) |
| Base | ATGAACAACTTTAATCTGCACACCCCAACCCGCATTCTGTTTGGTAAAGGCGCAAT |
| sequence | CGCTGGTTTACGCGAACAAATTCCTCACGATGCTCGCGTATTGATTACCTACGGCG |
| | GCGGCAGCGTGAAAAAAACCGGCGTTCTCGATCAAGTTCTGGATGCCCTGAAAGG |
| | CATGGACGTGCTGGAATTTGGCGGTATTGAGCCAAACCCGGCTTATGAAACGCTG |
| | ATGAACGCCGTGAAACTGGTTCGCGAACAGAAAGTGACTTTCCTGCTGGCGGTTG |
| | GCGGCGGTTCTGTACTGGACGGCACCAAATTTATCGCCGCAGCGGCTAACTATCCG |
| | GAAAATATCGATCCGTGGCACATTCTGCAAACGGGCGGTAAAGAGATTAAAAGCG |
| | CCATCCCGATGGGCTGTGTGCTGACGCTGCCAGCAACCGGTTCAGAATCCAACGC |
| | AGGCGCGGTGATCTCCCGTAAAACCACAGGCGACAAGCAGGCGTTCCATTCTGCC |
| | CATGTTCAGCCGGTATTTGCCGTGCTCGATCCGGTTTATACCTACACCCTGCCGCCG |
| | CGTCAGGTGGCTAACGGCGTAGTGGACGCCTTTGTACACACCGTGGAACAGTATG |
| | TTACCAAACCGGTTGATGCCAAAATTCAGGACCGTTTCGCAGAAGGCATTTTGCTG |
| | ACGCTAATCGAAGATGGTCCGAAAGCCCTGAAAGAGCCAGAAAACTACGATGTGC |
| | GCGCCAACGTCATGTGGGCGGCGACTCAGGCGCTGAACGGTTTGATTGGCGCTGG |
| | CGTACCGCAGGACTGGGCAACGCATATGCTGGGCCACGAACTGACTGCGATGCAC |
| | GGTCTGGATCACGCGCAAACACTGGCTATCGTCCTGCCTGCACTGTGGAATGAAA |
| | AACGCGATACCAAGCGCGCTAAGCTGCTGCAATATGCTGAACGCGTCTGGAACAT |
| | CACTGAAGGTTCCGATGATGAGCGTATTGACGCCGCGATTGCCGCAACCCGCAATT |
| | TCTTTGAGCAATTAGGCGTGCCGACCCACCTCTCCGACTACGGTCTGGACGGCAG |
| | CTCCATCCCGGCTTTGCTGAAAAAACTGGAAGAGCACGGCATGACCCAACTGGGC |
| | GAAAATCATGACATTACGTTGGATGTCAGCCGCCGTATATACGAAGCCGCCCGCTA |
| | A |
| | (SEQ ID NO: 2) |

TABLE 1-2
fucO/ACT44461.1/Escherichia coli BL21(DE3)

| | |
|---|---|
| Amino acid | MMANRMILNETAWFGRGAVGALTDEVKRRGYQKALIVTDKTLVQCGVVAKVTDKM |
| sequence | DAAGLAWAIYDGVVPNPTITVVKEGLGVFQNSGADYLIAIGGGSPQDTCKAIGIISNN |
| | PEFADVRSLEGLSPTNKPSVPILAIPTTAGTAAEVTINYVITDEEKRRKFVCVDPHDIPQ |
| | VAFIDADMMDGMPPALKAATGVDALTHAIEGYITRGAWALTDALHIKAIEIIAGALRG |
| | SVAGDKDAGEEIALGQYVAGMGFSNVGLGLVHGMAHPLGAFYNTPHGVANAILLPH |
| | VMRYNADFTGEKYRDIARVMGVKVEGMSLEEARNAAVEAVFALNRDVGIPPHLRDV |
| | GVRKEDIPALAQAALNDVCTGGNPREATLEDIVELYHTAW |
| | (SEQ ID NO: 3) |
| Base | ATGATGGCTAACAGAATGATTCTGAACGAAACGGCATGGTTTGGTCGGGGTGCTG |
| sequence | TTGGGGCTTTAACCGATGAGGTGAAACGCCGTGGTTATCAGAAGGCGCTGATCGT |
| | CACCGATAAAACGCTGGTGCAATGCGGCGTGGTGGCGAAAGTGACCGATAAGATG |
| | GATGCTGCAGGGCTGGCATGGGCGATTTACGACGGCGTAGTGCCCAACCCAACAA |
| | TTACTGTCGTCAAAGAAGGGCTCGGTGTATTCCAGAATAGCGGCGCGGATTACCTG |
| | ATCGCTATTGGTGGTGGTTCTCCACAGGATACTTGTAAAGCGATTGGCATTATCAGC |
| | AACAACCCGGAGTTTGCCGATGTGCGTAGCCTGGAAGGGCTTTCCCCGACCAATA |
| | AACCCAGTGTACCGATTCTGGCAATCCCCACCACAGCAGGCACTGCGGCAGAAGT |
| | GACCATTAACTACGTGATCACTGACGAAGAAAAACGGCGCAAGTTTGTTTGCGTT |
| | GATCCGCATGATATCCCGCAGGTGGCGTTTATTGACGCTGACATGATGGATGGTATG |
| | CCTCCAGCGCTGAAAGCTGCGACGGGTGTCGATGCGCTCACTCATGCTATTGAGG |
| | GGTATATTACCCGTGGCGCGTGGGCGCTAACCGATGCACTGCACATTAAAGCGATT |
| | GAAATCATTGCTGGGGCGCTGCGAGGATCGGTTGCTGGTGATAAGGATGCCGGAG |
| | AAGAAATAGCGCTCGGGCAGTATGTTGCGGGTATGGGCTTCTCGAATGTTGGGTTA |
| | GGGTTGGTGCATGGTATGGCGCATCCACTGGGCGCGTTTTATAACACTCCACACGG |
| | TGTTGCAAACGCCATCCTGCTACCGCATGTCATGCGCTATAACGCTGACTTTACCG |
| | GTGAGAAGTACCGCGATATCGCGCGCGTTATGGGCGTGAAAGTGGAAGGTATGAG |
| | CCTGGAAGAGGCGCGTAATGCCGCTGTTGAAGCGGTGTTTGCTCTCAACCGTGAT |
| | GTCGGTATTCCGCCACATTTGCGTGATGTTGGGGTACGCAAGGAAGACATTCCGGC |
| | ACTGGCGCAGGCGGCACTGAATGATGTTTGTACCGGTGGCAACCCGCGTGAAGCA |
| | ACGCTTGAGGATATTGTAGAGCTTTACCATACCGCCTGGTAA |
| | (SEQ ID NO: 4) |

TABLE 1-3
adhP/ACT43318.1/Escherichia coli BL21(DE3)

| | |
|---|---|
| Amino acid | MKAAVVTKDHHVDVTYKTLRSLKHGEALLKMECCGVCHTDLHVKNGDFGDKTGVI |
| sequence | LGHEGIGVVAEVGPGVTSLKPGDRASVAWFYEGCGHCEYCNSGNETLCRSVKNAGY |
| | SVDGGMAEECIVVADYAVKVPDGLDSAAASSITCAGVTTYKAVKLSKIRPGQWIAIY |
| | GLGGLGNLALQYAKNVFNAKVIAIDVNDEQLKLATEMGADLAINSHTEDAAKIVQE |
| | KTGGAHAAVVTAVAKAAFNSAVDAVRAGGRVVAVGLPPESMSLDIPRLVLDGIEVVG |
| | SLVGTRQDLTEAFQFAAEGKVVPKVALRPLADINTIFTEMEEGKIRGRMVIDFRH |
| | (SEQ ID NO: 5) |
| Base | ATGAAGGCTGCAGTTGTTACGAAGGATCATCATGTTGACGTTACGTATAAAACACT |
| sequence | GCGCTCACTGAAACATGGCGAAGCCCTGCTGAAAATGGAGTGTTGTGGTGTATGT |
| | CATACCGATCTTCATGTTAAGAATGGCGATTTTGGTGACAAAACCGGCGTAATTCT |
| | GGGCCATGAAGGCATCGGTGTGGTGGCAGAAGTGGGTCCAGGTGTCACCTCATTA |
| | AAACCAGGCGATCGTGCCAGCGTGGCGTGGTTCTACGAAGGATGCGGTCATTGCG |
| | AATACTGTAACAGTGGTAACGAAACGCTCTGCCGTTCAGTTAAAAATGCCGGATAC |
| | AGCGTTGATGGCGGGATGGCGGAAGAGTGCATCGTGGTCGCCGATTACGCGGTAA |
| | AAGTGCCAGATGGTCTGGACTCGGCGGCGGCCAGCAGCATTACCTGTGCGGGAGT |
| | CACCACCTACAAAGCCGTTAAGCTGTCAAAAATTCGTCCAGGGCAGTGGATTGCT |
| | ATCTACGGTCTTGGCGGTCTGGGTAACCTCGCCCTGCAATACGCGAAGAATGTCTT |
| | TAACGCCAAAGTGATCGCCATTGATGTCAATGATGAGCAGTTAAAACTGGCAACC |
| | GAAATGGGCGCAGATTTAGCGATTAACTCACACACCGAAGACGCCGCCAAAATTG |
| | TGCAGGAGAAAACTGGTGGCGCTCACGCTGCGGTGGTAACAGCGGTAGCTAAAG |
| | CTGCGTTTAACTCGGCAGTTGATGCTGTCCGTGCAGGCGGTCGTGTTGTGGCTGTC |
| | GGTCTACCGCCGGAGTCTATGAGCCTGGATATCCCACGTCTTGTGCTGGATGGTATT |
| | GAAGTGGTCGGTTCGCTGGTCGGCACGCGCCAGGATTTAACTGAAGCCTTCCAGT |
| | TTGCCGCCGAAGGTAAAGTGGTGCCGAAAGTCGCCCTGCGTCCGTTAGCGGACAT |
| | CAACACCATCTTTACTGAGATGGAAGAAGGCAAAATCCGTGGCCGCATGGTGATT |
| | GATTTCCGTCACTAA |
| | (SEQ ID NO: 6) |

TABLE 1-4
ybbO/ACT42343.1/Escherichia coli BL21(DE3)

| | |
|---|---|
| Amino acid | MTHKATEILTGKVMQKSVLITGCSSGIGLESALELKRQGFHVLAGCRKPDDVERMNN |
| sequence | MGFTGVLIDLDSPESVDRAADEVIALTDNCLYGIFNNAGFGMYGPLSTISRAQMEQQF |
| | SANFFGAHQLTMRLLPAMLPHGEGRIVMTSSVMGLISTPGRGAYAASKYALEAWSDA |
| | LRMELRHSGIKVSLIEPGPIRTRFTDNVNQTQSDKPVENPGIAARFTLGPEAVVDKVR |
| | HAFISEKPKMRYPVTLVTWAVMVLKRLLPGRVMDKILQG (SEQ ID NO: 7) |
| Base | ATGACTCATAAAGCAACGGAGATCCTGACAGGTAAAGTTATGCAAAAATCGGTCTT |
| sequence | AATTACCGGATGTTCCAGTGGAATTGGCCTGGAAAGCGCGCTCGAATTAAAACGC |
| | CAGGGTTTTCATGTGCTGGCAGGTTGCCGAAAACCGGATGATGTTGAGCGTATGA |
| | ACAACATGGGATTTACCGGCGTGTTGATCGATCTGGATTCACCAGAAAGTGTTGAT |
| | CGCGCAGCAGACGAGGTGATCGCCCTGACCGATAATTGTCTGTATGGGATCTTTAA |
| | CAATGCCGGATTCGGCATGTATGGCCCCCTTTCCACCATCAGCCGTGCGCAGATGG |
| | AACAGCAGTTTTCCGCCAACTTTTTCGGCGCACACCAGCTCACCATGCGCCTGTTA |
| | CCCGCGATGTTACCGCACGGTGAAGGGCGTATTGTGATGACATCATCGGTGATGGG |
| | ATTAATCTCCACGCCGGGTCGTGGCGCTTACGCGGCCAGTAAATATGCGCTGGAGG |
| | CGTGGTCAGACGCGCTGCGCATGGAGCTACGCCACAGCGGAATTAAAGTCAGCCT |
| | GATCGAACCCGGTCCCATTCGTACTCGCTTCACCGACAACGTCAACCAGACGCAA |
| | AGTGATAAACCAGTCGAAAATCCCGGCATCGCCGCCCGCTTTACGTTGGGACCGG |
| | AAGCGGTGGTGGACAAAGTACGCCATGCTTTTATTAGCGAGAAGCCGAAGATGCG |
| | CTATCCGGTAACGCTGGTGACCTGGGCAGTAATGGTGCTTAAGCGCCTGCTGCCGG |
| | GGCGCGTGATGGACAAAATATTGCAGGGGTGA |
| | (SEQ ID NO: 8) |

TABLE 1-5
eutG/ACT44165.1/Escherichia coli BL21(DE3)

| | |
|---|---|
| Amino acid | MQNELQTALFQAFDTLNLQRVKTFSVPPVTLCGPGAVSSCGQQAQTRGLKHLFVMA |
| sequence | DSFLHQAGMTAGLTRSLAVKGIAMTLWPCPVGEPCITDVCAAVAQLRESGCDGVIAF |
| | GGGSVLDAAKAVALLVTNPDSTLAEMSETSVLQPRLPLIAIPTTAGTGSETTNVTVIID |
| | AVSGRKQVLAHASLMPDVAILDAALTEGVPSHVTAMTGIDALTHAIEAYSALNATPFT |
| | DSLAIGAIAMIGKSLPKAVGYGHDLAARESMLLASCMAGMAFSSAGLGLCHAMAHQ |
| | PGAALHIPHGLANAMLLPTVMEFNRMVCRERFSQIGRALRTKKSDDRDAINAVSELI |
| | AEVGIGKRLGDVGATSAHYGAWAQAALEDICLRSNPRTASLEQIVGLYAAAQ |
| | (SEQ ID NO: 9) |
| Base | ATGCAAAATGAATTGCAGACCGCGCTCTTTCAGGCGTTCGATACCCTGAATCTGCA |
| sequence | ACGGGTAAAAACATTTAGCGTTCCACCGGTGACGCTTTGCGGTCCGGGCGCGGTG |
| | AGCAGTTGCGGGCAGCAAGCGCAAACGCGTGGGCTGAAACATCTGTTCGTGATG |
| | GCAGACAGCTTTTTGCATCAGGCGGGGATGACCGCCGGGCTGACGCGCAGCCTGG |
| | CTGTTAAAGGCATCGCCATGACGCTCTGGCCATGTCCGGTGGGCGAACCGTGCATT |
| | ACCGACGTGTGTGCAGCCGTGGCGCAGTTGCGTGAGTCAGGCTGTGATGGGGTGA |
| | TCGCATTTGGCGGCGGCTCGGTGCTGGATGCGGCGAAAGCCGTGGCGTTGCTGGT |
| | GACGAACCCCGATAGCACGCTGGCAGAGATGTCAGAAACCAGCGTTCTGCAACC |
| | GCGCTTGCCGCTGATTGCCATTCCAACGACCGCCGGAACCGGCTCTGAAACCACC |
| | AATGTAACGGTGATTATCGACGCGGTGAGCGGGCGCAAGCAGGTGTTAGCCCATG |
| | CCTCGCTGATGCCGGATGTGGCGATCCTCGACGCCGCATTGACCGAAGGTGTGCC |
| | GTCGCATGTCACGGCGATGACCGGCATTGATGCGTTAACCCATGCCATTGAAGCAT |
| | ACAGCGCCCTGAACGCTACACCGTTTACCGACAGCCTGGCGATTGGTGCCATTGC |
| | GATGATTGGCAAATCGCTGCCGAAAGCGGTGGGCTACGGTCACGACCTTGCCGCG |
| | CGCGAGAGCATGTTACTGGCTTCATGTATGGCGGGAATGGCGTTTTCCAGTGCGGG |
| | TCTTGGGTTGTGCCACGCGATGGCGCATCAGCCGGGCGCGGCGCTGCATATTCCGC |
| | ACGGTCTCGCGAACGCCATGTTGCTGCCAACGGTGATGGAATTTAACCGGATGGTT |
| | TGTCGTGAACGCTTTAGTCAGATTGGTCGGGCACTGCGAACTAAAAAATCCGACG |
| | ATCGTGACGCTATTAACGCGGTAAGTGAGCTGATTGCGGAAGTTGGGATTGGTAAA |
| | CGACTGGGCGATGTTGGTGCGACATCTGCGCATTACGGCGCATGGGCGCAGGCCG |
| | CGCTGGAAGATATTTGTCTGCGCAGTAACCCGCGTACCGCCAGCCTGGAGCAGATT |
| | GTCGGCCTGTACGCAGCGGCGCAATAA |
| | (SEQ ID NO: 10) |

TABLE 1-6
ahr/ACT45923.1/Escherichia coli BL21(DE3)

| | |
|---|---|
| Amino acid | MSMIKSYAAKEAGGELEVYEYDPGELRPQDVEVQVDYCGICHSDLSMIDNEWGFSQ |
| sequence | YPLVAGHEVIGRVVALGSAAQDKGLQVGQRVGIGWTARSCGHCDACISGNQINCEQG |
| | AVPTIMNRGGFAEKLRADWQWVIPLPENIDIESAGPLLCGGITVFKPLLMHHITATSRV |
| | GVIGIGGLGHIAIKLLHAMGCEVTAFSSNPAKEQEVLAMGADKVVNSRDPQALKALA |
| | GQFDLIINTVNVSLDWQPYFEALTYGGNFHTVGAVLTPLSVPAFTLIAGDRSVSGSATG |
| | TPYELRKLMRFAARSKVAPTTELFPMSKINDAIQHVRDGKARYRVVLKADY |
| | (SEQ ID NO: 11) |
| Base | ATGTCGATGATAAAAAGCTATGCCGCAAAAGAAGCGGGCGGAGAACTGGAAGTTT |
| sequence | ATGAGTACGATCCCGGTGAGCTGAGGCCACAAGATGTTGAAGTGCAGGTGGATTA |
| | CTGCGGGATCTGCCATTCCGATCTGTCGATGATCGATAACGAATGGGGATTTTCAC |
| | AATATCCGCTGGTTGCCGGGCATGAGGTGATTGGGCGCGTGGTGGCACTCGGGAG |
| | CGCCGCGCAGGATAAAGGTTTGCAGGTCGGTCAGCGTGTCGGGATTGGCTGGACG |
| | GCGCGTAGCTGTGGTCACTGCGACGCCTGTATTAGCGGTAATCAGATCAACTGCGA |
| | GCAAGGTGCGGTGCCGACGATTATGAATCGCGGTGGCTTTGCCGAGAAGTTGCGT |
| | GCGGACTGGCAATGGGTGATTCCACTGCCAGAAAATATTGATATCGAGTCCGCCGG |
| | GCCGCTGTTGTGCGGCGGTATCACGGTCTTTAAACCACTGTTGATGCACCATATCA |
| | CTGCTACCAGCCGCGTTGGGGTAATTGGTATTGGCGGGCTGGGGCATATCGCTATA |
| | AAACTTCTGCACGCAATGGGATGCGAGGTGACAGCCTTTAGTTCTAATCCGGCGA |
| | AAGAGCAGGAAGTGCTGGCGATGGGTGCCGATAAAGTGGTGAATAGCCGCGATCC |
| | GCAGGCACTGAAAGCACTGGCGGGGCAGTTTGATCTCATTATCAACACCGTCAAC |
| | GTCAGCCTCGACTGGCAGCCCTATTTTGAGGCGCTGACCTATGGCGGTAATTTCCA |
| | TACGGTCGGTGCGGTTCTCACGCCGCTGTCTGTTCCGGCCTTTACGTTAATTGCGG |
| | GCGATCGCAGCGTCTCTGGTTCTGCTACCGGCACGCCTTATGAGCTGCGTAAGCTG |
| | ATGCGTTTTGCCGCCCGCAGCAAGGTTGCGCCGACCACCGAACTGTTCCCGATGT |
| | CGAAAATTAACGACGCCATCCAGCATGTGCGCGACGGTAAGGCGCGTTACCGCGT |
| | GGTGTTGAAAGCCGATTATTGA |
| | (SEQ ID NO: 12) |

TABLE 1-7
yahK/ACT42179.1/Escherichia coli BL21(DE3)

| | |
|---|---|
| Amino acid | MKIKAVGAYSAKQPLEPMDITRREPGPNDVKIEIAYCGVCHSDLHQVRSEWAGTVYP |
| sequence | CVPGHEIVGRVVAVGDQVEKYAPGDLVGVGCIVDSCKHCEECEDGLENYCDHMTGT |
| | YNSPTPDEPGHTLGGYSQQIVVHERYVLRIRHPQEQLAAVAPLLCAGITTYSPLRHWQ |
| | AGPGKKVGVVGIGGLGHMGIKLAHAMGAHVVAFTTSEAKREAAKALGADEVVNSR |
| | NADEMAAHLKSFDFILNTVAAPHNLDDFTTLLKRDGTMTLVGAPATPHKSPEVFNLI |
| | MKRRAIAGSMIGGIPETQEMLDFCAEHGIVADIEMIRADQINEAYERMLRGDVKYRF |
| | VIDNRTLTD |
| | (SEQ ID NO: 13) |
| Base | ATGAAGATCAAAGCTGTTGGTGCATATTCCGCTAAACAACCACTTGAACCGATGGA |
| sequence | TATCACCCGGCGTGAACCGGGACCGAATGATGTCAAAATCGAAATCGCTTACTGTG |
| | GCGTTTGCCATTCCGATCTCCACCAGGTCCGTTCCGAGTGGGCGGGGACGGTTTAC |
| | CCCTGCGTGCCGGGTCATGAAATTGTGGGGCGTGTGGTAGCCGTTGGTGATCAGG |
| | TAGAAAAATATGCGCCGGGCGATCTGGTCGGTGTCGGCTGCATTGTCGACAGTTGT |
| | AAACATTGCGAAGAGTGTGAAGACGGGTTGGAAAACTACTGTGATCACATGACCG |
| | GCACCTATAACTCGCCGACGCCGGACGAACCGGGCCATACTCTGGGCGGCTACTC |
| | ACAACAGATCGTCGTTCATGAGCGATATGTTCTGCGTATTCGTCACCCGCAAGAGC |
| | AGCTGGCGGCGGTGGCTCCTTTGTTGTGTGCAGGGATCACCACGTATTCGCCGCTA |
| | CGTCACTGGCAGGCCGGGCCGGGTAAAAAAGTGGGCGTGGTCGGCATCGGCGGT |
| | CTGGGACATATGGGGATTAAGCTGGCCCACGCGATGGGGGCACATGTGGTGGCATT |
| | TACCACTTCTGAGGCAAAACGCGAAGCGGCAAAAGCCCTGGGGGCCGATGAAGT |
| | TGTTAACTCACGCAATGCCGATGAGATGGCGGCTCATCTGAAGAGTTTCGATTTCA |
| | TTTTGAATACAGTAGCTGCGCCACATAATCTCGACGATTTTACCACCTTGCTGAAG |
| | CGTGATGGCACCATGACGCTGGTTGGTGCGCCTGCGACACCGCATAAATCGCCGG |
| | AAGTTTTCAACCTGATCATGAAACGCCGTGCGATAGCCGGTTCTATGATTGGCGGC |
| | ATTCCAGAAACTCAGGAGATGCTCGATTTTTGCGCCGAACATGGCATCGTGGCTGA |
| | TATAGAGATGATTCGGGCCGATCAAATTAATGAAGCCTATGAGCGAATGCTGCGCG |
| | GTGATGTGAAATATCGTTTTGTTATCGATAATCGCACACTAACAGACTGA |
| | (SEQ ID NO: 14) |

TABLE 1-8
adhE/ACT43105.1/Escherichia coli BL21(DE3)

| | |
|---|---|
| Amino acid | MAVTNVAELNALVERVKKAQREYASFTQEQVDKIFRAAALAAADARIPLAKMAVAES |
| sequence | GMGIVEDKVIKNHFASEYIYNAYKDEKTCGVLSEDDTFGTITIAEPIGIICGIVPTTNPTS |
| | TAIFKSLISLKTRNAIIFSPHPRAKDATNKAADIVLQAAIAAGAPKDLIGWIDQPSVELS |
| | NALMHHPDINLILATGGPGMVKAAYSSGKPAIGVGAGNTPVVIDETADIKRAVASVL |
| | MSKTFDNGVICASEQSVVVVDSVYDAVRERFATHGGYLLQGKELKAVQDVILKNGA |
| | LNAAIVGQPAYKIAELAGFSVPENTKILIGEVTVVDESEPFAHEKLSPTLAMYRAKDFE |
| | DAVEKAEKLVAMGGIGHTSCLYTDQDNQPARVSYFGQKMKTARILINTPASQGGIGDL |
| | YNFKLAPSLTLGCGSWGGNSISENVGPKHLINKKTVAKRAENMLWHKLPKSIYFRRG |
| | SLPIALDEVITDGHKRALIVTDRFLFNNGYADQITSVLKAAGVETEVFFEVEADPTLSI |
| | VRKGAELANSFKPDVIIALGGGSPMDAAKIMWVMYEHPETHFEELALRFMDIRKRIY |
| | KFPKMGVKAKMIAVTTTSGTGSEVTPFAVVTDDATGQKYPLADYALTPDMAIVDANL |
| | VMDMPKSLCAFGGLDAVTHAMEAYVSVLASEFSDGQALQALKLLKEYLPASYHEGS |
| | KNPVARERVHSAATIAGIAFANAFLGVCHSMAHKLGSQFHIPHGLANALLICNVIRYN |
| | ANDNPTKQTAFSQYDRPQARRRYAEIADHLGLSAPGDRTAAKIEKLLAWLETLKAEL |
| | GIPKSIREAGVQEADFLANVDKLSEDAFDDQCTGANPRYPLISELKQILLDTYYGRDY |
| | VEGETAAKKEAAPAKAEKKAKKSA |
| | (SEQ ID NO: 15) |
| Base | ATGGCTGTTACTAATGTCGCTGAACTTAACGCACTCGTAGAGCGTGTAAAAAAAGC |
| sequence | CCAGCGTGAATATGCCAGTTTCACTCAAGAGCAAGTAGACAAAATCTTCCGCGCC |
| | GCCGCTCTGGCTGCTGCAGATGCTCGAATCCCACTCGCGAAAATGGCCGTTGCCG |
| | AATCCGGCATGGGTATCGTCGAAGATAAAGTGATCAAAAACCACTTTGCTTCTGAA |
| | TATATCTACAACGCCTATAAAGATGAAAAAACCTGTGGTGTTCTGTCTGAAGACGA |
| | CACTTTTGGTACCATCACTATCGCTGAACCAATCGGTATTATTTGCGGTATCGTTCC |
| | GACCACTAACCCGACTTCAACTGCTATCTTCAAATCGCTGATCAGTCTGAAGACCC |
| | GTAACGCCATTATCTTCTCCCCGCACCCGCGTGCAAAAGATGCCACCAACAAAGC |
| | GGCTGATATCGTTCTGCAGGCTGCTATCGCTGCCGGTGCTCCGAAAGATCTGATCG |
| | GCTGGATCGATCAACCTTCTGTTGAACTGTCTAACGCACTGATGCACCACCCAGAC |
| | ATCAACCTGATCCTCGCGACTGGTGGTCCGGGCATGGTTAAAGCCGCATACAGCTC |
| | CGGTAAACCAGCTATCGGTGTAGGCGCGGGCAACACTCCAGTTGTTATCGATGAA |
| | ACTGCTGATATCAAACGTGCAGTTGCATCTGTACTGATGTCCAAAACCTTCGACAA |
| | CGGCGTAATCTGTGCTTCTGAACAGTCTGTTGTTGTTGTTGACTCTGTTTATGACGC |
| | TGTACGTGAACGTTTTGCAACCCACGGCGGCTATCTGTTGCAGGGTAAAGAGCTG |
| | AAAGCTGTTCAGGATGTTATCCTGAAAAACGGTGCGCTGAACGCGGCTATCGTTG |
| | GTCAGCCAGCCTATAAAATTGCTGAACTGGCAGGCTTCTCTGTACCAGAAAACAC |
| | CAAGATTCTGATCGGTGAAGTGACCGTTGTTGATGAAAGCGAACCGTTCGCACAT |
| | GAAAAACTGTCCCCGACTCTGGCAATGTACCGCGCTAAAGATTTCGAAGACGCGG |
| | TAGAAAAAGCAGAGAAACTGGTTGCTATGGGCGGTATCGGTCATACCTCTTGCCTG |
| | TACACTGACCAGGATAACCAACCGGCTCGCGTTTCTTACTTCGGTCAGAAAATGA |
| | AAACGGCGCGTATCCTGATTAACACCCCAGCGTCTCAGGGTGGTATCGGTGACCTG |
| | TATAACTTCAAACTCGCACCTTCCCTGACTCTGGGTTGTGGTTCTTGGGGTGGTAA |
| | CTCCATCTCTGAAAACGTTGGTCCGAAACACCTGATCAACAAGAAAACCGTTGCT |
| | AAGCGAGCTGAAAACATGTTGTGGCACAAACTTCCGAAATCTATCTACTTCCGCCG |
| | TGGCTCCCTGCCAATCGCGCTGGATGAAGTGATTACTGATGGCCACAAACGTGCG |
| | CTCATCGTGACTGACCGCTTCCTGTTCAACAATGGTTATGCTGATCAGATCACTTCC |
| | GTACTGAAAGCAGCAGGCGTTGAAACTGAAGTCTTCTTCGAAGTAGAAGCGGAC |
| | CCGACCCTGAGCATCGTTCGTAAAGGTGCAGAACTGGCAAACTCCTTCAAACCAG |
| | ACGTGATTATCGCGCTGGGTGGTGGTTCCCCGATGGACGCCGCGAAGATCATGTGG |
| | GTTATGTACGAACATCCGGAAACTCACTTCGAAGAGCTGGCGCTGCGCTTTATGGA |
| | TATCCGTAAACGTATCTACAAGTTCCCGAAAATGGGCGTGAAAGCGAAAATGATCG |
| | CTGTCACCACCACTTCTGGTACAGGTTCTGAAGTCACTCCGTTTGCGGTTGTAACT |
| | GACGACGCTACTGGTCAGAAATATCCGCTGGCAGACTATGCGCTGACTCCGGATAT |
| | GGCGATTGTCGACGCCAACCTGGTTATGGACATGCCGAAGTCCCTGTGTGCTTTCG |
| | GTGGTCTGGACGCAGTAACTCACGCCATGGAAGCTTATGTTTCTGTACTGGCATCT |
| | GAGTTCTCTGATGGTCAGGCTCTGCAGGCACTGAAACTGCTGAAAGAATATCTGC |
| | CAGCGTCCTACCACGAAGGGTCTAAAAATCCGGTAGCGCGTGAACGTGTTCACAG |
| | TGCAGCGACTATCGCGGGTATCGCGTTTGCGAACGCCTTCCTGGGTGTATGTCACT |
| | CAATGGCGCACAAACTGGGTTCCCAGTTCCATATTCCGCACGGTCTGGCAAACGC |
| | CCTGCTGATTTGTAACGTTATTCGCTACAATGCGAACGACAACCCGACCAAGCAGA |
| | CTGCATTCAGCCAGTATGACCGTCCGCAGGCTCGCCGTCGTTATGCTGAAATTGCC |
| | GACCACTTGGGTCTGAGCGCACCGGGCGACCGTACTGCTGCTAAGATCGAGAAAC |
| | TGCTGGCATGGCTGGAAACGCTGAAAGCTGAACTGGGTATTCCGAAATCTATCCGT |
| | GAAGCTGGCGTTCAGGAAGCAGACTTCCTGGCGAACGTGGATAAACTGTCTGAA |
| | GATGCATTCGATGACCAGTGCACCGGCGCTAACCCGCGTTACCCGCTGATCTCCGA |
| | GCTGAAACAGATCCTGCTGGATACCTACTACGGTCGTGATTATGTAGAAGGTGAAA |
| | CTGCAGCGAAAAAAGAAGCCGCTCCGGCTAAAGCTGAGAAAAAAGCGAAAAAAT |
| | CCGCTTAA |
| | (SEQ ID NO: 16) |

TABLE 1-9
ybdR/ACT42455.1/Escherichia coli BL21(DE3)

| | |
|---|---|
| Amino acid | MKALTYHGPHHVQVENVPDPGIEQADDIILRITATAICGSDLHLYRGKIPQVKHGDIFG |
| sequence | HEFMGEVVETGKDVKNLQKGDRVVIPFVIACGDCFFCRLQQYAACENTNAGKGAAL |
| | NKKQIPAPAALFGYSHLYGGVPGGQAEYVRVPKGNVGPFKVPPLLSDDKALFLSDILP |
| | TAWQAAKNAQIQQGSSVAVYGAGPVGLLTIACARLLGAEQIFVVDHHPYRLHFAADR |
| | YGAIPINFDEDSDPAQSIIEQTAGHRGVDAVIDAVGFEAKGSTTETVLTNLKLEGSSGK |
| | ALRQCIAAVRRGGIVSVPGVYAGFIHGFLFGDAFDKGLSFKMGQTHVHAWLGELLPL |
| | IEKGLLKPEEIVTHYMPFEEAARGYEIFEKREEECRKVILVPGAQSAEAAQKAVSGLV |
| | NAMPGGTI (SEQ ID NO: 17) |
| Base | ATGAAAGCATTGACTTATCACGGCCCACATCACGTTCAGGTAGAAAATGTTCCCGA |
| sequence | TCCGGGCATTGAACAGGCAGATGATATTATTCTGCGTATTACGGCAACGGCGATCT |
| | GTGGCTCTGACCTCCATCTTTATCGAGGCAAAATACCCCAGGTTAAACATGGCGAT |
| | ATTTTTGGTCATGAATTTATGGGGGAAGTCGTTGAAACCGGAAAGGACGTAAAAA |
| | ATTTGCAAAAAGGCGACCGGGTGGTAATTCCGTTTGTCATTGCTTGTGGCGACTGT |
| | TTTTTCTGTCGATTACAGCAATATGCCGCCTGCGAAAATACCAATGCGGGTAAAGG |
| | CGCTGCGCTCAATAAAAAACAGATACCAGCTCCCGCGGCATTGTTTGGTTATAGTC |
| | ACCTGTATGGCGGCGTTCCTGGTGGGCAGGCGGAATATGTCCGCGTCCCTAAAGG |
| | GAATGTGGGGCCGTTTAAAGTACCGCCTTTGCTTTCAGATGATAAAGCGCTTTTCC |
| | TTTCTGATATTCTGCCAACGGCATGGCAGGCAGCAAAAAATGCGCAGATCCAACA |
| | AGGTTCAAGCGTTGCAGTCTATGGTGCTGGTCCTGTGGGATTGTTGACAATCGCCT |
| | GTGCACGGTTGCTCGGTGCGGAACAGATTTTTGTTGTTGATCATCATCCCTACCGC |
| | TTGCATTTCGCCGCCGACCGCTACGGCGCGATCCCGATTAATTTTGATGAAGACAG |
| | CGATCCGGCACAGTCAATTATTGAACAAACGGCAGGTCACCGGGGCGTGGATGCA |
| | GTAATAGACGCCGTCGGTTTTGAAGCGAAAGGCAGCACCACGGAAACGGTGCTG |
| | ACTAACCTGAAACTGGAGGGCAGCAGCGGTAAAGCGTTGCGTCAGTGTATTGCGG |
| | CGGTCAGGCGTGGCGGCATTGTTAGCGTACCGGGCGTCTACGCTGGATTTATTCAC |
| | GGTTTCCTGTTTGGCGACGCCTTTGATAAAGGGTTGTCGTTTAAAATGGGACAGAC |
| | CCACGTTCACGCATGGCTGGGAGAATTATTACCGTTAATTGAGAAAGGATTACTGA |
| | AACCAGAAGAAATTGTTACCCACTATATGCCGTTTGAAGAGGCCGCCCGGGGATAT |
| | GAGATTTTCGAAAAACGTGAAGAGGAGTGCCGTAAGGTGATTCTGGTACCCGGTG |
| | CACAAAGCGCAGAGGCGGCGCAGAAGGCGGTTTCAGGTCTAGTGAATGCGATGC |
| | CGGGGGGAACAATATGA (SEQ ID NO: 18) |

TABLE 1-10
dkgA/ACT44689.1/Escherichia coli BL21(DE3)

| | |
|---|---|
| Amino acid | MANPTVIKLQDGNVMPQLGLGVWQASNEEVITAIQKALEVGYRSIDTAAAYKNEEG |
| sequence | VGKALKNASVNREELFITTKLWNDDHKRPREALLDSLKKLQLDYIDLYLMHWPVPAI |
| | DHYVEAWKGMIELQKEGLIKSIGVCNFQIHHLQRLIDETGVTPVINQIELHPLMQQRQ |
| | LHAWNATHKIQTESWSPLAQGGKGVFDQKVIRDLADKYGKTPAQIVIRWHLDSGLV |
| | VIPKSVTPSRIAENFDVWDFRLDKDELGEIAKLDQGKRLGPDPDQFGG (SEQ ID NO: |
| | 19) |
| Base | ATGGCTAATCCAACCGTTATTAAGCTACAGGATGGCAATGTCATGCCCCAGCTGGG |
| sequence | ACTGGGCGTCTGGCAAGCAAGTAATGAGGAAGTAATCACCGCCATTCAAAAAGCG |
| | TTAGAAGTGGGTTATCGCTCGATTGATACCGCCGCGGCCTACAAGAACGAAGAAG |
| | GTGTCGGCAAAGCCCTGAAAAATGCCTCAGTCAACAGAGAAGAACTGTTCATCAC |
| | CACTAAGCTGTGGAACGACGACCACAAGCGCCCCCGCGAAGCCCTGCTCGACAG |
| | CCTGAAAAAACTCCAGCTTGATTATATCGACCTCTACTTAATGCACTGGCCCGTTCC |
| | CGCTATCGACCATTATGTCGAAGCATGGAAAGGCATGATCGAATTGCAAAAAGAG |
| | GGATTAATCAAAAGCATCGGCGTGTGCAACTTCCAGATCCATCACCTGCAACGCCT |
| | GATTGATGAAACTGGCGTGACGCCTGTGATAAACCAGATCGAACTTCATCCGCTGA |
| | TGCAACAACGCCAGCTACACGCCTGGAACGCGACACACAAAATCCAGACCGAAT |
| | CCTGGAGCCCATTAGCGCAAGGAGGGAAAGGCGTTTTCGATCAGAAAGTCATTCG |
| | CGATCTGGCAGATAAATACGGCAAAACCCCGGCGCAGATTGTTATCCGCTGGCATC |
| | TGGATAGCGGCCTGGTGGTGATCCCGAAATCGGTCACACCTTCACGTATTGCCGAA |
| | AACTTTGATGTCTGGGATTTCCGTCTCGACAAAGACGAACTCGGCGAAATTGCAA |
| | AACTCGATCAGGGCAAGCGTCTCGGTCCCGATCCTGACCAGTTCGGCGGCTAA |
| | (SEQ ID NO: 20) |

TABLE 1-11
yiaY/ACT45243.1/Escherichia coli BL21(DE3)

| | |
|---|---|
| Amino acid | MASSTFFIPSVNVIGADSLTDAMNMMADYGFTRTLIVTDNMLTKLGMAGDVQKALE |
| sequence | ERNIFSVIYDGTQPNPTTENVAAGLKLLKENNCDSVISLGGGSPHDCAKGIALVAANG |
| | GDIRDYEGVDRSAKPQLPMIAINTTAGTASEMTRFCIITDEARHIKMAIVDKHVTPLLS |
| | VNDSSLMIGMPKSLTAATGMDALTHAIEAYVSIAATPITDACALKAVTMIAENLPLAVE |
| | DGSNAKAREAMAYAQFLAGMAFNNASLGYVHAMAHQLGGFYNLPHGVCNAVLLP |
| | HVQVFNSKVAAARLRDCAAAMGVNVTGKNDAEGAEACINAIRELAKKVDIPAGLRD |
| | LNVKEEDFAVLATNALKDACGFTNPIQATHEEIVAIYRAAM |
| | (SEQ ID NO: 21) |
| Base | ATGGCATCTTCAACTTTCTTTATTCCTTCTGTGAATGTCATCGGCGCTGATTCATTGA |
| sequence | CTGATGCAATGAATATGATGGCAGATTATGGATTTACCCGTACCTTAATTGTCACTG |
| | ACAATATGTTAACGAAATTAGGTATGGCGGGTGATGTGCAAAAAGCACTGGAAGA |
| | ACGCAATATTTTTAGCGTTATTTATGATGGCACCCAACCTAACCCAACCACGGAAA |
| | ACGTCGCCGCAGGTTTGAAATTACTTAAAGAAAATAATTGCGATAGCGTGATTTCC |
| | TTAGGCGGTGGTTCTCCGCATGACTGTGCAAAAGGTATTGCGCTGGTGGCAGCCA |
| | ATGGTGGTGATATCCGTGATTATGAAGGCGTTGACCGCTCTGCAAAACCGCAGCTG |
| | CCGATGATCGCCATCAATACCACTGCGGGTACAGCATCAGAAATGACTCGTTTCTG |
| | CATCATCACCGACGAAGCGCGTCACATCAAAATGGCGATTGTTGATAAGCACGTG |
| | ACTCCGCTGCTTTCTGTCAATGACTCCTCGCTGATGATCGGTATGCCGAAGTCACT |
| | GACCGCCGCCACTGGTATGGACGCCTTAACGCACGCTATCGAAGCGTATGTTTCTA |
| | TTGCCGCCACGCCGATCACTGACGCTTGTGCACTGAAAGCCGTGACCATGATTGC |
| | CGAAAACCTGCCGTTAGCCGTTGAAGATGGCAGTAATGCGAAAGCGCGTGAAGCA |
| | ATGGCTTATGCCCAGTTCCTCGCCGGTATGGCGTTCAATAATGCTTCTCTGGGTTAT |
| | GTTCATGCGATGGCGCACCAGCTGGGCGGTTTCTACAACCTGCCACACGGTGTATG |
| | TAACGCCGTTTTGCTGCCGCATGTTCAGGTATTCAACAGCAAAGTCGCCGCCGCAC |
| | GTCTGCGTGACTGTGCCGCTGCAATGGGCGTGAACGTGACAGGTAAAAACGATGC |
| | GGAAGGTGCTGAAGCCTGCATTAACGCCATCCGTGAACTGGCGAAGAAAGTGGAT |
| | ATCCCGGCAGGCCTACGCGACCTGAACGTGAAAGAAGAAGATTTCGCGGTTCTGG |
| | CGACTAATGCCCTGAAAGATGCCTGTGGTTTTACTAACCCGATCCAGGCAACTCAC |
| | GAAGAAATTGTGGCGATTTATCGCGCAGCGATGTAA |
| | (SEQ ID NO: 22) |

TABLE 1-12
frmA/ACT42209.1/Escherichia coli BL21(DE3)

| | |
|---|---|
| Amino acid | MKSRAAVAFAPGKPLEIVEIDVAPPKKGEVLIKVTHTGVCHTDAFTLSGDDPEGVFPV |
| sequence | VLGHEGAGVVVEVGEGVTSVKPGDHVIPLYTAECGECEFCRSGKTNLCVAVRETQGK |
| | GLMPDGTTRFSYNGQPLYHYMGCSTFSEYTVVAEVSLAKINPEANHEHVCLLGCGVT |
| | TGIGAVHNTAKVQPGDSVAVFGLGAIGLAVVQGARQAKAGRIIAIDTNPKKFDLARRF |
| | GATDCINPNDYDKPIKDVLLDINKWGIDHTFECIGNVNVMRAALESAHRGWGQSVII |
| | GVAGAGQEISTRPFQLVTGRVWKGSAFGGVKGRSQLPGMVEDAMKGDIDLEPFVTH |
| | TMSLDEINDAFDLMHEGKSIRTVIRY (SEQ ID NO: 23) |
| Base | ATGAAATCACGTGCTGCCGTTGCATTTGCTCCCGGTAAACCGCTGGAAATCGTTGA |
| sequence | AATTGACGTTGCACCACCGAAAAAAGGTGAAGTGCTGATTAAAGTCACCCATACC |
| | GGCGTTTGCCATACCGACGCATTTACCCTCTCCGGGGATGACCCGGAAGGTGTATT |
| | CCCGGTGGTTCTCGGTCACGAAGGGGCCGGCGTTGTGGTTGAAGTCGGTGAAGG |
| | CGTAACCAGCGTCAAACCTGGCGACCATGTGATCCCGCTTTACACCGCGGAGTGC |
| | GGCGAGTGTGAGTTCTGTCGTTCTGGCAAAACTAACCTCTGTGTTGCGGTTCGCG |
| | AAACCCAGGGTAAAGGCTTGATGCCAGACGGCACCACCCGTTTTTCTTACAACGG |
| | GCAGCCGCTTTATCACTACATGGGATGCTCAACATTCAGTGAATACACCGTGGTCG |
| | CGGAAGTGTCTCTGGCCAAAATTAATCCAGAAGCAAACCATGAACACGTCTGCCT |
| | GCTGGGCTGTGGCGTGACCACCGGTATTGGCGCGGTGCACAACACAGCTAAAGTC |
| | CAGCCAGGTGATTCTGTTGCCGTGTTTGGTCTTGGCGCGATTGGTCTGGCAGTGGT |
| | TCAGGGCGCGCGTCAGGCGAAAGCGGGACGGATTATCGCTATCGATACCAACCCG |
| | AAGAAATTCGATCTGGCTCGTCGCTTCGGTGCTACCGACTGCATTAACCCGAATGA |
| | CTACGACAAACCGATTAAAGATGTCCTGCTGGATATCAACAAATGGGGTATCGACC |
| | ATACCTTTGAATGCATCGGTAACGTCAACGTGATGCGTGCGGCGCTGGAAAGTGC |
| | GCACCGCGGCTGGGGTCAGTCGGTGATCATCGGGGTAGCAGGTGCCGGTCAGGA |
| | AATCTCCACCCGACCATTCCAGTTGGTCACCGGTCGCGTATGGAAAGGTTCCGCGT |
| | TTGGCGGCGTGAAAGGTCGTTCCCAGTTACCGGGTATGGTTGAAGATGCGATGAA |
| | AGGTGATATCGATCTGGAACCGTTTGTCACGCATACCATGAGCCTTGATGAAATTA |
| | ATGACGCCTTCGACCTGATGCATGAAGGCAAATCCATTCGAACCGTAATTCGTTAC |
| | TGA(SEQ ID NO: 24) |

TABLE 1-13
dkgB/ACT42101.1/Escherichia coli BL21(DE3)

| | |
|---|---|
| Amino acid | MAIPAFGLGTFRLKDDVVISSVKTALELGYRAIDTAQIYDNEAAVGQAIAESGVPRHE |
| sequence | LYITTKIWIENLSKDKLIPSLKESLQKLRTDYVDLTLIHWPSPNDEVSVEEFMQELLEA |
| | KKEGLTREIGISNFTIPLMEKAIAAVGAENIATNQIELSPYLQNRKVVAWAKQHGIHITS |
| | YMTLAYGKALKDEVIARIAAKHNATPAQVILAWAMGEGYSVIPSSTKRKNLESNLKA |
| | QNLQLDAEDKKAIAALDCNDRLVSPEGLAPEWD (SEQ ID NO: 25) |
| Base | ATGGCTATCCCTGCATTTGGTTTAGGTACTTTCCGTCTGAAAGACGACGTTGTTATT |
| sequence | TCATCTGTGAAAACGGCGCTTGAACTTGGTTATCGCGCAATTGATACTGCACAAAT |
| | CTATGATAACGAAGCCGCAGTAGGTCAGGCGATTGCAGAAAGTGGCGTGCCACGT |
| | CATGAACTCTACATCACCACTAAAATCTGGATTGAAAATCTCAGCAAAGACAAATT |
| | GATCCCGAGTCTGAAAGAGAGCCTGCAAAAATTGCGTACTGATTATGTTGATCTGA |
| | CTCTAATCCACTGGCCGTCACCAAACGATGAAGTCTCTGTTGAAGAGTTTATGCAG |
| | GAGCTGCTGGAAGCCAAAAAAGAAGGGTTGACGCGTGAGATCGGTATTTCCAACT |
| | TCACGATCCCATTGATGGAAAAGGCGATTGCTGCTGTTGGCGCTGAAAACATCGCT |
| | ACTAACCAGATTGAACTCTCTCCTTATCTGCAAAACCGTAAAGTGGTTGCCTGGGC |
| | TAAACAGCACGGCATCCATATTACTTCCTATATGACGCTGGCGTATGGTAAGGCCCT |
| | GAAAGATGAGGTTATTGCTCGTATCGCAGCTAAACACAATGCGACTCCGGCACAA |
| | GTGATTCTGGCGTGGGCTATGGGGGAAGGTTACTCAGTAATTCCTTCTTCTACTAA |
| | ACGTAAAAACCTGGAAAGTAATCTTAAGGCACAAAATTTACAGCTTGATGCCGAA |
| | GATAAAAAAGCGATCGCCGCACTGGATTGCAACGACCGCCTGGTTAGCCCGGAAG |
| | GTCTGGCTCCTGAATGGGATTAA (SEQ ID NO: 26) |

TABLE 1-14
yghA/ACT44682.1/Escherichia coli BL21(DE3)

| | |
|---|---|
| Amino | MSHLKDPTTQYYTGEYPKQKQPTPGIQAKMTPVPDCGEKTY |
| acid | VGSGRLKDRKALVTGGDSGIGRAAAIAYAREGADVAISYLP |
| sequence | VEEEDAQDVKKIIEECGRKAVLLPGDLSDEKFARSLVHEAH |
| | KALGGLDIMALVAGKQVAIPDIADLTSEQFQKTFAINVFAL |
| | FWLTQEAIPLLPKGASIITTSSIQAYQPSPHLLDYAATKAA |
| | ILNYSRGLAKQVAEKGIRVNIVAPGPIWTALQISGGQTQDK |
| | IPQFGQQTPMKRAGQPAELAPVYVYLASQESSYVTAEVHGV |
| | CGGEHLG |
| | (SEQ ID NO: 27) |
| Base | ATGTCTCATTTAAAAGACCCGACCACGCAGTATTACACTGG |
| sequence | TGAATATCCCAAACAGAAACAACCGACGCCAGGCATCCAGG |
| | CGAAGATGACACCGGTACCGGATTGCGGCGAGAAAACCTAT |
| | GTTGGTAGCGGTCGCCTGAAAGATCGTAAAGCACTGGTGAC |
| | AGGGGGCGATTCCGGAATAGGTCGCGCTGCCGCCATCGCTT |
| | ACGCGCGTGAAGGGGCTGACGTGGCGATCAGTTATCTTCCC |
| | GTGGAAGAAGAAGACGCTCAGGATGTGAAAAAGATCATTGA |
| | AGAATGCGGACGCAAAGCCGTTCTGCTGCCAGGCGATTTAA |
| | GCGATGAGAAATTTGCCCGTTCGCTGGTTCACGAAGCGCAC |
| | AAGGCGTTAGGCGGGCTGGATATTATGGCGCTGGTCGCCGG |
| | GAAACAGGTTGCCATTCCGGATATTGCAGACCTCACCAGCG |
| | AACAGTTTCAAAAGACCTTTGCCATTAACGTTTTCGCGCTG |
| | TTCTGGCTAACCCAGGAAGCGATCCCCCTGCTACCGAAAGG |
| | TGCAAGTATCATCACCACTTCGTCAATCCAGGCATACCAGC |
| | CAAGTCCGCATTTACTGGACTATGCGGCTACGAAGGCGGCG |
| | ATTCTGAACTACAGCCGTGGCTTGGCAAAACAGGTCGCGGA |
| | GAAAGGTATTCGGGTGAATATTGTCGCGCCAGGCCCGATCT |
| | GGACAGCACTGCAAATTTCCGGCGGACAAACGCAGGATAAG |
| | ATCCCGCAGTTTGGTCAGCAAACGCCGATGAAACGTGCGGG |
| | GCAACCGGCGGAACTGGCCCCTGTATATGTTTATCTGGCAA |
| | GTCAGGAGTCGAGCTACGTCACCGCAGAAGTGCACGGCGTG |
| | TGCGGCGGCGAGCATTTAGGTTAA |
| | (SEQ ID NO: 28) |

TABLE 1-15
ydjG/ACT43594.1/Escherichia coli BL21(DE3)

| | |
|---|---|
| Amino | MKKIPLGTTDITLSRMGLGTWAIGGGPAWNGDLDRQICIDT |
| acid | ILEAHRCGINLIDTAPGYNFGNSEVIVGQALKKLPREQVVV |
| sequence | ETKCGIVWERKGSLFNKVGDRQLYKNLSPESIREEVEASLQ |
| | RLGIDYIDIYMTHWQSVPPFFTPIAETVAVLNELKAEGKIR |
| | AIGAANVDADHIREYLQYGELDIIQAKYSILDRAMENELLP |
| | LCRDNGIVVQVYSPLEQGLLTGTITRDYVPGGARANKVWFQ |
| | RENMLKVIDMLEQWQPLCARYQCTIPTLALAWILKQSDLIS |
| | ILSGATAPEQVRENVAALNINLSDADATLMREMAEALER |
| | (SEQ ID NO: 29) |
| Base | ATGAAAAAAATACCTTTAGGCACAACGGATATTACGCTTTC |
| sequence | GCGAATGGGGTTGGGGACATGGGCCATTGGCGGCGGTCCTG |
| | CATGGAATGGCGATCTCGATCGGCAAATATGTATTGATACG |
| | ATTCTTGAAGCCCATCGTTGCGGCATTAATCTGATTGATAC |
| | TGCACCAGGATATAACTTTGGCAATAGTGAAGTTATCGTCG |
| | GTCAGGCGTTAAAAAAACTGCCCCGTGAACAGGTTGTAGTA |
| | GAAACCAAATGCGGCATTGTCTGGGAACGAAAAGGAAGTTT |
| | ATTCAACAAAGTTGGCGATCGGCAGTTGTATAAAAACCTTT |
| | CCCCGGAATCTATCCGCGAAGAGGTAGAAGCCAGCTTGCAA |
| | CGTCTGGGTATTGATTACATCGATATCTACATGACGCACTG |
| | GCAGTCGGTGCCGCCATTTTTTACGCCGATAGCTGAAACTG |
| | TCGCAGTGCTTAATGAGTTAAAAGCCGAAGGGAAAATTCGC |
| | GCGATAGGCGCTGCTAACGTCGATGCTGACCATATCCGCGA |
| | GTATCTGCAATATGGTGAACTGGATATTATTCAGGCGAAAT |
| | ACAGTATCCTCGACCGGGCAATGGAAAACGAACTGCTGCCG |
| | CTATGTCGTGATAATGGCATTGTGGTTCAGGTTTATTCCCC |
| | GCTAGAGCAGGGATTGTTGACCGGCACCATCACTCGTGATT |
| | ACGTTCCGGGCGGCGCTCGGGCAAATAAAGTCTGGTTCCAG |
| | CGTGAAAACATGCTGAAAGTGATTGATATGCTTGAACAGTG |
| | GCAGCCACTTTGTGCTCGTTATCAGTGCACAATTCCCACTC |
| | TGGCACTGGCGTGGATATTAAAACAGAGTGATTTAATCTCC |
| | ATTCTTAGTGGGGCTACTGCACCGGAACAGGTACGCGAAAA |
| | TGTCGCGGCACTGAATATCAACTTATCGGATGCAGACGCAA |
| | CATTGATGAGGGAAATGGCAGAGGCCCTGGAGCGTTAA |
| | (SEQ ID NO: 30) |

TABLE 1-16
gldA/ACT45624.1/Escherichia coli BL21(DE3)

| | |
|---|---|
| Amino | MDRIIQSPGKYIQGADVINRLGEYLKPLAERWLVVGDKFVL |
| acid | GFAQSTVEKSFKDAGLVVEIAPFGGECSQNEIDRLRGIAET |
| sequence | AQCGAILGIGGGKTLDTAKALAHFMGVPVAIAPTIASTDAP |
| | CSALSVIYTDEGEFDRYLLLPNNPNMVIVDTKIVAGAPARL |
| | LAAGIGDALATWFEARACSRSGATTMAGGKCTQAALALAEL |
| | CYNTLLEEGEKAMLAAEQHVVTPALERVIEANTYLSGVGFE |
| | SGGLAAAHAVHNGLTAIPDAHHYYHGEKVAFGTLTQLVLEN |
| | APVEEIETVAALSHAVGLPITLAQLDIKEDVPAKMRIVAEA |
| | ACAEGETIHNMPGGATPDQVYAALLVADQYGQRFLQEWE |
| | (SEQ ID NO: 31) |
| Base | ATGGACCGCATTATTCAATCACCGGGTAAATACATCCAGGG |
| sequence | CGCTGATGTGATTAATCGTCTGGGCGAATACCTGAAGCCGC |
| | TGGCAGAACGCTGGTTAGTGGTGGGTGACAAATTTGTTTTA |
| | GGTTTTGCTCAATCCACTGTCGAGAAAAGCTTTAAAGATGC |
| | TGGACTGGTAGTAGAAATTGCGCCGTTTGGCGGTGAATGTT |
| | CGCAAAATGAGATCGACCGTCTGCGTGGCATCGCGGAGACT |
| | GCGCAGTGTGGCGCAATTCTCGGTATCGGTGGCGGAAAAAC |
| | CCTCGATACTGCCAAAGCACTGGCACATTTCATGGGTGTTC |
| | CGGTAGCGATCGCACCGACTATCGCCTCTACCGATGCACCG |
| | TGCAGCGCATTGTCTGTTATCTACACCGATGAGGGTGAGTT |
| | TGACCGCTATCTGCTGTTGCCAAATAACCCGAATATGGTCA |
| | TTGTCGACACCAAAATCGTCGCTGGCGCACCTGCACGTCTG |
| | TTAGCGGCGGGTATCGGCGATGCGCTGGCAACCTGGTTTGA |
| | AGCGCGTGCCTGCTCTCGTAGCGGCGCGACCACCATGGCGG |
| | GCGGCAAGTGCACCCAGGCTGCGCTGGCACTGGCTGAACTG |
| | TGCTACAACACCCTGCTGGAAGAAGGCGAAAAAGCGATGCT |
| | TGCTGCCGAACAGCATGTAGTGACTCCGGCGCTGGAGCGCG |
| | TGATTGAAGCGAACACCTATTTGAGCGGTGTTGGTTTTGAA |
| | AGTGGTGGTCTGGCTGCGGCGCACGCAGTGCATAACGGCCT |
| | GACCGCTATCCCGGACGCGCATCACTATTATCACGGTGAAA |
| | AAGTGGCATTCGGTACGCTGACGCAGCTGGTTCTGGAAAAC |
| | GCGCCGGTGGAGGAAATCGAAACCGTAGCTGCGCTTAGCCA |
| | TGCGGTAGGTTTGCCAATAACTCTCGCTCAACTGGATATTA |
| | AAGAAGATGTCCCGGCGAAAATGCGAATTGTGGCAGAAGCG |
| | GCATGTGCAGAAGGTGAAACCATCCACAACATGCCTGGCGG |
| | CGCGACGCCAGATCAGGTTTACGCCGCTCTGCTGGTAGCCG |
| | ACCAGTACGGTCAGCGTTTCCTGCAAGAGTGGGAATAA |
| | (SEQ ID NO: 32) |

TABLE 1-17
yohF/ACT43891.1/Escherichia coli BL21(DE3)

| | |
|---|---|
| Amino | MAQVAIITASDSGIGKECALLLAQQGFDIGITWHSDEEGAK |
| acid | DTAREVVSHGVRAEIVQLDLGNLPEGALALEKLIQRLGRID |
| sequence | VLVNNAGAMTKAPFLDMAFDEWRKIFTVDVDGAFLCSQIAA |
| | RQMVKQGQGGRIINITSVHEHTPLPDASAYTAAKHALGGLT |
| | KAMALELVRHKILVNAVAPGAIATPMNGMDDSDVKPDAEPS |
| | IPLRRFGATHEIASLVVWLCSEGANYTTGQSLIVDGGFMLA |
| | NPQFNPE |
| | (SEQ ID NO: 33) |
| Base | ATGGCACAGGTTGCGATTATTACCGCCTCCGATTCGGGGAT |
| sequence | CGGCAAAGAGTGCGCGTTATTACTGGCGCAGCAGGGGTTTG |
| | ATATTGGTATTACCTGGCACTCAGATGAAGAAGGGGCAAAA |
| | GATACCGCGCGTGAGGTAGTTAGCCACGGCGTACGTGCGGA |
| | GATCGTGCAGCTGGATCTCGGCAATCTACCAGAAGGGGCAC |
| | TGGCGCTGGAGAAACTCATTCAACGGCTGGGGCGCATTGAT |
| | GTGCTGGTGAATAATGCGGGTGCAATGACCAAAGCGCCGTT |
| | TCTTGATATGGCTTTTGATGAGTGGCGCAAGATTTTTACCG |
| | TTGATGTCGATGGTGCATTCTTATGCTCGCAAATTGCGGCT |
| | CGTCAGATGGTGAAACAAGGGCAGGGCGGTCGCATCATCAA |
| | CATTACGTCGGTACATGAACATACGCCGCTGCCGGATGCCA |
| | GCGCCTACACAGCCGCTAAACATGCGCTCGGTGGGTTAACC |
| | AAAGCGATGGCGCTGGAGCTGGTCAGGCATAAGATTTTGGT |
| | GAACGCAGTCGCGCCTGGGGCGATCGCCACGCCAATGAATG |
| | GCATGGATGACAGCGACGTGAAGCCCGACGCGGAGCCTTCG |
| | ATTCCCTTGCGGCGTTTTGGCGCAACGCATGAGATTGCCAG |
| | CCTGGTGGTGTGGCTTTGTTCGGAGGGCGCAAATTACACCA |
| | CCGGGCAGTCGTTGATAGTGGATGGCGGCTTTATGTTGGCG |
| | AATCCACAGTTCAACCCAGAATAG |
| | (SEQ ID NO: 34) |

TABLE 1-18
yeaE/ACT43604.1/Escherichia coli BL21(DE3)

| | |
|---|---|
| Amino | MQQKMIQFSGDVSLPAVGQGTWYMGEDASQRKTEVAALRAG |
| acid | IELGLTLIDTAEMYADGGAEKVVGEALTGLREKVFLVSKVY |
| sequence | PWNAGGQKAINACEASLRRLNTDYLDLYLLHWSGSFAFEET |
| | VAAMEKLIAQGKIRRWGVSNLDYADMQELWQLPGGNQCATN |
| | QVLYHLGSRGIEYDLLPWCQQQQMPVMAYSPLAQAGRLRNG |
| | LLKNAVVNEIAHAHNISAAQVLLAWVISHQGVMAIPKAATI |
| | AHVQQNAAVLEVELSSAELAMLDKAYPAPKGKTALDMV |
| | (SEQ ID NO: 35) |
| Base | ATGCAACAAAAAATGATTCAATTTAGTGGCGATGTCTCACT |
| sequence | GCCAGCCGTAGGGCAGGGAACATGGTATATGGGCGAAGATG |
| | CCAGTCAGCGCAAAACAGAAGTTGCTGCACTACGCGCGGGC |
| | ATTGAACTCGGTTTAACCCTCATTGATACCGCCGAAATGTA |
| | TGCCGATGGCGGTGCCGAAAAGGTGGTTGGGGAAGCATTAA |
| | CCGGTCTGCGAGAGAAGGTCTTTCTCGTCTCTAAAGTCTAT |
| | CCGTGGAATGCTGGCGGGCAAAAAGCGATAAATGCATGCGA |
| | AGCCAGTTTACGCCGTCTCAATACTGATTATCTCGATCTTT |
| | ACTTATTACACTGGTCTGGCAGTTTCGCTTTTGAAGAGACT |
| | GTCGCAGCGATGGAAAAATTGATCGCCCAGGGAAAAATCCG |
| | CCGCTGGGGCGTTTCTAACCTTGATTATGCTGATATGCAGG |
| | AACTCTGGCAGCTGCCGGGGGGAAATCAGTGTGCCACTAAT |
| | CAGGTGCTTTACCATCTCGGTTCACGAGGAATTGAGTACGA |
| | TCTACTCCCCTGGTGCCAGCAACAGCAGATGCCGGTGATGG |
| | CTTACAGTCCGTTAGCCCAGGCCGGGCGGTTGCGCAATGGA |
| | CTGTTAAAAAACGCGGTAGTCAACGAAATTGCACATGCTCA |
| | CAATATCAGCGCGGCACAAGTATTGTTGGCGTGGGTGATCA |
| | GTCATCAGGGTGTGATGGCGATTCCAAAAGCGGCCACGATT |
| | GCCCATGTCCAACAAAATGCGGCTGTGCTTGAGGTCGAACT |
| | TTCTTCAGCGGAATTAGCTATGCTGGATAAGGCATATCCGG |
| | CACCAAAAGGAAAAACTGCGCTGGATATGGTGTGA |
| | (SEQ ID NO: 36) |

TABLE 1-19
ADH1/NP_014555.1/Saccharomyces cerevisiae S288C

| | |
|---|---|
| Amino | MSIPETQKGVIFYESHGKLEYKDIPVPKPKANELLINVKYS |
| acid | GVCHTDLHAWHGDWPLPVKLPLVGGHEGAGVVVGMGENVKG |
| sequence | WKIGDYAGIKWLNGSCMACEYCELGNESNCPHADLSGYTHD |
| | GSFQQYATADAVQAAHIPQGTDLAQVAPILCAGITVYKALK |
| | SANLMAGHWVAISGAAGGLGSLAVQYAKAMGYRVLGIDGGE |
| | GKEELFRSIGGEVFIDFTKEKDIVGAVLKATDGGAHGVINV |
| | SVSEAAIEASTRYVRANGTTVLVGMPAGAKCCSDVFNQVVK |
| | SISIVGSYVGNRADTREALDFFARGLVKSPIKVVGLSTLPE |
| | IYEKMEKGQIVGRYVVDTSK |
| | (SEQ ID NO: 37) |
| Base | ATGTCTATCCCAGAAACTCAAAAAGGTGTTATCTTCTACGA |
| sequence | ATCCCACGGTAAGTTGGAATACAAAGATATTCCAGTTCCAA |
| | AGCCAAAGGCCAACGAATTGTTGATCAACGTTAAATACTCT |
| | GGTGTCTGTCACACTGACTTGCACGCTTGGCACGGTGACTG |
| | GCCATTGCCAGTTAAGCTACCATTAGTCGGTGGTCACGAAG |
| | GTGCCGGTGTCGTTGTCGGCATGGGTGAAAACGTTAAGGGC |
| | TGGAAGATCGGTGACTACGCCGGTATCAAATGGTTGAACGG |
| | TTCTTGTATGGCCTGTGAATACTGTGAATTGGGTAACGAAT |
| | CCAACTGTCCTCACGCTGACTTGTCTGGTTACACCCACGAC |
| | GGTTCTTTCCAACAATACGCTACCGCTGACGCTGTTCAAGC |
| | CGCTCACATTCCTCAAGGTACCGACTTGGCCCAAGTCGCCC |
| | CCATCTTGTGTGCTGGTATCACCGTCTACAAGGCTTTGAAG |
| | TCTGCTAACTTGATGGCCGGTCACTGGGTTGCTATCTCCGG |
| | TGCTGCTGGTGGTCTAGGTTCTTTGGCTGTTCAATACGCCA |
| | AGGCTATGGGTTACAGAGTCTTGGGTATTGACGGTGGTGAA |
| | GGTAAGGAAGAATTATTCAGATCCATCGGTGGTGAAGTCTT |
| | CATTGACTTCACTAAGGAAAAGGACATTGTCGGTGCTGTTC |
| | TAAAGGCCACTGACGGTGGTGCTCACGGTGTCATCAACGTT |
| | TCCGTTTCCGAAGCCGCTATTGAAGCTTCTACCAGATACGT |
| | TAGAGCTAACGGTACCACCGTTTTGGTCGGTATGCCAGCTG |
| | GTGCCAAGTGTTGTTCTGATGTCTTCAACCAAGTCGTCAAG |
| | TCCATCTCTATTGTTGGTTCTTACGTCGGTAACAGAGCTGA |
| | CACCAGAGAAGCTTTGGACTTCTTCGCCAGAGGTTTGGTCA |
| | AGTCTCCAATCAAGGTTGTCGGCTTGTCTACCTTGCCAGAA |
| | ATTTACGAAAAGATGGAAAAGGGTCAAATCGTTGGTAGATA |
| | CGTTGTTGACACTTCTAAATAA |
| | (SEQ ID NO: 38) |

TABLE 1-20
ADH2/NP_014032.1/Saccharomyces cerevisiae S288C

| | |
|---|---|
| Amino | MSIPETQKAIIFYESNGKLEHKDIPVPKPKPNELLINVKYS |
| acid | GVCHTDLHAWHGDWPLPTKLPLVGGHEGAGVVVGMGENVKG |
| sequence | WKIGDYAGIKWLNGSCMACEYCELGNESNCPHADLSGYTHD |
| | GSFQEYATADAVQAAHIPQGTDLAEVAPILCAGITVYKALK |
| | SANLRAGHWAAISGAAGGLGSLAVQYAKAMGYRVLGIDGGP |
| | GKEELFTSLGGEVFIDFTKEKDIVSAVVKATNGGAHGIINV |
| | SVSEAAIEASTRYCRANGTVVLVGLPAGAKCSSDVFNHVVK |
| | SISIVGSYVGNRADTREALDFFARGLVKSPIKVVGLSSLPE |
| | IYEKMEKGQIAGRYVVDTSK |
| | (SEQ ID NO: 39) |
| Base | ATGTCTATTCCAGAAACTCAAAAAGCCATTATCTTCTACGA |
| sequence | ATCCAACGGCAAGTTGGAGCATAAGGATATCCCAGTTCCAA |
| | AGCCAAAGCCCAACGAATTGTTAATCAACGTCAAGTACTCT |
| | GGTGTCTGCCACACCGATTTGCACGCTTGGCATGGTGACTG |
| | GCCATTGCCAACTAAGTTACCATTAGTTGGTGGTCACGAAG |
| | GTGCCGGTGTCGTTGTCGGCATGGGTGAAAACGTTAAGGGC |
| | TGGAAGATCGGTGACTACGCCGGTATCAAATGGTTGAACGG |
| | TTCTTGTATGGCCTGTGAATACTGTGAATTGGGTAACGAAT |
| | CCAACTGTCCTCACGCTGACTTGTCTGGTTACACCCACGAC |
| | GGTTCTTTCCAAGAATACGCTACCGCTGACGCTGTTCAAGC |
| | CGCTCACATTCCTCAAGGTACTGACTTGGCTGAAGTCGCGC |
| | CAATCTTGTGTGCTGGTATCACCGTATACAAGGCTTTGAAG |
| | TCTGCCAACTTGAGAGCAGGCCACTGGGCGGCCATTTCTGG |
| | TGCTGCTGGTGGTCTAGGTTCTTTGGCTGTTCAATATGCTA |
| | AGGCGATGGGTTACAGAGTCTTAGGTATTGATGGTGGTCCA |
| | GGAAAGGAAGAATTGTTTACCTCGCTCGGTGGTGAAGTATT |
| | CATCGACTTCACCAAAGAGAAGGACATTGTTAGCGCAGTCG |
| | TTAAGGCTACCAACGGCGGTGCCCACGGTATCATCAATGTT |
| | TCCGTTTCCGAAGCCGCTATCGAAGCTTCTACCAGATACTG |
| | TAGGGCGAACGGTACTGTTGTCTTGGTTGGTTTGCCAGCCG |
| | GTGCAAAGTGCTCCTCTGATGTCTTCAACCACGTTGTCAAG |
| | TCTATCTCCATTGTCGGCTCTTACGTGGGGAACAGAGCTGA |
| | TACCAGAGAAGCCTTAGATTTCTTTGCCAGAGGTCTAGTCA |
| | AGTCTCCAATAAAGGTAGTTGGCTTATCCAGTTTACCAGAA |
| | ATTTACGAAAAGATGGAGAAGGGCCAAATTGCTGGTAGATA |
| | CGTTGTTGACACTTCTAAATAA |
| | (SEQ ID NO: 40) |

TABLE 1-21
ADH3/NP_013800.1/Saccharomyces cerevisiae S288C

| | |
|---|---|
| Amino | MLRTSTLFTRRVQPSLFSRNILRLQSTAAIPKTQKGVIFYE |
| acid | NKGKLHYKDIPVPEPKPNEILINVKYSGVCHTDLHAWHGDW |
| sequence | PLPVKLPLVGGHEGAGVVVKLGSNVKGWKVGDLAGIKWLNG |
| | SCMTCEFCESGHESNCPDADLSGYTHDGSFQQFATADAIQA |
| | AKIQQGTDLAEVAPILCAGVTVYKALKEADLKAGDWVAISG |
| | AAGGLGSLAVQYATAMGYRVLGIDAGEEKEKLFKKLGGEVF |
| | IDFTKTKNMVSDIQEATKGGPHGVINVSVSEAAISLSTEYV |
| | RPCGTVVLVGLPANAYVKSEVFSHVVKSINIKGSYVGNRAD |
| | TREALDFFSRGLIKSPIKIVGLSELPKVYDLMEKGKILGRY |
| | VVDTSK |
| | (SEQ ID NO: 41) |
| Base | ATGTTGAGAACGTCAACATTGTTCACCAGGCGTGTCCAACC |
| sequence | AAGCCTATTTTCTAGAAACATTCTTAGATTGCAATCCACAG |
| | CTGCAATCCCTAAGACTCAAAAAGGTGTCATCTTTTATGAG |
| | AATAAGGGGAAGCTGCATTACAAAGATATCCCTGTCCCCGA |
| | GCCTAAGCCAAATGAAATTTTAATCAACGTTAAATATTCTG |
| | GTGTATGTCACACCGATTTACATGCTTGGCACGGCGATTGG |
| | CCATTACCTGTTAAACTACCATTAGTAGGTGGTCATGAAGG |
| | TGCTGGTGTAGTTGTCAAACTAGGTTCCAATGTCAAGGGCT |
| | GGAAAGTCGGTGATTTAGCAGGTATCAAATGGCTGAACGGT |
| | TCTTGTATGACATGCGAATTCTGTGAATCAGGTCATGAATC |
| | AAATTGTCCAGATGCTGATTTATCTGGTTACACTCATGATG |
| | GTTCTTTCCAACAATTTGCGACCGCTGATGCTATTCAAGCC |
| | GCCAAAATTCAACAGGGTACCGACTTGGCCGAAGTAGCCCC |
| | AATATTATGTGCTGGTGTTACTGTATATAAAGCACTAAAAG |
| | AGGCAGACTTGAAAGCTGGTGACTGGGTTGCCATCTCTGGT |
| | GCTGCAGGTGGCTTGGGTTCCTTGGCCGTTCAATATGCAAC |
| | TGCGATGGGTTACAGAGTTCTAGGTATTGATGCAGGTGAGG |
| | AAAAGGAAAAACTTTTCAAGAAATTGGGGGGTGAAGTATTC |
| | ATCGACTTTACTAAAACAAAGAATATGGTTTCTGACATTCA |
| | AGAAGCTACCAAAGGTGGCCCTCATGGTGTCATTAACGTTT |
| | CCGTTTCTGAAGCCGCTATTTCTCTATCTACGGAATATGTT |
| | AGACCATGTGGTACCGTCGTTTTGGTTGGTTTGCCCGCTAA |
| | CGCCTACGTTAAATCAGAGGTATTCTCTCATGTGGTGAAGT |
| | CCATCAATATCAAGGGTTCTTATGTTGGTAACAGAGCTGAT |
| | ACGAGAGAAGCCTTAGACTTCTTTAGCAGAGGTTTGATCAA |
| | ATCACCAATCAAAATTGTTGGATTATCTGAATTACCAAAGG |
| | TTTATGACTTGATGGAAAAGGGCAAGATTTTGGGTAGATAC |
| | GTCGTCGATACTAGTAAATAA |
| | (SEQ ID NO: 42) |

TABLE 1-22
ADH4/NP_011258.2/Saccharomyces cerevisiae S288C

| | |
|---|---|
| Amino | MSSVTGFYIPPISFFGEGALEETADYIKNKDYKKALIVTDP |
| acid | GIAAIGLSGRVQKMLEERDLNVAIYDKTQPNPNIANVTAGL |
| sequence | KVLKEQNSEIVVSIGGGSAHDNAKAIALLATNGGEIGDYEG |
| | VNQSKKAALPLFAINTTAGTASEMTRFTIISNEEKKIKMAI |
| | IDNNVTPAVAVNDPSTMFGLPPALTAATGLDALTHCIEAYV |
| | STASNPITDACALKGIDLINESLVAAYKDGKDKKARTDMCY |
| | AEYLAGMAFNNASLGYVHALAHQLGGFYHLPHGVCNAVLLP |
| | HVQEANMQCPKAKKRLGEIALHFGASQEDPEETIKALHVLN |
| | RTMNIPRNLKELGVKTEDFEILAEHAMHDACHLTNPVQFTK |
| | EQVVAIIKKAYEY |
| | (SEQ ID NO: 43) |
| Base | ATGTCTTCCGTTACTGGGTTTTACATTCCACCAATCTCTTT |
| sequence | CTTTGGTGAAGGTGCTTTAGAAGAAACCGCTGATTACATCA |
| | AAAACAAGGATTACAAAAAGGCTTTGATCGTTACTGATCCT |
| | GGTATTGCAGCTATTGGTCTCTCCGGTAGAGTCCAAAAGAT |
| | GTTGGAAGAACGTGACTTAAACGTTGCTATCTATGACAAAA |
| | CTCAACCAAACCCAAATATTGCCAATGTCACAGCTGGTTTG |
| | AAGGTTTTGAAGGAACAAAACTCTGAAATTGTTGTTTCCAT |
| | TGGTGGTGGTTCTGCTCACGACAATGCTAAGGCCATTGCTT |
| | TATTGGCTACTAACGGTGGGGAAATCGGAGACTATGAAGGT |
| | GTCAATCAATCTAAGAAGGCTGCTTTACCACTATTTGCCAT |
| | CAACACTACTGCTGGTACTGCTTCCGAAATGACCAGATTCA |
| | CTATTATCTCTAATGAAGAAAAGAAAATCAAGATGGCTATC |
| | ATTGACAACAACGTCACTCCAGCTGTTGCTGTCAACGATCC |
| | ATCTACCATGTTTGGTTTGCCACCTGCTTTGACTGCTGCTA |
| | CTGGTCTAGATGCTTTGACTCACTGTATCGAAGCTTATGTT |
| | TCCACCGCCTCTAACCCAATCACCGATGCCTGTGCTTTGAA |
| | GGGTATTGATTTGATCAATGAAAGCTTAGTCGCTGCATACA |
| | AAGACGGTAAAGACAAGAAGGCCAGAACTGACATGTGTTAC |
| | GCTGAATACTTGGCAGGTATGGCTTTCAACAATGCTTCTCT |
| | AGGTTATGTTCATGCCCTTGCTCATCAACTTGGTGGTTTCT |
| | ACCACTTGCCTCATGGTGTTTGTAACGCTGTCTTGTTGCCT |
| | CATGTTCAAGAGGCCAACATGCAATGTCCAAAGGCCAAGAA |
| | GAGATTAGGTGAAATTGCTTTGCATTTCGGTGCTTCTCAAG |
| | AAGATCCAGAAGAAACCATCAAGGCTTTGCACGTTTTAAAC |
| | AGAACCATGAACATTCCAAGAAACTTGAAAGAATTAGGTGT |
| | TAAAACCGAAGATTTTGAAATTTTGGCTGAACACGCCATGC |
| | ATGATGCCTGCCATTTGACTAACCCAGTTCAATTCACCAAA |
| | GAACAAGTGGTTGCCATTATCAAGAAAGCCTATGAATATTA |
| | A |
| | (SEQ ID NO: 44) |

TABLE 1-23
ADH5/NP_009703.3/Saccharomyces cerevisiae S288C

| | |
|---|---|
| Amino | MPSQVIPEKQKAIVFYETDGKLEYKDVTVPEPKPNEILVHV |
| acid | KYSGVCHSDLHAWHGDWPFQLKFPLIGGHEGAGVVVKLGSN |
| sequence | VKGWKVGDFAGIKWLNGTCMSCEYCEVGNESQCPYLDGTGF |
| | THDGTFQEYATADAVQAAHIPPNVNLAEVAPILCAGITVYK |
| | ALKRANVIPGQWVTISGACGGLGSLAIQYALAMGYRVIGID |
| | GGNAKRKLFEQLGGEIFIDFTEEKDIVGAIIKATNGGSHGV |
| | INVSVSEAAIEASTRYCRPNGTVVLVGMPAHAYCNSDVFNQ |
| | VVKSISIVGSCVGNRADTREALDFFARGLIKSPIHLAGLSD |
| | VPEIFAKMEKGEIVGRYWETSK |
| | (SEQ ID NO: 45) |
| Base | ATGCCTTCGCAAGTCATTCCTGAAAAACAAAAGGCTATTGT |
| sequence | CTTTTATGAGACAGATGGAAAATTGGAATATAAAGACGTCA |
| | CAGTTCCGGAACCTAAGCCTAACGAAATTTTAGTCCACGTT |
| | AAATATTCTGGTGTTTGTCATAGTGACTTGCACGCGTGGCA |
| | CGGTGATTGGCCATTTCAATTGAAATTTCCATTAATCGGTG |
| | GTCACGAAGGTGCTGGTGTTGTTGTTAAGTTGGGATCTAAC |
| | GTTAAGGGCTGGAAAGTCGGTGATTTTGCAGGTATAAAATG |
| | GTTGAATGGGACTTGCATGTCCTGTGAATATTGTGAAGTAG |
| | GTAATGAATCTCAATGTCCTTATTTGGATGGTACTGGCTTC |
| | ACACATGATGGTACTTTTCAAGAATACGCAACTGCCGATGC |
| | CGTTCAAGCTGCCCATATTCCACCAAACGTCAATCTTGCTG |
| | AAGTTGCCCCAATCTTGTGTGCAGGTATCACTGTTTATAAG |
| | GCGTTGAAAAGAGCCAATGTGATACCAGGCCAATGGGTCAC |
| | TATATCCGGTGCATGCGGTGGCTTGGGTTCTCTGGCAATCC |
| | AATACGCCCTTGCTATGGGTTACAGGGTCATTGGTATCGAT |
| | GGTGGTAATGCCAAGCGAAAGTTATTTGAACAATTAGGCGG |
| | AGAAATATTCATCGATTTCACGGAAGAAAAAGACATTGTTG |
| | GTGCTATAATAAAGGCCACTAATGGCGGTTCTCATGGAGTT |
| | ATTAATGTGTCTGTTTCTGAAGCAGCTATCGAGGCTTCTAC |
| | GAGGTATTGTAGGCCCAATGGTACTGTCGTCCTGGTTGGTA |
| | TGCCAGCTCATGCTTACTGCAATTCCGATGTTTTCAATCAA |
| | GTTGTAAAATCAATCTCCATCGTTGGATCTTGTGTTGGAAA |
| | TAGAGCTGATACAAGGGAGGCTTTAGATTTCTTCGCCAGAG |
| | GTTTGATCAAATCTCCGATCCACTTAGCTGGCCTATCGGAT |
| | GTTCCTGAAATTTTTGCAAAGATGGAGAAGGGTGAAATTGT |
| | TGGTAGATATGTTGTTGAGACTTCTAAATGA |
| | (SEQ ID NO: 46) |

TABLE 1-24
ADH6/NP_014051.3/Saccharomyces cerevisiae S288C

| | |
|---|---|
| Amino | MSYPEKFEGIAIQSHEDWKNPKKTKYDPKPFYDHDIDIKIE |
| acid | ACGVCGSDIHCAAGHWGNMKMPLVVGHEIVGKVVKLGPKSN |
| sequence | SGLKVGQRVGVGAQVFSCLECDRCKNDNEPYCTKFVTTYSQ |
| | PYEDGYVSQGGYANYVRVHEHFVVPIPENIPSHLAAPLLCG |
| | GLTVYSPLVRNGCGPGKKVGIVGLGGIGSMGTLISKAMGAE |
| | TYVISRSSRKREDAMKMGADHYIATLEEGDWGEKYFDTFDL |
| | IVVCASSLTDIDFNIMPKAMKVGGRIVSISIPEQHEMLSLK |
| | PYGLKAVSISYSALGSIKELNQLLKLVSEKDIKIWVETLPV |
| | GEAGVHEAFERMEKGDVRYRFTLVGYDKEFSD |
| | (SEQ ID NO: 47) |
| Base | ATGTCTTATCCTGAGAAATTTGAAGGTATCGCTATTCAATC |
| sequence | ACACGAAGATTGGAAAAACCCAAAGAAGACAAAGTATGACC |
| | CAAAACCATTTTACGATCATGACATTGACATTAAGATCGAA |
| | GCATGTGGTGTCTGCGGTAGTGATATTCATTGTGCAGCTGG |
| | TCATTGGGGCAATATGAAGATGCCGCTAGTCGTTGGTCATG |
| | AAATCGTTGGTAAAGTTGTCAAGCTAGGGCCCAAGTCAAAC |
| | AGTGGGTTGAAAGTCGGTCAACGTGTTGGTGTAGGTGCTCA |
| | AGTCTTTTCATGCTTGGAATGTGACCGTTGTAAGAATGATA |
| | ATGAACCATACTGCACCAAGTTTGTTACCACATACAGTCAG |
| | CCTTATGAAGACGGCTATGTGTCGCAGGGTGGCTATGCAAA |
| | CTACGTCAGAGTTCATGAACATTTTGTGGTGCCTATCCCAG |
| | AGAATATTCCATCACATTTGGCTGCTCCACTATTATGTGGT |
| | GGTTTGACTGTGTACTCTCCATTGGTTCGTAACGGTTGCGG |
| | TCCAGGTAAAAAAGTTGGTATAGTTGGTCTTGGTGGTATCG |
| | GCAGTATGGGTACATTGATTTCCAAAGCCATGGGGGCAGAG |
| | ACGTATGTTATTTCTCGTTCTTCGAGAAAAAGAGAAGATGC |
| | AATGAAGATGGGCGCCGATCACTACATTGCTACATTAGAAG |
| | AAGGTGATTGGGGTGAAAAGTACTTTGACACCTTCGACCTG |
| | ATTGTAGTCTGTGCTTCCTCCCTTACCGACATTGACTTCAA |
| | CATTATGCCAAAGGCTATGAAGGTTGGTGGTAGAATTGTCT |
| | CAATCTCTATACCAGAACAACACGAAATGTTATCGCTAAAG |
| | CCATATGGCTTAAAGGCTGTCTCCATTTCTTACAGTGCTTT |
| | AGGTTCCATCAAAGAATTGAACCAACTCTTGAAATTAGTCT |
| | CTGAAAAAGATATCAAAATTTGGGTGGAAACATTACCTGTT |
| | GGTGAAGCCGGCGTCCATGAAGCCTTCGAAAGGATGGAAAA |
| | GGGTGACGTTAGATATAGATTTACCTTAGTCGGCTACGACA |
| | AAGAATTTTCAGACTAG |
| | (SEQ ID NO: 48) |

TABLE 1-25
ADH7/NP_010030.1/Saccharomyces cerevisiae S288C

| | |
|---|---|
| Amino | MLYPEKFQGIGISNAKDWKHPKLVSFDPKPFGDHDVDVEIE |
| acid | ACGICGSDFHIAVGNWGPVPENQILGHEIIGRVVKVGSKCH |
| sequence | TGVKIGDRVGVGAQALACFECERCKSDNEQYCTNDHVLTMW |
| | TPYKDGYISQGGFASHVRLHEHFAIQIPENIPSPLAAPLLC |
| | GGITVFSPLLRNGCGPGKRVGIVGIGGIGHMGILLAKAMGA |
| | EVYAFSRGHSKREDSMKLGADHYIAMLEDKGWTEQYSNALD |
| | LLVVCSSSLSKVNFDSIVKIMKIGGSIVSIAAPEVNEKLVL |
| | KPLGLMGVSISSSAIGSRKEIEQLLKLVSEKNVKIWVEKLP |
| | ISEEGVSHAFTRMESGDVKYRFTLVDYDKKFHK |
| | (SEQ ID NO: 49) |
| Base | ATGCTTTACCCAGAAAAATTTCAGGGCATCGGTATTTCCAA |
| sequence | CGCAAAGGATTGGAAGCATCCTAAATTAGTGAGTTTTGACC |
| | CAAAACCCTTTGGCGATCATGACGTTGATGTTGAAATTGAA |
| | GCCTGTGGTATCTGCGGATCTGATTTTCATATAGCCGTTGG |
| | TAATTGGGGTCCAGTCCCAGAAAATCAAATCCTTGGACATG |
| | AAATAATTGGCCGCGTGGTGAAGGTTGGATCCAAGTGCCAC |
| | ACTGGGGTAAAAATCGGTGACCGTGTTGGTGTTGGTGCCCA |
| | AGCCTTGGCGTGTTTTGAGTGTGAACGTTGCAAAAGTGACA |
| | ACGAGCAATACTGTACCAATGACCACGTTTTGACTATGTGG |
| | ACTCCTTACAAGGACGGCTACATTTCACAAGGAGGCTTTGC |
| | CTCCCACGTGAGGCTTCATGAACACTTTGCTATTCAAATAC |
| | CAGAAAATATTCCAAGTCCGCTAGCCGCTCCATTATTGTGT |
| | GGTGGTATTACAGTTTTCTCTCCACTACTAAGAAATGGCTG |
| | TGGTCCAGGTAAGAGGGTAGGTATTGTTGGCATCGGTGGTA |
| | TTGGGCATATGGGGATTCTGTTGGCTAAAGCTATGGGAGCC |
| | GAGGTTTATGCGTTTTCGCGAGGCCACTCCAAGCGGGAGGA |
| | TTCTATGAAACTCGGTGCTGATCACTATATTGCTATGTTGG |
| | AGGATAAAGGCTGGACAGAACAATACTCTAACGCTTTGGAC |
| | CTTCTTGTCGTTTGCTCATCATCTTTGTCGAAAGTTAATTT |
| | TGACAGTATCGTTAAGATTATGAAGATTGGAGGCTCCATCG |
| | TTTCAATTGCTGCTCCTGAAGTTAATGAAAAGCTTGTTTTA |
| | AAACCGTTGGGCCTAATGGGAGTATCAATCTCAAGCAGTGC |
| | TATCGGATCTAGGAAGGAAATCGAACAACTATTGAAATTAG |
| | TTTCCGAAAAGAATGTCAAAATATGGGTGGAAAAACTTCCG |
| | ATCAGCGAAGAAGGCGTCAGCCATGCCTTTACAAGGATGGA |
| | AAGCGGAGACGTCAAATACAGATTTACTTTGGTCGATTATG |
| | ATAAGAAATTCCATAAATAG |
| | (SEQ ID NO: 50) |

TABLE 1-26
SFA1/NP_010113.1/Saccharomyces cerevisiae S288C

| | |
|---|---|
| Amino acid | MSAATVGKPIKCIAAVAYDAKKPLSVEEITVDAPKAHEVRIKIEYTAVCHTDAYTLSGS |
| sequence | DPEGLFPCVLGHEGAGIVESVGDDVITVKPGDHVIALYTAECGKCKFCTSGKTNLCG |
| | AVRATQGKGVMPDGTTRFHNAKGEDIYHFMGCSTFSEYTVVADVSVVAIDPKAPLDA |
| | ACLLGCGVTTGFGAALKTANVQKGDTVAVFGCGTVGLSVIQGAKLRGASKIIAIDINN |
| | KKKQYCSQFGATDFVNPKEDLAKDQTIVEKLIEMTDGGLDFTFDCTGNTKIMRDALE |
| | ACHKGWGQSIIIGVAAAGEEISTRPFQLVTGRVWKGSAFGGIKGRSEMGGLIKDYQK |
| | GALKVEEFITHRRPFKEINQAFEDLHNGDCLRTVLKSDEIK (SEQ ID NO: 51) |
| Base | ATGTCCGCCGCTACTGTTGGTAAACCTATTAAGTGCATTGCTGCTGTTGCGTATGAT |
| sequence | GCGAAGAAACCATTAAGTGTTGAAGAAATCACGGTAGACGCCCCAAAAGCGCAC |
| | GAAGTACGTATCAAAATTGAATATACTGCTGTATGCCACACTGATGCGTACACTTTA |
| | TCAGGCTCTGATCCAGAAGGACTTTTCCCTTGCGTTCTGGGCCACGAAGGAGCCG |
| | GTATCGTAGAATCTGTAGGCGATGATGTCATAACAGTTAAGCCTGGTGATCATGTTA |
| | TTGCTTTGTACACTGCTGAGTGTGGCAAATGTAAGTTCTGTACTTCCGGTAAAACC |
| | AACTTATGTGGTGCTGTTAGAGCTACTCAAGGGAAAGGTGTAATGCCTGATGGGAC |
| | CACAAGATTTCATAATGCGAAAGGTGAAGATATATACCATTTCATGGGTTGCTCTAC |
| | tttttccgaatatactgtggtggcagatgtctctgtggttgccatcgatccaaaagc |
| | TCCCTTGGATGCTGCCTGTTTACTGGGTTGTGGTGTTACTACTGGTTTTGGGGCGG |
| | CTCTTAAGACAGCTAATGTGCAAAAAGGCGATACCGTTGCAGTATTTGGCTGCGGG |
| | ACTGTAGGACTCTCCGTTATCCAAGGTGCAAAGTTAAGGGGCGCTTCCAAGATCAT |
| | TGCCATTGACATTAACAATAAGAAAAAACAATATTGTTCTCAATTTGGTGCCACGG |
| | ATTTTGTTAATCCCAAGGAAGATTTGGCCAAAGATCAAACTATCGTTGAAAAGTTA |
| | ATTGAAATGACTGATGGGGGTCTGGATTTTACTTTTGACTGTACTGGTAATACCAAA |
| | ATTATGAGAGATGCTTTGGAAGCCTGTCATAAAGGTTGGGGTCAATCTATTATCATT |
| | GGTGTGGCTGCCGCTGGTGAAGAAATTTCTACAAGGCCGTTCCAGCTGGTCACTG |
| | GTAGAGTGTGGAAAGGCTCTGCTTTTGGTGGCATCAAAGGTAGATCTGAAATGGG |
| | CGGTTTAATTAAAGACTATCAAAAAGGTGCCTTAAAAGTCGAAGAATTTATCACTC |
| | ACAGGAGACCATTCAAAGAAATCAATCAAGCCTTTGAAGATTTGCATAACGGTGA |
| | TTGCTTAAGAACCGTCTTGAAGTCTGATGAAATAAAATAG (SEQ ID NO: 52) |

TABLE 1-27
AAD3/NP_010032.1/Saccharomyces cerevisiae S288C

| | |
|---|---|
| Amino acid | MIGSASDSSSKLGRLRFLSETAAIKVSPLILGEVSYDGARSDFLKSMNKNRAFELLDTF |
| sequence | YEAGGNFIDAANNCQNEQSEEWIGEWIQSRRLRDQIVIATKFIKSDKKYKAGESNTAN |
| | YCGNHKRSLHVSVRDSLRKLQTDWIDILYVHWWDYMSSIEEFMDSLHILVQQGKVL |
| | YLGVSDTPAWVVSAANYYATSYGKTPFSIYQGKWNVLNRDFERDIIPMARHFGMAL |
| | APWDVMGGGRFQSKKAMEERRKNGEGIRSFVGASEQTDAEIKISEALAKIAEEHGTE |
| | SVTAIAIAYVRSKAKNFFPSVEGGKIEDLKENIKALSIDLTPDNIKYLESIVPFDIGFPNN |
| | FIVLNSLTQKYGTNNV (SEQ ID NO: 53) |
| Base | ATGATTGGGTCCGCGTCCGACTCATCTAGCAAGTTAGGACGCCTCCGATTTCTTTCT |
| sequence | GAAACTGCCGCTATTAAAGTATCCCCGTTAATCCTAGGAGAAGTCTCATACGATGG |
| | AGCACGTTCGGATTTTCTCAAATCAATGAACAAGAATCGAGCTTTTGAATTGCTTG |
| | ATACTTTTTACGAGGCAGGTGGAAATTTCATTGATGCCGCAAACAACTGCCAAAAC |
| | GAGCAATCAGAAGAATGGATTGGTGAATGGATACAGTCCAGAAGGTTACGTGATC |
| | AAATTGTCATTGCAACCAAGTTTATAAAAAGCGATAAAAAGTATAAAGCAGGTGA |
| | AAGTAACACTGCCAACTACTGTGGTAATCACAAGCGTAGTTTACATGTGAGTGTGA |
| | GGGATTCTCTCCGCAAATTGCAAACTGATTGGATTGATATACTTTACGTTCACTGGT |
| | GGGATTATATGAGTTCAATCGAAGAATTTATGGATAGTTTGCATATTCTGGTCCAGC |
| | AGGGCAAGGTCCTCTATTTGGGTGTATCTGATACACCTGCTTGGGTTGTTTCTGCG |
| | GCAAACTACTACGCTACATCTTATGGTAAAACTCCCTTTAGTATCTACCAAGGTAAA |
| | TGGAACGTGTTGAACAGAGATTTTGAGCGTGATATTATTCCAATGGCTAGGCATTT |
| | CGGTATGGCCCTCGCCCCATGGGATGTCATGGGAGGTGGAAGATTTCAGAGTAAA |
| | AAAGCAATGGAGGAACGGAGGAAGAATGGAGAGGGTATTCGTTCTTTCGTTGGCG |
| | CCTCCGAACAAACAGATGCAGAAATCAAGATTAGTGAAGCATTGGCCAAGATTGC |
| | TGAGGAACATGGCACTGAGTCTGTTACTGCTATTGCTATTGCCTATGTTCGCTCTAA |
| | GGCGAAAAATTTTTTTCCGTCGGTTGAAGGAGGAAAAATTGAGGATCTCAAAGAG |
| | AACATTAAGGCTCTCAGTATCGATCTAACGCCAGACAATATAAAATACTTAGAAAG |
| | TATAGTTCCTTTTGACATCGGATTTCCTAATAATTTTATCGTGTTAAATTCCTTGACT |
| | CAAAAATATGGTACGAATAATGTTTAG (SEQ ID NO: 54) |

TABLE 1-28
AAD4/NP_010038.1/Saccharomyces cerevisiae S288C

| | |
|---|---|
| Amino acid | MGSMNKEQAFELLDAFYEAGGNCIDTANSYQNEESEIWIGEWMKSRKLRDQIVIATK |
| sequence | FTGDYKKYEVGGGKSANYCGNHKHSLHVSVRDSLRKLQTDWIDILYVHWWDYMSS |
| | IEEVMDSLHILVQQGKVLYLGVSDTPAWVVSAANYYATSHGKTPFSIYQGKWNVLNR |
| | DFERDIIPMARHFGMALAPWDVMGGGRFQSKKAMEERKKNGEGLRTVSGTSKQTD |
| | KEVKISEALAKVAEEHGTESVTAIAIAYVRSKAKNVFPLVGGRKIEHLKQNIEALSIKL |
| | TPEQIEYLESIIPFDVGFPTNFIGDDPAVTKKASLLTAMSAQISFD (SEQ ID NO: 55) |
| Base | ATGGGCTCTATGAATAAGGAACAGGCTTTTGAACTTCTTGATGCTTTTTATGAAGC |
| sequence | AGGAGGTAATTGCATTGATACTGCAAACAGTTACCAAAATGAAGAGTCAGAGATT |
| | TGGATAGGTGAATGGATGAAATCAAGAAAGTTGCGTGACCAAATTGTAATTGCCAC |
| | CAAGTTTACCGGAGATTATAAGAAGTATGAAGTAGGTGGCGGTAAAAGTGCCAAC |
| | TATTGTGGTAATCACAAGCATAGTTTACATGTGAGTGTGAGGGATTCTCTCCGCAA |
| | ATTGCAAACTGATTGGATTGATATACTTTACGTTCACTGGTGGGATTATATGAGTTC |
| | AATCGAAGAAGTTATGGATAGTTTGCATATTTTAGTTCAGCAGGGCAAAGTCCTCT |
| | ATTTGGGTGTGTCTGATACACCTGCTTGGGTTGTTTCTGCGGCAAACTACTACGCC |
| | ACATCTCATGGGAAAACTCCTTTTAGTATCTATCAAGGTAAATGGAATGTGTTGAA |
| | CAGGGACTTTGAGCGCGATATCATTCCAATGGCCAGACATTTTGGTATGGCTCTAG |
| | CCCCATGGGATGTTATGGGAGGTGGAAGATTTCAGAGTAAAAAAGCAATGGAGGA |
| | ACGGAAGAAGAATGGAGAGGGTCTGCGTACTGTTTCGGGTACTTCTAAACAGACG |
| | GATAAAGAGGTTAAGATCAGTGAAGCATTGGCCAAGGTTGCTGAGGAACATGGCA |
| | CTGAGTCTGTTACTGCTATTGCTATTGCCTATGTTCGCTCTAAGGCGAAAAATGTTT |
| | TCCCATTGGTTGGTGGAAGGAAAATTGAACACCTCAAACAGAACATTGAGGCTTT |
| | AAGTATCAAACTGACACCAGAACAGATAGAATACTTAGAAAGTATTATTCCTTTTG |
| | ATGTTGGTTTTCCTACTAATTTTATCGGTGATGATCCGGCTGTTACCAAGAAGGCTT |
| | CACTTCTCACGGCAATGTCTGCGCAGATTTCCTTCGATTAA (SEQ ID NO: 56) |

TABLE 1-29
AAD10/NP_012689.1/Saccharomyces cerevisiae S288C

| | |
|---|---|
| Amino acid | MASRKLRDQIVIATKFTTDYKGYDVGKGKSANFCGNHKRSLHVSVRDSLRKLQTDW |
| sequence | IDILYVHWWDYMSSIEEVMDSLHILVQQGKVLYLGVSDTPAWVVSAANYYATSHGKT |
| | PFSIYQGKWNVLNRDFERDIIPMARHFGMALAPWDVMGGGRFQSKKAVEERKKKGE |
| | GLRTFFGTSEQTDMEVKISEALLKVAEEHGTESVTAIAIAYVRSKAKHVFPLVGGRKIE |
| | HLKQNIEALSIKLTPEQIKYLESIVPFDVGFPTNFIGDDPAVTKKPSFLTEMSAKISFED |
| | (SEQ ID NO: 57) |
| Base | ATGGCATCAAGAAAACTGCGTGACCAGATTGTAATTGCCACTAAATTTACCACGGA |
| sequence | TTATAAGGGGTATGATGTAGGCAAGGGGAAGAGTGCCAATTTCTGTGGGAATCACA |
| | AGCGCAGTTTGCATGTAAGTGTGAGAGATTCCCTTCGTAAGTTGCAAACTGATTGG |
| | ATTGATATTCTTTACGTTCACTGGTGGGATTATATGAGCTCCATTGAGGAAGTTATG |
| | GATAGTTTGCACATTCTTGTGCAGCAGGGCAAAGTACTCTATCTAGGTGTGTCTGA |
| | TACTCCTGCCTGGGTTGTTTCTGCAGCAAATTACTACGCCACATCTCATGGTAAAA |
| | CTCCCTTTAGTATCTATCAAGGTAAATGGAATGTATTGAACAGGGACTTTGAACGT |
| | GATATCATTCCAATGGCTAGGCATTTTGGTATGGCTCTTGCTCCATGGGATGTTATG |
| | GGAGGCGGGAGATTTCAGAGTAAAAAGGCAGTGGAAGAGCGGAAGAAGAAAGG |
| | AGAAGGCTTGCGTACCTTTTTTGGTACTTCGGAACAGACGGATATGGAGGTTAAAA |
| | TCAGCGAAGCATTGTTAAAAGTTGCGGAAGAACATGGCACTGAGTCTGTCACTGC |
| | TATTGCCATAGCTTATGTTCGGTCTAAAGCGAAACATGTTTTCCCATTAGTGGGAGG |
| | AAGAAAGATCGAACATCTCAAACAGAACATTGAGGCTTTGAGCATTAAATTAACA |
| | CCAGAACAAATAAAGTACTTAGAAAGTATTGTTCCTTTTGATGTCGGATTTCCCAC |
| | TAATTTTATTGGAGATGACCCAGCTGTTACCAAGAAACCTTCATTTCTCACCGAAAT |
| | GTCTGCCAAGATTAGCTTCGAAGATTAG (SEQ ID NO: 58) |

TABLE 1-30
AAD14/NP_014068.1/Saccharomyces cerevisiae S288C

| | |
|---|---|
| Amino acid | MTDLFKPLPEPPTELGRLRVLSKTAGIRVSPLILGGASIGDAWSGFMGSMNKEQAFEL |
| sequence | LDAFYEAGGNCIDTANSYQNEESEIWIGEWMASRKLRDQIVIATKFTGDYKKYEVGG |
| | GKSANYCGNHKRSLHVSVRDSLRKLQTDWIDILYIHWWDYMSSIEEVMDSLHILVQQ |
| | GKVLYLGVSDTPAWVVSAANYYATSHGKTPFSVYQGKWNVLNRDFERDIIPMARHF |
| | GMALAPWDVMGGGRFQSKKAMEERKKNGEGLRTFVGGPEQTELEVKISEALTKIAE |
| | EHGTESVTAIAIAYVRSKAKNVFPLIGGRKIEHLKQNIEALSIKLTPEQIEYLESIVPFDV |
| | GFPKSLIGDDPAVTKKLSPLTSMSARIAFDN (SEQ ID NO: 59) |
| Base | ATGACTGACTTGTTTAAACCTCTACCTGAACCACCTACCGAATTGGGACGTCTCAG |
| sequence | GGTTCTTTCTAAAACTGCCGGCATAAGGGTTTCACCGCTAATTCTGGGAGGAGCTT |
| | CAATCGGCGACGCATGGTCAGGCTTTATGGGCTCTATGAATAAGGAACAGGCCTTT |
| | GAACTTCTTGATGCTTTTTATGAAGCTGGAGGTAATTGTATTGATACTGCAAACAGT |
| | TACCAAAATGAAGAGTCAGAGATTTGGATAGGTGAATGGATGGCATCAAGAAAAC |
| | TGCGTGACCAGATTGTAATTGCCACCAAGTTTACCGGAGATTATAAGAAGTATGAA |
| | GTAGGTGGTGGTAAAAGTGCCAACTACTGTGGTAATCACAAGCGTAGTTTACATGT |
| | GAGTGTGAGGGATTCTCTCCGCAAATTGCAAACTGATTGGATTGATATACTTTACAT |
| | TCACTGGTGGGATTATATGAGTTCAATCGAAGAAGTTATGGATAGTTTGCATATTTT |
| | AGTTCAGCAGGGCAAGGTCCTATATTTAGGAGTATCTGATACACCTGCTTGGGTTG |
| | TTTCTGCGGCAAATTACTACGCTACATCTCATGGTAAAACTCCTTTTAGCGTCTATC |
| | AAGGTAAATGGAATGTATTGAACAGGGACTTTGAGCGTGATATTATTCCAATGGCT |
| | AGGCATTTTGGTATGGCTCTAGCCCCATGGGATGTCATGGGAGGTGGAAGATTTCA |
| | GAGTAAAAAAGCAATGGAAGAACGGAAGAAGAATGGAGAGGGTCTGCGTACTTT |
| | TGTGGGTGGCCCCGAACAAACAGAATTGGAGGTTAAAATCAGCGAAGCATTGACT |
| | AAAATTGCTGAGGAACATGGAACAGAGTCTGTTACTGCTATCGCTATTGCCTATGT |
| | TCGCTCTAAAGCGAAAAATGTTTTCCCATTGATTGGAGGAAGGAAAATTGAACATC |
| | TCAAGCAGAACATTGAGGCTTTGAGTATTAAATTAACACCGGAACAAATAGAATAC |
| | CTGGAAAGTATTGTTCCTTTTGATGTTGGCTTTCCCAAAAGTTTAATAGGAGATGA |
| | CCCAGCGGTAACCAAGAAGCTTTCACCCCTCACATCGATGTCTGCCAGGATAGCTT |
| | TTGACAATTAG (SEQ ID NO: 60) |

TABLE 1-31
AAD15/NP_014477.1/Saccharomyces cerevisiae S288C

| | |
|---|---|
| Amino acid | MARHFGMALAPWDVMGGGRFQSKKAMEERRKNGECIRSFVGASEQTDAEIKISEAL |
| sequence | AKVAEEHGTESVTAIAIAYVRSKAKNVFPSVEGGKIEDLKENIKALSIDLTPDNIKYLE |
| | NVVPFDIGFPNTFIVLNSLTQKYGTNNV (SEQ ID NO: 61) |
| Base | ATGGCTAGGCATTTCGGTATGGCCCTCGCCCCATGGGATGTCATGGGAGGTGGAAG |
| sequence | ATTTCAGAGTAAAAAAGCAATGGAGGAACGGAGGAAGAATGGAGAGTGTATTCGT |
| | TCTTTCGTTGGCGCCTCCGAACAAACAGATGCAGAAATCAAGATTAGTGAAGCAT |
| | TAGCCAAGGTTGCTGAGGAACATGGCACTGAGTCTGTTACTGCTATTGCTATTGCC |
| | TATGTTCGCTCTAAGGCGAAAAATGTTTTTCCGTCGGTTGAAGGAGGAAAAATTGA |
| | GGATCTCAAAGAGAACATTAAGGCTCTCAGTATCGATCTAACGCCGGACAATATAA |
| | AATACTTGGAAAATGTAGTTCCTTTTGACATCGGATTTCCTAACACTTTTATCGTGT |
| | TAAATTCCTTGACTCAAAAATATGGTACGAATAATGTTTAG (SEQ ID NO: 62) |

TABLE 1-32
YPR1/NP_010656.1/Saccharomyces cerevisiae S288C

| | |
|---|---|
| Amino acid | MPATLKNSSATLKLNTGASIPVLGFGTWRSVDNNGYHSVIAALKAGYRHIDAAAIYL |
| sequence | NEEEVGRAIKDSGVPREEIFITTKLWGTEQRDPEAALNKSLKRLGLDYVDLYLMHWP |
| | VPLKTDRVTDGNVLCIPTLEDGTVDIDTKEWNFIKTWELMQELPKTGKTKAVGVSNF |
| | SINNIKELLESPNNKVVPATNQIEIHPLLPQDELIAFCKEKGIVVEAYSPFGSANAPLLK |
| | EQAIIDMAKKHGVEPAQLIISWSIQRGYVVLAKSVNPERIVSNFKIFTLPEDDFKTISNL |
| | SKVHGTKRVVDMKWGSFPIFQ (SEQ ID NO: 63) |
| Base | ATGCCTGCTACGTTAAAGAATTCTTCTGCTACATTAAAACTAAATACTGGTGCCTCC |
| sequence | ATTCCAGTGTTGGGTTTCGGCACTTGGCGTTCCGTTGACAATAACGGTTACCATTC |
| | TGTAATTGCAGCTTTGAAAGCTGGATACAGACACATTGATGCTGCGGCTATCTATTT |
| | GAATGAAGAAGAAGTTGGCAGGGCTATTAAAGATTCCGGAGTCCCTCGTGAGGAA |
| | ATTTTTATTACTACTAAGCTTTGGGGTACGGAACAACGTGATCCGGAAGCTGCTCT |
| | AAACAAGTCTTTGAAAAGACTAGGCTTGGATTATGTTGACCTATATCTGATGCATTG |
| | GCCAGTGCCTTTGAAAACCGACAGAGTTACTGATGGTAACGTTCTGTGCATTCCAA |
| | CATTAGAAGATGGCACTGTTGACATCGATACTAAGGAATGGAATTTTATCAAGACG |
| | TGGGAGTTGATGCAAGAGTTGCCAAAGACGGGCAAAACTAAAGCCGTTGGTGTC |
| | TCTAATTTTTCTATTAACAACATTAAAGAATTATTAGAATCTCCAAATAACAAGGTG |
| | GTACCAGCTACTAATCAAATTGAAATTCATCCATTGCTACCACAAGACGAATTGATT |
| | GCCTTTTGTAAGGAAAAGGGTATTGTTGTTGAAGCCTACTCACCATTTGGGAGTGC |
| | TAATGCTCCTTTACTAAAAGAGCAAGCAATTATTGATATGGCTAAAAAGCACGGCG |
| | TTGAGCCAGCACAGCTTATTATCAGTTGGAGTATTCAAAGAGGCTACGTTGTTCTG |
| | GCCAAATCGGTTAATCCTGAAAGAATTGTATCCAATTTTAAGATTTTCACTCTGCCT |
| | GAGGATGATTTCAAGACTATTAGTAACCTATCCAAAGTGCATGGTACAAAGAGAGT |
| | CGTTGATATGAAGTGGGGATCCTTCCCAATTTTCCAATGA (SEQ ID NO: 64) |

TABLE 1-33
NCgl0324/NP_599582.1/Corynebacterium glutamicum ATCC 13032

| | |
|---|---|
| Amino acid | MSISVKALQKSGPEAPFEVKIIERRDPRADDVVIDIKAAGICHSDIHTIRNEWGEAHFP |
| sequence | LTVGHEIAGVVSAVGSDVTKWKVGDRVGVGCLVNSCGECEQCVAGFENNCLRGNV |
| | GTYNSNDVDGTITQGGYAEKVVVNERFLCSIPEELNFDVAAPLLCAGITTYSPIARWN |
| | VKEGDKVAVMGLGGLGHMGVQIAAAKGAEVTVLSRSLRKAELAKELGAARTLATS |
| | DEDFFTEHAGEFDFILNTISASIPVDKYLSLLKPHGVMAVVGLPPEKQPLSFGALIGGG |
| | KVLTGSNIGGIPETQEMLDFCAKHGLGAMIETVGVNDVDAAYDRVVAGDVQFRVVID |
| | TASFAEVEAV (SEQ ID NO: 65) |
| Base | GTGAGTATCTCAGTAAAAGCACTACAAAAGTCCGGCCCAGAAGCACCTTTCGAGG |
| sequence | TCAAGATCATTGAACGCCGTGACCCACGCGCAGATGATGTGGTTATTGATATCAAA |
| | GCTGCGGGCATCTGCCACAGCGATATCCACACCATCCGCAACGAATGGGGCGAGG |
| | CGCACTTCCCGCTCACCGTCGGCCACGAAATCGCAGGCGTTGTCTCTGCGGTTGG |
| | ATCCGATGTAACCAAATGGAAAGTCGGCGACCGCGTGGGCGTCGGCTGCCTCGTT |
| | AACTCCTGCGGCGAATGCGAACAGTGCGTCGCAGGATTTGAAAACAACTGCCTTC |
| | GCGGAAACGTCGGAACCTACAACTCTAACGACGTCGACGGCACCATCACCCAAG |
| | GCGGCTACGCTGAAAAGGTAGTGGTCAACGAACGTTTCCTGTGCAGCATCCCAGA |
| | GGAACTTAACTTCGATGTCGCAGCACCACTGCTGTGCGCAGGCATCACCACCTACT |
| | CCCCAATCGCTCGCTGGAACGTTAAAGAAGGCGACAAAGTAGCAGTCATGGGCCT |
| | CGGCGGACTCGGACACATGGGTGTCCAGATCGCTGCAGCCAAGGGTGCTGAGGTT |
| | ACCGTTCTGTCCCGTTCCCTGCGCAAGGCAGAACTTGCCAAGGAACTCGGCGCAG |
| | CTCGCACGCTTGCGACTTCTGATGAGGATTTCTTCACCGAACACGCCGGTGAATTC |
| | GACTTCATCCTCAACACCATTAGCGCATCCATCCCAGTCGACAAGTACCTGAGCCT |
| | TCTCAAGCCACACGGTGTCATGGCTGTTGTCGGTCTGCCACCAGAGAAGCAGCCA |
| | CTGAGCTTCGGTGCGCTCATCGGCGGCGGAAAAGTCCTCACCGGATCCAACATTG |
| | GCGGCATCCCTGAAACCCAGGAAATGCTCGACTTCTGTGCAAAACACGGCCTCGG |
| | TGCGATGATCGAAACTGTCGGCGTCAACGATGTTGATGCAGCCTACGACCGTGTTG |
| | TTGCCGGCGACGTTCAGTTCCGCGTTGTCATTGATACTGCTTCGTTTGCTGAGGTT |
| | GAGGCGGTTTAG (SEQ ID NO: 66) |

TABLE 1-34
NCgl0313/NP_599571.1/Corynebacterium glutamicum ATCC 13032

| | |
|---|---|
| Amino acid | MSTVVPGIVALSKGAPVEKVNVVVPDPGANDVIVKIQACGVCHTDLAYRDGDISDEF |
| sequence | PYLLGHEAAGIVEEVGESVTHVEVGDFVILNWRAVCGECRACKKGEPKYCFNTHNA |
| | SKKMTLEDGTELSPALGIGAFLEKTLVHEGQCTKVNPEEDPAAAGLLGCGIMAGLGA |
| | AVNTGDIKRGESVAVFGLGGVGMAAIAGAKIAGASKIIAVDIDEKKLEWAKEFGATHT |
| | INSSGLGGEGDASEVVAKVRELTDGFGTDVSIDAVGIMPTWQQAFYSRDHAGRMVM |
| | VGVPNLTSRVDVPAIDFYGRGGSVRPAWYGDCLPERDFPTYVDLHLQGRFPLDKFVS |
| | ERIGLDDVEEAFNTMKAGDVLRSVVEI (SEQ ID NO: 67) |
| Base | ATGAGCACTGTAGTGCCTGGAATTGTCGCACTGTCCAAGGGTGCACCGGTAGAAA |
| sequence | AAGTAAACGTTGTTGTCCCTGATCCAGGTGCTAACGATGTCATCGTCAAGATTCAG |
| | GCCTGCGGTGTGTGCCACACCGACTTGGCCTACCGCGATGGCGATATTTCAGATGA |
| | GTTCCCTTACCTCCTCGGCCACGAGGCAGCAGGCATTGTTGAGGAGGTAGGCGAG |
| | TCCGTCACCCACGTTGAGGTCGGCGATTTCGTCATCTTGAACTGGCGTGCAGTGTG |
| | CGGCGAGTGCCGTGCATGTAAGAAGGGCGAGCCAAAGTACTGCTTTAACACCCAC |
| | AACGCCTCTAAGAAGATGACCCTGGAAGACGGCACCGAGCTGTCCCCAGCACTG |
| | GGTATTGGCGCGTTCTTGGAAAAGACCCTGGTCCACGAAGGCCAGTGCACCAAGG |
| | TTAACCCTGAGGAAGATCCAGCAGCAGCTGGCCTTCTGGGTTGTGGCATCATGGC |
| | AGGCCTTGGCGCTGCGGTGAACACCGGTGATATTAAGCGTGGCGAGTCCGTAGCA |
| | GTCTTCGGCCTTGGTGGCGTGGGCATGGCAGCTATTGCTGGCGCCAAGATTGCTGG |
| | CGCTTCCAAGATCATTGCTGTTGATATCGATGAGAAGAAGCTGGAGTGGGCGAAG |
| | GAATTCGGCGCAACCCACACCATTAATTCCTCTGGTCTTGGTGGCGAAGGTGATGC |
| | CTCTGAGGTCGTGGCAAAGGTTCGTGAGCTCACCGATGGTTTCGGCACCGATGTC |
| | TCCATCGATGCGGTAGGCATCATGCCGACCTGGCAGCAGGCGTTTTACTCCCGTGA |
| | CCATGCAGGCCGCATGGTGATGGTGGGCGTTCCAAACCTGACGTCTCGCGTAGAT |
| | GTTCCTGCGATTGATTTTTACGGTCGCGGTGGATCCGTGCGCCCTGCATGGTACGG |
| | CGACTGCCTGCCTGAGCGTGATTTCCCAACTTATGTGGATCTGCACCTGCAGGGTC |
| | GTTTCCCACTGGATAAGTTTGTTTCTGAGCGTATTGGTCTTGATGATGTTGAAGAG |
| | GCTTTCAACACCATGAAGGCTGGCGACGTGCTGCGTTCTGTGGTGGAGATCTAA |
| | (SEQ ID NO: 68) |

TABLE 1-35
NCgl0219/NP_599475.1/Corynebacterium glutamicum ATCC 13032

| | |
|---|---|
| Amino acid | MPKYIAMQVSESGAPLAANLVQPAPLKSREVRVEIAASGVCHADIGTAAASGKHTVF |
| sequence | PVTPGHEIAGTIAEIGENVSRWTVGDRVAIGWFGGNCGDCAFCRAGDPVHCRERKIP |
| | GVSYAGGWAQNIVVPAEALAAIPDGMDFYEAAPMGCAGVTTFNALRNLKLDPGAAV |
| | AVFGIGGLVRLAIQFAAKMGYRTITIARGLEREELARQLGANHYIDSNDLHPGQALFE |
| | LGGADLILSTASTTEPLSELSTGLSIGGQLTIIGVDGGDITVSAAQLMMNRQIITGHLTG |
| | SANDTEQTMKFAHLHGVKPLIERMPLDQANEAIARISAGKPRFRIVLEPNS (SEQ ID |
| | NO: 69) |
| Base | ATGCCCAAATACATTGCCATGCAGGTATCCGAATCCGGTGCACCGTTAGCCGCGAA |
| sequence | TCTCGTGCAACCTGCTCCGTTGAAATCGAGGGAAGTCCGCGTGGAAATCGCTGCT |
| | AGTGGTGTGTGCCATGCAGATATTGGCACGGCAGCAGCATCGGGGAAGCACACTG |
| | TTTTTCCTGTTACCCCTGGTCATGAGATTGCAGGAACCATCGCGGAAATTGGTGAA |
| | AACGTATCTCGGTGGACGGTTGGTGATCGCGTTGCAATCGGTTGGTTTGGTGGCAA |
| | TTGCGGTGACTGCGCTTTTTGTCGTGCAGGTGATCCTGTGCATTGCAGAGAGCGG |
| | AAGATTCCTGGCGTTTCTTATGCGGGTGGTTGGGCACAGAATATTGTTGTTCCAGC |
| | GGAGGCTCTTGCTGCGATTCCAGATGGCATGGACTTTTACGAGGCCGCCCCGATGG |
| | GCTGCGCAGGTGTGACAACATTCAATGCGTTGCGAAACCTGAAGCTGGATCCCGG |
| | TGCGGCTGTCGCGGTCTTTGGAATCGGCGGTTTAGTGCGCCTAGCTATTCAGTTTG |
| | CTGCGAAAATGGGTTATCGAACCATCACCATCGCCCGCGGTTTAGAGCGTGAGGA |
| | GCTAGCTAGGCAACTTGGCGCCAACCACTACATCGATAGCAATGATCTGCACCCTG |
| | GCCAGGCGTTATTTGAACTTGGCGGGGCTGACTTGATCTTGTCTACTGCGTCCACC |
| | ACGGAGCCTCTTTCGGAGTTGTCTACCGGTCTTTCTATTGGCGGGCAGCTAACCAT |
| | TATCGGAGTTGATGGGGGAGATATCACCGTTTCGGCAGCCCAATTGATGATGAACC |
| | GTCAGATCATCACAGGTCACCTCACTGGAAGTGCGAATGACACGGAACAGACTAT |
| | GAAATTTGCTCATCTCCATGGCGTGAAACCGCTTATTGAACGGATGCCTCTCGATC |
| | AAGCCAACGAGGCTATTGCACGTATTTCAGCTGGTAAACCACGTTTCCGTATTGTC |
| | TTGGAGCCGAATTCATAA (SEQ ID NO: 70) |

TABLE 1-36
NCgl2709/NP_601999.1/Corynebacterium glutamicum ATCC 13032

| | |
|---|---|
| Amino acid | MTTAAPQEFTAAVVEKFGHDVTVKDIDLPKPGPHQALVKVLTSGICHTDLHALEGDW |
| sequence | PVKPEPPFVPGHEGVGEVVELGPGEHDVKVGDIVGNAWLWSACGTCEYCITGRETQ |
| | CNEAEYGGYTQNGSFGQYMLVDTRYAARIPDGVDYLEAAPILCAGVTVYKALKVSE |
| | TRPGQFMVISGVGGLGHIAVQYAAAMGMRVIAVDIADDKLELARKHGAEFTVNARN |
| | EDSGEAVQKYTNGGAHGVLVTAVHEAAFGQALDMARRAGTIVFNGLPPGEFPASVF |
| | NIVFKGLTIRGSLVGTRQDLAEALDFFARGLIKPTVSECSLDEVNGVLDRMRNGKIDG |
| | RVAIRF (SEQ ID NO: 71) |
| Base | ATGACCACTGCTGCACCCCAAGAATTTACCGCTGCTGTTGTTGAAAAATTCGGTCA |
| sequence | TGACGTGACCGTGAAGGATATTGACCTTCCAAAGCCAGGGCCACACCAGGCATTG |
| | GTGAAGGTACTCACCTCCGGCATCTGCCACACCGACCTCCACGCCTTGGAGGGCG |
| | ATTGGCCAGTAAAGCCGGAACCACCATTCGTACCAGGACACGAAGGTGTAGGTGA |
| | AGTTGTTGAGCTCGGACCAGGTGAACACGATGTGAAGGTCGGCGATATTGTCGGC |
| | AATGCGTGGCTCTGGTCAGCGTGTGGCACCTGCGAATACTGCATCACCGGCAGGG |
| | AAACTCAGTGCAACGAAGCTGAGTATGGTGGCTACACCCAAAATGGATCCTTCGG |
| | CCAGTACATGCTGGTGGATACCCGTTACGCCGCTCGCATCCCAGACGGCGTGGACT |
| | ACCTCGAAGCAGCACCAATTCTGTGTGCAGGCGTGACTGTCTACAAGGCACTCAA |
| | AGTCTCTGAAACCCGCCCGGGCCAATTCATGGTGATCTCCGGTGTCGGCGGACTT |
| | GGCCACATCGCAGTCCAATACGCAGCGGCGATGGGCATGCGTGTCATTGCGGTAG |
| | ATATTGCCGATGACAAGCTGGAACTTGCCCGTAAGCACGGTGCGGAATTTACCGTG |
| | AATGCGCGTAATGAAGATTCAGGCGAAGCTGTACAGAAGTACACCAACGGTGGCG |
| | CACACGGCGTGCTTGTGACTGCAGTTCACGAGGCAGCATTCGGCCAGGCACTGGA |
| | TATGGCTCGACGTGCAGGAACAATTGTGTTCAACGGTCTGCCACCGGGAGAGTTC |
| | CCAGCATCCGTGTTCAACATCGTATTCAAGGGCCTGACCATCCGTGGATCCCTCGT |
| | GGGAACCCGCCAAGACTTGGCCGAAGCGCTCGATTTCTTTGCACGCGGACTAATC |
| | AAGCCAACCGTGAGTGAGTGCTCCCTCGATGAGGTCAATGGTGTGCTTGACCGCA |
| | TGCGAAACGGCAAGATCGATGGTCGTGTGGCGATTCGTTTCTAA (SEQ ID NO: 72) |

TABLE 1-37
NCgl1112/NP_600385.1/Corynebacterium glutamicum ATCC 13032

| | |
|---|---|
| Amino acid | MSLQFDHETLGQRVLFGSGEAAQNLAAEISRLDAKNVMVVAGDFELPMARQVAADI |
| sequence | DVKVWHSNVVMHVPIETAEEARSVAKENDIDVVVCVGGGSTTGLAKAIAMTTALPII |
| | AVPTTYAGSEATNVWGLTEAARKTTGVDNKVLPVTVIYDSALTMSLPVEMSVASGLN |
| | GLAHCIDSLWGPKADPINAAMAAEGIRALSAGLPKIVADAQDVDGRDEALYGAYLA |
| | AVSFASAGSGLHHKICHVLGGTFNLPHAQTHATVLPYVLAFNAPYAPQAEQRAAAAF |
| | GSATALEGLQQLRAQVGAPQRLSDYGFTAAGIPEAVEIILEKVPANNPRTVTEENLTAL |
| | LTTALNGDDPATLN (SEQ ID NO: 73) |
| Base | ATGTCTTTACAGTTCGATCATGAAACCCTCGGTCAACGAGTTCTGTTCGGTTCAGG |
| sequence | TGAGGCGGCGCAAAATCTCGCCGCTGAAATTAGCCGACTCGATGCCAAAAACGTC |
| | ATGGTGGTTGCCGGTGATTTCGAGCTTCCCATGGCACGGCAAGTAGCAGCAGATAT |
| | TGATGTCAAGGTGTGGCATTCAAATGTCGTGATGCATGTGCCCATCGAAACAGCAG |
| | AAGAAGCACGCAGTGTTGCGAAAGAAAACGACATTGATGTTGTGGTGTGTGTGG |
| | GCGGTGGATCCACAACAGGTCTAGCTAAAGCGATTGCCATGACCACCGCATTGCC |
| | GATCATTGCGGTACCCACTACTTATGCAGGTTCTGAAGCAACAAATGTGTGGGGAT |
| | TGACCGAAGCCGCGCGCAAAACAACTGGTGTTGATAACAAAGTGCTGCCAGTGA |
| | CAGTTATCTACGATTCAGCGTTAACCATGTCTTTGCCGGTAGAAATGTCGGTTGCTT |
| | CTGGTCTCAATGGTTTGGCTCACTGCATTGATTCTTTGTGGGGACCGAAGGCGGAT |
| | CCCATCAATGCGGCTATGGCTGCTGAGGGAATTCGAGCACTTTCTGCTGGCCTTCC |
| | CAAGATTGTGGCAGATGCTCAGGACGTAGATGGTCGCGATGAAGCGCTCTACGGT |
| | GCCTACCTGGCTGCGGTGTCTTTTGCCTCTGCTGGCTCTGGTCTCCACCACAAGAT |
| | CTGCCACGTGTTGGGTGGAACTTTTAACCTTCCACACGCGCAAACCCATGCAACA |
| | GTACTGCCTTATGTTCTTGCCTTCAACGCGCCATATGCGCCACAGGCAGAACAACG |
| | CGCAGCGGCAGCTTTCGGTTCTGCGACAGCACTTGAAGGATTGCAACAGCTGCGT |
| | GCCCAAGTGGGAGCACCACAGCGACTATCCGATTACGGATTCACCGCAGCAGGAA |
| | TCCCAGAGGCAGTGGAAATCATCTTGGAGAAAGTACCGGCGAATAATCCACGGAC |
| | GGTCACAGAAGAAAACCTCACTGCGCTGCTTACCACAGCGCTCAACGGCGACGAT |
| | CCAGCAACTTTGAATTAA (SEQ ID NO: 74) |

TABLE 1-38
NCgl2382/NP_601669.1/Corynebacterium glutamicum ATCC 13032

| | |
|---|---|
| Amino acid | MQTLAAIVRATKQPFEITTIDLDAPRPDEVQIRVIAAGVRHTDAIVRDQIYPTFLPAVFG |
| sequence | HEGAGVVVAVGSAVTSVKPDDKVVLGFNSCGQCLKCLGGKPAYCEKFYDRNFACTR |
| | DAGHTTLFTRATKEQAEAIIDTLDDVFYDADAGFLAYPATPPEASGVSVLVVAAGTSD |
| | LPQAKEALHTASYLGRSTSLIVDFGVAGIHRLLSYEEELRAAGVLIVAAGMDGALPGV |
| | VAGLVSAPVVALPTSVGYGAGAGGIAPLLTMLNACAPGVGVVNIDNGYGAGHLAAQ |
| | IAAR (SEQ ID NO: 75) |
| Base | ATGCAAACCCTTGCTGCTATTGTTCGTGCCACGAAGCAACCTTTTGAGATCACCAC |
| sequence | CATTGATCTGGATGCACCACGACCAGATGAAGTTCAAATCCGTGTTATTGCTGCCG |
| | GAGTGCGCCACACTGACGCAATTGTTCGTGATCAGATTTACCCAACTTTTCTTCCC |
| | GCAGTTTTCGGCCACGAAGGCGCCGGAGTAGTTGTCGCCGTGGGTTCTGCAGTCA |
| | CCTCGGTGAAACCAGATGACAAGGTAGTGCTGGGATTCAACTCTTGTGGCCAGTG |
| | CTTGAAGTGTTTGGGCGGTAAGCCTGCGTACTGTGAGAAATTCTATGACCGCAACT |
| | TCGCATGCACCCGCGATGCCGGGCACACTACTTTGTTTACCCGTGCAACAAAAGA |
| | GCAGGCAGAGGCCATCATCGACACCCTTGATGATGTTTTCTACGATGCGGATGCGG |
| | GTTTCCTGGCATACCCAGCAACTCCCCCAGAGGCTTCGGGAGTAAGCGTGTTGGT |
| | TGTCGCGGCTGGTACCTCTGATCTCCCCCAAGCAAAGGAAGCACTACACACTGCC |
| | TCCTACTTGGGGCGCTCCACCTCACTGATTGTTGATTTTGGAGTGGCTGGCATCCA |
| | CCGCCTGCTTTCATACGAAGAAGAACTCCGCGCTGCGGGCGTGCTCATCGTTGCC |
| | GCTGGAATGGATGGTGCGCTACCCGGAGTTGTCGCAGGCTTAGTGTCCGCACCTG |
| | TCGTCGCACTGCCAACCTCCGTGGGATACGGCGCAGGTGCTGGAGGAATCGCACC |
| | ACTTCTGACCATGCTTAACGCCTGCGCGCCGGGAGTTGGAGTGGTCAACATTGATA |
| | ACGGCTATGGAGCAGGACACCTGGCTGCGCAGATTGCGGCGAGGTAA (SEQ ID |
| | NO: 76) |

TABLE 1-39
NCgl0186/NP_599442.1/Corynebacterium glutamicum
ATCC 13032

| | |
|---|---|
| Amino | MLNAVGKAQNILLLGGTSEIGISIVSRFLKQGPSHVTLAA |
| acid | RKDSPRVDAAVAEIKAAGAASVAVVDFDALDTESHPAAID |
| sequence | AAFENGDVDVAIVAFGILGDNEAQWRDQALAVEATTVNYT |
| | AGVSVGVLLGQKFEQQGHGTIVALSSVAGQRVRRSNFVYG |
| | SAKAGFDGFYTQLGEALRGSGANVLVVRPGQVRTKMSADG |
| | GEAPLTVNREDVADAVYDAVVNKKDIIFVHPLFQYVSFAF |
| | QFIPRAIFRKLPF (SEQ ID NO: 77) |
| Base | ATGCTTAACGCAGTGGGCAAAGCCCAAAACATTCTCCTTC |
| sequence | TTGGTGGAACCTCTGAGATCGGTATTTCCATTGTCTCCCG |
| | CTTCCTCAAGCAGGGTCCATCCCATGTGACCTTGGCAGCG |
| | CGTAAAGATTCCCCACGCGTGGACGCAGCAGTCGCAGAGA |
| | TCAAAGCAGCTGGCGCTGCTTCCGTTGCTGTTGTTGATTT |
| | CGATGCGCTCGACACCGAATCCCACCCTGCAGCCATCGAC |
| | GCAGCCTTTGAAAACGGCGACGTTGACGTAGCAATCGTGG |
| | CTTTCGGCATCCTCGGCGACAACGAAGCACAGTGGCGCGA |
| | CCAAGCACTAGCAGTGGAAGCAACCACCGTGAACTACACC |
| | GCCGGCGTTTCCGTAGGTGTACTGCTGGGCCAGAAATTTG |
| | AGCAGCAGGGCCACGGCACCATCGTGGCATTGTCCTCTGT |
| | GGCAGGCCAGCGAGTCCGCCGCTCCAACTTTGTCTACGGC |
| | TCCGCCAAGGCAGGTTTCGACGGTTTCTACACCCAGCTCG |
| | GCGAAGCCCTGCGTGGATCCGGTGCCAACGTATTGGTGGT |
| | TCGCCCAGGCCAGGTACGCACCAAGATGTCCGCAGATGGT |
| | GGCGAAGCCCCACTGACCGTCAACCGCGAAGACGTGGCAG |
| | ATGCTGTTTATGATGCAGTGGTGAACAAGAAGGACATCAT |
| | CTTTGTCCACCCACTGTTCCAGTACGTCTCTTTTGCGTTC |
| | CAATTCATTCCGCGAGCAATCTTCCGCAAGCTGCCGTTC |
| | TAA (SEQ ID NO: 78) |

TABLE 1-40
NCgl0099/NP_599352.1/Corynebacterium glutamicum
ATCC 13032

| | |
|---|---|
| Amino | MEHGVTVIKGTEFDVFPLNLGGNTFGWTSNREQTFAVLDA |
| acid | FVAAGGNFVDTADSYSAWVEGNEGGESERELGAWIKERGA |
| sequence | DKLIIATKSGALEPVAGRSREATFKAVEGSLERLGVESID |
| | IFYYHYDDEAVSIDEQVAIANDLIAQGKIKHLALSNYSAE |
| | RLAEFFEKSVGTPAQPVALQPHYNLVSRKDYEENVQPLAE |
| | KHGVAVFPYFALAAGLLTGKYTSKEDISGKARAGQLDRYA |
| | SDEAFAVVTELRAVADELGVAPTTVALAWLVAHGVTAPIA |
| | SVSKVEQLKDLMAVKDVELSAEQLARLDKVSEPFA |
| | (SEQ ID NO: 79) |
| Base | ATGGAGCACGGCGTGACCGTTATTAAAGGCACTGAATTTG |
| sequence | ATGTTTTCCCACTAAACCTCGGTGGAAATACCTTTGGCTG |
| | GACCTCGAATAGGGAACAGACCTTCGCGGTTTTGGATGCA |
| | TTCGTGGCAGCGGGAGGAAACTTTGTTGACACCGCCGATT |
| | CTTATTCTGCATGGGTTGAAGGCAATGAGGGTGGCGAGTC |
| | GGAGCGGGAGCTCGGCGCGTGGATTAAGGAACGTGGCGCA |
| | GACAAGCTGATCATTGCTACCAAGTCTGGTGCGTTGGAGC |
| | CTGTTGCTGGTCGTTCCCGTGAGGCAACTTTCAAGGCTGT |
| | CGAGGGTTCCCTGGAGCGTTTGGGCGTGGAATCGATCGAT |
| | ATTTTTTACTACCACTACGACGATGAGGCAGTCAGCATTG |
| | ATGAGCAGGTTGCTATCGCTAATGATCTGATTGCACAGGG |
| | CAAGATTAAGCACCTCGCATTGTCTAACTACAGCGCGGAG |
| | CGTTTAGCTGAGTTCTTTGAGAAGTCTGTAGGCACTCCAG |
| | CGCAGCCGGTTGCTCTGCAACCGCACTACAACCTGGTGTC |
| | GAGGAAGGATTATGAGGAGAACGTGCAGCCACTCGCCGAG |
| | AAGCATGGCGTTGCAGTCTTCCCTTATTTCGCGCTTGCCG |
| | CGGGTCTTTTGACCGGAAAGTACACCTCCAAGGAGGATAT |
| | TTCGGGTAAAGCGCGTGCGGGGCAGTTGGATCGTTACGCC |
| | AGCGATGAGGCGTTTGCCGTGGTGACAGAGTTGCGTGCTG |
| | TTGCCGATGAGTTGGGTGTTGCGCCAACGACTGTGGCGCT |
| | TGCGTGGTTGGTTGCGCATGGTGTGACCGCACCGATTGCG |
| | TCCGTGTCCAAGGTAGAGCAGTTGAAGGATTTGATGGCTG |
| | TGAAGGATGTGGAGCTGAGCGCTGAGCAGCTTGCACGTTT |
| | GGATAAGGTTTCGGAGCCTTTCGCTTAA |
| | (SEQ ID NO: 80) |

TABLE 1-41
NCgl2952/NP_602249.1/Corynebacterium glutamicum
ATCC 13032

| | |
|---|---|
| Amino | MNNSLAFNHDTLPQKVMFGYGKSSAFLKQEVERRGSAKVM |
| acid | VIAGEREMSIAHKVASEIEVAIWHDEVVMHVPIEVAERAR |
| sequence | AVATDNEIDLLVCVGGGSTIGLAKAIAMTTALPIVAIPTT |
| | YAGSEATNVWGLTEAARKTTGVDLKVLPETVIYDSELTMS |
| | LPVEMSVASGLNGLAHCIDSLWGPNADPINAVLAAEGIRA |
| | LNQGLPKIVANPHSIEGRDEALYGAYLAAVSFASAGSGLH |
| | HKICHTLGGTFNLPHAQTHATVLPYVLAFNAGDAPEAERR |
| | AAAAFGTDTALEGLQRLRLSVNAPKRLSDYGFEASGIAEA |
| | VDVTLEKVPANNPRPVTRENLSRLLEAALNGEDPAVLSAV |
| | LSN (SEQ ID NO: 81) |
| Base | GTGAACAACTCACTCGCATTCAACCACGACACCCTCCCAC |
| sequence | AGAAAGTCATGTTTGGATATGGCAAGTCCAGTGCATTCTT |
| | AAAGCAGGAAGTTGAACGCCGCGGCTCAGCCAAGGTCATG |
| | GTCATTGCGGGTGAACGAGAAATGTCGATCGCCCATAAGG |
| | TGGCCTCAGAAATTGAGGTGGCGATCTGGCACGACGAAGT |
| | TGTCATGCACGTGCCCATCGAAGTAGCCGAACGTGCGCGT |
| | GCAGTGGCAACCGACAATGAGATTGATCTGCTGGTGTGTG |
| | TTGGCGGCGGATCCACCATAGGTTTGGCCAAAGCAATTGC |
| | CATGACCACTGCCCTGCCCATCGTCGCGATCCCCACCACC |
| | TACGCAGGATCGGAAGCAACCAACGTGTGGGGTCTGACGG |
| | AAGCAGCGCGCAAAACAACCGGTGTTGATCTGAAGGTGCT |
| | CCCCGAAACAGTCATTTACGATTCCGAACTCACCATGTCG |
| | CTTCCAGTGGAGATGTCCGTGGCATCCGGACTCAACGGCC |
| | TGGCGCACTGCATTGATTCTTTGTGGGGACCCAACGCCGA |
| | TCCCATCAACGCAGTGCTTGCAGCCGAAGGAATCCGCGCA |
| | CTCAACCAGGGACTGCCGAAAATTGTTGCGAACCCGCACA |
| | GCATCGAAGGACGCGACGAAGCCCTCTACGGCGCCTACCT |
| | CGCAGCAGTATCCTTCGCCTCCGCAGGCTCCGGACTACAC |
| | CACAAAATCTGCCACACCTTGGGAGGCACCTTCAACCTCC |
| | CCCACGCCCAAACCCACGCAACCGTGCTGCCGTATGTTTT |
| | GGCATTCAACGCAGGCGACGCACCAGAAGCTGAACGCCGC |
| | GCAGCCGCAGCCTTTGGAACTGACACCGCACTAGAAGGCC |
| | TGCAACGCCTCCGCTTGTCAGTCAACGCACCGAAACGACT |
| | TTCCGACTACGGCTTCGAGGCTTCAGGAATTGCTGAGGCA |
| | GTGGACGTCACGTTGGAGAAAGTTCCCGCCAACAATCCTC |
| | GCCCAGTGACCCGGGAAAACCTCAGCAGATTGCTCGAAGC |
| | AGCACTCAACGGTGAGGATCCGGCAGTTCTTAGCGCAGTA |
| | CTCAGTAACTAA (SEQ ID NO: 82) |

TABLE 1-42
NCgl1459/NP_600732.2/Corynebacterium glutamicum
ATCC 13032

| | |
|---|---|
| Amino | MKQRMVGSSGLRVSRLGLGTSTWGSGTELAEAGDIFKAFI |
| acid | NSGGTLIDVSPNYTTGVAEEMLGTMLDAEVSRSAVVISSS |
| sequence | AGVNPALPLGRRVDCSRRNLIAQLDVTLRALNTDYLDLWS |
| | VGYWDEGTPPHEVADTLDYAVRTGRVRYAGVRGYSGWQLA |
| | VTHAASNHAAASARPVVVAQNEYSLLERRAEQELLPATQH |
| | LGVGFFAGAPLGQGVLTAKYRSEIPHDSRAASTGRDAEVQ |
| | SYLDNRGRIIVDALDTAAKGLGISPAVTATTWVRDRPGVT |
| | AVIVGARTHEQLSHLLKAESVTLPTPITQALDDVSL |
| | (SEQ ID NO: 83) |
| Base | GTGAAACAGCGAATGGTCGGTTCAAGTGGTTTGCGGGTAT |
| sequence | CCAGGCTCGGTTTGGGCACCTCAACATGGGGCTCGGGCAC |
| | CGAGCTGGCTGAGGCAGGCGATATCTTTAAGGCGTTCATC |
| | AATTCTGGTGGCACGCTTATCGACGTCTCCCCCAACTACA |
| | CCACCGGCGTCGCGGAAGAAATGCTCGGCACGATGTTGGA |
| | TGCGGAAGTCTCTCGTTCGGCTGTCGTCATTTCCTCCAGC |
| | GCAGGTGTCAACCCCGCTCTGCCGCTCGGCCGACGTGTGG |
| | ATTGCTCCCGCCGCAATTTGATTGCCCAATTAGATGTCAC |
| | CCTGCGGGCATTAAACACTGACTATTTGGATTTGTGGTCT |
| | GTGGGCTATTGGGATGAGGGCACCCCACCGCATGAGGTGG |
| | CCGATACTTTGGATTACGCCGTGCGCACCGGCCGAGTCCG |
| | ATATGCCGGTGTCCGAGGATATTCCGGTTGGCAGTTAGCG |
| | GTCACCCACGCTGCATCCAATCATGCAGCGGCCTCCGCCC |
| | GCCCCGTGGTCGTTGCACAAAATGAATACAGCCTGCTGGA |
| | ACGCCGCGCAGAACAAGAACTCCTCCCTGCCACCCAACAC |
| | CTAGGTGTCGGATTCTTTGCTGGCGCTCCGCTGGGGCAAG |
| | GCGTGCTGACTGCTAAATACCGCTCCGAAATTCCCCATGA |
| | TTCCAGAGCTGCATCCACAGGACGCGACGCAGAAGTCCAA |
| | AGCTACCTAGATAATCGAGGCCGCATCATTGTCGATGCTC |
| | TTGATACTGCAGCCAAAGGATTAGGCATTAGCCCCGCTGT |
| | CACAGCCACCACCTGGGTGCGTGATCGTCCCGGAGTGACA |
| | GCTGTCATCGTGGGCGCTCGCACACATGAACAGCTGTCAC |
| | ATCTTCTCAAGGCGGAATCGGTGACTTTGCCAACACCAAT |
| | CACACAAGCCCTTGATGATGTCTCCCTGTGA |
| | (SEQ ID NO: 84) |

TABLE 1-43
yogA/NP_389725.1/Bacillus subtilis subsp.
subtilis str. 168

| | |
|---|---|
| Amino | MKAVIHNGKAGLLGLSVQDVPSTKPGYGEVKVKLKSAGLN |
| acid | HRDLFLMKNKSEQDPHMILGSDGAGIIEEIGEGVKNVTVQ |
| sequence | TEVVIFPTLNWDLTENVPPVPEILGGPSDGTLAEYVIIPS |
| | QNAIKKPAYLSWEEAGVLPLSALTAYRALFTKGQLKKGEH |
| | LLIPGIGSGVATYALFMAKAIGATVSVTSRSEEKRKKALK |
| | LGADYAFDSYSNWDEQLQGKKIDVVLDSIGPALFSEYFRH |
| | VKPNGRIVSFGASSGDNLSFPVRSLFFPQVNVLGTSMGSG |
| | EEFQAMLAFIDKHKLRPVIDRIYPLEKACEAYKRMQEGRQ |
| | FGNIGIVME (SEQ ID NO: 85) |
| Base | ATGAAAGCTGTAATTCACAACGGAAAAGCCGGTCTTCTGG |
| sequence | GGTTATCAGTTCAGGACGTTCCATCAACAAAGCCTGGATA |
| | CGGAGAGGTAAAGGTTAAATTAAAATCTGCAGGCCTGAAT |
| | CATCGTGACTTGTTTCTTATGAAAAACAAATCTGAACAAG |
| | ATCCTCACATGATACTGGGTTCTGACGGCGCGGGTATCAT |
| | CGAAGAGATTGGTGAAGGCGTGAAAAATGTTACTGTTCAG |
| | ACAGAAGTAGTCATTTTCCCGACATTGAACTGGGATTTGA |
| | CAGAAAATGTTCCACCTGTACCTGAGATTCTGGGAGGTCC |
| | TTCGGACGGAACACTTGCTGAATATGTGATCATTCCTTCA |
| | CAAAATGCAATCAAAAAACCTGCTTATTTATCTTGGGAAG |
| | AAGCGGGCGTTTTACCTTTATCCGCTTTAACTGCATATCG |
| | GGCTCTGTTTACAAAGGGGCAATTAAAAAAAGGCGAGCAT |
| | CTATTGATACCCGGCATCGGCAGCGGTGTAGCAACCTACG |
| | CTTTATTTATGGCTAAGGCGATTGGGGCAACAGTAAGCGT |
| | GACCTCCCGCAGTGAGGAGAAAAGAAAAAAGGCGCTGAAA |
| | TTAGGTGCTGATTACGCATTTGACAGCTACAGCAATTGGG |
| | ATGAGCAGTTGCAGGGAAAAAAGATAGATGTTGTTCTTGA |
| | CAGCATAGGACCTGCCCTCTTTTCGGAATACTTCCGCCAT |
| | GTAAAACCAAATGGCCGTATTGTCAGCTTTGGGGCAAGCT |
| | CAGGGGATAATCTCAGTTTTCCGGTGCGTTCTTTATTCTT |
| | TCCTCAGGTCAATGTTTTGGGAACCTCGATGGGAAGCGGT |
| | GAGGAATTTCAAGCTATGCTCGCTTTCATTGACAAACATA |
| | AGCTGCGGCCTGTAATTGACCGGATATATCCTTTAGAAAA |
| | AGCATGTGAAGCATATAAAAGAATGCAGGAAGGCAGACAG |
| | TTTGGAAACATCGGGATCGTAATGGAATAA |
| | (SEQ ID NO: 86) |

TABLE 1-44
bdhK/NP_391014.1/Bacillus subtilis subsp.
subtilis str. 168

| | |
|---|---|
| Amino | MENFTYYNPTKLIFGKGQLEQLRKEFKRYGKNVLLVYGGG |
| acid | SIKRNGLYDQVTGILKEEGAVVHELSGVEPNPRLATVEKG |
| sequence | IGLCREHDIDFLLAVGGGSVIDCTKAIAAGVKYDGDAWDI |
| | FSKKVTAEDALPFGTVLTLAATGSEMNPDSVITNWETNEK |
| | FVWGSNVTHPRFSILDPENTFTVPENQTVYGMVDMMSHVF |
| | EQYFHNVENTPLQDRMCFAVLQTVIETAPKLLEDLENYEL |
| | RETILYAGTIALNGTLQMGYFGDWASHTMEHAVSAVYDIP |
| | HAGGLAILFPNWMRYTLDTNVGRFKNLMLNMFDIDTEGKT |
| | DKEIALEGIDKLSAFWTSLGAPSRLADYNIGEEKLELIAD |
| | IAAKEMEHGGFGNFQKLNKDDVLAILRASL |
| | (SEQ ID NO: 87) |
| Base | ATGGAAAATTTCACTTATTATAATCCGACAAAGCTGATTT |
| sequence | TTGGAAAAGGTCAGCTTGAACAATTAAGAAAAGAATTCAA |
| | ACGATACGGCAAGAATGTACTGCTTGTTTACGGGGGCGGC |
| | AGCATTAAACGCAACGGCCTTTATGATCAAGTCACAGGCA |
| | TTTTAAAAGAAGAGGGCGCTGTTGTTCATGAGCTGTCAGG |
| | TGTAGAGCCAAACCCGCGTCTTGCGACAGTGGAAAAAGGC |
| | ATAGGACTTTGCAGAGAGCATGACATTGATTTTCTGCTTG |
| | CTGTCGGCGGAGGCAGTGTGATTGACTGTACAAAGGCAAT |
| | CGCAGCTGGCGTCAAATATGACGGTGACGCTTGGGATATT |
| | TTCAGCAAAAAAGTAACAGCGGAGGATGCGCTGCCGTTTG |
| | GCACTGTTTTAACTCTTGCTGCAACAGGGTCTGAAATGAA |
| | CCCTGATTCCGTGATTACAAACTGGGAAACAAACGAGAAA |
| | TTTGTATGGGGCAGCAATGTCACTCATCCGCGTTTCTCTA |
| | TTTTAGACCCTGAAAATACGTTCACCGTTCCAGAAAATCA |
| | AACAGTATACGGCATGGTTGACATGATGAGCCACGTATTC |
| | GAACAATACTTCCATAATGTTGAAAACACGCCGCTTCAGG |
| | ATAGAATGTGCTTTGCTGTTTTGCAGACGGTCATCGAAAC |
| | AGCTCCTAAGCTTCTTGAAGATCTGGAAAACTACGAACTT |
| | CGTGAAACGATTTTGTATGCTGGTACTATTGCTTTAAACG |
| | GCACGCTCCAAATGGGATACTTCGGTGACTGGGCTTCTCA |
| | TACAATGGAACACGCTGTTTCAGCTGTATATGATATTCCG |
| | CACGCGGGCGGTTTGGCAATACTGTTCCCAAATTGGATGA |
| | GATACACGCTTGATACAAATGTAGGCCGTTTTAAAAACCT |
| | TATGCTCAACATGTTTGACATTGATACTGAAGGCAAAACA |
| | GATAAAGAAATTGCGCTTGAAGGAATCGATAAACTGTCTG |
| | CGTTCTGGACAAGCCTCGGTGCACCTTCTCGTCTTGCTGA |
| | TTACAATATTGGAGAAGAAAAGCTTGAGCTGATTGCTGAT |
| | ATCGCAGCCAAGGAAATGGAACACGGCGGCTTCGGCAACT |
| | TCCAAAAACTGAACAAAGATGACGTGCTTGCCATCCTTCG |
| | CGCGTCTCTATAA (SEQ ID NO: 88) |

TABLE 1-45
bdhJ/NP_391015.1/Bacillus subtilis subsp.
subtilis str. 168

| | |
|---|---|
| Amino | MQNFTYWNPTKLIFGRGEVERLPEELKPYGKNVLLVYGGG |
| acid | SIKRSGLYDQVIEQLNKAGATVHELAGVEPNPRVSTVNKG |
| sequence | VAICKEQNIDFLLAVGGGSVIDCTKAIAAGAKYDGDAWDI |
| | VTKKHQPKDALPFGTVLTLAATGSEMNSGSVITNWETKEK |
| | YGWGSPLVFPKFSILDPVNTFTVPKNHTIYGMVDMMSHVF |
| | EQYFHHVSNTPYQDRMCESLLRTVIETAPKLINDLENYEL |
| | RETILYTGTIALNGMLSMGARGDWATHNIEHAVSAVYDIP |
| | HAGGLAILFPNWMRHTLSENPARMKQLAVRVFDVEEAGKT |
| | DEEIALEGIDKLSAFWTSLGAPNRLADYDINDEQLDTIAD |
| | KAMANGTFGQFKSLNKEDVLSILKASL |
| | (SEQ ID NO: 89) |
| Base | ATGCAAAACTTTACATACTGGAATCCGACCAAATTAATTT |
| sequence | TCGGGCGCGGCGAAGTGGAAAGACTTCCGGAGGAACTCAA |
| | ACCTTACGGAAAAAACGTATTGCTTGTGTACGGAGGCGGC |
| | AGCATTAAACGCAGCGGCCTGTATGATCAAGTGATTGAAC |
| | AGCTGAATAAAGCCGGAGCGACCGTGCATGAATTAGCAGG |
| | TGTGGAACCGAATCCTCGTGTGTCGACTGTTAATAAAGGT |
| | GTTGCCATCTGTAAAGAACAAAACATTGATTTCTTGCTGG |
| | CTGTCGGAGGCGGAAGCGTAATCGACTGTACAAAAGCGAT |
| | TGCCGCAGGAGCGAAGTATGATGGTGATGCGTGGGATATC |
| | GTTACGAAAAAGCATCAGCCAAAAGATGCTTTGCCATTCG |
| | GAACGGTATTGACTCTCGCTGCAACTGGTTCAGAAATGAA |
| | CTCAGGATCTGTTATTACAAACTGGGAAACAAAAGAAAAA |
| | TACGGCTGGGGCAGCCCGCTCGTATTCCCTAAATTCTCGA |
| | TTCTTGATCCGGTGAATACATTCACCGTACCTAAAAACCA |
| | CACGATCTACGGGATGGTTGACATGATGAGCCACGTATTC |
| | GAACAATACTTCCATCATGTATCAAACACGCCGTATCAGG |
| | ACCGCATGTGTGAATCACTTTTGCGTACAGTCATTGAAAC |
| | AGCGCCTAAGCTGATCAATGATCTCGAAAATTACGAATTG |
| | CGTGAGACGATCCTGTACACAGGAACAATCGCATTAAACG |
| | GCATGCTTTCTATGGGGGCAAGAGGGGATTGGGCTACTCA |
| | TAATATTGAACATGCAGTATCAGCCGTTTATGATATTCCG |
| | CATGCCGGCGGACTGGCGATTCTGTTCCCGAATTGGATGA |
| | GACACACATTGTCTGAAAACCCTGCCCGCATGAAACAGCT |
| | TGCAGTTCGCGTGTTTGATGTTGAAGAAGCAGGTAAAACG |
| | GATGAAGAAATTGCCCTTGAAGGTATCGATAAGCTGTCCG |
| | CATTCTGGACAAGTCTTGGCGCTCCGAACCGTCTTGCTGA |
| | TTATGATATTAATGATGAGCAGCTTGACACAATCGCTGAC |
| | AAGGCAATGGCTAACGGTACATTCGGCCAATTTAAATCAC |
| | TCAACAAAGAAGATGTGCTGTCAATTTTGAAAGCATCACT |
| | ATAA (SEQ IDNO: 90) |

TABLE 1-46
akrN/NP_388834.1/Bacillus subtilis subsp.
subtilis str. 168

| | |
|---|---|
| Amino | MEYTSIADTGIEASRIGLGTWAIGGTMWGGTDEKTSIETI |
| acid | RAALDQGITLIDTAPAYGFGQSEEIVGKAIKEYGKRDQVI |
| sequence | LATKTALDWKNNQLFRHANRARIVEEVENSLKRLQTDYID |
| | LYQVHWPDPLVPIEETAEVMKELYDAGKIRAIGVSNFSIE |
| | QMDTFRAVAPLHTIQPPYNLFEREMEESVLPYAKDNKITT |
| | LLYGSLCRGLLTGKMTEEYTFEGDDLRNHDPKFQKPRFKE |
| | YLSAVNQLDKLAKTRYGKSVIHLAVRWILDQPGADIALWG |
| | ARKPGQLEALSEITGWTLNSEDQKDINTILENTISDPVGP |
| | EFMAPPTREEI (SEQ ID NO: 91) |
| Base | ATGGAATATACCAGTATAGCAGATACAGGAATAGAAGCCT |
| sequence | CCAGAATCGGCCTCGGCACATGGGCCATTGGCGGAACGAT |
| | GTGGGGAGGCACTGACGAAAAAACATCGATTGAAACAATC |
| | CGCGCCGCTCTTGATCAGGGGATTACACTGATTGACACCG |
| | CACCGGCTTACGGCTTCGGGCAGTCCGAGGAAATTGTCGG |
| | AAAGGCAATCAAAGAGTACGGCAAAAGAGACCAGGTGATT |
| | CTCGCAACGAAAACGGCTCTGGACTGGAAGAACAACCAGC |
| | TGTTCCGCCATGCGAACAGAGCGAGAATTGTAGAGGAAGT |
| | TGAGAATTCTTTGAAGCGGCTTCAAACAGACTATATTGAT |
| | CTTTATCAGGTGCATTGGCCCGATCCGCTTGTGCCAATTG |
| | AAGAAACGGCTGAAGTCATGAAGGAATTATATGATGCGGG |
| | AAAAATCCGGGCGATTGGCGTCAGCAATTTTTCAATTGAG |
| | CAAATGGATACATTTCGCGCCGTCGCACCTCTCCATACGA |
| | TTCAGCCTCCATATAATCTGTTTGAAAGAGAGATGGAAGA |
| | GAGTGTCCTTCCTTATGCGAAAGATAACAAGATAACAACA |
| | TTATTATACGGCAGTTTATGCAGAGGGCTGTTAACAGGCA |
| | AAATGACTGAAGAATATACATTTGAGGGCGATGATCTGCG |
| | TAATCACGATCCAAAATTCCAGAAGCCCCGCTTTAAAGAG |
| | TATCTTTCTGCTGTGAATCAATTGGATAAGCTGGCGAAGA |
| | CACGTTATGGAAAATCAGTGATTCACTTGGCTGTCAGATG |
| | GATCTTAGATCAGCCGGGAGCGGATATCGCTCTTTGGGGA |
| | GCAAGAAAGCCTGGGCAGCTTGAGGCCCTATCTGAGATTA |
| | CAGGCTGGACGCTGAACAGTGAAGATCAGAAAGATATCAA |
| | TACTATATTGGAAAATACGATATCAGACCCTGTCGGACCG |
| | GAGTTTATGGCCCCGCCGACCAGAGAGGAAATATAA |
| | (SEQ ID NO: 92) |

TABLE 1-47
yqkF/NP_390243.1/Bacillus subtilis subsp.
subtilis str. 168

| | |
|---|---|
| Amino | MRKRKLGTSDLDISEVGLGCMSLGTEKNKALSILDEAIEL |
| acid | GINYLDTADLYDRGRNEEIVGDAIQNRRHDIILATKAGNR |
| sequence | WDDGSEGWYWDPSKAYIKEAVKKSLTRLKTDYIDLYQLHG |
| | GTIEDNIDETIEAFEELKQEGVIRYYGISSIRPNVIKEYV |
| | KKSNIVSIMMQFSLFDRRPEEWLPLLEEHQISVVARGPVA |
| | KGLLTEKPLDQASESMKQNGYLSYSFEELTNARKAMEEVA |
| | PDLSMTEKSLQYLLAQPAVASVITGASKIEQLRENIQAAN |
| | ARRLTEEEIKALQSHTKQDIYKAHRS |
| | (SEQ ID NO: 93) |
| Base | ATGAGAAAGCGCAAATTGGGTACATCTGATTTAGACATTA |
| sequence | GCGAAGTCGGACTCGGCTGTATGTCTCTTGGAACTGAAAA |
| | AAACAAAGCATTGTCCATTCTGGATGAAGCGATCGAGCTT |
| | GGCATCAACTATTTGGACACAGCGGATTTGTATGACCGGG |
| | GACGCAATGAAGAAATTGTCGGTGATGCGATCCAAAACAG |
| | ACGCCATGATATTATTCTGGCAACAAAAGCGGGAAACCGT |
| | TGGGATGACGGAAGCGAGGGCTGGTATTGGGACCCTTCAA |
| | AAGCTTACATAAAAGAGGCGGTAAAAAAGAGCCTTACACG |
| | TCTGAAAACAGATTATATCGACCTTTATCAGCTCCACGGC |
| | GGCACGATAGAGGACAACATTGATGAAACGATTGAAGCGT |
| | TTGAGGAATTAAAACAAGAAGGTGTCATCCGCTACTACGG |
| | CATTTCTTCCATCCGCCCGAATGTGATTAAAGAATATGTA |
| | AAAAAATCAAACATCGTCAGCATTATGATGCAATTCAGCC |
| | TGTTTGACAGACGCCCTGAGGAATGGCTCCCGCTTTTAGA |
| | GGAACATCAAATCAGCGTAGTCGCCAGAGGTCCTGTTGCC |
| | AAAGGGCTCTTAACTGAAAAACCGCTTGATCAAGCTTCAG |
| | AAAGTATGAAACAAAACGGGTACTTGTCCTATTCATTCGA |
| | GGAACTGACAAATGCCCGTAAGGCAATGGAGGAAGTGGCT |
| | CCCGATCTTTCCATGACAGAAAAGTCGCTGCAGTATCTGC |
| | TAGCACAGCCGGCTGTCGCTTCAGTGATTACAGGCGCCAG |
| | TAAGATTGAGCAGTTACGGGAAAATATTCAGGCAGCAAAT |
| | GCACGGCGTTTAACCGAAGAGGAAATCAAAGCGCTCCAAT |
| | CTCATACGAAACAAGACATTTACAAAGCTCATCGCTCATA |
| | G (SEQ ID NO: 94) |

TABLE 1-48
yccK/NP_388159.1/Bacillus subtilis subsp.
subtilis str. 168

| | |
|---|---|
| Amino | MDQTRTLGKTKLKVKRIGFGANAVGGHNLFPNLNDETGKD |
| acid | LVRTALDGGVNFIDTAFIYGLGRSEELIGEVVQERGVRNE |
| sequence | LIIATKGAHKEVDGSIELDNSREFLRSEVEKSLKRLKTDY |
| | IDLYYVHFPDGKTPLAEVAGTLKELKDEGKIKAIGASNLD |
| | YQQLQDFNADGYLEVFQAEYSLIQRDAEKELLPYCEKQGI |
| | SFIPYFPLASGLLTGKFTQDTVFDDFRKDKPQFQGETFIH |
| | NLKKVDKLKAVAEEKQADTAHVALAWLLTRPAIDAIIPGA |
| | KRPEQLQDNLKTLNIELTEDEVNFISDIFK |
| | (SEQ ID NO: 95) |
| Base | ATGGATCAAACACGTACACTCGGCAAAACGAAGCTGAAGG |
| sequence | TGAAGCGGATCGGATTCGGCGCGAATGCGGTCGGCGGGCA |
| | TAATCTATTTCCAAATCTAAATGATGAAACAGGGAAGGAT |
| | TTAGTGCGCACGGCATTGGATGGCGGCGTCAATTTTATCG |
| | ATACCGCCTTTATATATGGATTGGGGCGATCTGAAGAATT |
| | AATCGGCGAAGTCGTACAGGAACGCGGCGTGCGGAATGAG |
| | CTCATCATCGCCACCAAAGGAGCTCATAAAGAAGTGGACG |
| | GCAGCATTGAATTAGACAATAGCCGGGAGTTTCTTCGCAG |
| | CGAGGTGGAGAAGAGCCTGAAGCGGCTGAAAACAGATTAC |
| | ATTGATTTGTATTATGTTCACTTTCCGGATGGAAAAACAC |
| | CTCTCGCTGAAGTGGCGGGCACTTTGAAAGAGCTGAAGGA |
| | TGAGGGGAAAATCAAAGCGATCGGCGCTTCGAACCTCGAT |
| | TATCAGCAATTGCAGGATTTTAATGCTGACGGCTATTTGG |
| | AGGTCTTCCAGGCCGAATATTCTCTCATACAGCGTGATGC |
| | CGAGAAAGAGCTTCTTCCATACTGTGAAAAACAAGGCATC |
| | TCCTTTATTCCTTACTTTCCGCTTGCGTCCGGACTGCTGA |
| | CAGGAAAATTCACGCAAGACACAGTCTTTGATGATTTCAG |
| | AAAGGATAAACCTCAATTTCAGGGTGAAACGTTTATCCAC |
| | AATCTCAAAAAAGTAGATAAGCTGAAAGCAGTAGCGGAGG |
| | AAAAACAAGCGGATACGGCACATGTCGCCTTGGCGTGGCT |
| | GTTAACGAGACCGGCGATTGATGCCATTATTCCAGGAGCT |
| | AAACGACCGGAGCAATTACAGGATAACCTGAAAACCTTGA |
| | ACATTGAACTGACCGAAGATGAAGTGAATTTCATCAGCGA |
| | CATTTTCAAATAA (SEQ ID NO: 96) |

TABLE 1-49
iolS/NP_391857.1/Bacillus subtilis subsp.
subtilis str. 168

| | |
|---|---|
| Amino | MKKAKLGKSDLQVFPIGLGTNAVGGHNLYPNLNEETGKEL |
| acid | VREAIRNGVTMLDTAYIYGIGRSEELIGEVLREFNREDVV |
| sequence | IATKAAHRKQGNDFVFDNSPDFLKKSVDESLKRLNTDYID |
| | LFYIHFPDEHTPKDEAVNALNEMKKAGKIRSIGVSNFSLE |
| | QLKEANKDGLVDVLQGEYNLLNREAEKTFFPYTKEHNISF |
| | IPYFPLVSGLLAGKYTEDTTFPEGDLRNEQEHFKGERFKE |
| | NIRKVNKLAPIAEKHNVDIPHIVLAWYLARPEIDILIPGA |
| | KRADQLIDNIKTADVTLSQEDISFIDKLFA |
| | (SEQ ID NO: 97) |
| Base | ATGAAAAAAGCGAAGCTCGGAAAATCAGACTTGCAGGTAT |
| sequence | TCCCTATCGGATTAGGAACAAATGCTGTCGGAGGACATAA |
| | CCTCTACCCGAACCTAAATGAAGAAACCGGAAAAGAATTG |
| | GTGCGCGAGGCGATCCGTAATGGCGTGACAATGTTAGACA |
| | CCGCTTACATTTACGGGATCGGCCGTTCCGAAGAATTAAT |
| | TGGTGAAGTGCTGCGTGAATTCAACCGTGAAGATGTTGTC |
| | ATCGCGACAAAAGCCGCTCACAGAAAACAAGGCAATGACT |
| | TTGTCTTTGATAATTCACCAGATTTTCTTAAAAAATCAGT |
| | TGATGAAAGCCTGAAGCGCTTGAATACCGATTATATTGAT |
| | TTGTTCTACATTCACTTCCCTGACGAACATACGCCTAAGG |
| | ATGAAGCCGTTAACGCGCTGAATGAGATGAAGAAAGCCGG |
| | AAAAATCCGTTCCATCGGTGTATCCAACTTCTCTTTAGAG |
| | CAATTGAAAGAAGCAAACAAAGACGGTTTGGTAGATGTAT |
| | TGCAAGGCGAATACAACCTGTTAAACCGTGAAGCGGAAAA |
| | AACATTCTTCCCGTATACGAAGGAGCATAATATTTCATTT |
| | ATCCCTTACTTCCCTCTCGTATCAGGTTTATTGGCAGGAA |
| | AGTATACAGAAGATACAACGTTCCCAGAAGGCGACCTGCG |
| | AAACGAACAGGAACACTTCAAGGGTGAGCGTTTCAAAGAA |
| | AATATCAGAAAGGTCAACAAGCTTGCGCCGATTGCCGAAA |
| | AACACAACGTGGATATCCCTCACATCGTATTGGCCTGGTA |
| | TTTAGCAAGACCGGAAATTGATATTTTAATCCCAGGAGCA |
| | AAACGTGCCGATCAGCTGATTGATAACATCAAAACAGCTG |
| | ACGTGACGCTTTCTCAAGAGGATATTTCATTTATTGATAA |
| | GCTGTTCGCATAA (SEQ ID NO: 98) |

TABLE 1-50
yrpG/NP_390562.2/Bacillus subtilis subsp.
subtilis str. 168

| | |
|---|---|
| Amino | MEYTYLGRTGLRVSRLCLGTMNFGVDTDEKTAFRIMDEAL |
| acid | DNGIQFFDTANIYGWGKNAGLTESIIGKWFAQGGQRREKV |
| sequence | VLATKVYEPISDPNDGPNDMRGLSLYKIRRHLEGSLKRLQ |
| | TDHIELYQMHHIDRRTPWDEIWEAFETQVRSGKVDYIGSS |
| | NFAGWHLVKAQAEAEKRRFMGLVTEQHKYSLLERTAEMEV |
| | LPAARDLGLGVVAWSPLAGGLLGGKALKSNAGTRTAKRAD |
| | LIEKHRLQLEKFSDLCKELGEKEANVALAWVLANPVLTAP |
| | IIGPRTVEQLRDTIKAVEISLDKEILRMLNDIFPGPGGET |
| | PEAYAW (SEQ ID NO: 99) |
| Base | GTGGAGTATACCTATTTAGGGAGAACAGGATTGCGGGTGA |
| sequence | GCCGTTTATGTTTAGGCACGATGAATTTTGGAGTTGATAC |
| | AGACGAAAAGACTGCGTTCCGTATCATGGATGAAGCACTT |
| | GATAACGGCATTCAATTTTTTGATACTGCCAATATTTACG |
| | GCTGGGGCAAAAACGCAGGATTGACAGAGAGCATCATTGG |
| | AAAATGGTTTGCACAAGGAGGACAGCGCCGCGAGAAAGTT |
| | GTTCTGGCGACAAAAGTATATGAACCGATTTCTGATCCGA |
| | ATGACGGACCAAATGATATGAGGGGCTTGTCTCTATACAA |
| | AATCAGACGTCATCTGGAAGGATCACTGAAGCGGCTTCAG |
| | ACAGATCATATCGAATTGTACCAAATGCATCATATCGATA |
| | GGCGGACACCGTGGGATGAGATATGGGAAGCTTTTGAGAC |
| | TCAGGTTCGCTCCGGCAAAGTAGACTATATTGGATCCAGT |
| | AATTTTGCAGGCTGGCATTTAGTTAAAGCGCAAGCTGAAG |
| | CTGAAAAACGGCGATTCATGGGACTCGTCACTGAACAGCA |
| | TAAGTATAGTTTATTAGAACGAACAGCTGAAATGGAAGTG |
| | CTGCCGGCTGCACGGGATCTTGGTTTAGGAGTAGTGGCGT |
| | GGAGTCCCCTTGCAGGAGGGCTTCTTGGCGGGAAGGCATT |
| | GAAAAGCAATGCCGGAACTCGTACAGCAAAAAGAGCAGAT |
| | TTAATTGAAAAACATCGTTTGCAACTCGAGAAATTTTCAG |
| | ATTTATGCAAAGAACTAGGAGAAAAAGAAGCAAATGTGGC |
| | TTTGGCATGGGTGCTGGCAAATCCAGTTTTAACTGCGCCG |
| | ATCATCGGACCACGAACGGTTGAGCAGCTGCGTGATACGA |
| | TAAAAGCCGTTGAAATCAGTCTGGATAAGGAGATTCTCCG |
| | CATGTTAAATGATATCTTTCCCGGACCTGGAGGAGAGACA |
| | CCTGAGGCATACGCCTGGTGA (SEQ ID NO: 100) |

In the present invention, an activity of one kind of ADH may be reduced, and activities of two or more kinds of ADH may be reduced. It is preferable that activities of two or more kinds of ADH are reduced from the viewpoint of further reducing production of an alcohol form as a by-product.

In the present invention, “ADH non-reduced strain” (alternatively, also simply referred to as a “non-reduced strain”) refers to a strain which is not modified such that the ADH activity is reduced. Examples of the ADH non-reduced strain include, but are not limited to, a wild-type strain or a reference strain of each microbial strain and a derivative strain including a strain obtained by breeding. Examples of E. coli strains include, but are not limited to, K-12 strain, B strain, C strain, W strain, and derivative strains thereof such as BL21 (DE3) strain, W3110 strain, MG1655 strain, JM109 strain, DH5α strain, and HB101 strain.

The genetically modified microorganism “modified to reduce an activity of an alcohol dehydrogenase” means that a modification is performed at least to suppress expression of a gene encoding an alcohol dehydrogenase. The “modification to reduce an activity of an alcohol dehydrogenase” includes a modification to suppress an activity of the enzyme, in addition to the modification to suppress expression of a gene encoding an alcohol dehydrogenase. That is, the modified microorganism of the present invention contains a modification to suppress expression of a gene encoding an alcohol dehydrogenase compared to a non-reduced strain (for example, a host microorganism) or a modification to suppress an activity of the enzyme. More specifically, the “modification to reduce an activity of an alcohol dehydrogenase” means that a modification is performed at least to suppress expression of a gene encoding an alcohol dehydrogenase, and a modification is preferably performed to suppress expression of a gene encoding an alcohol dehydrogenase and to suppress an activity of an alcohol dehydrogenase. Since a host microorganism may have plural genes each encoding an alcohol dehydrogenase and plural alcohol dehydrogenases exhibiting an activity against the same substrate may be present, even in the case where “the modification is performed to reduce an activity of an alcohol dehydrogenase”, the activity of the alcohol dehydrogenase may be maintained in a decomposition activity of alcohol species compared to a non-reduced strain. Therefore, as described above, the case where “the modification is performed to reduce an activity of an alcohol dehydrogenase” includes a case where an activity of an alcohol dehydrogenase is maintained compared to a non-reduced strain even though the modification aimed to reduce the activity is performed. In the present invention, with regard to the gene encoding an enzyme, “suppression of expression” also includes “reduction in expression”. In addition, with regard to the enzyme, “suppression of activity” is synonymous with “suppression of function”, “reduction in function”, and “reduction in activity”, and these are used interchangeably.

The genetically modified microorganism of the present invention preferably contains a modification to suppress expression of two or more genes encoding an alcohol dehydrogenase. In the production of the diamine compound, the production amount of the diamine compound can be significantly increased, and production of an alcohol form that is a by-product can also be suppressed, by modifying the microorganism to suppress expression of a plurality of kinds of genes.

The modification performed to reduce the activity of an ADH can be achieved by, for example, reducing expression of a gene encoding an ADH. More specifically, the reduction in the expression of the gene may mean that a transcription amount of a gene (mRNA amount) is reduced and/or that a translation amount of a gene (protein amount) is reduced. The reduction in the expression of the gene also includes a case where a gene is not expressed at all.

The reduction in the expression of the gene may be, for example, a reduction in transcription amount, a reduction in translation amount, or a combination thereof. The reduction in the transcription amount can be achieved by, for example, a method of modifying an expression regulatory region such as a promoter region or a ribosome binding site (RBS) of an ADH gene. The reduction in the transcription amount of the gene can be evaluated by a method known to those skilled in the art, and examples of the method include a quantitative RT-PCR method and a Northern blotting method. The transcription amount of the gene may be reduced to, for example, 50% or less, 20% or less, 10% or less, 5% or less, or 0% compared to the ADH non-reduced strain.

An example of a method of reducing the translation amount includes a method of suppressing translation by inserting a riboswitch region into the upstream of a gene. The riboswitch refers to an RNA that selectively binds to a specific low-molecular compound, and the low-molecular compound refers to a ligand. A secondary structure is formed by RNA base pairs in the absence of a ligand to affect nucleic acids around the riboswitch. In particular, in a case where a ribosome binding site is included in the downstream of the riboswitch, the access of the ribosome to the ribosome binding site is blocked, which interferes with translation of mRNA of a gene located further downstream. On the other hand, the ribosome can access the ribosome binding site in the presence of a ligand through secondary-structure elimination according to ligand binding. Therefore, when a ligand is not added, mRNA of a gene is not translated, and expression of a target gene is suppressed. The reduction in the translation amount of the gene can be evaluated by a method known to those skilled in the art, and an example of the method includes a Western blotting method. The translation amount of the gene may be reduced to, for example, 50% or less, 20% or less, 10% or less, 5% or less, or 0% compared to the ADH non-reduced strain.

In addition, the modification to reduce the activity of the ADH is achieved by disrupting a gene encoding an ADH. The disruption of the ADH gene means that a gene is modified so that a protein having an ADH activity is not expressed, and includes a case where a protein is not produced at all and a case where a protein with reduced or eliminated ADH activity is produced. For example, the disruption can be achieved by deficiency of a part or all of a coding region of a gene on a chromosome. Furthermore, the entire gene containing sequences before and after a gene on a chromosome may be deleted. As long as the reduction of the ADH activity can be achieved, the deleted region may be any one of the N-terminus region, the internal region, and the C-terminus region.

In addition, the disruption of the ADH gene can be achieved by a method of introducing amino acid substitution (missense mutation) or introducing a stop codon (nonsense mutation) into a coding region of an ADH gene on a chromosome, or a method of introducing a frameshift mutation that adds or deletes one or two bases.

Furthermore, the disruption of the ADH gene can be achieved by inserting another sequence into a coding region of a gene on a chromosome. Examples of other sequences include an antibiotic-resistant gene or a transposon, but are not particularly limited as long as the ADH activity is reduced.

The disruption of the ADH gene can be performed by using a method of homologous recombination, and examples of the method include, but are not limited to, a method of using Red recombinase of A-phage (Datsenko, Kirill A., and Barry L. Wanner. “One-step inactivation of chromosomal genes in Escherichia coli K-12 using PCR products.” Proceedings of the National Academy of Sciences 97.12 (2000): 6640-6645), a method of using a Suicide vector containing a temperature sensitive origin of replication (Blomfield et al., Molecular microbiology 5.6 (1991): 1447-1457), and a method of using a CRISPR-Cas9 system (Jiang, Yu, et al. “Multigene editing in the Escherichia coli genome via the CRISPR-Cas9 system.” Appl. Environ. Microbiol. 81.7 (2015): 2506-2514).

In addition, the disruption of the ADH gene can be performed by a mutation treatment. Examples of the mutation treatment include physical treatments such as an X-ray treatment, an ultraviolet ray treatment, and a γ-ray treatment, and chemical treatments with mutagens such as N-methyl-N′-nitro-N-nitrosoguanidine, ethyl methanesulfonate, and methyl methanesulfonate, but are not particularly limited as long as the ADH activity is reduced.

The ADH activity can be evaluated by a method known to those skilled in the art. For example, an example of the evaluation method includes a method of monitoring oxidation of NAD(P)H by incubating a substrate (an aldehyde or a ketone) and NAD(P)H and measuring an absorbance at 340 nm (Pick, et al., Applied microbiology and biotechnology 97.13 (2013): 5815-5824). The ADH activity may be reduced to, for example, 50% or less, 20% or less, 10% or less, 5% or less, or 0% compared to an ADH of an ADH non-reduced strain.

In the genetically modified microorganism according to the present invention, the enzyme involved in synthesis of a diamine compound may have an endogenous property, an exogenous property, or a combination thereof.

The genetically modified microorganism according to the present invention preferably expresses a carboxylic acid reductase as an enzyme gene involved in synthesis of a diamine compound. The carboxylic acid reductase (CAR) generally refers to an arbitrary protein having an activity of reducing a carboxylic acid to convert into an aldehyde. In the present invention, the carboxylic acid reductase has an activity of converting, for example, a carboxyl group of a carboxylic acid semialdehyde, a dicarboxylic acid, or an aminocarboxylic acid into an aldehyde. Examples of the carboxylic acid reductase include, but are not limited to, enzymes classified as EC 1.2.1.30, EC 1.2.1.31, EC 1.2.1.95, EC 1.2.99.6 or the like.

A source of a gene encoding the enzyme is not particularly limited as long as it has a carboxylic acid reducing activity, and examples thereof include, but are not limited to, Nocardia iowensis, Nocardia asteroides, Nocardia brasiliensis, Nocardia farcinica, Segniliparus rugosus, Segniliparus rotundus, Tsukamurella paurometabola, Mycobacterium marinum, Mycobacterium neoaurum, Mycobacterium abscessus, Mycobacterium avium, Mycobacterium chelonae, Mycobacterium immunogenum, Mycobacterium smegmatis, Serpula lacrymans, Heterobasidion annosum, Coprinopsis cinerea, Aspergillus flavus, Aspergillus terreus, Neurospora crassa, and Saccharomyces cerevisiae. In the present invention, for example, a gene encoding a protein consisting of an amino acid sequence set forth in any one of SEQ ID NOs: 101 to 104 is used. A gene encoding a carboxylic acid reductase MaCar derived from Mycobacterium abscessus is preferably used. A base sequence of a coding region of a MaCar gene is set forth in SEQ ID NO: 105, and an amino acid sequence of MaCar is set forth in SEQ ID NO: 103.

TABLE 2-1

| Accession No./ | |
|---|---|
| origin (name | |
| of protein) | Amino acid sequence |
| AAR91681/ | MAVDSPDERLQRRIAQLFAEDEQVKAARPLEAVSAAVSAPGMRLAQIAATVMAGY |
| Nocardia | ADRPAAGQRAFELNTDDATGRTSLRLLPRFETITYRELWQRVGEVAAAWHHDPENP |
| iowensis | LRAGDFVALLGFTSIDYATLDLADIHLGAVTVPLQASAAVSQLIAILTETSPRLLASTP |
| | EHLDAAVECLLAGTTPERLVVFDYHPEDDDQRAAFESARRRLADAGSSVIVETLDA |
| | VRARGRDLPAAPLFVPDTDDDPLALLIYTSGSTGTPKGAMYTNRLAATMWQGNS |
| | MLQGNSQRVGINLNYMPMSHIAGRISLFGVLARGGTAYFAAKSDMSTLFEDIGLVR |
| | PTEIFFVPRVCDMVFQRYQSELDRRSVAGADLDTLDREVKADLRQNYLGGRFLVAV |
| | VGSAPLAAEMKTFMESVLDLPLHDGYGSTEAGASVLLDNQIQRPPVLDYKLVDVP |
| | ELGYFRTDRPHPRGELLLKAETTIPGYYKRPEVTAEIFDEDGFYKTGDIVAELEHDR |
| | LVYVDRRNNVLKLSQGEFVTVAHLEAVFASSPLIRQIFIYGSSERSYLLAVIVPTDDA |
| | LRGRDTATLKSALAESIQRIAKDANLQPYEIPRDFLIETEPFTIANGLLSGIAKLLRPN |
| | LKERYGAQLEQMYTDLATGQADELLALRREAADLPVLETVSRAAKAMLGVASAD |
| | MRPDAHFTDLGGDSLSALSFSNLLHEIFGVEVPVGVVVSPANELRDLANYIEAERN |
| | SGAKRPTFTSVHGGGSEIRAADLTLDKFIDARTLAAADSIPHAPVPAQTVLLTGANG |
| | YLGRFLCLEWLERLDKTGGTLICVVRGSDAAAARKRLDSAFDSGDPGLLEHYQQL |
| | AARTLEVLAGDIGDPNLGLDDATWQRLAETVDLIVHPAALVNHVLPYTQLFGPNV |
| | VGTAEIVRLAITARRKPVTYLSTVGVADQVDPAEYQEDSDVREMSAVRVVRESYAN |
| | GYGNSKWAGEVLLREAHDLCGLPVAVFRSDMILAHSRYAGQLNVQDVFTRLILSLV |
| | ATGIAPYSFYRTDADGNRQRAHYDGLPADFTAAAITALGIQATEGFRTYDVLNPYD |
| | DGISLDEFVDWLVESGHPIQRITDYSDWFHRFETAIRALPEKQRQASVLPLLDAYRN |
| | PCPAVRGAILPAKEFQAAVQTAKIGPEQDIPHLSAPLIDKYVSDLELLQLL |
| | (SEQ ID NO: 101) |
| ACC40567/ | MSPITREERLERRIQDLYANDPQFAAAKPATAITAAIERPGLPLPQIIETVMTGYADRP |
| Mycobacterium | ALAQRSVEFVTDAGTGHTTLRLLPHFETISYGELWDRISALADVLSTEQTVKPGDR |
| marinum | VCLLGFNSVDYATIDMTLARLGAVAVPLQTSAAITQLQPIVAETQPTMIAASVDALA |
| | DATELALSGQTATRVLVFDHHRQVDAHRAAVESARERLAGSAVVETLAEAIARGD |
| | VPRGASAGSAPGTDVSDDSLALLIYTSGSTGAPKGAMYPRRNVATFWRKRTWFEG |
| | GYEPSITLNFMPMSHVMGRQILYGTLCNGGTAYFVAKSDLSTLFEDLALVRPTELTF |
| | VPRVWDMVFDEFQSEVDRRLVDGADRVALEAQVKAEIRNDVLGGRYTSALTGSAP |
| | ISDEMKAWVEELLDMHLVEGYGSTEAGMILIDGAIRRPAVLDYKLVDVPDLGYFLT |
| | DRPHPRGELLVKTDSLFPGYYQRAEVTADVFDADGFYRTGDIMAEVGPEQFVYLD |
| | RRNNVLKLSQGEFVTVSKLEAVFGDSPLVRQIYIYGNSARAYLLAVIVPTQEALDAV |
| | PVEELKARLGDSLQEVAKAAGLQSYEIPRDFIIETTPWTLENGLLTGIRKLARPQLK |
| | KHYGELLEQIYTDLAHGQADELRSLRQSGADAPVLVTVCRAAAALLGGSASDVQP |
| | DAHFTDLGGDSLSALSFTNLLHEIFDIEVPVGVIVSPANDLQALADYVEAARKPGSS |
| | RPTFASVHGASNGQVTEVHAGDLSLDKFIDAATLAEAPRLPAANTQVRTVLLTGAT |
| | GFLGRYLALEWLERMDLVDGKLICLVRAKSDTEARARLDKTFDSGDPELLAHYRA |
| | LAGDHLEVLAGDKGEADLGLDRQTWQRLADTVDLIVDPAALVNHVLPYSQLFGP |
| | NALGTAELLRLALTSKIKPYSYTSTIGVADQIPPSAFTEDADIRVISATRAVDDSYANG |
| | YSNSKWAGEVLLREAHDLCGLPVAVFRCDMILADTTWAGQLNVPDMFTRMILSLA |
| | ATGIAPGSFYELAADGARQRAHYDGLPVEFIAEAISTLGAQSQDGFHTYHVMNPY |
| | DDGIGLDEFVDWLNESGCPIQRIADYGDWLQRFETALRALPDRQRHSSLLPLLHNY |
| | RQPERPVRGSIAPTDRFRAAVQEAKIGPDKDIPHVGAPIIVKYVSDLRLLGLL |
| | (SEQ ID NO: 102) |
| CAM64782/ | MTETISTAAVPTTDLEEQVKRRIEQVVSNDPQLAALLPEDSVTEAVNEPDLPLVEVI |
| Mycobacterium | RRLLEGYGDRPALGQRAFEFVTGDDGATVIALKPEYTTVSYRELWERAEAIAAAW |
| abcessus | HEQGIRDGDFVAQLGFTSTDFASLDVAGLRLGTVSVPLQTGASLQQRNAILEETRPA |
| (MaCar) | VFAASIEYLDAAVDSVLATPSVRLLSVFDYHAEVDSQREALEAVRARLESAGRTIVV |
| | EALAEALARGRDLPAAPLPSADPDALRLLIYTSGSTGTPKGAMYPQWLVANLWQK |
| | KWLTDDVIPSIGVNFMPMSHLAGRLTLMGTLSGGGTAYYIASSDLSTFFEDIALIRPS |
| | EVLFVPRVVEMVFQRFQAELDRSLAPGESNSEIAERIKVRIREQDFGGRVLSAGSGS |
| | APLSPEMTEFMESLLQVPLRDGYGSTEAGGVWRDGVLQRPPVTDYKLVDVPELGY |
| | FTTDSPHPRGELRLKSETMFPGYYKRPETTADVFDDEGYYKTGDVVAELGPDHLK |
| | YLDRVKNVLKLAQGEFVAVSKLEAAYTGSPLVRQIFVYGNSERSFLLAVVVPTPEV |
| | LERYADSPDALKPLIQDSLQQVAKDAELQSYEIPRDFIVETVPFTVESGLLSDARKLL |
| | RPKLKDHYGERLEALYAELAESQNERLRQLAREAATRPVLETVTDAAAALLGASSS |
| | DLAPDVRFIDLGGDSLSALSYSELLRDIFEVDVPVGVINSVANDLAAIARHIEAQRT |
| | GAATQPTFASVHGKDATVITAGELTLDKFLDESLLKAAKDVQPATADVKTVLVTGG |
| | NGWLGRWLVLDWLERLAPNGGKVYALIRGADAEAARARLDAVYESGDPKLSAH |
| | YRQLAQQSLEVIAGDFGDQDLGLSQEVWQKLAKDVDLIVHSGALVNHVLPYSQLF |
| | GPNVAGTAEIIKLAISERLKPVTYLSTVGIADQIPVTEFEEDSDVRVMSAERQINDGY |
| | ANGYGNSKWAGEVLLREAHDLAGLPVRVFRSDMILAHSDYHGQLNVTDVFTRSIQ |
| | SLLLTGVAPASFYELDADGNRQRAHYDGVPGDFTAASITAIGGVNVVDGYRSFDVF |
| | NPHHDGVSMDTFVDWLIDAGYKIARIDDYDQWLARFELALKGLPEQQRQQSVLP |
| | LLKMYEKPQPAIDGSALPTAEFSRAVHEAKVGDSGEIPHVTKELILKYASDIQLLGL |
| | V (SEQ ID NO: 103) |
| AFP42026/ | MHQLTVTGMNICEVQRLFPRMTSDVHDATDGVTETALDDEQSTRRIAELYATDPEF |
| Mycobacterium | AAAAPLPAVVDAAHKPGLRLAEILQTLFTGYGDRPALGYRARELATDEGGRTVTRL |
| smegmatis | LPRFDTLTYAQVWSRVQAVAAALRHNFAQPIYPGDAVATIGFASPDYLTLDLVCAYL |
| | GLVSVPLQHNAPVSRLAPILAEVEPRILTVSAEYLDLAVESVRDVNSVSQLVVFDHH |
| | PEVDDHRDALARAREQLAGKGIAVTTLDAIADEGAGLPAEPIYTADHDQRLAMILY |
| | TSGSTGAPKGAMYTEAMVARLWTMSFITGDPTPVINVNFMPLNHLGGRIPISTAVQ |
| | NGGTSYFVPESDMSTLFEDLALVRPTELGLVPRVADMLYQHHLATVDRLVTQGADE |
| | LTAEKQAGAELREQVLGGRVITGFVSTAPLAAEMRAFLDITLGAHIVDGYGLTETG |
| | AVTRDGVIVRPPVIDYKLIDVPELGYFSTDKPYPRGELLVRSQTLTPGYYKRPEVTA |
| | SVFDRDGYYHTGDVMAETAPDHLVYVDRRNNVLKLAQGEFVAVANLEAVFSGAA |
| | LVRQIFVYGNSERSFLLAVVVPTPEALEQYDPAALKAALADSLQRTARDAELQSYE |
| | VPADFIVETEPFSAANGLLSGVGKLLRPNLKDRYGQRLEQMYADIAATQANQLREL |
| | RRAAATQPVIDTLTQAAATILGTGSEVASDAHFTDLGGDSLSALTLSNLLSDFFGFE |
| | VPVGTIVNPATNLAQLAQHIEAQRTAGDRRPSFTTVHGADATEIRASELTLDKFIDA |
| | ETLRAAPGLPKVTTEPRTVLLSGANGWLGRFLTLQWLERLAPVGGTLITIVRGRDD |
| | AAARARLTQAYDTDPELSRRFAELADRHLRVVAGDIGDPNLGLTPEIWHRLAAEVD |
| | LVVHPAALVNHVLPYRQLFGPNVVGTAEVIKLALTERIKPVTYLSTVSVAMGIPDFE |
| | EDGDIRTVSPVRPLDGGYANGYGNSKWAGEVLLREAHDLCGLPVATFRSDMILAHP |
| | RYRGQVNVPDMFTRLLLSLLITGVAPRSFYIGDGERPRAHYPGLTVDFVAEAVTTLG |
| | AQQREGYVSYDVMNPHDDGISLDVFVDWLIRAGHPIDRVDDYDDWVRRFETALT |
| | ALPEKRRAQTVLPLLHAFRAPQAPLRGAPEPTEVFHAAVRTAKVGPGDIPHLDEALI |
| | DKYIRDLREFGLI (SEQ ID NO: 104) |

TABLE 2-2
Base sequence of a region encoding MaCar

ATGACTGAAACGATCTCCACAGCGGCTGTCCCCACTACGGATCTCGAAGA
GCAGGTGAAGCGACGCATCGAGCAGGTCGTGTCCAACGATCCGCAGCTGG
CGGCGCTTCTCCCGGAAGATTCGGTCACCGAGGCGGTCAACGAGCCCGAT
CTACCGCTGGTCGAGGTGATCAGGCGACTGCTGGAGGGCTACGGTGACCG
CCCGGCACTCGGCCAGCGCGCCTTCGAGTTCGTCACCGGGGACGACGGTG
CGACCGTGATCGCGCTGAAGCCCGAATACACCACCGTCTCCTACCGCGAG
TTGTGGGAACGTGCCGAGGCTATCGCTGCCGCGTGGCACGAGCAGGGCAT
CCGTGACGGCGACTTCGTCGCTCAGTTGGGTTTCACCAGCACGGACTTCG
CGTCGCTCGACGTCGCGGGATTGCGTCTGGGCACCGTCTCGGTGCCCCTG
CAGACGGGCGCGTCGCTGCAGCAGCGCAACGCGATTCTCGAAGAGACCCG
GCCCGCAGTCTTTGCCGCGAGTATCGAATACCTTGATGCCGCCGTCGATT
CGGTGCTTGCGACCCCCTCGGTGCGACTCCTCTCGGTTTTCGACTATCAC
GCGGAGGTCGACAGCCAGCGCGAAGCGCTGGAGGCTGTGCGGGCCCGGCT
TGAGAGTGCCGGCCGGACGATCGTCGTCGAGGCCCTGGCGGAGGCTCTCG
CGCGGGGGCGGGACCTGCCCGCCGCGCCGCTGCCCAGTGCAGATCCCGAT
GCCTTGCGTCTGCTCATCTACACCTCCGGCAGCACCGGTACCCCCAAGGG
CGCCATGTATCCGCAATGGCTGGTCGCCAACTTGTGGCAGAAGAAGTGGC
TCACCGACGATGTGATTCCGTCCATAGGCGTGAACTTCATGCCCATGAGC
CACCTGGCGGGTCGCCTCACTCTCATGGGCACCCTTTCCGGTGGCGGAAC
CGCCTACTACATCGCTTCGAGCGATCTTTCGACTTTCTTCGAGGACATCG
CGCTCATCCGCCCCTCCGAAGTGCTCTTCGTGCCGCGTGTGGTGGAGATG
GTGTTCCAGCGTTTTCAGGCAGAATTGGACCGGTCCCTTGCCCCGGGTGA
GAGCAACTCCGAGATCGCGGAGCGAATCAAGGTCCGCATCCGGGAACAGG
ACTTCGGCGGGCGTGTGCTCAGTGCTGGCTCCGGGTCGGCCCCGTTGTCT
CCTGAGATGACGGAGTTCATGGAGTCGCTGCTGCAGGTGCCGTTGCGCGA
CGGGTATGGGTCCACCGAGGCCGGTGGTGTGTGGCGTGACGGAGTCCTGC
AGCGTCCGCCCGTCACCGACTACAAGCTGGTTGACGTTCCGGAACTCGGA
TACTTCACCACAGATTCGCCGCATCCCCGTGGCGAGCTGCGGTTGAAGTC
GGAGACGATGTTCCCCGGCTACTACAAGCGCCCGGAGACCACTGCCGATG
TCTTCGATGACGAGGGGTACTACAAGACCGGTGACGTGGTCGCCGAGCTC
GGGCCGGATCACCTCAAGTACCTCGACCGCGTCAAGAACGTCCTCAAGCT
CGCGCAGGGAGAGTTTGTCGCGGTGTCAAAGCTGGAGGCCGCTTACACCG
GCAGCCCGCTGGTCCGGCAGATCTTTGTGTACGGGAACAGTGAACGCTCG
TTCCTGCTGGCTGTCGTGGTCCCGACACCCGAAGTCCTTGAGCGGTACGC
AGATTCGCCAGATGCGCTCAAGCCCTTGATCCAGGATTCGCTGCAGCAGG
TCGCCAAGGACGCGGAGCTGCAATCCTATGAGATACCGCGCGACTTCATC
GTTGAGACGGTGCCGTTCACCGTCGAGTCCGGATTGCTATCGGACGCGCG
AAAGCTGCTGCGCCCCAAGCTGAAGGATCACTACGGAGAGAGGCTGGAGG
CGCTGTACGCCGAACTGGCGGAAAGCCAGAATGAGCGGCTGCGCCAGTTG
GCCAGGGAGGCAGCCACGCGCCCGGTCCTGGAGACGGTGACCGATGCGGC
CGCCGCGCTGCTGGGCGCATCGTCCTCGGATCTGGCTCCTGATGTGCGAT
TCATCGACCTCGGTGGCGACTCACTGTCGGCGCTGTCGTACTCCGAGCTG
CTGCGCGACATCTTTGAGGTGGACGTTCCGGTGGGCGTCATCAACAGCGT
CGCCAACGACCTTGCCGCGATCGCCCGGCACATCGAGGCGCAGCGGACCG
GCGCCGCTACGCAGCCGACCTTTGCGTCGGTCCACGGCAAGGACGCGACG
GTCATCACCGCCGGTGAACTCACCCTCGACAAGTTCTTGGACGAGTCACT
GTTGAAAGCGGCCAAGGACGTTCAGCCGGCAACGGCCGATGTCAAGACCG
TTCTAGTGACCGGCGGCAACGGCTGGTTGGGTCGTTGGCTGGTGCTCGAT
TGGCTGGAGCGGTTGGCACCCAATGGTGGCAAGGTCTACGCCCTCATTCG
TGGCGCCGATGCCGAAGCAGCCCGGGCACGGTTGGACGCCGTGTACGAAT
CGGGTGATCCCAAGCTGTCCGCGCATTATCGTCAGCTGGCGCAACAGAGT
CTGGAAGTTATCGCCGGCGATTTCGGCGACCAGGATCTCGGTCTATCCCA
GGAAGTTTGGCAGAAGCTGGCCAAGGACGTGGACCTGATCGTGCACTCCG
GTGCCTTGGTGAACCACGTGCTGCCGTACAGCCAGTTGTTCGGTCCGAAT
GTGGCGGGTACCGCCGAGATCATCAAGCTGGCAATTTCGGAGCGGCTCAA
GCCGGTCACCTACCTGTCGACGGTGGGCATCGCCGACCAGATTCCGGTGA
CGGAGTTCGAGGAAGACTCCGATGTTCGTGTGATGTCGGCCGAGCGCCAG
ATCAATGACGGCTACGCGAACGGATACGGCAACTCAAAATGGGCCGGCGA
GGTGCTGTTGCGGGAGGCTCATGACCTAGCGGGGCTGCCGGTGCGTGTGT
TCCGCTCCGACATGATCCTGGCGCACAGTGACTACCACGGACAGCTCAAC
GTCACCGACGTGTTCACCCGGAGCATCCAGAGTCTGCTGCTCACCGGTGT
TGCACCGGCCAGCTTCTATGAATTGGATGCCGACGGCAATCGGCAGCGCG
CTCACTATGACGGTGTGCCCGGCGATTTCACCGCCGCATCGATCACCGCC
ATCGGCGGTGTGAACGTGGTAGACGGTTACCGCAGCTTCGACGTGTTCAA
CCCGCACCATGACGGTGTCTCGATGGATACCTTCGTCGACTGGCTGATCG
ACGCAGGCTACAAGATCGCGCGGATCGACGATTACGACCAGTGGCTCGCC
CGGTTCGAGCTGGCCCTCAAGGGATTGCCCGAGCAGCAGCGGCAACAGTC
GGTGTTGCCACTTCTCAAGATGTACGAGAAGCCGCAACCGGCGATCGACG
GAAGTGCACTTCCGACCGCAGAATTCAGTCGCGCCGTGCACGAGGCGAAG
GTCGGAGACAGCGGTGAGATACCGCACGTCACCAAGGAGCTGATCCTCAA
GTACGCCAGCGATATTCAGCTGTTGGGCCTGGTGTAG
(SEQ ID NO: 105)

The activity of the carboxylic acid reductase can be evaluated by a method known to those skilled in the art, and examples of the evaluation method include a method of monitoring oxidation of NADPH by incubating a substrate (a carboxylic acid) and an enzyme in the presence of ATP and NADPH and measuring an absorbance at 340 nm, and a method of quantifying a consumption amount of a substrate and/or a production amount of a product (an aldehyde) (Venkitasubramanian et al., Journal of Biological Chemistry, Vol. 282, No. 1, 478-485 (2007)).

In addition, the carboxylic acid reductase can be converted into an active holoenzyme by being phosphopantetinylated (Venkitasubramanian et al., Journal of Biological Chemistry, Vol. 282, No. 1, 478-485 (2007)). The phosphopantetinylation is catalyzed by a phosphopantetheinyl transferase (PT) (for example, an example thereof includes an enzyme classified as EC 2.7.8.7). Therefore, the microorganism of the present invention may be further modified such that an activity of the phosphopantetheinyl transferase is increased. Examples of a method of increasing the activity of the phosphopantetheinyl transferase include, but are not limited to, a method of introducing an exogenous phosphopantetheinyl transferase and a method of enhancing expression of an endogenous phosphopantetheinyl transferase. An example of a donor of the phosphopantetheinyl group includes a coenzyme A (CoA).

A source of a PT gene is not particularly limited as long as it has a phosphopantetheinyl group transfer activity, and examples of a gene encoding a typical phosphopantetheinyl transferase include Sfp of Bacillus subtilis, Npt of Nocardia iowensis (Venkitasubramanian et al., Journal of Biological Chemistry, Vol. 282, No. 1,478-485 (2007)), and Lys5 of Saccharomyces cerevisiae (Ehmann et al., Biochemistry 38.19 (1999): 6171-6177). In the present invention, for example, a gene encoding a protein consisting of an amino acid sequence set forth in any one of SEQ ID NOs: 106 to 108 is used. An Npt gene of Nocardia iowensis derived from Nocardia iowensis is preferably used. A base sequence of a coding region of the Npt gene is set forth in SEQ ID NO: 109, and an amino acid sequence of Npt is set forth in SEQ ID NO: 107.

TABLE 3-1

| Accession | |
|---|---|
| No./ | |
| origin | |
| (name of | |
| protein) | Amino acid sequence |
| CAA44858/ | MKIYGIYMDRPLSQEENERFMTFISPEKREKCRRF |
| derived | YHKEDAHRTLLGDVLVRSVISRQYQLDKSDIRFST |
| from | QEYGKPCIPDLPDAHFNISHSGRWVIGAFDSQPIG |
| Bacillus | IDIEKTKPISLEIAKRFFSKTEYSDLLAKDKDEQT |
| subtilis/ | DYFYHLWSMKESFIKQEGKGLSLPLDSFSVRLHQD |
| (Sfp) | GQVSIELPDSHSPCYIKTYEVDPGYKMAVCAAHPD |
| | FPEDITMVSYEELL (SEQ ID NO: 106) |
| ABI83656/ | MIETILPAGVESAELLEYPEDLKAHPAEEHLIAKS |
| derived | VEKRRRDFIGARHCARLALAELGEPPVAIGKGERG |
| from | APIWPRGVVGSLTHCDGYRAAAVAHKMRFRSIGID |
| Nocardia | AEPHATLPEGVLDSVSLPPEREWLKTTDSALHLDR |
| iowensis | LLFCAKEATYKAWWPLTARWLGFEEAHITFEIEDG |
| (Npt) | SADSGNGTFHSELLVPGQTNDGGTPLLSFDGRWLI |
| | ADGFILTAIAYA (SEQ ID NO: 107) |
| CAA96866/ | MVKTTEVVSEVSKVAGVRPWAGIFVVEIQEDILAD |
| derived | EFTFEALMRTLPLASQARILNKKSFHDRCSNLCSQ |
| from | LLQLFGCSIVTGLNFQELKFDKGSFGKPFLDNNRF |
| Sacharomyces | LPFSMTIGEQYVAMFLVKCVSTDEYQDVGIDIASP |
| cerevisiae | CNYGGREELELFKEVFSEREFNGLLKASDPCTIFT |
| (Lys5) | YLWSLKESYTKFTGTGLNTDLSLIDFGAISFFPAE |
| | GASMCITLDEVPLIFHSQWFNNEIVTICMPKSISD |
| | KINTNRPKLYNISLSTLIDYFIENDGL |
| | (SEQ ID NO: 108) |

TABLE 3-2
Base sequence of a region encoding Npt

ATGATCGAGACAATTTTGCCTGCTGGTGTCGAGTCGGCTGAGCTGCTGGA
GTATCCGGAGGACCTGAAGGCGCATCCGGCGGAGGAGCATCTCATCGCGA
AGTCGGTGGAGAAGCGGCGCCGGGACTTCATCGGGGCCAGGCATTGTGCC
CGGCTGGCGCTGGCTGAGCTCGGCGAGCCGCCGGTGGCGATCGGCAAAGG
GGAGCGGGGTGCGCCGATCTGGCCGCGCGGCGTCGTCGGCAGCCTCACCC
ATTGCGACGGATATCGGGCCGCGGCGGTGGCGCACAAGATGCGCTTCCGT
TCGATCGGCATCGATGCCGAGCCGCACGCGACGCTGCCCGAAGGCGTGCT
GGATTCGGTCAGCCTGCCGCCGGAGCGGGAGTGGTTGAAGACCACCGATT
CCGCACTGCACCTGGACCGTTTACTGTTCTGCGCCAAGGAAGCCACCTAC
AAGGCGTGGTGGCCGCTGACCGCGCGCTGGCTCGGCTTCGAGGAAGCGCA
CATCACCTTCGAGATCGAAGACGGCTCCGCCGATTCCGGCAACGGCACCT
TTCACAGCGAGCTGCTGGTGCCGGGACAGACGAATGACGGTGGGACGCCG
CTGCTTTCGTTCGACGGCCGGTGGCTGATCGCCGACGGGTTCATC
CTCACCGCGATCGCGTACGCCTGA (SEQ ID NO: 109)

As another aspect of producing an aldehyde, the genetically modified microorganism of the present invention may express acyl-(acyl carrier protein (ACP)) reductase (AAR). AAR is an enzyme that converts acyl-ACP into an aldehyde. A gene encoding AAR is not particularly limited, and an example of a typical AAR gene includes AAR of Synechococcus elongatus (Schirmer, Andreas, et al., Science 329.5991 (2010): 559-562).

In addition, as another aspect, an enzyme that produces an aldehyde from acyl-CoA may be expressed. Examples of a gene encoding an enzyme that catalyzes this reaction include, but are not limited to, acr1 of Acinetobacter baylyi encoding fatty acid acyl-CoA dehydrogenase (ZHENG, Yan-Ming, et al., Microbial cell factories, 2012, 11.1:65) and an sucD gene of Clostridium kluyveri encoding succinic acid semialdehyde dehydrogenase (Sohling, B., and Gerhard Gottschalk, Journal of bacteriology 178.3 (1996): 871-880).

Furthermore, an enzyme that produces an aldehyde from acyl phosphate may be expressed, and for example, aspartic acid semialdehyde dehydrogenase (ASD; EC 1.2.1.11) that catalyzes a reaction of aspartic acid semialdehyde from NADPH-dependent 4-aspartyl phosphate can catalyze the same reaction, and an asd gene of E. coli or the like can be used.

The genetically modified microorganism according to the present invention expresses an aminotransferase as an enzyme gene involved in synthesis of a diamine compound.

The aminotransferase refers to an arbitrary enzyme that catalyzes an amino group transfer reaction in the presence of an amino group donor and a receptor. An example of the aminotransferase includes an enzyme classified as EC 2.6.1.p (wherein, p is an integer of 1 or more). Examples of the amino group donor include, but are not limited to, L-glutamic acid, L-alanine, and glycine.

A gene encoding the aminotransferase is not particularly limited as long as it has an amino group transfer activity, and putrescine aminotransferase or other diamine transferases can be preferably used. Examples thereof include a ygjG gene encoding putrescine aminotransferase of E. coli, which is reported to perform amino group transfer on cadaverine and spermidine (Samsonova, et al., BMC microbiology 3.1 (2003): 2), an SpuC gene encoding putrescine aminotransferase of the genus Pseudomonas (Lu et al., Journal of bacteriology 184.14 (2002): 3765-3773, Galman et al., Green Chemistry 19.2 (2017): 361-366), and a gabT gene and a puuE gene encoding GABA aminotransferase of E. coli. Furthermore, it is reported that an ω-transaminase derived from Ruegeria pomeroyi, Chromobacterium violaceum, Arthrobacter citreus, Sphaerobacter thermophilus, Aspergillus fischeri, Vibrio fluvialis, Agrobacterium tumefaciens, Mesorhizobium loti, or the like also has an amino group transfer activity to a diamine compound such as 1,8-diaminooctane and 1,10-diaminodecane, and can be preferably used (Sung et al., Green Chemistry 20.20 (2018): 4591-4595, Sattler et al., Angewandte Chemie 124.36 (2012): 9290-9293).

In the present invention, as the gene encoding an aminotransferase, for example, a gene encoding a protein consisting of an amino acid sequence set forth in any one of SEQ ID NOs: 110 to 114 is used. A putrescine aminotransferase ygjG gene derived from E. coli is preferably used. A base sequence of a coding region of the ygjG gene is set forth in SEQ ID NO: 115, and an amino acid sequence of ygjG is set forth in SEQ ID NO: 110.

TABLE 4-1

| Accession | |
|---|---|
| No./ | |
| origin | |
| (name of | |
| protein) | Amino acid sequence |
| BAE77123/ | MIREPPEHILNRLPSSASALACSAHALNLIEKRTLD |
| derived | HEEMKALNREVIEYFKEHVNPGFLEYRKSVTAGGDY |
| from | GAVEWQAGSLNTLVDTQGQEFIDCLGGFGIFNVGHR |
| Escherichia | NPVVVSAVQNQLAKQPLHSQELLDPLRAMLAKTLAA |
| coli | LTPGKLKYSFFCNSGTESVEAALKLAKAYQSPRGKF |
| K-12 | TFIATSGAFHGKSLGALSATAKSTFRKPFMPLLPGF |
| (ygiG) | RHVPFGNIEAMRTALNECKKTGDDVAAVILEPIQGE |
| | GGVILPPPGYLTAVRKLCDEFGALMILDEVQTGMGR |
| | TGKMFACEHENVQPDILCLAKALGGGVMPIGATIAT |
| | EEVFSVLFDNPFLHTTTFGGNPLACAAALATINVLL |
| | EQNLPAQAEQKGDMLLDGFRQLAREYPDLVQEARGK |
| | GMLMAIEFVDNEIGYNFASEMFRQRVLVAGTLNNAK |
| | TIRIEPPLTLTIEQCELVIKAARKALAAMRVSVEEA |
| | (SEQ ID NO: 110) |
| AAG03688/ | MNSQITNAKTREWQALSRDHHLPPFTDYKQLNEKGA |
| derived | RIITKAEGVYIWDSEGNKILDAMAGLWCVNVGYGRE |
| from | ELVQAATRQMRELPFYNLFFQTAHPPVVELAKAIAD |
| Pseudomonas | VAPEGMNHVFFTGSGSEANDTVLRMVRHYWATKGQP |
| aeruginosa | QKKVVIGRWNGYHGSTVAGVSLGGMKALHEQGDFPI |
| PAO1 | PGIVHIAQPYWYGEGGDMSPDEFGVWAAEQLEKKIL |
| | EVGEENVAAFIAEPIQGAGGVIVPPDTYWPKIREIL |
| | AKYDILFIADEVICGFGRTGEWFGSQYYGNAPDLMP |
| | IAKGLTSGYIPMGGVVVRDEIVEVLNQGGEFYHGFT |
| | YSGHPVAAAVALENIRILREEKIIEKVKAETAPYLQ |
| | KRWQELADHPLVGEARGVGMVAALELVKNKKTRERF |
| | TDKGVGMLCREHCFRNGLIMRAVGDTMIISPPLVID |
| | PSQIDELITLARKCLDQTAAAVLA |
| | (SEQ ID NO: 111) |
| BAA16525/ | MNSNKELMQRRSQAIPRGVGQIHPIFADRAENCRVW |
| derived | DVEGREYLDFAGGIAVLNTGHLHPKVVAAVEAQLKK |
| from | LSHTCFQVLAYEPYLELCEIMNQKVPGDFAKKTLLV |
| Escherichia | TTGSEAVENAVKIARAATKRSGTIAFSGAYHGRTHY |
| coli | TLALTGKVNPYSAGMGLMPGHVYRALYPCPLHGISE |
| K-12 | DDAIASIHRIFKNDAAPEDIAAIVIEPVQGEGGFYA |
| (gabT) | SSPAFMQRLRALCDEHGIMLIADEVQSGAGRTGTLF |
| | AMEQMGVAPDLTTFAKSIAGGFPLAGVTGRAEVMDA |
| | VAPGGLGGTYAGNPIACVAALEVLKVFEQENLLQKA |
| | NDLGQKLKDGLLAIAEKHPEIGDVRGLGAMIAIELF |
| | EDGDHNKPDAKLTAEIVARARDKGLILLSCGPYYNV |
| | LRILVPLTIEDAQIRQGLEIISQCFDEAKQ |
| | (SEQ ID NO: 112) |
| BAA14871/ | MSNNEFHQRRLSATPRGVGVMCNFFAQSAENATLKD |
| derived | VEGNEYIDFAAGIAVLNTGHRHPDLVAAVEQQLQQF |
| from | THTAYQIVPYESYVTLAEKINALAPVSGQAKTAFFT |
| Escherichia | TGAEAVENAVKIARAHTGRPGVIAFSGGFHGRTYMT |
| coli | MALTGKVAPYKIGFGPFPGSVYHVPYPSDLHGISTQ |
| K-12 | DSLDAIERLFKSDIEAKQVAAIIFEPVQGEGGFNVA |
| (puuE) | PKELVAAIRRLCDEHGIVMIADEVQSGFARTGKLFA |
| | MDHYADKPDLMTMAKSLAGGMPLSGVVGNANIMDAP |
| | APGGLGGTYAGNPLAVAAAHAVLNIIDKESLCERAN |
| | QLGQRLKNTLIDAKESVPAIAAVRGLGSMIAVEFND |
| | PQTGEPSAAIAQKIQQRALAQGLLLLTCGAYGNVIR |
| | FLYPLTIPDAQFDAAMKILQDALSD |
| | (SEQ ID NO: 113) |
| WP_ | MATITNHMPTAELQALDAAHHLHPFSANNALGEEGT |
| 011049154/ | RVITRARGVWLNDSEGEEILDAMAGLWCVNIGYGRD |
| derived | ELAEVAARQMRELPYYNTFFKTTHVPAIALAQKLAE |
| from | LAPGDLNHVFFAGGGSEANDTNIRMVRTYWQNKGQP |
| Ruegeria | EKTVIISRKNAYHGSTVASSALGGMAGMHAQSGLIP |
| pomeroyi | DVHHINQPNWWAEGGDMDPEEFGLARARELEEAILE |
| | LGENRVAAFIAEPVQGAGGVIVAPDSYWPEIQRICD |
| | KYDILLIADEVICGFGRTGNWFGTQTMGIRPHIMTI |
| | AKGLSSGYAPIGGSIVCDEVAHVIGKDEFNHGYTYS |
| | GHPVAAAVALENLRILEEENILDHVRNVAAPYLKEK |
| | WEALTDHPLVGEAKIVGMMASIALTPNKASRAKFAS |
| | EPGTIGYICRERCFANNLIMRHVGDRMIISPPLVIT |
| | PAEIDEMFVRIRKSLDEAQAEIEKQGLMKSAA |
| | (SEQ ID NO: 114) |

TABLE 4-2
Base sequence of a region encoding ygjG

ATGATACGCGAGCCTCCGGAGCATATTTTGAACAGGTTACCTTCGAGCGC
ATCGGCTTTAGCGTGCAGCGCCCACGCCCTGAATCTCATTGAGAAGCGAA
CGCTGGATCATGAGGAGATGAAAGCACTTAACCGAGAGGTGATTGAATAC
TTCAAAGAGCATGTCAATCCGGGGTTTTTAGAGTATCGCAAATCTGTTAC
CGCCGGCGGGGATTACGGAGCCGTAGAGTGGCAAGCGGGAAGTTTAAATA
CGCTTGTCGACACCCAGGGCCAGGAGTTTATCGACTGCCTGGGAGGTTTT
GGAATTTTCAACGTGGGGCACCGTAATCCAGTTGTGGTTTCCGCCGTACA
GAATCAACTTGCGAAACAACCGCTGCACAGCCAGGAGCTGCTCGATCCGT
TACGGGCGATGTTGGCGAAAACCCTTGCTGCGCTAACGCCCGGTAAACTG
AAATACAGCTTCTTCTGTAATAGCGGCACCGAGTCCGTTGAAGCAGCGCT
GAAGCTGGCGAAAGCTTACCAGTCACCGCGCGGCAAGTTTACTTTTATTG
CCACCAGCGGCGCGTTCCACGGTAAATCACTTGGCGCGCTGTCGGCCACG
GCGAAATCGACCTTCCGCAAACCGTTTATGCCGTTACTGCCGGGCTTCCG
TCATGTGCCGTTTGGCAATATCGAAGCCATGCGCACGGCTCTTAACGAGT
GCAAAAAAACCGGTGATGATGTGGCTGCGGTGATCCTCGAACCGATTCAG
GGTGAAGGTGGCGTAATTCTGCCGCCGCCGGGCTATCTCACCGCCGTACG
TAAGCTATGCGATGAGTTCGGCGCACTGATGATCCTCGATGAAGTACAAA
CGGGCATGGGGCGCACGGGCAAGATGTTCGCCTGCGAGCATGAGAACGTA
CAGCCGGATATCCTCTGCCTTGCCAAAGCGCTCGGCGGCGGCGTGATGCC
GATTGGCGCGACCATCGCCACTGAAGAGGTGTTTTCAGTTCTGTTCGACA
ACCCATTCCTGCATACCACCACCTTTGGCGGCAACCCGCTGGCCTGTGCG
GCGGCGCTGGCGACCATCAATGTGTTGCTGGAGCAGAACTTACCGGCTCA
GGCTGAGCAAAAAGGCGATATGTTGCTGGACGGTTTCCGTCAACTGGCGC
GGGAATATCCCGATCTGGTACAGGAAGCGCGTGGTAAAGGGATGTTGATG
GCGATTGAGTTTGTTGATAACGAAATCGGCTATAACTTTGCCAGCGAGAT
GTTCCGCCAGCGCGTACTGGTGGCCGGAACGCTCAATAACGCCAAAACGA
TCCGCATTGAACCGCCACTGACACTGACCATTGAACAGTGTGAACTGGTG
ATCAAAGCGGCGCGTAAGGCGCTGGCGGCCATGCGAGTAAGTGTCGAAGA
AGCGTAA (SEQ ID NO: 115)

The gene encoding the enzyme that can be used in the present invention may be derived from an organism other than the exemplified organism or artificially synthesized, and may be any gene that can express a substantial enzyme activity in a host microorganism cell.

In addition, the enzyme gene that can be used for the present invention may have all naturally occurring mutations or artificially introduced mutations and modifications as long as it can express a substantial enzyme activity in the host microorganism cell. For example, it is known that extra codons are present in various codons encoding a specific amino acid. Therefore, in the present invention, alternative codons that are to be finally translated into the same amino acid may also be used. That is, since a genetic code degenerates, a plurality of codons can be used to encode a particular amino acid, so that the amino acid sequence can be encoded by an arbitrary set of similar DNA oligonucleotides. Only the member of the set is identical to the gene sequence of the natural enzyme; however, even mismatched DNA oligonucleotides can hybridize to natural sequences under an appropriate stringent condition (for example, hybridize at 3×SSC and 68° C. and wash at 2×SSC and 0.1% SDS and 68° C.), DNA encoding a natural sequence can by identified and isolated, and such a gene can also be used in the present invention. In particular, since most organisms are known to preferentially use a subset of a specific codon (optimal codon) (Gene, Vol. 105, pp. 61-72, 1991, and the like), performing of “codon optimization” depending on a host microorganism can also be useful in the present invention.

Therefore, the genetically modified microorganism according to the present invention can contain a base sequence having, for example, 80%, 85%, 90%, 95%, 97%, 98%, or 99% or more of sequence identity with the base sequence of the enzyme gene under a condition in which a substantial enzyme activity can be expressed. Alternatively, the genetically modified microorganism according to the present invention can contain a base sequence having, for example, 80%, 85%, 90%, 95%, 97%, 98%, or 99% or more of sequence identity with the base sequence encoding the amino acid sequence of the enzyme.

In the present invention, when the diamine compound synthetase gene group is introduced in a host microorganism cell as an “expression cassette”, a more stable and high level of enzyme activity can be obtained. In the present specification, the “expression cassette” refers to a nucleotide containing a nucleic acid sequence that is functionally linked to a nucleic acid to be expressed or a gene to be expressed and regulates transcription and translation. Typically, the expression cassette of the present invention contains a promoter sequence on the 5′ upstream from the coding sequence, a terminator sequence on the 3′ upstream, and an optionally additional normal regulatory element in a functionally linked state, and in such a case, a nucleic acid to be expressed or a gene to be expressed is introduced into a host microorganism.

The promoter is defined as a DNA sequence that allows RNA polymerase to bind to DNA to initiate RNA synthesis, regardless of whether the promoter is a constitutive expression promoter or an inductive expression promoter. A strong promoter is a promoter that initiates mRNA synthesis at a high frequency, and is also preferably used in the present invention. In E. coli, a major operator and promoter region of a lac system, a trp system, a tac or trc system, or A-phage, a control region for fd coat protein, a promoter for a glycolytic enzyme (for example, 3-phosphoglycerate kinase or glyceraldehyde-3-phosphate dehydrogenase), glutamate decarboxylase A, or serine hydroxymethyltransferase, a promoter region of RNA polymerase derived from T7 phage, and the like can be used. In Corynebacterium glutamicum, a high-level constitutive expression (HCE) promoter, a cspB promoter, a sodA promoter, an elongation factor Tu (EF-Tu) promoter, and the like can be used. As the terminator, a T7 terminator, an rrnBT1T2 terminator, a lac terminator, and the like can be used. In addition to the promoter and terminator sequences, other examples of regulatory elements may include a selection marker, an amplification signal, and a replication point. A preferred regulatory sequence is described in, for example, “Gene Expression Technology: Methods in Enzymology 185” Academic Press (1990)”.

The expression cassette described above is incorporated into a vector consisting of, for example, a plasmid, a phage, a transposon, an IS element, a fosmid, a cosmid, or a linear or cyclic DNA and is inserted into a host microorganism. A plasmid and a phage are preferred. The vector may be self-replicated in a host microorganism or may be replicated by a chromosome. Examples of a preferred plasmid include pLG338, pACYC184, pBR322, pUC18, pUC19, pKC30, pRep4, pHS1, pKK223-3, pDHE19.2, pHS2, pPLc236, pMBL24, pLG200, pUR290, pIN-III113-B1, λgt11, or pBdCI of E. coli; pUB110, pC194, or pBD214 of a Bacillus; and pSA77 or pAJ667 of the genus Corynebacterium. Other plasmids that can also be used are described in “Cloning Vectors”, Elsevier, 1985. The expression cassette can be introduced into the vector by a conventional method of including cutting with an appropriate restriction enzyme, cloning, and ligation. Each expression cassette may be located on one vector or on two or more vectors.

After the vector having the expression cassette of the present invention is constructed as described above, a conventional method can be used as a method that can be applied when the vector is introduced into a host microorganism. Examples of the method include, but are not limited to, a calcium chloride method, an electroporation method, a conjugation transfer method, and a protoplast fusion method, and a method suitable for a host microorganism can be selected.

The modified microorganism obtained as described above is cultured and maintained under conditions suitable for growth and/or maintenance of the modified microorganism for production of the diamine compound of the present invention. A medium composition, culture conditions, and culture time suitable for the modified microorganisms derived from various host microorganisms can be easily set by those skilled in the art.

Another embodiment of the present invention relates to a method of producing a diamine compound using the modified microorganism described above. The method of producing a diamine includes, for example, the following steps.

(a) Culture Step

The method of producing a diamine compound includes a culture step of culturing the modified microorganism according to the embodiment described above. For example, the modified microorganism is cultured in a medium containing a carbon source and a nitrogen source to obtain a culture medium containing bacterial cells.

The present production method may include bringing the genetically modified microorganism into contact with a precursor of a diamine compound. Examples of a method of supplying the precursor of the diamine compound to the modified microorganism include a method of producing a precursor of a diamine compound in a modified microorganism and a method of supplying a precursor of a diamine compound from the outside of a cell without relying on a modified microorganism.

In the culture step, in a case where the modified microorganism is brought into contact with a precursor of a diamine compound, the medium may further contain a precursor, or a precursor may be added to the medium during the culture step.

The medium may be a natural, semi-synthetic, or synthetic medium containing one or more carbon sources, nitrogen sources, inorganic salts, vitamins, and optionally a trace component such as a trace element or vitamin. However, the medium to be used is required to appropriately satisfy nutritional requirements of microorganisms to be cultured.

Examples of the carbon source include D-glucose, sucrose, lactose, fructose, maltose, oligosaccharides, polysaccharides, starch, cellulose, rice bran, waste molasses, fats and oils (for example, soybean oil, sunflower oil, peanut oil, palm oil, and the like), fatty acids (for example, palmitic acid, linoleic acid, oleic acid, linolenic acid, and the like), alcohols (for example, glycerol, ethanol, and the like), and organic acids (for example, acetic acid, lactic acid, succinic acid, and the like). Furthermore, the carbon source may be biomass containing D-glucose. Examples of preferred biomass include a corn decomposition liquid and a cellulose decomposition liquid. These carbon sources can be used alone or as a mixture.

The diamine compound produced using a raw material derived from biomass can be clearly distinguished from a synthetic raw material derived from, for example, petroleum, natural gas, or coal, by measurement of a biomass carbon content based on Carbon-14 (radiocarbon) analysis defined in ISO 16620-2 or ASTM D6866.

Examples of the nitrogen source include nitrogen-containing organic compounds (for example, peptone, casamino acid, tryptone, a yeast extract, a meat extract, a malt extract, a corn steep liquor, soy flour, an amino acid, urea, and the like) and inorganic compounds (for example, an aqueous ammonia solution, ammonium sulfate, ammonium chloride, ammonium phosphate, ammonium carbonate, sodium nitrate, ammonium nitrate, and the like). These nitrogen sources can be used alone or as a mixture.

In addition, the medium may contain a corresponding antibiotic in a case where a modified microorganism expresses a useful additional trait, for example, in a case where a modified microorganism contains a marker resistant to an antibiotic. Therefore, a risk of contamination by various bacteria during culture is reduced. Examples of the antibiotic include, but are not limited to, a β-lactam antibiotic such as ampicillin, an aminoglycoside antibiotic such as kanamycin, a macrolide antibiotic such as erythromycin, a tetracycline antibiotic, and chloramphenicol.

The “precursor” of the diamine compound refers to a compound that can induce a diamine compound by the enzyme involved in synthesis of a diamine compound of the present invention. Examples of the precursor include, but are not limited to, a dicarboxylic acid, a carboxylic acid semialdehyde, a dialdehyde, an aminocarboxylic acid, an aminoaldehyde, acyl-ACP, acyl-CoA, and acyl phosphate.

As a specific precursor that can induce hexamethylenediamine, for example, adipic acid, adipic acid semialdehyde, adipaldehyde, 6-aminohexanoic acid, 6-aminohexanal, adipyl-CoA, adipyl phosphate, or the like can be used.

For example, in a case where a diamine compound is hexamethylenediamine, the modified microorganism is brought into contact with adipic acid that is a precursor, such that the adipic acid is converted into hexamethylenediamine by a carboxylic acid reductase and an aminotransferase produced by the modified microorganism.

In addition, for example, in a case where a diamine compound is 1,10-decanediamine, the modified microorganism is brought into contact with sebacic acid that is a precursor, such that the sebacic acid is converted into 1,10-decanediamine by a carboxylic acid reductase and an aminotransferase produced by the modified microorganism.

As the precursor, one precursor may be used, or two or more precursors may be combined. In addition, in a case where the compound is a compound that can adopt a salt form, the precursor may be used as a salt, a free form, or a mixture thereof.

The method of producing a precursor is not particularly limited, and a precursor can be produced by, for example, a chemical synthesis method, an enzyme method, a bioconversion method, a fermentation method, or a combination thereof.

In the culture step, the genetically modified microorganism of the present invention can be brought into contact with a precursor of a diamine compound to generate and accumulate a diamine compound in a medium, thereby producing a diamine compound. In addition, as described below, in a reaction step, the genetically modified microorganism of the present invention may be allowed to act in an aqueous solution containing a precursor of a diamine compound to produce and accumulate a diamine compound in the reaction solution.

(b) Reaction Step

The present step is a step of bringing a precursor of a diamine compound into contact with the modified microorganism to produce a target diamine compound from the precursor of the diamine compound. The contact of the precursor of the diamine compound may be performed, for example, in the culture step described above, or may be performed after the culture step. In a case where the present reaction step is performed after the culture step, the culture medium and/or bacterial cells obtained in the culture step can be brought into contact with an aqueous solution containing a precursor of a diamine compound to obtain a reaction solution containing a diamine compound. The diamine compound is produced and accumulated in the reaction solution by being brought into contact with such a precursor.

In one aspect, in the present step, the culture medium containing the bacterial cells obtained in the culture step and/or the bacterial cells from which a supernatant is removed by centrifugation or the like from the culture medium obtained in the culture step are brought into contact with an aqueous solution containing a precursor to obtain a reaction solution.

In addition, in another aspect, bacteria that produce a precursor by fermentation and the modified microorganism according to the present invention may be co-cultured. By co-culturing the bacteria and the modified microorganism, the precursor produced by the bacteria can be efficiently converted into a target diamine compound by the enzyme produced by the modified composition according to the present invention.

In still another aspect, the genetically modified microorganism according to the present invention is allowed to have an ability of producing a precursor of a diamine compound so that a diamine compound may be produced and accumulated from components in the medium.

As an example of a precursor production pathway by the genetically modified microorganism, a method of producing 6-aminohexanoic acid using glucose as a starting material (Turk et al., ACS synthetic biology 5.1 (2015): 65-73) and a method of producing adipic acid using glucose or glycerol as a starting material (Zhao et al., Metabolic engineering 47 (2018): 254-262) are disclosed, and the method is not limited as long as a precursor can be produced.

For example, the modified microorganism has an ability of producing a dicarboxylic acid, a carboxylic acid semialdehyde, or an aminocarboxylic acid, and further expresses an aminotransferase and a carboxylic acid reductase so that a diamine compound can be produced.

In addition, for example, the modified microorganism has an ability of producing adipic acid, adipic acid semialdehyde, or 6-aminohexanoic acid, and further expresses an aminotransferase and a carboxylic acid reductase so that hexamethylenediamine can be produced.

According to the genetically modified microorganism and the method of producing a diamine compound using the microorganism of the present invention, production of a by-product can be suppressed, and a diamine compound can be more efficiently produced. Specifically, for example, the microorganism that expresses an enzyme required to produce a diamine compound is modified to reduce an activity of an alcohol dehydrogenase compared to a non-reduced strain, thereby suppressing production of an alcohol form that is a by-product and efficiently producing a diamine compound.

Hereinafter, examples are given for the purpose of further description, and the present invention is not limited to the examples. In the present specification, unless otherwise specified, nucleotide sequences are described from 5′ directions to 3′ direction.

### EXAMPLES

Hereinafter, the present invention will be explained based on examples, but the present invention is not limited to these examples.

### 1: Construction of ADH Gene-Disrupted Strain

### 1-a Construction of Plasmid for Disrupting Gene

Disruption of an ADH gene was performed by a homologous recombination method using pHAK1 (deposited with biotechnology division of National Institute of Technology and Evaluation (NITE), Patent Microorganisms Depositary (NPMD) (address: #122, 2-5-8, Kazusakamatari, Kisarazu-shi, Chiba, Japan) on Mar. 18, 2019, under the accession number NITE P-02919). pHAK1 contains a temperature-sensitive variant repA gene, a kanamycin resistant gene, and a levansucrase gene SacB derived from Bacillus subtilis. The levansucrase gene lethally acts on a host microorganism under the presence of sucrose. The PCR fragment was amplified using PrimeSTAR Max DNA Polymerase (trade name, manufactured by TAKARA BIO INC.), and the plasmid was prepared using an E. coli HST08 strain. Using the genomic DNA of the E. coli BL21 (DE3) strain as a template, a PCR product containing an upstream region, a coding region, and a downstream region of a disruption target gene was obtained. The combinations of the target gene and the primer sequence are shown in the following table.

TABLE 5

| Target | Forward primer | Reverse primer |
|---|---|---|
| gene | (SEQ ID NO) | (SEQ ID NO) |
| yqhD | TTGTTGAATAAATCGT | TATCGCATGCAGATCT |
| | CTGTTTGCCGAGAATA | GCGCTAATGGGCTCCA |
| | CGCG | GGA |
| | (SEQ ID NO: 116) | (SEQ ID NO: 117) |
| fucO | TTGTTGAATAAATCGC | TATCGCATGCAGATCC |
| | GCGCCAGCGCTGGCTG | CGCCAATGCCGGAAGA |
| | TTT | GTT |
| | (SEQ ID NO: 118) | (SEQ ID NO: 119) |
| adhP | TTGTTGAATAAATCGC | TATCGCATGCAGATCG |
| | GATCGTGATGCCGCTG | ACAACGTAGGCTTTGT |
| | TCT | TCA |
| | (SEQ ID NO: 120) | (SEQ ID NO: 121) |
| eutG | TTGTTGAATAAATCGC | TATCGCATGCAGATCG |
| | GCCATCTCGACACTCT | CGAACATCGATGGGTT |
| | TGA | AGC |
| | (SEQ ID NO: 122) | (SEQ ID NO: 123) |
| ybbO | TTGTTGAATAAATCGT | TATCGCATGCAGATCG |
| | GGCATTTGCCCTTCCT | TCCTGATCCTGCAACG |
| | GTT | GAA |
| | (SEQ ID NO: 124) | (SEQ ID NO: 125) |
| ahr | TTGTTGAATAAATCGC | TATCGCATGCAGATCT |
| | TCATAACGGTACTGCA | CGCAGCAGGTAAGATG |
| | AAC | ATT |
| | (SEQ ID NO: 126) | (SEQ ID NO: 127) |
| yahK | TTGTTGAATAAATCGA | TATCGCATGCAGATCT |
| | TATTCGTCCTAACGAA | TTTTGATTTTCAAGTA |
| | CAG | TGT |
| | (SEQ ID NO: 128) | (SEQ ID NO: 129) |

Next, the present PCR product was inserted into the pHAK1 plasmid fragment amplified using primers of SEQ ID NOs: 130 and 131 using an In-Fusion HD cloning kit (trade name, manufactured by Clontech Laboratories, Inc.) to circularize it.

TABLE 6
GATCTGCATGCGATATCTCTAGAACGCGTAAGCTT
(SEQ ID NO: 130)
TCTCGAGCCGATTTATTCAACAAAGCCGC
(SEQ ID NO: 131)

PCR was performed using the pHAK1 plasmid into which the DNA fragment containing the upstream region, the coding region, and the downstream region of the obtained disruption target gene as a template and using primers shown in the following table, thereby obtaining a plasmid fragment in which the coding region of the disruption target gene was partially or entirely removed.

TABLE 7

| Target | Forward primer | Reverse primer |
|---|---|---|
| gene | (SEQ ID NO) | (SEQ ID NO) |
| yqhD | ACTTTCGTTTTCGGGC | CCAATATGAGGGCAG |
| | ATTTCGTCC | AGAACGATC |
| | (SEQ ID NO: 132) | SEQ ID NO: 133) |
| fucO | ATGCGCTGATGTGATA | CCTTCTCCTTGTTGC |
| | ATGC | TTTA |
| | (SEQ ID NO: 134) | (SEQ ID NO: 135) |
| adhP | GAGGCCTTTGCTGCGA | AGTTCCTCCTTTTCGG |
| | CTGC | ATGAT |
| | (SEQ ID NO: 136) | (SEQ ID NO: 137) |
| eutG | ATGCCGGATGCGACGC | TCATTTTGCATATAGC |
| | TT | CCCT |
| | (SEQ ID NO: 138) | (SEQ ID NO: 139) |
| ybbO | TGACCTGGGCAGTAAT | CAGGATCTCCGTTGCT |
| | GGTG | TTATGAGTC |
| | (SEQ ID NO: 140) | (SEQ ID NO: 141) |
| ahr | CGTGGTGTTGAAAGCC | CATAAACTTCCAGTTC |
| | GATTATTG | TCCGCCC |
| | (SEQ ID NO: 142) | (SEQ ID NO: 143) |
| yahK | TCGCACACTAACAGAC | TGTGTTTACTCCTGAT |
| | TGAA | TAGC |
| | (SEQ ID NO: 144) | (SEQ ID NO: 145) |

The obtained plasmid fragment was subjected to terminal phosphorylation and circularization by self-ligation to obtain a plasmid for disrupting a gene.

### 1-b Construction of ADH Gene-Disrupted E. coli Strain

A plasmid for disrupting a desired gene was transformed into the E. coli BL21 (DE3) strain by a calcium chloride method (refer to Genetic Engineering Laboratory Notebook, by Takaaki Tamura, Yodosha), and then applied to an LB agar medium (10 g/L of tryptone, 5 g/L of yeast extract, 5 g/L of sodium chloride, and 15 g/L of agar powder) containing 100 mg/L of kanamycin sulfate, and culture was performed at 30° C. overnight to obtain a single colony, thereby obtaining a transformant. The present transformant was inoculated into 1 mL of an LB liquid medium (10 g/L of tryptone, 5 g/L of yeast extract, and 5 g/L of sodium chloride) containing 100 mg/L of kanamycin sulfate with a platinum loop, and shaking culture was performed at 30° C. The obtained culture medium was applied to an LB agar medium containing 100 mg/L of kanamycin sulfate, and culture was performed at 42° C. overnight. In the obtained colony, a plasmid was inserted into the genome by single crossover. The colony was inoculated into 1 mL of an LB liquid medium with a platinum loop, and shaking culture was performed at 30° C. The obtained culture medium was applied to an LB agar medium containing 10% sucrose, and culture was performed overnight. Disruption of a desired gene in the obtained colony was observed by colony direct PCR using the primer set shown in Table 8. The constructed ADH gene-disrupted E. coli strain is shown in Table 9. In the table, A indicates that the enzyme gene is deficient.

TABLE 8

| Target | Forward primer | Reverse primer |
|---|---|---|
| gene | (SEQ ID NO) | (SEQ ID NO) |
| yqhD | TATTCTCAATCCGTTT | TTCGGGATCACCACCA |
| | CAGCACGCG | GGCCG |
| | (SEQ ID NO: 146) | (SEQ ID NO: 147) |
| fucO | CGGAAATGGACGAACA | CGTCATCAGCGTTTAC |
| | GTGG | CAGATT |
| | (SEQ ID NO: 148) | (SEQ ID NO: 149) |
| adhP | GCATAAACACTGTCCG | GAAATCGAGAAGGCAG |
| | CGTC | AAGCGAAA |
| | (SEQ ID NO: 150) | (SEQ ID NO: 151) |
| eutG | GGTGCGGTCACCATTG | CATATCGCACGCCAGC |
| | TTCG | AGTG |
| | (SEQ ID NO: 152) | (SEQ ID NO: 153) |
| ybbO | GGTGAGGATGGAGAGT | CAGTTCGATTTGCGCC |
| | TCATG | ACCAG |
| | (SEQ ID NO: 154) | (SEQ ID NO: 155) |
| ahr | TCCGCTAGTGTGATTT | GAAATTATTATGCCGC |
| | CAGG | CAGGCGT |
| | (SEQ ID NO: 156) | (SEQ ID NO: 157) |
| yahK | TTATGGTCTGGGCGAC | GCATCATCCTGGTCAT |
| | ATGC | ATACCC |
| | (SEQ ID NO: 158) | (SEQ ID NO: 159) |

TABLE 9

BL21(DE3) ΔyqhD
BL21(DE3) ΔfucO
BL21(DE3) ΔadhP
BL21(DE3) ΔeutG
BL21(DE3) ΔybbO
BL21(DE3) Δahr
BL21(DE3) ΔyahK
BL21(DE3) ΔyqhD ΔfucO
BL21(DE3) ΔyqhD ΔadhP
BL21(DE3) ΔyqhD ΔeutG
BL21(DE3) ΔyqhD ΔybbO
BL21(DE3) ΔyqhD Δahr
BL21(DE3) ΔyqhD ΔyahK
BL21(DE3) ΔyqhD ΔfucO ΔadhP
BL21(DE3) ΔyqhD ΔfucO ΔadhP ΔeutG
BL21(DE3) ΔyqhD ΔfucO ΔadhP ΔeutG ΔybbO
BL21(DE3) ΔyqhD ΔfucO ΔadhP ΔeutG ΔybbO Δahr
BL21(DE3) ΔyqhD ΔfucO ΔadhP ΔeutG ΔybbO Δahr ΔyahK

### 1-c Test for Reducing Decomposition Activity of 1,6-Hexanediol

The reduction in decomposition activity of 1,6-hexanediol of the constructed ADH gene-disrupted E. coli strain was confirmed by the progress of the oxidation reaction of 1,6-hexanediol. 1,6-Hexanediol is one of alcohol forms that are by-products of the production reaction of hexamethylenediamine. In this test, based on the fact that the reaction between the aldehyde and the alcohol catalyzed by ADH illustrated in FIG. 1 is a reversible reaction, a conversion reaction from the alcohol (here, 1,6-hexanediol) to the aldehyde was focused on, and the consumption of 1,6-hexanediol was used as an index of the decomposition activity of 1,6-hexanediol by ADH. In this test, the bacterial cells of each of the ADH gene-disrupted E. coli strains were inoculated into 2 mL of an LB liquid medium with a platinum loop, and shaking culture was performed at 37° C. overnight as pre-culture. The obtained pre-culture medium was inoculated into 2 mL of an LB liquid medium containing 10 mM 1,6-hexanediol in an amount corresponding to 1%, and shaking culture was performed at 37° C. for 48 hours as a main culture. The culture medium was separated into the bacterial cells and the supernatant by centrifugation, and a concentration of 1,6-hexanediol in the supernatant was analyzed.

The analysis of the concentration of 1,6-hexanediol was performed using gas chromatograph.

The conditions are as follows.

GC system: GC-2010 (manufactured by Shimadzu Corporation)

Detector: Hydrogen flame ionization detector

Column: DB-WAX (manufactured by Agilent Technologies, column length: 30 m, inner diameter: 0.25 mm, film thickness: 0.25 mm)

Carrier gas: He

Gas pressure: 100 kPa

Column temperature: 50° C.—(25° C./min)—230° C.—(holding for 20 min)

Detector temperature: 250° C.

Inlet temperature: 250° C.

Injection amount: 1 μL

Injection method: Split injection method (split ratio: 36.3)

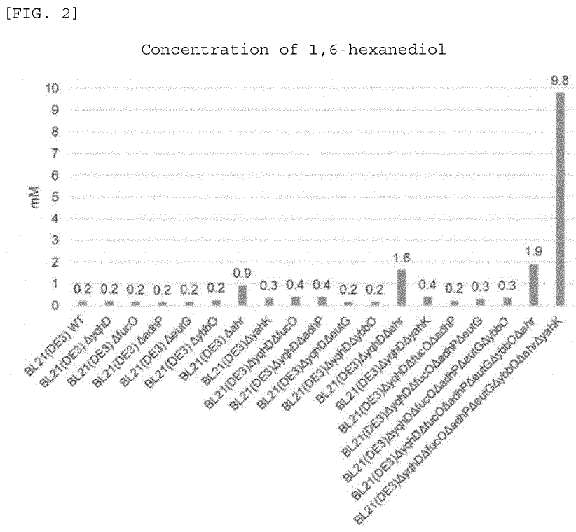

The concentration of 1,6-hexanediol in the culture supernatant 48 hours after the main culture is shown in FIG. 2. In the wild-type strain (BL21(DE3) strain, WT is an abbreviation of Wild type and indicates wild type) in which the ADH gene was not disrupted, 1,6-hexanediol was consumed by the action of the ADH, whereas in the ADH gene-disrupted strain, particularly regarding two genes: the ahr gene and the yahK gene, consumption of 1,6-hexanediol was suppressed by single gene disruption. It was confirmed from the present results that the decomposition activity of 1,6-hexanediol was reduced in the ADH gene-disrupted strain.

### 2: Production of Diamine Compound in ADH Gene-Disrupted Strain

### 2-a Construction of MaCar Gene, Npt Gene, and ygjG Gene Expression Plasmids

The PCR fragment was amplified using PrimeSTAR Max DNA Polymerase (trade name, manufactured by TAKARA BIO INC.), and the plasmid was prepared using an E. coli JM109 strain. PCR was performed using the genome DNA of the Escherichia coli W3110 strain (NBRC12713) as a template, and using oligonucleotides of SEQ ID NOs: 160 and 161 as primers, thereby obtaining a PCR product containing a coding region of a ygjG gene. Next, the present PCR product was inserted between restriction enzymes NcoI and HindIII cleavage sites of plasmid pACYCDuet (trademark)-1 (trade name, manufactured by Merck & Co., Inc.) using an In-Fusion HD cloning kit (trade name, manufactured by Clontech Laboratories, Inc.), and the PCR product was named “pDA50”.

TABLE 10
ATAAGGAGATATACCATGATACGCGAGCCTCCGGA
(SEQ ID NO: 160)
ATGCGGCCGCAAGCTTTACGCTTCTTCGACACTTA
(SEQ ID NO: 161)

PCR was performed using the genome DNA of the Mycobacterium abscessus JCM13569 strain (provided by RIKEN BRC through Ministry of Education, Culture, Sports, Science and Technology/the National BioResource Project of Japan Agency for Medical Research and Development) as a template and using oligonucleotides of SEQ ID NOs: 162 and 163 as primers, thereby obtaining a PCR product containing a coding region of an MaCar gene. Next, the PCR product was inserted between restriction enzymes NdeI and AvrII cleavage sites of pDA50 using an In-Fusion HD cloning kit (trade name, manufactured by Clontech Laboratories, Inc.), and the PCR product was named “pDA52”.

TABLE 11
TAAGAAGGAGATATACATATGACTGAAACGATCTC
(SEQ ID NO: 162)
GTGGCAGCAGCCTAGCTACACCAGGCCCAACAGCT
(SEQ ID NO: 163)

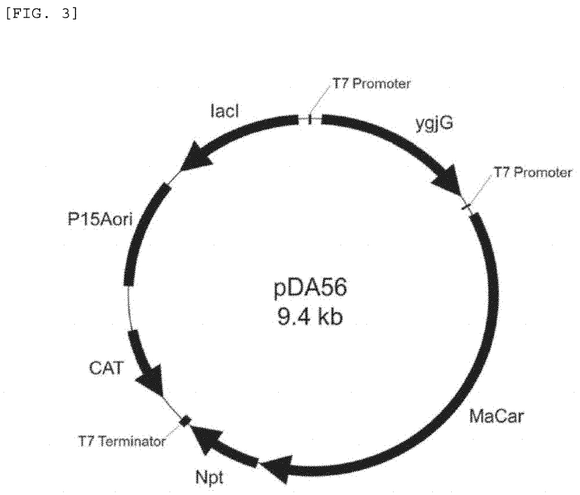

PCR was performed using the genome DNA of the Nocardia iowensis JCM18299 strain (provided by RIKEN BRC through Ministry of Education, Culture, Sports, Science and Technology/the National BioResource Project of Japan Agency for Medical Research and Development) as a template and using oligonucleotides of SEQ ID NOs: 164 and 165 as primers, thereby obtaining a PCR product containing a coding region of an Npt gene. Next, PCR was performed using pDA52 as a template and using oligonucleotides of SEQ ID NOs: 166 and 167 as primers, thereby obtaining a pDA52 fragment. The PCR products were connected to each other using an In-Fusion HD cloning kit (trade name, manufactured by Clontech Laboratories, Inc.). The plasmid was extracted from the obtained transformant, and the product into which the Npt gene was inserted was named “pDA56”. The plasmid map of pDA56 is illustrated in FIG. 3.

TABLE 12
TTTAAGGAGTTCGATATGATCGAGACAATTTTGCC
(SEQ ID NO: 164)
GGTGGCAGCAGCCTAGTCAGGCGTACGCGATCGCG
(SEQ ID NO: 165)
CTAGGCTGCTGCCACCGCTG
(SEQ ID NO: 166)
ATCGAACTCCTTAAATTTATCTACACCAGGCCCAACAGCT
(SEQ ID NO: 167)

### 2-b Preparation of Transformant

pDA56 was introduced into an ADH gene non-disrupted E. coli strain or an ADH gene-disrupted strain by a calcium chloride method (refer to Genetic Engineering Laboratory Notebook, by Takaaki Tamura, Yodosha), and culture was performed in an LB agar medium containing 34 mg/L of chloramphenicol overnight, thereby obtaining a transformant. The obtained transformants were named transformants A to S as shown in the following table. As shown in the table, the transformant A is an ADH gene non-disrupted strain, the transformants B to H are strains in which any one of genes encoding an ADH are disrupted, and the transformants I to S are strains in which at least two of genes encoding ADH are disrupted (multiply disrupted).

TABLE 13

| Name of | | |
|---|---|---|
| transformant | Microorganism strain | Plasmid |
| A | BL21 (DE3) | pDA56 |
| B | BL21 (DE3) ΔyqhD | |
| C | BL21 (DE3) ΔfucO | |
| D | BL21 (DE3) ΔadhP | |
| E | BL21 (DE3) ΔeutG | |
| F | BL21 (DE3) ΔybbO | |
| G | BL21 (DE3) Δahr | |
| H | BL21 (DE3) ΔyahK | |
| I | BL21 (DE3) ΔyqhDΔfucO | |
| J | BL21 (DE3) ΔyqhDΔadhP | |
| K | BL21 (DE3) ΔyqhDΔeutG | |
| L | BL21 (DE3) ΔyqhDΔybbO | |
| M | BL21 (DE3) ΔyqhDΔahr | |
| N | BL21 (DE3) ΔyqhDΔyahK | |
| O | BL21 (DE3) ΔyqhDΔfucOΔadhP | |
| P | BL21 (DE3) ΔyqhDΔfucOΔadhPΔeutG | |
| Q | BL21 (DE3) ΔyqhDΔfucOΔadhPΔeutGΔybbO | |
| R | BL21 (DE3) ΔyqhDΔfucOΔadhPΔeutGΔybbOΔahr | |
| S | BL21 (DE3) ΔyqhDΔfucOΔadhPΔeutGΔybbOΔahrΔyahK | |

### 2-c Production of Hexamethylenediamine from Adipic Acid (Comparative Example 1 and Examples 1 to 18)

The bacterial cells of the transformants A to S were inoculated in 2 mL of an LB liquid medium containing 34 mg/L of chloramphenicol with a platinum loop, and shaking culture was performed at 37° C. overnight as pre-culture. The obtained pre-culture medium was inoculated in 1 mL of an SOB liquid medium (20 g/L of tryptone, 5 g/L of yeast extract, 0.5 g/L of sodium chloride, 2.5 mM calcium chloride, 10 mM magnesium sulfate, and 10 mM magnesium chloride) containing 50 mM diammonium adipate, 34 mg/L of chloramphenicol, and 2% glucose in an amount corresponding to 1%, and shaking culture was performed at 37° C. After 2 hours of culture, isopropyl β-thiogalactopyranoside (IPTG) was added so that the final concentration was 0.2 mM, and shaking culture was performed at 30° C. for 48 hours. The culture medium was separated into the bacterial cells and the supernatant by centrifugation, and a concentration of hexamethylenediamine and a concentration of 1,6-hexanediol in the supernatant were analyzed.

The analysis of the concentration of hexamethylenediamine was performed using ion chromatograph. The conditions are as follows.

Apparatus: ICS-3000 (manufactured by Dionex Corporation)

Detector: Electrical conductivity detector

Column: IonPac CG19 (2×50 mm)/CS19(2×250 mm) (manufactured by Thermo Fisher Scientific)

Oven temperature: 30° C.

Mobile phase: 8 mM aqueous methanesulfonic acid solution (A), 70 mM aqueous methanesulfonic acid solution (B)

Gradient condition: (A: 100%, B: 0%)—(10 min)-(A: 0%, B: 100%)—(holding for 1 min)

Flow rate: 0.35 mL/min

Injection amount: 20 μL

The concentration of 1,6-hexanediol was performed using gas chromatograph under the same conditions as in (1-c).

The concentration of hexamethylenediamine and the concentration of 1,6-hexanediol in each of the culture media are shown in Table 14. First, as for the concentration of hexamethylenediamine, an increase in production amount of hexamethylenediamine was observed in the ADH gene-disrupted strains of Examples 1, 3, and 8 to 18 compared to the ADH gene non-disrupted strain (Comparative example 1). In addition, in the ADH gene-disrupted strains of Examples 1, 2, and 4 to 18, the production amount of 1,6-hexanediol was reduced. In addition, the production amount of hexamethylenediamine was further increased by multiple disruption of the ADH gene (Examples 8 to 18). Regarding the concentration of 1,6-hexanediol, it was observed that the production was suppressed compared to the ADH gene non-disrupted strain (Comparative example 1).

TABLE 14

| | | Concentration of | Concentration of |
|---|---|---|---|
| | | hexamethylenediamine | 1,6-hexanediol |
| | Transformant | (μM) | (mM) |
| Comparative | A | 2 | 1.70 |
| example 1 | | | |
| Example 1 | B | 10 | 0.22 |
| Example 2 | C | 2 | 1.41 |
| Example 3 | D | 4 | 2.01 |
| Example 4 | E | 2 | 1.57 |
| Example 5 | F | 1 | 1.18 |
| Example 6 | G | 2 | 1.30 |
| Example 7 | H | 2 | 1.28 |
| Example 8 | I | 38 | 0.27 |
| Example 9 | J | 81 | 0.38 |
| Example 10 | K | 48 | 0.29 |
| Example 11 | L | 18 | 0.18 |
| Example 12 | M | 69 | 0.08 |
| Example 13 | N | 63 | 0.26 |
| Example 14 | O | 68 | 0.31 |
| Example 15 | P | 54 | 0.28 |
| Example 16 | Q | 42 | 0.24 |
| Example 17 | R | 38 | 0.09 |
| Example 18 | S | 47 | 0.05 |

### 2-d Production of 1,10-Decanediamine from Sebacic Acid (Comparative Example 2 and Examples 19 and 20)

The bacterial cells of the transformants A to S were inoculated in 2 mL of an LB liquid medium containing 34 mg/L of chloramphenicol with a platinum loop, and shaking culture was performed at 37° C. overnight as pre-culture. The obtained pre-culture medium was inoculated in 1 mL of an SOB liquid medium containing 50 mM sodium sebacate, 34 mg/L of chloramphenicol, and 2% glucose in an amount corresponding to 1%, and shaking culture was performed at 37° C. After 2 hours of culture, IPTG was added so that the final concentration was 0.2 mM, and shaking culture was performed at 30° C. for 48 hours. The culture medium was separated into the bacterial cells and the supernatant by centrifugation, and a concentration of 1,10-decanediamine and a concentration of 1,10-decanediol in the supernatant were analyzed.

The analysis of the concentration of 1,10-decanediamine was performed by ion chromatography under the same conditions as in (2-c). The concentration of 1,10-decanediamine in each of the culture media is shown in Table 15. It was observed that the production amount of 1,10-diaminodecane in the ADH gene-disrupted strain of each of Examples 19 and 20 was increased compared to the ADH gene non-disrupted strain (Comparative Example 2).

The concentration of 1,10-decanediol was performed using gas chromatograph under the same conditions as in (1-c). The concentration of 1,10-decanediamine in each of the culture media is shown in Table 15. It was observed that the production amount of 1,10-decanediol was suppressed in the ADH gene-disrupted strains of Examples 19 and 20 compared to the ADH gene non-disrupted strain (Comparative example 2).

TABLE 15

| | | Concentration of | Concentration of |
|---|---|---|---|
| | | 1,10-decanediamine | 1,10-decanediol |
| | Transformant | (μM) | (mM) |
| Comparative | A | 107 | 1.03 |
| example 2 | | | |
| Example 19 | B | 199 | 0.55 |
| Example 20 | S | 228 | 0.57 |

Deposit of Biological Material

The plasmid pHAK1 was deposited with biotechnology division of National Institute of Technology and Evaluation (MITE), Patent Microorganisms Depositary (NPMD) (address: #122, 2-5-8, Kazusakamatari, Kisarazu-shi, Chiba, Japan) on Jul. 21, 2020, under “NITE ABP-02919 (accession number)” (the demand for conversion from NITE P-02919 to the deposit under Budapest Treaty was filed).

### INDUSTRIAL APPLICABILITY

The genetically modified microorganism of the present invention can be suitably used in production of a diamine compound.

20200722111720202007161145140240 P1AP101_19_73.app

1. A genetically modified microorganism that expresses an enzyme involved in synthesis of a diamine compound, wherein
the diamine compound is represented by Formula: H₂N—R—NH₂
(wherein, R is a chain or cyclic organic group comprised of one or more atoms selected from the group consisting of C, H, O, N, and S), and
the genetically modified microorganism is modified to reduce an activity of an alcohol dehydrogenase compared to a non-reduced strain.

2. The genetically modified microorganism according to claim 1, wherein the modification performed to reduce the activity of the alcohol dehydrogenase compared to the non-reduced strain is
a modification to suppress expression of a gene encoding an alcohol dehydrogenase or
a modification to suppress expression of a gene encoding an alcohol dehydrogenase and to suppress an activity of an alcohol dehydrogenase.

3. The modified microorganism according to claim 1, wherein the alcohol dehydrogenase is a protein encoded by
DNA consisting of a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100,
DNA consisting of a base sequence having 85% or more of sequence identity with a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100 and encoding a protein having an alcohol dehydrogenase activity,
DNA consisting of a base sequence encoding a protein consisting of an amino acid sequence obtained by deleting, substituting, inserting, and/or adding 1 to 10 amino acids with respect to an amino acid sequence of a protein encoded by a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100 and encoding a protein having an alcohol dehydrogenase activity, or
DNA consisting of a degenerate isomer of a base sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, and 100.

4. The modified microorganism according to claim 1, wherein the alcohol dehydrogenase is a protein containing an amino acid sequence having 80% or more of sequence identity with an amino acid sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, and 99 and having an alcohol dehydrogenase activity.

5. The genetically modified microorganism according to claim 1, wherein the alcohol dehydrogenase is a protein encoded by at least one gene selected from the group consisting of yqhD, fucO, adhP, ybbO, eutG, ahr, yahK, adhE, ybdR, dkgA, yiaY, frmA, dkgB, yghA, ydjG, gldA, yohF, yeaE, ADH1, ADH2, ADH3, ADH4, ADH5, ADH6, ADH7, SFA1, AAD3, AAD4, AAD10, AAD14, AAD15, YPR1, NCg10324, NCg10313, NCg10219, NCg12709, NCg11112, NCg12382, NCg10186, NCg10099, NCg12952, NCg11459, yogA, bdhK, bdhJ, akrN, yqkF, yccK, iolS, and yrpG.

6. The genetically modified microorganism according to claim 1, wherein the alcohol dehydrogenase is a protein encoded by at least one gene selected from the group consisting of yqhD, fucO, adhP, eutG, ybbO, ahr, and yahK.

7. The genetically modified microorganism according to claim 1, wherein the alcohol dehydrogenase is a protein encoded by at least one gene selected from the group consisting of yqhD and adhP.

8. The genetically modified microorganism according to claim 7, wherein the alcohol dehydrogenase is a protein encoded by an adhP gene.

9. The genetically modified microorganism according to claim 1, wherein the alcohol dehydrogenase is a protein encoded by at least one gene selected from the group consisting of yqhD, fucO, eutG, ybbO, ahr, and yahK.

10. The genetically modified microorganism according to claim 9, wherein the alcohol dehydrogenase is a protein encoded by at least one gene selected from the group consisting of eutG, ybbO, ahr, and yahK.

11. The genetically modified microorganism according to claim 1, wherein the alcohol dehydrogenase is a protein encoded by two or more genes selected from the group consisting of yqhD, fucO, adhP, eutG, ybbO, ahr, and yahK.

12. The genetically modified microorganism according to claim 1, wherein the alcohol dehydrogenase is a protein encoded by a gene of one combination selected from the group consisting of:
yqhD and fucO,
yqhD and adhP,
yqhD and eutG,
yqhD and ybbO,
yqhD and ahr,
yqhD and yahK,
yqhD, fucO, and adhP,
yqhD, fucO, adhP, and eutG,
yqhD, fucO, adhP, eutG, and ybbO,
yqhD, fucO, adhP, eutG, ybbO, and ahr,
and
yqhD, fucO, adhP, eutG, ybbO, ahr, and yahK.

13. The modified microorganism according to claim 1, wherein the modification performed to reduce the activity of the alcohol dehydrogenase compared to the non-reduced strain is performed by one or more selected from the group consisting of
a reduction in transcription amount and/or translation amount of a gene encoding the alcohol dehydrogenase in the microorganism and
a disruption of a gene encoding the alcohol dehydrogenase in the microorganism.

14. The genetically modified microorganism according to claim 1, wherein the genetically modified microorganism belongs to a genus selected from the group consisting of the genus Escherichia, the genus Corynebacterium, the genus Bacillus, the genus Acinetobacter, the genus Burkholderia, the genus Pseudomonas, the genus Clostridium, the genus Saccharomyces, the genus Schizosaccharomyces, the genus Yarrowia, the genus Candida, the genus Pichia, and the genus Aspergillus.

15. The genetically modified microorganism according to claim 1, wherein the genetically modified microorganism is Escherichia coli.

16. The genetically modified microorganism according to claim 1, wherein the genetically modified microorganism expresses an aminotransferase as the enzyme involved in the synthesis of the diamine compound.

17. The genetically modified microorganism according to claim 1, wherein the genetically modified microorganism expresses a carboxylic acid reductase as the enzyme involved in the synthesis of the diamine compound.

18. The genetically modified microorganism according to claim 17, wherein the carboxylic acid reductase has an activity of converting a carboxyl group of a carboxylic acid semialdehyde, a dicarboxylic acid, or an aminocarboxylic acid into an aldehyde.

19. The genetically modified microorganism according to claim 1, wherein the genetically modified microorganism
has an ability of producing a dicarboxylic acid, a carboxylic acid semialdehyde, or an aminocarboxylic acid, and
further expresses an aminotransferase and a carboxylic acid reductase.

20. The genetically modified microorganism according to claim 1, wherein the genetically modified microorganism
has an ability of producing adipic acid, adipic acid semialdehyde, or 6-aminohexanoic acid, and
further expresses an aminotransferase and a carboxylic acid reductase.

21. The genetically modified microorganism according to claim 17, wherein the genetically modified microorganism is further modified to increase an activity of a phosphopantetheinyl transferase.

22. The genetically modified microorganism according to claim 16, wherein a gene encoding the aminotransferase is ygjG.

23. The genetically modified microorganism according to claim 17, wherein a gene encoding the carboxylic acid reductase is MaCar.

24. The genetically modified microorganism according to claim 21, wherein a gene encoding the phosphopantetheinyl transferase is Npt.

25. The modified microorganism according to claim 1, wherein the modified microorganism contains
a base sequence having 85% or more of sequence identity with a base sequence set forth in SEQ ID NO: 115 and encoding a protein having an aminotransferase activity or
a base sequence having 85% or more of sequence identity with a base sequence encoding an amino acid sequence set forth in any one of SEQ ID NOs: 110 to 114 and encoding a protein having an aminotransferase activity.

26. The modified microorganism according to claim 1, wherein the modified microorganism contains
a base sequence having 85% or more of sequence identity with a base sequence set forth in SEQ ID NO: 105 and encoding a protein having a carboxylic acid reductase activity or
a base sequence having 85% or more of sequence identity with a base sequence encoding an amino acid sequence set forth in any one of SEQ ID NOs: 101 to 104 and encoding a protein having a carboxylic acid reductase activity.

27. The modified microorganism according to claim 21, wherein the modified microorganism contains
a base sequence having 85% or more of sequence identity with a base sequence set forth in SEQ ID NO: 109 and encoding a protein having a phosphopantetheinyl transferase activity or
a base sequence having 80% or more of sequence identity with a base sequence encoding an amino acid sequence set forth in any one of SEQ ID NOs: 106 to 108 and encoding a protein having a phosphopantetheinyl transferase activity.

28. The genetically modified microorganism according to claim 1, wherein the genetically modified microorganism expresses one or more enzymes selected from the group consisting of
acyl-(acyl carrier protein (ACP)) reductase (AAR),
an enzyme that produces an aldehyde from acyl-CoA, and
an enzyme that produces an aldehyde from acyl phosphate.

29. A method of producing a diamine compound using the genetically modified microorganism according to claim 1.

30. The method of producing a diamine compound according to claim 29, wherein the method comprising a culture step of culturing the genetically modified microorganism in a medium containing a carbon source and a nitrogen source to obtain a culture medium containing bacterial cells.

31. The method of producing a diamine compound according to claim 30, wherein
the medium further contains a precursor of a diamine compound, or
in the culture step, the precursor is added to the medium.

32. The method of producing a diamine compound according to claim 30, further comprising a reaction step of bringing the culture medium and/or the bacterial cells into contact with an aqueous solution containing a precursor of a diamine compound to obtain a reaction solution containing a diamine compound.

33. The method of producing a diamine compound according to claim 31, wherein the precursor is selected from the group consisting of a dicarboxylic acid, a carboxylic acid semialdehyde, an aminocarboxylic acid, an aminoaldehyde, a dialdehyde, acyl-ACP, acyl-CoA, and acyl phosphate.

34. The method of producing a diamine compound according to claim 32, wherein the precursor is selected from the group consisting of a dicarboxylic acid, a carboxylic acid semialdehyde, an aminocarboxylic acid, an aminoaldehyde, a dialdehyde, acyl-ACP, acyl-CoA, and acyl phosphate.
